(19) United States
(12) Patent Application Publication
(10) Pub. No.: US 2026/0009047 A1
(43) Pub. Date: Jan. 8, 2026

(54) TRANSCRIPTION FACTORS TO IMPROVE RESISTANCE TO ENVIRONMENTAL STRESS IN PLANTS

(71) Applicant: BOARD OF TRUSTEES OF MICHIGAN STATE UNIVERSITY, East Lansing, MI (US)

(72) Inventors: Gregg A. Howe, East Lansing, MI (US); Marcelo Campos, Lago Norte (BR); Yuki Yoshida, Tokyo (JP)

(21) Appl. No.: 19/326,458

(22) Filed: Sep. 11, 2025

Related U.S. Application Data
(62) Division of application No. 18/309,524, filed on Apr. 28, 2023, now Pat. No. 12,428,651.
Application No. 18/309,524 is a continuation of application No. 16/323,737, now Pat. No. 11,674,151.
(60) Provisional application No. 62/379,773, filed on Aug. 26, 2016.

(51) Int. Cl. C12N 15/82

(52) U.S. Cl. CPC C12N 15/8286

(57) ABSTRACT

Plants, plant cells, and seeds are described herein that grow well and are resistant to environmental stresses such as drought and insects, where the plants have one or more mutations that reduce or eliminate the interaction of MYC transcription factors with the JAZ proteins. The plants can have an additional mutation that reduces or eliminates the function of the PHYB gene, and/or a heterologous PIF4 transgene or PIF4 expression cassette to improve the growth of the myc mutant plants. Methods of making and using such plants, plant cells, and seeds are also described.

### CROSS REFERENCE TO RELATED APPLICATIONS

This application is a divisional application of U.S. patent application Ser. No. 18/309,524, filed Apr. 28, 2023, which is a continuation of U.S. patent application Ser. No. 16/323,737, filed Feb. 6, 2019, issued as U.S. Pat. No. 11,674,151, issued on Jun. 13, 2023, which is a national stage application filed under 35 U.S.C. § 371 from International Application Serial No. PCT/US2017/048660, filed Aug. 25, 2017, and published as WO 2018/039590 on Mar. 1, 2018, which claims benefit of priority to the filing date of U.S. Provisional Application Ser. No. 62/379,773, filed Aug. 26, 2016, and the contents of each application are specifically incorporated herein by reference in their entirety.

### FEDERAL FUNDING

This invention was made with government support under DE-FG02-91ER20021 awarded by the U.S. Department of Energy, and under GM057795 awarded by the National Institutes of Health, and under IOS1139329 awarded by the National Science Foundation. The government has certain rights in the invention.

### INCORPORATION BY REFERENCE OF SEQUENCE LISTING

A Sequence Listing is provided herewith as an xml file, “2329982.xml” created on Apr. 28, 2023, and having a size of 181,631 bytes. The content of the xml file is incorporated by reference herein in its entirety.

### BACKGROUND OF THE INVENTION

Plants integrate developmental and environmental cues to prioritize the allocation of photosynthetic products to growth, defense and other physiological processes. Investments in defense often suppress growth, whereas rapid growth, such as that triggered by competition for light, attenuates defense.

### SUMMARY

In plants, investments in defense often suppress growth, reducing overall biomass yields. For example, dense planting of crops such as corn suppresses the plant immune system through active repression of the jasmonate signaling pathway. As described herein, selected jasmonate and phytochrome gene deletions can unlink growth and defense tradeoffs in plants. As also described herein, selected mutations in transcription factors can obviate jasmonate inhibition and improve plant resistance to environmental stresses, but plants with such transcription factor mutations may not grow optimally. By combining the transcription factor mutations with loss-of-function phytochrome gene mutations can improve plant growth while retaining environmental stress resistance. The resulting phenotype observed in plants includes robust growth and less insect infestation. Such modifications enhance biomass output, and allows crops to be densely planted. Such modified plants can have significant utility in agriculture.

Described herein are plants, plant cells, and plant seeds that can have a PhyB loss-of-function mutation, and (a) a modified MYC nucleic acid encoding a mutant MYC protein comprising at least one mutation within or outside of a JAZ-interacting domain (JID) polypeptide region, (b) a loss-of-function mutation in at least one gene encoding a transcriptional repressor of jasmonic acid response (JAZ) protein; or (c) a combination of (a) and (b).

Also described here are methods of making such plants, plant cells, and seeds. For example, one method can include (a) providing one or more plant cell that has a PhyB loss-of-function mutation; (b) introducing into at least one of the one or more plant cells at least one transgene or expression cassette encoding a mutant MYC nucleic acid segment that encodes a mutant MYC protein to generate one or more transformed plant cells; and (c) generating a plant from the one or more transformed plant cell(s). The mutant MYC nucleic acid can, for example, have a dominant MYC mutation. Such a mutant MYC protein can have reduced binding to a JAZ protein selected from a JAZ1 protein, JAZ2 protein, JAZ3 protein, JAZ4 protein, JAZ5 protein, JAZ6 protein, JAZ7 protein, JAZ8 protein, JAZ9 protein, JAZ10 protein, JAZ11 protein, JAZ12 protein, JAZ13 protein, or a combination thereof, where for example the binding is reduced by at least 20% compared to a corresponding wild type MYC protein that does not have the MYC mutation(s).

Another exemplary method can include (a) providing one or more plant cells with a loss-of-function mutation in at least one gene encoding a transcriptional repressor of jasmonic acid response (JAZ) protein; (b) introducing into the one or more plant cells a PhyB loss-of-function mutation to generate one or more modified plant cells; and (c) generating a plant from the one or more modified plant cell(s). For example, the method can involve providing one or more plant cells with a loss-of-function mutation in jaz1, jaz3, jaz4-1, jaz9, and jaz10 genes.

Such methods can provide plants that exhibit resistance to environmental stress compared to a wild type plant of the same species under the same environmental conditions.

For example, the modified plants and plants grown from the modified seeds described herein can have 5% less, or 10% less, or 20% less, or 30% less, or 40% less, or 50% less, or 60% less, or 70% less, or 80% less, or 90% less, or 100% less leaf damage from insect feeding than a wild type plant (without the mutations described herein) of the same species grown under the same conditions. In some cases, the modified plants and plants grown from the modified seeds described herein can have at least about 10%, or at least about 20%, or at least about 30%, or at least about 40%, or at least about 50%, or at least about 60%, or at least about 70%, or at least about 80%, or at least about 90% fewer insects or insect larvae than a wild type plant of the same species grown under the same conditions.

The modified plants and plants grown from the modified seeds described herein grow as well as or better than wild type plants. For example, the rosette dry weight of the modified plants and plants grown from the modified seeds described herein is about the same as the rosette dry weight of wild type plants (without the genetic modifications described herein) grown for the same time and under the same conditions. For example, the rosette dry weight of the modified plants, and plants grown from the modified seeds, described herein is about 80% to about 120%, or about 90% to about 110% of the rosette dry weight of wild type plants grown for the same time and under the same conditions. In some cases, the average primary root length of the modified plants and plants grown from the modified seeds described herein can be 1.5-fold longer, of 2-fold longer, or 2.3-fold longer, or 2.5-fold longer, or 2.7-fold longer, or 3-fold longer than the average primary root length of wild type plants grown for the same time and under the same conditions.

### DESCRIPTION OF FIGURES

FIGS. 1A-1I illustrate development of a JAZ quintuple mutant (jazQ) that exhibits reduced growth and enhanced defense. FIG. 1A shows a simple model of the jasmonate (JA)-gibberellic acid (GA) signaling network that governs growth and defense against environmental stress. FIG. 1B shows an image of wild-type (WT) and jazQ (IQ) seedlings grown in the absence or presence of 25 μM methyl-jasmonic acid (MJ or MeJA). FIG. 1C graphically illustrates accumulation of glucosinolates in WT (open bar) and jazQ mutant (shaded bar) seedlings. Compound abbreviations: 3MSP=3-methylsulfinylpropylglucosinolate; 4MSB=4-methylsulfinylbutylglucosinolate; 5MSP=5-methylsulfinylpentylyglucosinolate; 4OHI3M=4-hydroxyindol-3-ylmethylglucosinolate; 7MSH=7-methylsulfinylheptylglucosinolate; 4MTB=4-methylthiobutylglucosinolate; 8MSO=8-methylsulfinyloctylglucosinolate; I3M=indol-3-ylmethylglucosinolate; 4MI3M=4-methoxyindol-3-ylmethylglucosinolate; 1MI3M=1-methoxyindol-3-ylmethylglucosinolate; 7MTH=7-methylthioheptyl-glucosinolate; 8MTO, 8-methylthiooctylglucosinolate. FIG. 1D graphically illustrates anthocyanin accumulation in petioles of 4-week-old plants. FIG. 1E graphically illustrates Trichoplusia ni weight after feeding on WT (33 larvae) and mutant jazQ (38 larvae) plants for 10 days. FIG. 1F shows an image of 4-week old soil-grown WT and jazQ plants. Data in all graphs represent the mean±standard error (s.e.) of at least 10 biological replicates. Asterisks in FIGS. 1C, 1D, and 1E denote significant differences between WT and jazQ mutants at P<0.05 (Student's t-test). FIG. 1G is a schematic diagram showing T-DNA insertion lines used for construction of jazQ mutations. The organization of each JAZ gene is depicted by white and grey boxes representing untranslated regions (UTRs) and exons, respectively. The identity and position of the T-DNA insertion is shown. Arrows show the position of primers used to test expression by RT-PCR. FIG. 1H shows a gel illustrating RT-PCR analysis of JAZ gene expression in WT and mutant jazQ seedlings. RNA was obtained from seedlings grown for eight days on plates containing 25 μM MeJA. The ACTIN1 gene (ACT1, At2g37620) was used as a positive control. FIG. 1I graphically illustrates root length of WT, jaz10-1 mutant, and jazQ mutant seedlings grown for eight days on MS medium supplemented with 5, 10 or 25 μM MeJA. Control seedlings were grown in the absence of MeJA (0 μM). Data shown are the mean±s.e. of measurements on at least 12 seedlings per genotype. P-values are shown for two-way ANOVA comparisons (inset). Asterisks represent statistical difference between mutant and WT according to Tukey HSD test (P<0.05). Single asterisks denote a significant difference between mutant and WT, whereas double asterisks denote a significant difference between jaz10-1 and jazQ mutants at a given concentration of MeJA.

FIG. 2A-2H illustrate that mutant jazQ phyB plants simultaneously grow well and defend against insect infestation. FIG. 2A shows images of five week-old WT, jazQ mutant, and sjq11 mutant plants. The sjq11 plants have a mutation in the PHYB gene that suppresses the growth phenotype of jazQ. Hence, jazQ sjq11 mutant plants are about the same size as wild type plants. FIG. 2B illustrates Trichoplusia ni weight after feeding for 10 days on WT (31 larvae), mutant jazQ (31 larvae), and mutant sjg11 (37 larvae) plants. Data shown are the mean±standard error (s.e.) of at least 12 independent replicates. FIG. 2C shows images of four week-old plants grown in soil. FIG. 2D graphically illustrates rosette dry weight of WT, mutant jazQ, mutant phyB and mutant jazQ/phyB plants. FIG. 2E graphically illustrates anthocyanin accumulation in petioles of WT, mutant jazQ, mutant phyB and mutant jazQ/phyB plants. Data shown in FIG. 2D-2E are the mean±standard error (s.e.) of ten plants per genotype. FIG. 2F graphically illustrates T ni larval weight after feeding for 10 days on WT (23 larvae), mutant jazQ (29 larvae), and mutant jazQ phyB (27 larvae) plants. Data show the mean larval weight ±s.e. of insects reared on 12 plants per host genotype. Capital letters denote statistical differences according to Tukey HSD-test (P<0.05). Scale bars=1 cm. FIG. 2G shows a schematic diagram of the PHYB gene in sjg11. Sequence analysis identified a cytosine (C) to thymine (T) transition that creates a TGA nonsense mutation at the CGA codon for R322. This mutation truncates the PHYB apoprotein in the chromophore-binding GAF domain and is a null mutation. FIG. 2H graphically illustrates the number of days to bolting of wild type, jazQ, and jazQ sjq11 plants. Data show the mean±standard error (s.e.) of at least 12 independent replicates. Letters indicate statistical differences between genotypes (Tukey HSD-test, P<0.05).

FIG. 3 illustrates that the combination of jazQ and phyB mutations promotes expression of growth-related and defense-related genes. WT, mutant jazQ, mutant phyB and mutant jazQ/phyB seedlings were grown for 8 days in continuous white light prior to RNA extraction and analysis of gene expression by mRNA sequencing. The Venn diagram shows the number of genes upregulated in comparisons between WT and each of the three mutants. GO analysis of functional categories was performed with gene sets that are shared between mutant jazQ and mutant jazQ phyB (blue intersect), shared between mutant phyB and mutant jazQ phyB (yellow intersect), or unique to mutant jazQ phyB (green shade).

FIGS. 4A-4E illustrate that jazQ and phyB mutations interact to modulate photosynthesis and leaf architecture. FIG. 4A shows a heat map of photosystem II quantum efficiency (Φ_{II}) in response to varying light regimes. Chlorophyll fluorescence values for the indicated mutants were normalized to Col-0. Plants were exposed to three consecutive 16 hr/day light regimes: constant light (day 1, left panel); sinusoidal increase and decrease in light intensity (day 2, middle panel); and sinusoidal light with higher intensity pulses (day 3, right panel). FIG. 4B graphically illustrates the photosynthetic rate in response to increasing light as measured by gas exchange in 6-9 plants per genotype. The inset shows non-linear curve-fitting to model the maximum velocity of Rubisco determined from foliage photosynthetic rates in response to increasing CO₂. FIG. 4C graphically illustrates Rubisco concentration in leaves from 54-day-old plants (n=4). FIG. 4D graphically illustrates total chlorophyll concentration in leaves from 54-day-old plants (n=4). FIG. 4E graphically illustrates the thickness of 22-day-old rosette leaves (n=4). Data shown in FIGS. 4B-4E are the mean±s.e., and capital letters indicate statistical difference at P<0.05 (Tukey HSD-test). In d, WT and mutant jazQ phyB means are different at P<0.1.

FIGS. 5A-5E illustrate that overexpression of PIF4 in the mutant jazQ background leads to partial rescue of growth without compromising defense. FIG. 5A shows images of representative 21-d-old plants of the indicated genotype. Two independent T3 lines (#1-2 and #3-1) of jazQ 35S:PIF4 were characterized but only the latter is shown. Scale bars=1 cm. FIG. 5B graphically illustrates petiole length of the third true leaf of independent jazQ 35S:PIF4 T2 lines (n=6 plants per line). T2 lines #1 and #3 are parents of T3 lines #1-2 and #3-1 described in panels c-e below. As a control to demonstrate the expected effects of PIF4 overexpression on petiole length, WT Col-0 plants were also transformed with the 35S:PIF4 transgene. Data for four independent T2 lines is shown. FIG. 5C graphically illustrates petiole length of the third true leaf of 21-d-old jazQ 35S:PIF4 plants compared to WT and mutant jazQ (n=10). FIG. 5D graphically illustrates anthocyanin content in petioles of 21-d-old plants of the indicated genotype (n>10 plants). FIG. 5E graphically illustrates the weight of T. ni larvae recovered after 10 d feeding on 12 plants per genotype: WT plants (37 larvae), jazQ mutant plants (31 larvae), jazQ 35:PIF4 #1-2 mutant plants (27 larvae), and jazQ 35:PIF4 #3-1 mutant plants (25 larvae). Data show the mean s.e. Capitalized letters indicate statistical differences (Tukey HSD-test, P<0.05).

FIG. 6 shows a sequence alignment of the JAZ-interacting domain (JID) from various MYC transcription factors (SEQ ID NOs: 116 (AtMYC3), 117 (AtMYC4), 118 (Bradi3g34200), 119 (GRMZM2G049229), 120 (Os10g42430), 121 (GRMZM2G001930), 122 (Sobic.001G287600), 123 (Camelina sativa), 124 (AtMYC2), 125 (Solyc08g076930), 126 (SolycO8g005050), 127 (AtJAM1), 128 (AtJAM2)). Underlining identifies those conserved amino acids that interact directly with JAZ9. These amino acid residues are targets for site directed mutagenesis, to generate modified MYC transcription factors that are insensitive to inhibition by JAZ repressors.

FIG. 7A-7E illustrate structure-based design of dominant MYC transcription factors. FIG. 7A schematically illustrates the domain architecture of Arabidopsis thaliana MYC3 (AtMYC3) and two alternative splice forms of Arabidopsis thaliana JAZ10 (AtJAZ10). FIG. 7B shows schematic diagrams of X-ray crystal structures of MYC3 in complex with JAZ10's CMID (left) or Jas domain (center), and an overlay of the two structures (see, e.g., Zhang et al., 2017). FIG. 7C shows results of yeast two-hybrid analyses of MED25 and JAZ10.4 (bait) interactions with wild-type MYC3 and MYC3 point mutants (prey, identified at the top). Darker color (blue in the original) denotes protein-protein interaction. Asterisks (*) denote two novel MYC3 mutants that fail to interact with the CMID of JAZ10.4 but retain interaction with the MED25 co-activator. FIG. 7D shows that overexpression of the MYC3^{D94N} dominant transcription factor (but not wild-type MYC3) in the phyB mutant background confers resistance to 5-methyl-tryptophan (5-MT). Plants were grown for 3 weeks in MS medium containing 50 μM 5-methyl-tryptophan or in mock treatment without methyl-tryptophan. FIG. 7E graphically illustrates that overexpression of a dominant MYC2 mutant transcription factor (MYC2^{D105N/E165K}) in phyB-9-defective mutant Arabidopsis plants reduces primary root inhibition caused by treatment with 5-methyl-tryptophan. As shown, the non-transgenic (NT) phyB-9 mutant plants that do not express the MYC2^{D105N/E165K} protein exhibit smaller primary roots, indicating the presence of 5-methyl-tryptophan has inhibited growth. Transgenic expression of additional wild type MYC2 improves primary root length the phyB-9-defective mutant Arabidopsis plants. However, expression of the dominant MYC2 mutant transcription factor (MYC2^{D105N/E165K}) provides the best primary root growth in the phyB-9-defective mutant Arabidopsis plants.

### DETAILED DESCRIPTION

Plants and methods of making such plants are described herein that grow well and are resistant to environmental stresses such as drought and insects. The plants have mutations that reduce or eliminate the expression or function of proteins that modulate jasmonic acid responses (e.g., JAZ genes/proteins). Plants with such mutations are referred to herein as jaz mutants or jaz plants. Such reduction/elimination of jasmonic acid regulatory protein expression and/or function improves the insect resistance (compared to wild type plants) of jaz mutant plants. An additional mutation that reduces or eliminates the function of the PHYB gene improves the growth of jaz mutant plants. Hence, the combination of jaz and phyB loss-of-function mutations provides robustly growing plant lines that are also resistant to insects. Seeds of such jaz and phyB loss-of-function mutants, and methods of making and using such seeds and plants are also described herein.

Plants described herein can have one or more mutations that reduce or eliminate the interaction of MYC transcription factors with the JAZ proteins. MYC2 mediates stress responses through the action of plant stress hormones such as jasmonate (JA). In plant cells containing high levels of jasmonate, MYC transcription factors bind to the promoter region of JA-response genes to promote their transcriptional activation. However, simple overexpression of MYC2 is insufficient to constitutively activate defense responses. This is because MYC transcription factors are strongly repressed by direct binding of members of the JAZ family of repressor proteins. This application describes mutated MYC transcription factors that do not bind JAZ repressor proteins. Such mutations can be dominant MYC mutations. These plants that express mutant MYC proteins are capable of strongly activated defense responses in the presence of JAZ proteins. Such strong defense responses can reduce plant growth. By expressing mutant MYC proteins in a mutant phyB loss-of-function background, the plants exhibit strong defenses against environmental stress and also grow well.

Jasmonic acid and its various metabolites regulate plant responses to abiotic and biotic stresses as well as plant growth and development. The JAZ proteins typically inhibit the activation of defense responses that are controlled by jasmonic acid, and reduce the resistance of plants to environmental stresses such as drought, insects, and other environmental stresses. Reduction or elimination of JAZ functions tends to increase jasmonic acid expression and/or function, increase the activity of MYC transcription factors, and thereby improve drought and insect resistance (compared to wild type plants). Plants that produce mutated MYC proteins unable to bind JAZ proteins are phenotypically similar (increased resistance and reduced growth) to plants in which JAZ function is reduced or eliminated. An additional mutation that reduces or eliminates the function of the PHYB gene improves the growth of the myc mutant plants. Hence, the combination of myc and phyB loss-of-function mutations provides robustly growing plant lines that are also resistant to environmental stresses. Seeds of such myc and phyB loss-of-function mutants, and methods of making and using such seeds and plants are also described herein.

### Mutations

Plants and seeds have one or more genomic deletions, insertions, or substitutions in at least part of the MYC, JAZ, and PHYB genes. Such deletions, insertions, or substitutions can be generated by site-specific recombination-mediated methods. The mutations can range in size from one or two nucleotides to thousands of nucleotides (or any value therebetween). Deletions, insertions, and/or substitutions are created at a desired location in the genome. For example, borders (end points) of the deletions, insertions, or substitutions can be at defined locations to control the size of the deletions, insertions, or substitutions.

The mutation(s) can reduce or eliminate expression of endogenous JAZ and/or PhyB genes within plant cells, plants, and seeds. For example, the mutations can eliminate transcription and/or translation of from JAZ and PHYB genes encoding JAZ1, JAZ3, JAZ4, JAZ9, JAZ10, PHYB, and combinations thereof. The mutations can also eliminate transcription and/or translation of from genes related to the JAZ and PHYB genes encoding JAZ1, JAZ3, JAZ4, JAZ9, JAZ10, PHYB, and combinations thereof. For example, transcription and/or translation can be reduced by at least 20%, or at least 30%, or at least 40%, or at least 50%, or at least 60%, or at least 70%, or at least 80%, or at least 90%, or at least 95%, or at least 97%, or at least 99% compared to wild type plant cells, plants, and seeds of the same species (that do not have the JAZ and/or PhyB mutation(s)).

The mutation(s) can reduce or eliminate MYC protein interaction with one or more JAZ protein. For example, the mutation(s) can reduce or eliminate MYC protein interaction with JAZ1 protein, JAZ2 protein, JAZ3 protein, JAZ4 protein, JAZ5 protein, JAZ6 protein, JAZ7 protein, JAZ8 protein, JAZ9 protein, JAZ10 protein, JAZ11 protein, JAZ12 protein, JAZ13 protein, and combinations thereof. For example, interaction between a MYC protein and a JAZ protein, or binding between such mutant MYC protein and any of such JAZ proteins can be reduced by at least 20%, or at least 30%, or at least 40%, or at least 50%, or at least 60%, or at least 70%, or at least 80%, or at least 90%, or at least 95%, or at least 97%, or at least 99% compared to wild type plant cells, plants, and seeds of the same species (that do not have the MYC mutation(s)).

Non-limiting examples of methods of introducing a modification into the genome of a plant cell can include microinjection, viral delivery, recombinase technologies, homologous recombination, TALENS, CRISPR, and/or ZFN, see, e.g. Clark and Whitelaw Nature Reviews Genetics 4:825-833 (2003); which is incorporated by reference herein in its entirety.

For example, nucleases such as zinc finger nucleases (ZFNs), transcription activator like effector nucleases (TALENs), and/or meganucleases can be employed with guide nucleic acid that allows the nuclease to target the genomic MYC, JAZ and PHYB site(s). In some cases of the various aspects described herein, a targeting vector can be used to introduce a deletion or modification of the genomic MYC, JAZ and PHYB chromosomal sites.

A “targeting vector” is a vector generally has a 5′ flanking region and a 3′ flanking region homologous to segments of the gene of interest. The 5′ flanking region and a 3′ flanking region can surround a DNA sequence comprising a modification and/or a foreign DNA sequence to be inserted into the gene. For example, the genomic MYC, JAZ and PHYB site(s) can be disrupted by insertion of T-DNA. In another example, the foreign DNA to be inserted may encode a selectable marker, such as an antibiotics resistance gene. Examples for suitable selectable markers include chloramphenicol resistance, gentamycin resistance, kanamycin resistance, spectinomycin resistance (SpecR), neomycin resistance gene (NEO) and hygromycin β-phosphotransferase markers (genes). The 5′ flanking region and the 3′ flanking region can be homologous to regions within the gene, or such flanking regions can flank the coding region of gene to be deleted, mutated, or replaced with the unrelated DNA sequence. In some cases, the targeting vector does not comprise a selectable marker. DNA comprising the targeting vector and the native gene of interest are contacted under conditions that favor homologous recombination (e.g., by transforming plant cell(s) with the targeting vector).

A typical targeting vector contains nucleic acid fragments of not less than about 0.1 kb nor more than about 10.0 kb from both the 5′ and the 3′ ends of the genomic locus which encodes the gene to be modified (e.g. the genomic MYC, JAZ and/or PHYB site(s)). These two fragments can be separated by an intervening fragment of nucleic acid that includes the modification to be introduced. When the resulting construct recombines homologously with the chromosome at this locus, it results in the introduction of the modification, e.g. an insertion, substitution, or a deletion of a portion of the genomic MYC, JAZ and/or PHYB site(s).

In some cases, a Cas9/CRISPR system can be used to create a modification in genomic MYC, JAZ and/or PHYB site(s). Clustered regularly interspaced short palindromic repeats (CRISPR)/CRISPR-associated (Cas) systems are useful for, e.g. RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties). A CRISPR guide RNA can be used that can target a Cas enzyme to the desired location in the genome, where it generates a double strand break. This technique is available in the art and described, e.g. at Mali et al. Science 2013 339:823-6; which is incorporated by reference herein in its entirety and kits for the design and use of CRISPR-mediated genome editing are commercially available, e.g. the PRECISION X CAS9 SMART NUCLEASE™ System (Cat No. CAS900A-1) from System Biosciences, Mountain View, CA.

In other cases, a cre-lox recombination system of bacteriophage P1, described by Abremski et al. 1983. Cell 32:1301 (1983), Stemnberg et al., Cold Spring Harbor Symposia on Quantitative Biology, Vol. XLV 297 (1981) and others, can be used to promote recombination and alteration of the genomic MYC, JAZ and/or PHYB site(s). The cre-lox system utilizes the cre recombinase isolated from bacteriophage P1 in conjunction with the DNA sequences (termed lox sites) it recognizes. This recombination system has been effective for achieving recombination in plant cells (U.S. Pat. No. 5,658,772), animal cells (U.S. Pat. Nos. 4,959,317 and 5,801,030), and in viral vectors (Hardy et al., J Virology 71:1842 (1997).

The plant cells, plants, and plant seeds can have genomic mutations that alter one or more amino acids in the encoded MYC, JAZ and/or PHYB proteins. For example, plant cells, plants, and seeds can be modified so that at least one amino acid of a MYC, JAZ and/or PHYB polypeptide is deleted or mutated to reduce the function of MYC, JAZ and/or PHYB proteins. In some cases, a conserved amino acid or a conserved domain of the MYC, JAZ and/or PHYB polypeptide is modified. For example, a conserved amino acid or several amino acids in a conserved domain of the MYC, JAZ and/or PHYB polypeptide can be modified to change the physical and/or chemical properties of the conserved amino acid(s). For example, to change the physical and/or chemical properties of the conserved amino acid(s), the amino acid(s) can be deleted or replaced by amino acid(s) of another class, where the classes are identified in the following Table 1.

TABLE 1

| Classification | Genetically Encoded | Genetically Non-Encoded |
|---|---|---|
| Hydrophobic | | |
| Aromatic | F, Y, W | Phg, Nal, Thi, Tic, Phe(4- |
| | | Cl), Phe(2-F), Phe(3-F), |
| | | Phe(4-F), Pyridyl Ala, |
| | | Benzothienyl Ala |
| Apolar | M, G, P | |
| Aliphatic | A, V, L, I | t-BuA, t-BuG, MeIle, Nle, |
| | | MeVal, Cha, bAla, MeGly, |
| | | Aib |
| Hydrophilic | | |
| Acidic | D, E | |
| Basic | H, K, R | Dpr, Orn, hArg, Phe(p- |
| | | NH₂), DBU, A₂ BU |
| Polar | Q, N, S, T, Y | Cit, AcLys, MSO, hSer |
| Cysteine-Like | C | Pen, hCys, β-methyl Cys |

Different types of amino acids can be in the mutant myc, jazQ and/or phyB polypeptide(s), such as any of those listed in Table 2.

TABLE 2

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| N-Methylglycine | | MeGly |
| (sarcosine) | | |
| Ornithine | | Orn |
| Citrulline | | Cit |
| N-methylisoleucine | | MeIle |
| Phenylglycine | | Phg |
| Norleucine | | Nle |
| Penicillamine | | Pen |
| Homoarginine | | hArg |
| N-acetyl lysine | | AcLys |
| ρ-Aminophenylalanine | | Phe(pNH₂) |
| N-methylvaline | | MeVal |
| Homocysteine | | hCys |
| Homoserine | | hSer |

For example, modified MYC proteins can have any naturally occurring amino acid within the protein replaced with any of the amino acids listed in Tables 1 or 2. Positions within MYC protein that can have such replacements include, for example, amino acid positions 94, 97, 102, 105, 107, 125, 128, 129, 148, 152, 155, 163, 165, 169, and/or 172.

In some cases, myc, jaz and/or phyB mutations are introduced by insertion of foreign DNA into the gene of interest. For example, this can involve the use of either transposable elements (see, e.g., Parinov et al., Plant Cell 11, 2263-2270 (1999)) or T-DNA. The foreign DNA not only disrupts the expression of the gene into which it is inserted but also acts as a marker for subsequent identification of the mutation. Because some plant introns are small, and because there can be very little intergenic material in plant chromosomes, the insertion of a piece of T-DNA on the order of 5 to 25 kb in length generally produces a dramatic disruption of gene function. If a large enough population of T-DNA-transformed lines is available, one has a very good chance of finding a plant carrying a T-DNA insert within any gene of interest.

Mutations that are homozygous lethal can be maintained in the population in the form of heterozygous plants.

### MYC Proteins and Modifications Thereof

The bHLH-type transcription factor (TF) MYC2, together with related members of this family (e.g., MYC3 and MYC4 in Arabidopsis), promote myriad plant responses to biotic and abiotic stress (Kazan & Manners, 2013). MYC2 executes biotic and abiotic stress responses primarily through the stress hormones jasmonate and abscisic acid (ABA). For example, in some cases the MYC transcription factors can promote jasmonate-triggered defense responses against pathogen and insect pests. In plant cells containing high levels of jasmonate, MYC transcription factors bind to the promoter region of JA-response genes to promote their transcriptional activation.

Previous studies have shown that simple overexpression of MYC2 from a strong promoter such as the 35S cauliflower mosaic virus promoter is insufficient to constitutively activate defense responses in tomato/potato and Arabidopsis (Boter et al., 2004; Lorenzo et al., 2004). The reason for this is now clear: MYC transcription factors are strongly repressed via direct binding by members of the JAZ family of repressor proteins (of which there are thirteen in Arabidopsis, JAZ1-JAZ13).

JAZ proteins contain a C-terminal Jas motif that interacts directly with the JAZ-interacting domain (JID) of MYC transcription factors, thus inhibiting transcriptional activation of jasmonate response genes (Fernandez-Calvo et al., 2011; Zhang et al., 2015). Mutated derivatives of MYC2 (e.g. MYC2D105N) and MYC3 (e.g. MYC3D94N) fail to interact with most JAZ proteins, and are capable of activating jasmonate-responsive target genes in the presence of JAZs. This has been demonstrated from co-transfection assays (Goossens et al., 2015) and characterization of an Arabidopsis atr2D mutant (which harbors an Asp-to-Asn, MYC3D94N mutation) (Smolen et al., 2002). Based on these findings, it was stated that “Ultimately, the transferability of the Asp-to-Asn amino acid change might facilitate the design of hyperactive transcription factors for plant engineering” (Goossens et al., 2015). However, such mutations do not inhibit interactions with JAZ1 and JAZ10, which are potent repressors of MYC transcription factors.

Depending upon the location of a mutation, some mutations of MYC transcription factors may still be subject to repression by JAZ1 and JAZ10 because these two JAZ proteins harbor a cryptic MYC-interaction domain (CMID). By changing the MYC contact points with the CMID domain, which in some cases may be within the JID domain and in some cases outside of the JID domain, MYC transcription factors are generated that escape repression by all JAZ proteins. Such MYC transcription factors therefore are highly potent in their capacity to promote the expression of JA-response genes (i.e., MYC transcription factors that avoid repression by all JAZs).

This technology is useful not only for design of crops with increased resistance to pests, but also for enhancing the production of plant-derived medicinal compounds. One example is the anti-cancer drug taxol, whose production in taxus cells is promoted by the JA pathway via MYC transcription factors. Engineering of mutant (e.g., dominant mutant) MYC transcription factors into taxus cells could significantly increase taxol production. This same approach can be used to increase the production of any plant compound whose synthesis is controlled by MYC transcription factors (this includes many if not most plant secondary metabolites).

Examples of MYC protein sequences are provided herein that have one or more amino acid mutations, substitutions, replacements, insertions, or deletions within their JAZ-interacting domains (JIDs). In some cases, one or more mutations, substitutions, replacements, insertions, or deletions that are outside of the JAZ-interacting domain (JID) of the MYC proteins provided herein, for example, in regions that may interact with JAZ cryptic MYC-interaction domains (CMIDs).

For example, any of the MYC or MYC-related proteins described herein have at least one amino acid, or at least two amino acids, or at least three amino acids, or at least four amino acids, or at least five amino acid mutations, substitutions, replacements, insertions, or deletions in their JAZ-interacting domains (JIDs) on in regions that interact with JAZ cryptic MYC-interaction domains (CMIDs), or in both JID and CMID-interacting domains of a MYC protein. For example, MYC2 regions that interact with JAZ cryptic MYC-interaction domains (CMIDs) and/or MYC2 JAZ-interacting domains (JID) of a modified MYC protein can have less than 100%, or at less than 99.5%, or at less than 99%, or less than 98%, or at less than 97%, or less than 96%, or less than 95%, or less than 94%, or less than 93%, or less than 92%, or less than 91%, or less than 90% sequence identity with any of the CMID-interacting domains, JID, MYC or MYC related sequences described herein. However, in some cases the modified MYC protein have at least 40%, or at least 50%, or at least 60%, or at least 70%, or at least 80%, or at least 90%, or at least 95%, or at least 97%, or at least 99% sequence identity compared to the MYC or MYC-related sequences described herein.

For example, an Arabidopsis thaliana MYC2 sequence is shown below as SEQ ID NO:1, where the JAZ-interacting domain (JID) is shown in bold and with underlining.

| | |
|---|---|
| 1 | MTDYRLQPTM NLWTTDDNAS MMEAFMSSSD ISTLWPPAST |
| 41 | TTTTATTETT PTPAMEIPAQ AGFNQETLQQ RLQALIEGTH |
| 81 | EGWTYAIFWQ PSYDFSGASV LGWGDGYYKG EEDKANPRRR |
| 121 | SSSPPFSTPA DQEYRKKVLR ELNSLISGGV APSDDAVDEE |
| 161 | VTDTEWFFLV SMTQSFACGA GLAGKAFATG NAVWVSGSDQ |
| 201 | LSGSGCERAK QGGVFGMHTI ACIPSANGVV EVGSTEPIRQ |
| 241 | SSDLINKVRI LFNFDGGAGD LSGLNWNLDP DQGENDPSMW |
| 281 | INDPIGTPGS NEPGNGAPSS SSQLFSKSIQ FENGSSSTIT |
| 321 | ENPNLDPTPS PVHSQTQNPK FNNTFSRELN FSTSSSTLVK |
| 361 | PRSGEILNFG DEGKRSSGNP DPSSYSGQTQ FENKRKRSMV |
| 401 | LNEDKVLSFG DKTAGESDHS DLEASVVKEV AVEKRPKKRG |
| 441 | RKPANGREEP LNHVEAERQR REKLNQRFYA LRAVVPNVSK |
| 481 | MDKASLLGDA IAYINELKSK VVKTESEKLQ IKNQLEEVKL |
| 521 | ELAGRKASAS GGDMSSSCSS IKPVGMEIEV KIIGWDAMIR |
| 561 | VESSKRNHPA ARLMSALMDL ELEVNHASMS VVNDLMIQQA |
| 601 | TVKMGFRIYT QEQLRASLIS KIG |

The JAZ-interacting domain (JID) of the SEQ ID NO:1 Arabidopsis thaliana MYC2 protein is shown below as SEQ ID NO:2.

| | |
|---|---|
| 81 | YDFSGASV LGWGDGYYKG EEDKANPRRR |
| 121 | SSSPPFSTPA DQEYRKKVLR ELNSLISGGV APS |

In some cases, the MYC2 protein with SEQ ID NO:1 can have one or more mutations within the JID, and/or one or more mutations outside of the JID region. Thus modified MYC proteins described herein have less than 100% sequence identity to SEQ ID NO:1 and/or to SEQ ID NO:2.

Several amino acid positions of the MYC2 proteins can be modified, including for example, positions 94, 97, 102, 105, 107, 125, 128, 129, 148, 152, 155, 163, 165, 169, 172, or combinations thereof. MYC2 from different plant species can have variations in sequence. Hence, MYC2 from species other than Arabidopsis thaliana can have modifications at positions corresponding to positions 94, 97, 102, 105, 107, 125, 128, 129, 148, 152, 155, 163, 165, 169, and/or 172 from Arabidopsis thaliana.

For example, in some cases position 165 of the SEQ ID NO:1 MYC2 sequence can be a lysine rather than a glutamic acid (i.e., an E165K mutation). Such an E165K mutation is a gain-of-function allele of MYC2. The E165K mutation is in the transcriptional activation domain (TAD) of MYC2, which lies outside the JAZ-interacting domain, as described by Gasperini et al. PLOS Genetics 11(6): e1005300 (2015).

Other examples of MYC2 mutations include MYC2 D105N; MYC2 D105N+E165K; MYC2 M172A; MYC2 E165A+M172; and MYC2 L169A mutations.

An Arabidopsis thaliana MYC3 sequence is shown below as SEQ ID NO:3, where the JAZ-interacting domain (JID) is shown in bold and with underlining.

| | |
|---|---|
| 1 | MNGTTSSINF LTSDDDASAA AMEAFIGTNH HSSLFPPPPQ |
| 41 | QPPQPQFNED TLQQRLQALI ESAGENWTYA IFWQISHDFD |
| 81 | SSTGDNTVIL GWGDGYYKGE EDKEKKKNNT NTAEQEHRKR |
| 121 | VIRELNSLIS GGIGVSDESN DEEVTDTEWF FLVSMTQSFV |
| 161 | NGVGLPGESF LNSRVIWLSG SGALTGSGCE RAGQGQIYGL |
| 201 | KTMVCIATQN GVVELGSSEV ISQSSDLMHK VNNLFNFNNG |
| 241 | GGNNGVEASS WGFNLNPDQG ENDPALWISE PTNTGIESPA |
| 281 | RVNNGNNSNS NSKSDSHQIS KLEKNDISSV ENQNRQSSCL |
| 321 | VEKDLTFQGG LLKSNETLSF CGNESSKKRT SVSKGSNNDE |
| 361 | GMLSFSTVVR SAANDSDHSD LEASVVKEAI VVEPPEKKPR |
| 401 | KRGRKPANGR EEPLNHVEAE RQRREKLNQR FYSLRAVVPN |
| 441 | VSKMDKASLL GDAISYINEL KSKLQQAESD KEEIQKKLDG |
| 481 | MSKEGNNGKG CGSRAKERKS SNQDSTASSI EMEIDVKIIG |
| 521 | WDVMIRVQCG KKDHPGAREM EALKELDLEV NHASLSVVND |
| 561 | LMIQQATVKM GSQFFNHDQL KVALMTKVGE NY |

The JAZ-interacting domain (JID) of the SEQ ID NO:3 Arabidopsis thaliana MYC3 protein is shown below as SEQ ID NO:4.

| | |
|---|---|
| 81 | STGDNTVIL GWGDGYYKGE EDKEKKKNNT NTAEQEHRKR |
| 121 | VIRELNSLIS GGIGVS |

In some cases, the MYC3 protein with SEQ ID NO:3 can have one or more mutations within the JID, and/or one or more mutations outside of the JID region. Thus modified MYC proteins described herein have less than 100% sequence identity to SEQ ID NO:3 and/or to SEQ ID NO:4.

Several amino acid positions of the MYC3 proteins can be modified, including for example, positions 94, 97, 102, 105, 107, 125, 128, 129, 148, 152, 155, 163, 165, 169, 172, or combinations thereof. MYC3 from different plant species can have variations in sequence. Hence, MYC3 from species other than Arabidopsis thaliana can have modifications at positions corresponding to positions 94, 97, 102, 105, 107, 125, 128, 129, 148, 152, 155, 163, 165, 169, and/or 172 from Arabidopsis thaliana.

For example, the glutamic acid at position 148 of the MYC3 protein with SEQ ID NO:3 corresponds to the glutamic acid at position 165 of the MYC2 protein, and can be lysine rather than glutamic acid (E148K) or an alanine rather than glutamic acid (E148A). In addition the aspartic acid at position 94 of the MYC3 protein with SEQ ID NO:3 or SEQ ID NO:4 can be an asparagine (D94N). Modified MYC3 proteins can also have a combination of E148K and D94N mutations.

Other MYC3 modifications can include an MYC3 M155A mutation, an MYC3 L152A mutation, and combinations thereof. The MYC3 M155A mutation, and/or MYC3 L152A mutation can be combined with E148K, E148A, and/or D94N mutation.

An Arabidopsis thaliana MYC4 sequence is shown below as SEQ ID NO:5, where the JAZ-interacting domain (JID) is shown in bold and with underlining.

| | |
|---|---|
| 1 | MSPTNVQVTD YHLNQSKTDT TNLWSTDDDA SVMEAFIGGG |
| 41 | SDHSSLFPPL PPPPLPQVNE DNLQQRLQAL IEGANENWTY |
| 81 | AVFWQSSHGF AGEDNNNNNT VLLGWGDGYY KGEEEKSRKK |
| 121 | KSNPASAAEQ EHRKRVIREL NSLISGGVGG GDEAGDEEVT |
| 161 | DTEWFFLVSM TQSFVKGTGL PGQAFSNSDT IWLSGSNALA |
| 201 | GSSCERARQG QIYGLQTMVC VATENGVVEL GSSEIIHQSS |
| 241 | DLVDKVDTFF NFNNGGGEFG SWAFNLNPDQ GENDPGLWIS |
| 281 | EPNGVDSGLV AAPVMNNGGN DSTSNSDSQP ISKLCNGSSV |
| 321 | ENPNPKVLKS CEMVNFKNGI ENGQEEDSSN KKRSPVSNNE |
| 361 | EGMLSFTSVL PCDSNHSDLE ASVAKEAESN RVVVEPEKKP |
| 401 | RKRGRKPANG REEPLNHVEA ERQRREKLNQ RFYSLRAVVP |
| 441 | NVSKMDKASL LGDAISYISE LKSKLQKAES DKEELQKQID |
| 481 | VMNKEAGNAK SSVKDRKCLN QESSVLIEME VDVKIIGWDA |
| 521 | MIRIQCSKRN HPGAKFMEAL KELDLEVNHA SLSVVNDLMI |
| 561 | QQATVKMGNQ FFTQDQLKVA LTEKVGECP |

The JAZ-interacting domain (JID) of the SEQ ID NO:5 Arabidopsis thaliana MYC4 protein is shown below as SEQ ID NO:6.

| | |
|---|---|
| 81 | NNNNNT VLLGWGDGYY KGEEEKSRKK |
| 121 | KSNPASAAEQ EHRKRVIREL NSLISGGVGG G |

A comparison of the Arabidopsis thaliana MYC4 sequence with SEQ ID NO:5 and the Arabidopsis thaliana MYC2 sequence having SEQ ID NO:1 is shown below.

| 50.2% identity in 630 residues overlap; Score: 1225.0; Gap frequency: 12.4 | | |
|---|---|---|
| Seq 1 | 9 | TMNLWTTDDNASMMEAEMSS- |
| SDISTLWPPASTTTTTATTETTPTPAMEIPAQAGFNQET | | |
| Seq 5 | 20 | TTNLWSTDDDASVMEAFIGGGSDHSSLEPPLP-----------PPPLPQV------ |
| NEDN | | |
| | | * *** *** ** **** ** * * ** * * * |
| Seq 1 | 68 | LQQRLQALIEGTHEGWTYAIFWQPSYDFSGAS------- |
| VLGWGDGYYKGEEDKANPRRR | | |
| Seq 5 | 63 | LQQRLQALIEGANENWTYAVFWQSSHGFAGEDNNNNNTVLLGWGDGYYKGEEEKS-- |
| RKK | | |
| | | *********** * **** *** * * * ************ * * |
| Seq 1 | 121 | |
| SSSPPFSTPADQEYRKKVLRELNSLISGGVAPSDDAVDEEVTDTEWFFLVSMTQSFACGA | | |
| Seq 5 | 121 | KSNR-- |
| ASAAEQEHRKPVIRELNSLISGGVGGGDEAGDEEVTDTEWFFLVSMTQSFVKGT | | |
| * * * ** ** * *********** * * ******************* | | |
| * | | |
| Seq 1 | 181 | |
| GLAGKAFATGNAVWVSGSDQLSGSGCERAKQGGVFGMHTIACIPSANGVVEVGSTEPIRQ | | |
| Seq 5 | 179 | |
| GLPGQAFSNSDTIWLSGSNALAGSSCERARQGQIYGLQTMVCVATENGVVELGSSEIIHQ | | |
| ** * ** * *** * ** **** ** * * * ***** ** * ** | | |
| Seq 1 | 241 | |
| SSDLINKVRILFNFDGGAGDLSGLNWNLDPDQGENDPSMWINDPIGTPGSNEPGNGAPSS | | |
| Seq 5 | 239 | SSDLVDKVDTFFNFNNGGGEFGSWAFNLNPDQGENDPGLWISEPNGV------------- |
| | | **** ** *** * * ** ******** ** * * |
| Seq 1 | 301 | |
| SSQLFSKSIQFENGSSSTITENPNLDPTPSPVHSQTQNPKFNNTFSRELNFSTSSSTLVK | | |
| Seq 5 | 286 | DSGLVAAPVMNNGGNDSTSNSDSQ------PISKLCNGSSVENPNPKVL----------- |
| | | * * * ** * * * |
| Seq 1 | 361 | |
| PRSGEILNFGDEGKRSSGNPDPSSYSGQTQFENKRKRSMVLNEDKVLSFGDKTAGESDHS | | |
| Seq 5 | 329 | -KSCEMVN--KNGIENGQEEDSS-------- |
| NKKRSPVSNNEEGMLSFTSVLPCDSNHS | | |
| * * ** * ** ** ** *** * | | |
| ** | | |
| Seq 1 | 421 | DLEASVVKE------- |
| VAVEKRPKKRGRKPANGREEPLNHVEAERQRREKLNQRFYALPA | | |
| Seq 5 | 378 | |
| DLEASVAKEAESNRVVVEPEKKPRKRGRKPANGREEPLNHVEAERQRREKLNQRFYSLRA | | |
| ****** ** * ** * ******************************** | | |
| *** | | |
| Seq 1 | 474 | |
| VVPNVSKMDKASLLGDAIAYINELKSKVVKTESEKLQIKNQLEEVKLELAGRKASASGGD | | |
| Seq 5 | 438 | |
| VVPNVSKMDKASLLGDAISYISELKSKLQKAESDKEELQKQIDVMNKEAGNAKSSVKDRK | | |
| ****************** ** ***** * ** * * * * * | | |
| Seq 1 | 534 | |
| MSSSCSSIKPVGMEIEVKIIGWDAMIRVESSKRNHPAARLMSALMDLELEVNHASMSVVN | | |
| Seq 5 | 498 | CLNQESSVL- |
| IEMEVDVKIIGWDAMIRIQCSKRNHPGAKFMEALKELDLEVNHASLSVVN | | |
| ** ** *********** ****** * * ** * ******* | | |
| **** | | |
| Seq 1 | 594 | DLMIQQATVKMGFRIYTQEQLRASLISKIG |
| Seq 5 | 557 | DLMIQQATVKMGNQFFTQDQLKVALTEKVG |
| | | ************ ** ** * * * |

In some cases, the Arabidopsis thaliana MYC4 protein with SEQ ID NO:5 can have one or more mutations within the JID, and/or one or more mutations outside of the JID region. Thus modified MYC4 proteins described herein have less than 100% sequence identity to SEQ ID NO:5 and/or to SEQ ID NO:6.

Several amino acid positions of the MYC proteins can be modified, including for example, positions 94, 97, 102, 105, 107, 125, 128, 129, 148, 152, 155, 163, 165, 167, 169, 170, 172, or combinations thereof. MYC4 from different plant species can have variations in sequence. Hence, MYC4 from species other than Arabidopsis thaliana can have modifications at positions corresponding to positions 94, 97, 102, 105, 107, 125, 128, 129, 148, 152, 155, 163, 165, 167, 169, 170, and/or 172 from the Arabidopsis thaliana MYC2 protein (highlighted in bold and with underlining in the comparison above). In some cases the amino acid positions in the MYC4 protein can vary from those in the corresponding Arabidopsis thaliana MYC2 protein by 1-10 positions.

For example, modified MYC4 proteins can have D107N mutations, E163K mutations, M170A mutations, L167A mutations, or combinations thereof. Other MYC proteins can have the same types of mutations but the location can vary. For example, the aspartic acid (D) that is at position 107 of the MYC4 protein with SEQ ID NO:5, at be at different positions in other MYC proteins. For example, such an aspartic acid can be at ±5 positions from position 107. However, such an aspartic acid is readily identified by sequence comparisons such as those illustrated herein because a selected amino acid at a particular position can be aligned via its adjoining sequence with the sequence of a related protein, and even if there are sequence variations between the two proteins the skilled person can find the selected amino acid in the related protein.

Similarly, for example, a selected amino acid at a particular position, such an aspartic acid at position 107 in one protein can readily be identified in another protein as being at position 102 because that aspartic acid it is typically found within a sequence that is conserved between the two proteins. For example an aspartic acid at position 102 or 107 in different proteins can readily be identified because it is at the end of a conserved GWGD (SEQ ID NO:110) sequence. Other conserved segments of MYC protein sequences are illustrated in the sequence comparisons shown herein, including for example, the DFSG (SEQ ID NO:111) sequence, the RELNSLISGGV (SEQ ID NO:112) sequence, the DTEWFFLVSM (SEQ ID NO:113) sequence, the VVNDLMIQQATVKMG (SEQ ID NO:114) sequence, and/or the KRGRKPANGREEPLNHVEAERQRREKLNQRFY (SEQ ID NO:115) sequence. Such segments of conserved sequences facilitate alignment of related amino acid sequences so that corresponding amino acids can be identified despite position and some sequence variation.

MYC-related proteins can also be modified and expressed in a variety of plants, for example, instead of or in addition to a native MYC protein. An example of a MYC-related protein is a rice (Oryza sativa) MYC7E protein, which has at least 47% sequence identity to the MYC2 sequence with SEQ ID NO:1. This rice MYC7E protein sequence is shown below as SEQ ID NO:7, where the JAZ-interacting domain (JID) is shown in bold and with underlining.

| | |
|---|---|
| 1 | MWVLLSPLLT TKNPFHPIPI PTFPLLLFSS SLVGVLFQIK |
| 41 | SNLEEEEIEI KSMNLWTDDN ASMMEAFMAS ADLPAFPWGA |
| 81 | ASTPPPPPPP PHHHHQQQQQ QVLPPPAAAP AAAAFNQDTL |
| 121 | QQRLQSIIEG SRETWTYAIF WQSSIDVSTG ASLLGWGDGY |
| 161 | YKGCDDDKRK QRSSTPAAAA EQEHRKRVLR ELNSLIAGAG |
| 201 | AAPDEAVEEE VTDTEWFFLV SMTQSFPNGL GLPGQALFAA |
| 241 | QPTWIATGLS SAPCDRARQA YTFGLRTMVC LPLATGVLEL |
| 281 | GSTDVIFQTG DSIPRIRALF NLSAAAASSW PPHPDAASAD |
| 321 | PSVLWLADAP PMDMKDSISA ADISVSKPPP PPPHQIQHFE |
| 361 | NGSTSTLTEN PSPSVHAPTP SQPAAPPQRQ QQQQQSSQAQ |
| 401 | QGPFRRELNF SDFASNGGAA APPFFKPETG EILNFGNDSS |
| 441 | SGRRNPSPAP PAATASLTTA PGSLFSQHTP TLTAAANDAK |
| 481 | SNNQKRSMEA TSRASNTNNH PAATANEGML SFSSAPTTRP |
| 521 | STGTGAPAKS ESDHSDLEAS VREVESSRVV APPPEAEKRP |
| 561 | RKRGRKPANG REEPLNHVEA ERQRREKLNQ RFYALRAVVP |
| 601 | NVSKMDKASL LGDAISYINE LRGKLTALET DKETLQSQME |
| 641 | SLKKERDARP PAPSGGGGDG GARCHAVEIE AKILGLEAMI |
| 681 | RVQCHKRNHP AARLMTALRE LDLDVYHASV SVVKDLMIQQ |
| 721 | VAVKMASRVY SQDQLNAALY TRIAEPGTAA R |

A comparison of the rice MYC7E protein sequence having SEQ ID NO:7 with the MYC2 protein sequence having SEQ ID NO:1 is shown below, where the asterisks identify positions that are identical in the two proteins.

| 47.2% identity in 705 residues overlap; Score: 1039.0; Gap frequency: 14.8% | | |
|---|---|---|
| UserSeq 1 | 9 | TMNLWTTDDNASMMEAFMSSSDISTL-WPPASTTTTTATT---------ETTPTPAMEIP |
| UserSeq 7 | 52 | SMNLWT-DDNASMMEAFMASADLPAFTWGPASTPPPPPPPPHHHHQQQQQQVLPPPAAAP |
| | | ***** *********** * * *A *** * * |
| UserSeq 1 | 59 | AQAGFNQETLQQRLQALIEGTHEGWTYAIFWQPSYDFS-GASVLGWGDGYYKGEEDKANP |
| UserSeq 7 | 111 | AAAAFNQDTLQQRLQSIIEGSRETWTYAIFWQSSIDVSTGASLLGWGDGYYKGCDDDKR- |
| | | * * *** ******* *** * ******** * * * *** ********** * |
| UserSeq 1 | 118 | RRRSSSPPFSTPADQEYRKKVLRELNSLISGGVAPSDDAVDEEVTDTEWFFLVSMTQSFA |
| UserSeq 7 | 170 | KQRSSTP--AAAAEQEHRKRVLRELNSLIAGAGAAPDEAVEEEVTDTEWFFLVSMTQSFP |
| | | *** * * ** ** ********* * * * ** ****************** |
| UserSeq 1 | 178 | CGAGLAGKAFATGNAVWVSGSDQLSGSGCERAKQGGVFGMHTIACIPSANGVVEVGSTEP |
| UserSeq 7 | 228 | NGLGLPGQALFAAQPTWIATG--LSSAPCDRARQAYTFGLRTMVCLPLATGVLELGSTDV |
| | | * ** * * * ** * ** * ** * * * * ** * *** |
| UserSeq 1 | 238 | IRQSSDLINKVRILFNFDGGAGDLSGLNWNLDPDQGENDPS-MWIND--PIGTPGSNEPG |
| UserSeq 7 | 286 | IFQTGDSIPRIRALFNLSAAAAS----SWPPHPDAASADPSVLWLADAPPMDMKDSISAA |
| | | * * * * * *** * * ** *** * * * * |
| UserSeq 1 | 295 | NGAPSSSSQLFSKSIQ-FENGSSSTITENPNID---PTPS----PVHSQTQNPKFNNT-- |
| UserSeq 7 | 342 | DISVSKPPPPPPHQIQHFENGSTSTLTENPSPSVHAPTPSQPAAPPQRQQQQQQSSQAQQ |
| | | * ** ***** ** **** **** * * * |
| UserSeq 1 | 345 | --FSRELFSTSSST-------LVKPRSGEILNFGDEGKRSSGNRDPSSYSGQTQF---- |
| UserSeq 7 | 402 | GPFRRELNFSDFASNGGAAAPPFFKPETGEILNFGNDSSSGRRNPSPAPPAATASLTTAP |
| | | * ****** * ** ******* ** * |
| UserSeq 1 | 392 | -------------------ENKRKRSMVLNE--------------DKVLSF--------- |
| UserSeq 7 | 462 | GSLFSQHTPTLTAAANDAKSNNQKRSMEATSRASNTNNHPAATANEGMLSFSSAPTTRRS |
| | | * **** *** |
| UserSeq 1 | 410 | ---GDKTAGESDHSDLEASVVKEVA---------VEKRPKKRGRKPANGREEPLNHVEAE |
| UserSeq 7 | 522 | TGTGAPAKSESDHSDLEASVREVESSRVVAPPPEAEKRPRKRGRKPANGREEPLNHVEAE |
| | | * *********** **** ******************** |
| UserSeq 1 | 458 | RQRREKLNQRFYALRTAVVPNVSKMDKASLLGFAIAYINELKSKVVKTESEKLQIKNQLEE |
| UserSeq 7 | 582 | RQRREKLNQRFYALRVVPNVSKMDKASLLGDAISYINELRGKLTALETDKETLQSQMES |
| | | ********************************** ***** * * * * * |
| UserSeq 1 | 518 | VKLELAGRKASASGGDMSSSCSSIKPVGMEIEVKIIGWDAMIRVESSKRNHPAARLMSAL |
| UserSeq 7 | 642 | LKKERDARPPAPSGG---GGDGGARCHAVEIEAKILGLEAMIRVQCHKRNHPAARLMTAL |
| | | * * * *** *** ** * ***** ********** ** |
| UserSeq 1 | 578 | MDLELEVNHASMSVVNDLMIQQATVKMGFRIYTQEQLRASLISKI |
| UserSeq 7 | 699 | RELDLDVYHASVSVVKDLMIQQVAVKMASPVYSQDQLNAALYTRI |
| | | * * * *** *** ****** *** * * * ** * * * |

The JAZ-interacting domain (JID) of the rice MYC7E protein sequence having SEQ ID NO:7 is shown below as SEQ ID NO:8.

| | |
|---|---|
| 121 | STG ASLLGWGDGY |
| 161 | YKGCDDDKRK QRSSTPAAAA EQEHRKRVLR ELNSLIAGA |

In some cases, the MYC7E protein with SEQ ID NO:7 can have one or more mutations within the JID, and/or one or more mutations outside of the JID region. Thus modified MYC proteins described herein have less than 100% sequence identity to SEQ ID NO:7 and/or to SEQ ID NO:8.

For example, MYC7E from Oryza sativa can have modifications at positions corresponding to positions 94, 97, 102, 105, 107, 125, 128, 129, 148, 152, 155, 163, 165, 167, 169, 170, and/or 172 from the Arabidopsis thaliana MYC2 protein (highlighted in bold and with underlining in the comparison above). In some cases the amino acid positions in the endogenous Oryza sativa MYC protein can vary from those in the corresponding Arabidopsis thaliana MYC2 protein by 1-10 positions.

An example of another MYC protein is a maize (Zea mays) MYC4 protein, which has at least 47% sequence identity to the Arabidopsis MYC2 sequence with SEQ ID NO:1. This maize MYC4 protein sequence is shown below as SEQ ID NO:9, where the JAZ-interacting domain (JID) is shown in bold and with underlining.

| | |
|---|---|
| 1 | MNLWTDDNAS MMEAFMASAD LPTFPWGAPA GGGNSSAAAA |
| 41 | SPPPPQMPAA TAPGFNQDTL QQRLQAMIEG SRETWTYAIF |
| 81 | WQSSLDSATG ASLLGWGDGY YKGCDEDKRK QKPLTPSAQA |
| 121 | EQEHRKRVLR ELNSLISGAA AAPDEAVEEE VTDTEWFFLV |
| 161 | SMTQSFLNGS GLPGQALFAG QPTWIASGLS SAPCERARQA |
| 201 | YNFGLRTMVC FPVGTGVLEL GSTDVVFKTA ESMAKIRSLF |
| 241 | GGGAGGGSWP PVQPQAPSSQ QPAAGADHAE TDPSMLWLAD |
| 281 | APVMDIKDSL SHPSAEISVS KPPPHPPQIH FENGSTSTLT |
| 321 | ENPSPSVHAP PPPPAPAAPQ QRQHQHQNQA HQGPFRRELN |
| 361 | FSDFASTPSL AATPPFFKPE SGEILSFGAD SMARRNPSPV |
| 401 | PPAATASLTT APGSLFSQHT ATMTAAAAND AKNNNKRSME |
| 441 | ATSRASNTNH HPAATANEGM LSFSSAPTTR PSTGTGAPAK |
| 481 | SESDHSDLDA SVREVESSRV VAPPPEAEKR PRKRGRKPAN |
| 521 | GREEPLNHVE AERQRREKLN QRFYALRAVV PNVSKMDKAS |
| 561 | LLGDAISYIN ELRGKLTSLE TDKETLQTQV EALKKERDAR |
| 601 | PPSHSAGLGG HDGGPRCHAV EIDAKILGLE AMIRVQCHKR |
| 641 | NHPSARLMTA LRELDLDVYH ASVSVVKDLM IQQVAVKMAS |
| 681 | RVYTQDQLSA ALYSRLAEPG SAMGR* |

A comparison of the maize MYC4 protein sequence having SEQ ID NO:9 with the MYC2 protein sequence having SEQ ID NO:1 is shown below.

| 47.2% identity in 703 residues overlap; Score: 1048.0; Gap frequency: 13.8% | | |
|---|---|---|
| UserSeq 1 | 10 | MNLWTTDDNASMMEAFMSSSDISTL-W-PPASTTTTTATTETTPTPAMEIPAQAGFNQET |
| UserSeq 9 | 1 | MNLMT-DDNASMMEAFMASADLPTFPWGAPAGGGNSSAAAASPPPPQMPAATAPGFNQDT |
| | | ***** *********** * * * * ** * * * * **** * |
| UserSeq 1 | 68 | LQQRLQALIEGTHEGWTYAIFWQPSYDFS-GASVLGWGDGYYKGEEDKANPRRRSSSPPF |
| UserSeq 9 | 60 | LQQRLQAMIEGSRETWTYAIFWQSSLDSATGASLLGWGDGYYKGCDED---KRKQKPLTP |
| | | ******* *** * ******** * * *** ********** * |
| UserSeq 1 | 127 | STPADQEYRKKVLRELNSLISGGVAPSDDAVDEEVTDTEWFFLVSMTQSFACGAGLAGKA |
| UserSeq 9 | 117 | SAQAEQEHRKRVLRELNSLISGAAAAPDEAVEEEVTDTEWFFLVSMTQSFLNGSGLPGQA |
| | | * * ** ** *********** * * ** ****************** * ** * * |
| UserSeq 1 | 187 | FATGNAVWVSGSDQLSGSGCERAKQGGVFGMHTIACIPSANGVVEVGSTEPIRQSDLIN |
| UserSeq 9 | 177 | LFAGQPTWIASG--LSSAPCERARQAYNFGLRTMVCFPVGTGVLELGSTDVVFKTAESMA |
| | | * * ** **** * ** * * * ** * *** |
| UserSeq 1 | 247 | KVRILFNFDGGAGDL--------SGLNWNLDPDQGENDPSM-WIND-PIGT--PGSNEPG |
| UserSeq 9 | 235 | KIRSLFGGGAGGGSWPPVQPQAPSSQQPAAGADHAETDPSMLWLADAPVMDIKDSLSHPS |
| | | * * ** * * * * * **** * * * * |
| UserSeq 1 | 295 | NGAPSSSSQLFSKSIQFENGSSSTITENPNLD-----PTPSPV-----HSQTQNPKFNNT |
| UserSeq 9 | 295 | AEISVSKPPPHPPQIHFENGSTSTLTENPSPSVHAPPPPPAPAAPQQRQHQHQNQAHQGP |
| | | * * ***** ** **** * * * * ** |
| UserSeq 1 | 345 | FSRELNFSTSSST--------LVKPRSGEILNFG-DEGKRSSGNPDP------------S |
| UserSeq 9 | 355 | FRRELNFSDFASTPSLAATPPFFKPESGEILSFGADSNARRNPSPVPPAATASLTTAPGS |
| | | * ****** ** ** ***** ** * * * * * |
| UserSeq 1 | 384 | SYSGQT---------QFENKRKRSMVLNE--------------DKVLSF----------- |
| UserSeq 9 | 415 | LFSQHTATMTAAAANDAKNNNKRSMEATSRASNTNHHPAATANEGMLSFSSAPTTRPSTG |
| | | * * * **** *** |
| UserSeq 1 | 410 | -GDKTAGESDHSDLEASVVKEVA---------VEKRPKKRGRKPANGREEPLNHVEAERQ |
| UserSeq 9 | 475 | TGAPAKSESDHSDLDASVREVESSRVVAPPPEAEKRPRKRGRKPANGREEPLNHVEAERQ |
| | | * ******* *** **** ********************** |
| UserSeq 1 | 460 | RREKLNQRFYALRAVVPNVSKMDKASLLGDAIAYINELKSKVVKTESEKLQIKNQLEEVK |
| UserSeq 9 | 535 | RREKLNQRFYALRAVVPNVSKMDKASLLGDAISYINELRGKLTSLETDKETLQTQVEALK |
| | | ******************************** ***** * * * * * * |
| UserSeq 1 | 520 | LELAGRKASASGGDMSSSCSSIKPVGMEIEVKIIGWDAMIRVESSKRNHPAARLMSALMD |
| UserSeq 9 | 595 | KERDARPPSHSAG-LGGHDGGPRCHAVEIDAKILGLEAMIRVQCHKRNHPSARLMTALRE |
| | | * * * * * ** ** * ***** ***** **** ** |
| UserSeq 1 | 580 | LELEVNHASMSVVNDLMIQQATVKMGFRIYTQEQLRASLISKI |
| UserSeq 9 | 654 | LDLDVYHASVSVVKDLMIQQVAVKMASRVYTQDQLSAALYSRL |
| | | * * * *** *** ****** *** * *** ** * * * |

The JAZ-interacting domain (JID) of the maize MYC4 protein sequence having SEQ ID NO:9 is shown below as SEQ ID NO:10.

| | |
|---|---|
| 81 | ATG ASLLGWGDGY YKGCDEDKRK QKPLTPSAQA |
| 121 | EQEHRKRVLR ELNSLISGA |

In some cases, the maize MYC4 protein with SEQ ID NO:9 can have one or more mutations within the JID, and/or one or more mutations outside of the JID region. Thus modified MYC proteins described herein have less than 100% sequence identity to SEQ ID NO:9 and/or to SEQ ID NO:10.

For example, such a MYC4 from Zea mays can have modifications at positions corresponding to positions 94, 97, 102, 105, 107, 125, 128, 129, 148, 152, 155, 163, 165, 167, 169, 170, and/or 172 from the Arabidopsis thaliana MYC2 protein (highlighted in bold and with underlining in the comparison above). In some cases the amino acid positions in the endogenous Zea mays MYC4 protein can vary from those in the corresponding Arabidopsis thaliana MYC2 protein by 1-10 positions.

An example of another MYC-related protein is a maize (Zea mays) MYC4-like protein, shown below as SEQ ID NO:11, where the JAZ-interacting domain (JID) is shown in bold and with underlining.

| | |
|---|---|
| 1 | MNLWTDDNAS MMEAFMASAD LPAYPWGAPA GGGNPPPPQM |
| 41 | PPAMAMAPGF NQDTLQQRLQ AMIEGSRETW TYAIFWQSSL |
| 81 | DAATGASLLG WGDGYYKGCD DDKRRHRPPL TPAAQAEQEH |
| 121 | RKRVLRELNS LISGGASAAP APAPDEAVEE EVTDTEWFFL |
| 161 | VSMTQSFLNG SGLPGQALFA GHHTWIAAGL SSAPCDRARQ |
| 201 | AYNFGLRTMV CFPVGTGVLE LGSTDVVFQT AETMAKIRSL |
| 241 | FGGGPGGGSW PPVQPQAAPQ QQHAAEADQA AETDPSVLWL |
| 281 | ADAPVVDIKD SYSHPSAAEI SVSKPPPPPP PPQIHFENGS |
| 321 | TSTLTENPSP SVHAPPAPPA PPQRQQQNQG PFRRELNFSD |
| 361 | FASNPSLAAA PPFFKPESGE ILSFGVDSNA QRNPSPAPPA |
| 401 | SLTTAPGSLF SQSQHTATAA ANDAKNNNNN NKRSMEATSL |
| 441 | ASNTNHHPAA AANEGMLSFS SAPTARPSAG TGAPAKSESD |
| 481 | HSDLDASVRE VESSRVVAPP PEAEKRPRKR GRKPANGREE |
| 521 | PLNHVEAERQ RREKLNQRFY ALRAVVPNVS KMDKASLLGD |
| 561 | AISYINELRG KLTSLESDRE TLQAQVEALK KERDARPHPH |
| 601 | PAAGLGGHDA GGPRCHAVEI DAKILGLEAM IRVQCHKRNH |
| 641 | PSARLMTALR ELDLDVYHAS VSVVKDLMIQ QVAVKMASRM |
| 681 | YSQDQLSAAL YSRLAEPGSV MGR |

The JAZ-interacting domain (JID) of the maize MYC4-like protein sequence having SEQ ID NO:11 is shown below as SEQ ID NO:12.

| | |
|---|---|
| 81 | ATGASLLG WGDGYYKGCD DDKRRHRPPL TPAAQAEQEH |
| 121 | RKRVLRELNS LISGG |

A comparison of the Arabidopsis MYC4 protein with SEQ ID NO:5 with the Zea mays MYC4-like having SEQ ID NO:11 is shown below.

| 41.5% identity In 689 residues overlap; Score: 710.0; Gap frequency: 19.7% | | |
|---|---|---|
| Seq 5 | 25 | |
| STDDDASVMEAFIGGGSDHSSLFPPLPPPPLPQVNEDNLQQRLQALIEGANENWTYAVFW | | |
| Seq 11 | 18 | SADLPAYPWGAPAGGGNPPPPQMPPAMAMA- |
| PGFNQDTLQQRLQAMIEGSRETWTYAIFW | | |
| * * * * *** ** * * * ******* *** * **** | | |
| ** | | |
| Seq 5 | 85 | QSSHGFAGEDNNNNNTVLLGWGDGYYKGEEEKSRKKKS-- |
| NPASAAEQHRKRVIRELNS | | |
| Seq 11 | 77 | QSSLDAA------ |
| TGASLLGWGDGYYKGCDDDKRRHRPPLTPAAQAEQEHRKRVLRELNS | | |
| *** * *********** * ** ********* | | |
| ***** | | |
| Seq 5 | 143 | LISGGVGGG----- |
| DEAGDEEVTDTEWFFLVSMTQSFVKGTGLPGQAFSNSDTIWLSGSN | | |
| Seq 11 | 131 | |
| LISGGASAAPAPAPDEAVEEEVTDTEWFFLVSMTQSFLNGSGLPGQALFAGHHTWIAAG- | | |
| ***** *** ****************** * ****** * | | |
| Seq 5 | 198 | |
| ALAGSSCERARQGQIYGLQTMVCVATENGVVELGSSEIIHQSSDLVDKVDTFFNFNNGGG | | |
| Seq 11 | 190 | - |
| LSSAPCDRARQAYNFGLRTMVCFPVGTGVLELGSTDVVFQTAETMAKIRSLFGGGPGGG | | |
| * * **** ** **** ** **** * * * | | |
| *** | | |
| Seq 5 | 258 | EFGSWAFNLNPDQ---------GENDPG-LWI------------ |
| SEPNGVDSGLVAAP-- | | |
| Seq 11 | 249 | |
| SWPPVQPQAAPQQQHAAEADQAAETDPSVLWLADAPVVDIKDSYSHPSAAEISVSKPPPP | | |
| * * * ** ** * * * | | |
| Seq 5 | 294 | -----VMNNGGNDSTSNSDSQP------------------------------------ |
| -- | | |
| Seq 11 | 309 | |
| PPPPQIHFENGSTSTLTENPSPSVHAPPAPPAPPQRQQQNQGPFRRELNFSDFASNPSLA | | |
| * ** * | | |
| Seq 5 | 311 | -------------ISKLCNGSSVENPNPKVLKSC--------- |
| EMVNFKNGIEGQEEDS | | |
| Seq 11 | 369 | |
| AAPPFFKPESGEILSFGVDSNAQRNPSPAPPASLTTAPGSLFSQSQHTATAANDAKNNN | | |
| * ** * * * | | |
| Seq 5 | 349 | SNKKRS-------------PVSNNEEGMLSFTSV------------ |
| LPCDSNHSDLEASV | | |
| Seq 11 | 429 | |
| NNNKRSMEATSLASNTNHHPAAAANEGMLSFSSAPTARPSAGTGAPAKSESDHSDLDASV | | |
| * *** * ****** * * **** | | |
| *** | | |
| Seq 5 | 384 | AKEAESNRVVVEP--- |
| EKKPRKRGRKPANGREEPLNHVEAERQPREKLNQRFYSLRAVVP | | |
| Seq 11 | 489 | - |
| REVESSRVVAPPPEAEKRPRKRGRKPANGREEPLNHVEAERQRREKLNQRFYALRAVVP | | |
| * ** *** * ** ********************************** | | |
| ****** | | |
| Seq 5 | 441 | |
| NVSKMDKASLLGDAISYISELKSKLQKAESDKEELQKQIDVMNKEAGNAKSSVKDRKCLN | | |
| Seq 11 | 548 | |
| NVSKMDKASLLGDAISYINELRGKLTSLESDRETLQAQVEALKKERDARPHPHPAAGLGG | | |
| ****************** ** ** *** * ** * ** | | |
| Seq 5 | 501 | QESSV-- |
| LIEMEVDVKIIGWDAMIRIQCSKRNHPGAKFMEALKELDLEVNHASLSVVNDL | | |
| Seq 11 | 608 | |
| HDAGGPRCHAVEIDAKILGLEAMIRVQCHKRNHPSARLMTALRELDLDVYHASVSVVKDL | | |
| * * ** * **** ** ***** * * ** **** * *** *** | | |
| ** | | |
| Seq 5 | 559 | MIQQATVKMGNQFFTQDQLKVALTEKVGE |
| Seq 11 | 668 | MIQQVAVKMASRMYSQDQLSAALYSRLAE |
| | | **** *** **** ** * |

In some cases, the MYC4-like protein with SEQ ID NO:11 can have one or more mutations within the JID, and/or one or more mutations outside of the JID region. Thus modified MYC proteins described herein have less than 100% sequence identity to SEQ ID NO:11 and/or to SEQ ID NO:12.

For example, such a Zea mays MYC4-like protein can have modifications at positions corresponding to positions 94, 97, 102, 105, 107, 125, 128, 129, 148, 152, 155, 163, 165, 167, 169, 170, and/or 172 from the Arabidopsis thaliana MYC2 protein (highlighted in bold and with underlining in the comparison above). In some cases the amino acid positions in the endogenous Zea mays MYC4 protein can vary from those in the corresponding Arabidopsis thaliana MYC2 protein by 1-10 positions.

An example of another MYC-related protein is a Brachypodium distachyon MYC4-like protein, shown below as SEQ ID NO:13, where the JAZ-interacting domain (JID) is shown in bold and with underlining.

| | |
|---|---|
| 1 | MNLWTDDNAS MMEAFMASAA DLPTFPWGAA AATPPPPAAV |
| 41 | MPQQPAFNQD TLQQRLQAII EGSRETWTYA IFWQSSTDAG |
| 81 | AGASLLGWGD GYYKGCDDAD KRARQQPTPA SAAEQEHRKR |
| 121 | VLRELNSLIA GGGAAAPDEA VEEEVTDTEW FFLVSMTQSF |
| 161 | PNGMGLPGQA LYTRQPTWIA SGLASAPCER ARQAYTFGLR |
| 201 | TMVCIPVGTG VLELGATEVI FQTADSLGRI RSLFNLNGGG |
| 241 | GGGGAGSSWP PVAPHQQHGG DQAETDPSVL WLTDAPVGDM |
| 281 | KESPSVEISV SKPPPPPQIH HFENGSTSTL TENAGPSLHA |
| 321 | HQQPATLAPA APPRQNQHPH QLQLQHQQSQ QQQQQQQGPF |
| 361 | RRELNFSDFA TNASVTVTPP FFKPESGEIL NFGADSTSRR |
| 401 | NPSPAPPAAA ASLTTAPGSL FSQHTATVTA PTNEAKNNPK |
| 441 | RSMEATSRAS NTNHHPSATA NEGMLSFSSA PTTRPSTGTG |
| 481 | APAKSESDHS DLEASVREVE SSRVVPPPEE KRPRKRGRKP |
| 521 | ANGREEPLNH VEAERQRREK LNQRFYALRA VVPNVSKMDK |
| 561 | ASLLGDAISY INELRGKMTA LESDKDTLHS QIEALKKERD |
| 601 | ARPVAPLSGV HDSGPRCHAV EIEAKILGLE AMIRVQCHKR |
| 641 | NHPAAKLMTA LRELDLDVYH ASVSVVKDIM IQQVAVKMPN |
| 681 | RVYSQDQLNA ALYSRLAEPG APVPIR |

The JAZ-interacting domain (JID) of the Brachypodium distachyon MYC4-like protein sequence having SEQ ID NO:13 is shown below as SEQ ID NO: 14.

| | |
|---|---|
| 80 | G |
| 81 | AGASLLGWGD GYYKGCDDAD KRARQQPTPA SAAEQEHRKR |
| 121 | VLRELNSLIA GG |

In some cases, the Brachypodium distachyon MYC4-like protein with SEQ ID NO:13 can have one or more mutations within the JID, and/or one or more mutations outside of the JID region. Thus modified MYC proteins described herein have less than 100% sequence identity to SEQ ID NO:13 and/or to SEQ ID NO:14.

For example, such a MYC4-like from Brachypodium distachyon can have modifications at positions corresponding to positions 94, 97, 102, 105, 107, 125, 128, 129, 148, 152, 155, 163, 165, 167, 169, 170, and/or 172 from Arabidopsis thaliana.

An example of another MYC-related protein is a Sorghum bicolor MYC-like protein, shown below as SEQ ID NO:15, where the JAZ-interacting domain (JID) is shown in bold and with underlining.

| | |
|---|---|
| 1 | MNLWTDDNAS MMEAFMASAD LPTFPWGATA GGGNSSAAAA |
| 41 | TPPPPPQMPA AAMAPGFNQD TLQQRLQAMI EGSSETWTYA |
| 81 | IFWQSSLDAA TGASLLGWGD GYYKGCDDDK RKQRPLTPAA |
| 121 | QAEQEHRKRV LRELNSLISG AAAAPDEAVE EEVTDTEWFF |
| 161 | LVSMTQSFLN GSGLPGQALF AGQPTWIASG LSSAPCERAR |
| 201 | QAYNFGLRTM VCFPVGTGVL ELGSTDVVFQ TAESMAKIRS |
| 241 | LFGGGAGGGS WPPPQAPSHQ QPAAGPDQAE TDLWLADAPV |
| 281 | MDIKDSMSHP SAEISVSKPP PPPPPPQIHF ENASTSTLTE |
| 321 | NPSPSVHAAP PQPAPAAAPQ RQHQHQNQAH QGPFRRELNF |
| 361 | SDFASTNPSS LAATPPFFKP ESGEILSFGA DSNARRNPSP |
| 401 | APPAATASLT TAPGSLFSQH TATMTQAAAA NDAKNNNKRS |
| 441 | MEATSRASNT NHHPAATANE GMLSFSSAPT TRPSTGTGAP |
| 481 | AKSESDHSDL DASVREVESS RVVAPPPEAE KRPRKRGRKP |
| 521 | ANGREEPLNH VEAERQRREK LNQRFYALRA VVPNVSKMDK |
| 561 | ASLLGDAISY INELRGKLTS LESDKDTLQA QIEALKKERD |
| 601 | ARPPAHAAGL GGHDGGPRCH AVEIDAKILG LEAMIRVQCH |
| 641 | KRNHPSARLM TALRELDLDV YHASVSVVKD LMIQQVAVKM |
| 681 | ASRIYSQDQL NAALYSRLAE PGSAMGR |

The JAZ-interacting domain (JID) of the Sorghum bicolor MYC-like protein sequence having SEQ ID NO:15 is shown below as SEQ ID NO:16.

| | |
|---|---|
| 81 | A TGASLLGWGD GYYKGCDDDK RKQRPLTPAA |
| 121 | QAEQEHRKRV LRELNSLISG |

In some cases, the Sorghum bicolor MYC-like protein with SEQ ID NO: 15 can have one or more mutations within the JID, and/or one or more mutations outside of the JID region. Thus modified MYC proteins described herein have less than 100% sequence identity to SEQ ID NO:15 and/or to SEQ ID NO:16. For example, although any of the amino acids in the SEQ ID NO: 15 or 16 protein be modified, modification of the amino acids in the JAZ-interacting domain (JID), and/or modification of the serine at position 136 of the SEQ ID NO:15 or at the corresponding position of SEQ ID NO:16 can be useful to reduce interaction of the MYC-related protein with one or more JAZ proteins.

In other cases, such a Sorghum bicolor MYC-like protein can have modifications at positions corresponding to positions 94, 97, 102, 105, 107, 125, 128, 129, 148, 152, 155, 163, 165, 167, 169, 170, and/or 172 from Arabidopsis thaliana.

An example of another MYC-related protein is a Camelina sativa MYC2-like protein, shown below as SEQ ID NO:17, where the JAZ-interacting domain (JID) is shown in bold and with underlining.

| | |
|---|---|
| 1 | MTDYRLQPTM NLWTTDDNAS MMEAFISSSD ISTLWPMATT |
| 41 | TTTTTTATTS APATAMDIPA PAGFNQETLQ QRLQALIEGT |
| 81 | NEGWTYAIFW QPSYDFSGAS VLGWGDGYYK GEEDKAKPRQ |
| 121 | RSSSPPFSTP ADQEYRKKVL RELNSLISGG VAPSDDAVDE |
| 161 | EVTDTEWFFL VSMTQSFACG AGLAGRAFST GNAVWVSGSD |
| 201 | QLSGSGCERA KQGGVFGMQT IACIPSANGV VEVGSTEQIR |
| 241 | QSSDLINKVR VLFNLDGGAG DLSGLDWNLD PDQGENDPSM |
| 281 | WINDPIGAPG SNEPGNGAPS SSSQLFSKSI QFENGSSSTI |
| 321 | TENPNPDPTP SPVHSQTQNP KFSNNFSREL NFSTSSSTLV |
| 361 | KPRSGEILSF GDDGKRGSGN PDPSSYSGQT QFENKRKKSP |
| 401 | NEDKVLSFGD KTTGESDHSD LEASVVKEVA VEKRPKKRGR |
| 441 | KPANGREEPL NXMIYVIHSP NP |

The JAZ-interacting domain (JID) of the Camelina sativa MYC2-like protein sequence having SEQ ID NO:17 is shown below as SEQ ID NO:18.

| | |
|---|---|
| 41 | QRLQALIEGT |
| 81 | NEGWTYAIFW QPSYDFSGAS VLGWGDGYYK GEEDKAKPRQ |
| 121 | RSSSPPFSTP ADQEYRKKVL RELNSLISGG |

In some cases, the Camelina sativa MYC2-like protein with SEQ ID NO: 17 can have one or more mutations within the JID, and/or one or more mutations outside of the JID region. Thus modified MYC proteins described herein have less than 100% sequence identity to SEQ ID NO:17 and/or to SEQ ID NO:18.

For example, such a Camelina sativa MYC2-like protein can have modifications at positions corresponding to positions 94, 97, 102, 105, 107, 125, 128, 129, 148, 152, 155, 163, 165, 167, 169, 170, and/or 172 from Arabidopsis thaliana.

An example of another MYC-related protein is a Solanum lycopersicum MYC2-like protein, shown below as SEQ ID NO:19, where the JAZ-interacting domain (JID) is shown in bold and with underlining.

| | |
|---|---|
| 1 | MTEYSLPTMN LWNNSTSDDN VSMMEAFMSS DLSFWATNNS |
| 41 | TSAAVVGVNS NLPHASSNTP SVFAPSSSTS ASTLSAAATV |
| 81 | DASKSMPFFN QETLQQRLQA LIDGARETWT YAIFWQSSVV |
| 121 | DFSSPSVLGW GDGYYKGEED KAKRKLSVSS PAYIAEQEHR |
| 161 | KKVLRELNSL ISGAPPGTDD AVDEEVTDTE WFFLISMTQS |
| 201 | FVNGSGLPGQ ALYSSSPIWV AGTEKLAASH CERVRQAQGF |
| 241 | GLQTIVCIPS ANGVVELGST ELIVQSSDLM NKVRVLFNFS |
| 281 | NDLGSGSWAV QPESDPSALW LTDPSSSGME VRESLNTVQT |
| 321 | NSVPSSNSNK QIAYGNENNH PSGNGQSCYN QQQQKNPPQQ |
| 361 | QTQGFFTREL NFSEFGFDGS SNRNGNSSVS CKPESGEILN |
| 401 | FGDSTKKSAS SANVNLFTGQ SQFGAGEENN NKNKKRSATS |
| 441 | RGSNEEGMLS FVSGTVLPSS GMKSGGGGGE DSEHSDLEAS |
| 481 | VVKEADSSRV VEPEKRPRKR GRKPANGREE PLNHVEAERQ |
| 521 | RREKLNQRFY ALRAVVPNVS KMDKASLLGD AISYINELKS |
| 561 | KLQNTESDKE DLKSQIEDLK KESRRPGPPP PPNQDLKMSS |
| 601 | HTGGKIVDVD IDVKIIGWDA MIRIQCNKKN HPAARLMAAL |
| 641 | MELDLDVHHA SVSVVNDLMI QQATVKMGSR HYTEEQLRVA |
| 681 | LTSKIAETH |

The JAZ-interacting domain (JID) of the Solanum lycopersicum MYC2-like protein sequence having SEQ ID NO:19 is shown below as SEQ ID NO:20.

| | |
|---|---|
| 121 | FSSPSVLGW GDGYYKGEED KAKRKLSVSS PAYIAEQEHR |
| 161 | KKVLRELNSL ISGA |

A comparison of the Arabidopsis thaliana MYC2 sequence having SEQ ID NO:1 with the Solanum lycopersicum MYC2-like protein with SEQ ID NO:19 is shown below.

| 54.0% identity in 641 residues overlap; Score: 1333.0; Gap frequency: 8.7% | | |
|---|---|---|
| Seq 1 | 27 | SSSDISTLWPPASTTTTTATTETTPTPAMEIPAQAGFNQETLQQRLQALIEGTHEGWTYA |
| Seq 19 | 56 | SSNTPSVFAPSSSTSASTLSAAATVDASKSMPF---FNQETLQQRLQALIDGARETWTYA |
| | | ** * * ** * * * ************** * * **** |
| Seq 1 | 87 | IFWQPSY-DFSGASVLGWGDGYYKGEEDKANPRRRSSSPPFSTPADQEYRKKVLRELNSL |
| Seq 19 | 113 | IFWQSSVVDFSSPSVLGWGDGYYKGEEDKAKRKLSVSSPAYI--AEQEHRKKVLRELNSL |
| | | **** * *** ***************** *** * ** *********** |
| Seq 1 | 146 | ISGGVAPSDDAVDEEVTDTEWFFLVSMTQSFACGAGLAGKAFATGNAVWVSGSDQLSGSG |
| Seq 19 | 171 | ISGAPPGTDDAVDEEVTDTEWFFLISMTQSFVNGSGLPGQALYSSSPIWVAGTEKLAASH |
| | | *** **************** ****** * ** * * ** * * * |
| Seq 1 | 206 | CERAKQGGVFGMHTIACIPSANGVVEVGSTEPIRQSSDLINKVRILFNFDGGAGDLSGLN |
| Seq 19 | 231 | CERVPQAQGFGLQTIVCIPSANGVVELGSTELIVQSSDLMNKVRVLFNF---SNDLGSGS |
| | | *** * ** ** ********** **** * ***** **** **** ** |
| Seq 1 | 266 | WNLDPDQGENDPS-MWINDPIGTPGS-NEPGNGAPSSSSQLFSKSIQFENGSSSTITENP |
| Seq 19 | 288 | WAVQP---ESDPSALWLTDPSSSGMEVRESLNTVQTNSVPSSNSNKQIAYGNENNHPSGN |
| | | * * * *** * ** * * * * * |
| Seq 1 | 324 | NLDPTPSPVHSQTQNPKFNNTFSRELNFST-----------SSSTLVKPRSGEILNFGDE |
| Seq 19 | 345 | GQSCYNQQQQKNPPQQQTQGFFTRELNFSEFGFDGSSNRNGNSSVSCKPESGEILNFGDS |
| | | * ****** ** ** ********* |
| Seq 1 | 373 | GKRSSGNPDPSSYSGQTQF------ENKRKR----SMVLNEDKVLSF---------GDKT |
| Seq 19 | 405 | TKKSASSANVNLFTGQSQFGAGEENNNKNKKRSATSRGSNEEGMLSFVSGTVLPSSGMKS |
| | | * * ** ** ** * * ** *** * * |
| Seq 1 | 414 | AG----ESDHSDLEASVVKE------VAVEKRPKKRGRKPANGREEPLNHVEAERQRREK |
| Seq 19 | 465 | GGGGGEDSEHSDLEASVVKEADSSRVVEPEKRPRKRGRKPANGREEPLNHVEAERQRREK |
| | | * * *********** * **** ************************** |
| Seq 1 | 464 | LNQRFYALRAVVPNVSKMDKASLLGDAIAYINELKSKVVKTESEKLQIKNQLEEVKLELA |
| Seq 19 | 525 | LNQRFYALRAVVPNVSKMDKASLLGDAISYINELKSKLQNTESDKEDLKSQIEDLKKESR |
| | | **************************** ******** *** * * * * * * |
| Seq 1 | 524 | --GRKASASGGDMSSSCSSIKPVGMEIEVKIIGWDAMIRVESSKRNHPAARLMSALMDLE |
| Seq 19 | 585 | RPGPPPPPNQDLKMSSHTGGKIVDVDIDVKIIGWDAMIRIQCNKKNHPAARLMAALMELD |
| | | * ** * * * *********** * ******** *** * |
| Seq 1 | 582 | LEVNHASMSVVNDLMIQQATVKMGFRIYTQEQLRASLISKI |
| Seq 19 | 645 | LDVHHASVSVVNDLMIQQATVKMGSRHYTEEQLRVALTSKI |
| | | * * *** **************** * ** **** * *** |

In some cases, the Solanum lycopersicum MYC2-like protein with SEQ ID NO:19 can have one or more mutations within the JID, and/or one or more mutations outside of the JID region. For example, Solanum lycopersicum MYC2-like proteins can have mutations at positions 132, 190, 194, 197, or combinations thereof. In some cases, the positions of mutations can be at one position on either side of positions 132, 190, 194, or 197. Examples of mutations in Solanum lycopersicum MYC2-like proteins include mutations such as D132N, E190K, M197A, L194A, and combinations thereof. Thus modified MYC proteins described herein have less than 100% sequence identity to SEQ ID NO:19 and/or to SEQ ID NO:20.

In other examples, such a Solanum lycopersicum MYC2-like protein can have modifications at positions corresponding to positions 94, 97, 102, 105, 107, 125, 128, 129, 132, 148, 152, 155, 163, 165, 167, 169, 170, and/or 172 from Arabidopsis thaliana MYC2 protein (highlighted in bold and with underlining in the comparison above). In some cases the amino acid positions in the endogenous Zea mays MYC4 protein can vary from those in the corresponding Arabidopsis thaliana MYC2 protein by 1-10 positions.

An example of another MYC-related protein is a Solanum lycopersicum protein, shown below as SEQ ID NO:21, where the JAZ-interacting domain (JID) is shown in bold and with underlining.

| | |
|---|---|
| 1 | MTDYRLWSNT NTTNTCDDTM MMDSFLSSDP SSFWPASTPN |
| 41 | RPTPVNGVGE TMPFFNQESL QQRLQALIDG ARESWAYAIF |
| 81 | WQSSVVDFAS QTVLGWGDGY YKGEEDKNKR RGSSSSAANF |
| 121 | VAEQEHRKKV LRELNSLISG VQASAGNGTD DAVDEEVTDT |
| 161 | EWFFLISMTQ SFVNGNGLPG LAMYSSSPIW VTGTEKLAAS |
| 201 | QCERARQAQG FGLQTIVCIP SPESREILNF GDSSKRFSGQ |
| 241 | SQLGPGPGLM EENKNKNKNK KRSLGSRGNN EEGMLSFVSG |
| 281 | VILPTSTMGK SGDSDHSDLE ASVVKEAVVE PEKKPRKRGR |
| 321 | KPANGREEPL NHVEAERQRR EKLNQRFYEL RSQIECLRKE |
| 361 | LTNKGSSNYS ASPPLNQDVK IVDMDIDVKV IGWDAMIRIQ |
| 401 | CSKKNHPAAR LMAALKDLDL DVHHASVSVV NDLMIQQATV |
| 441 | KMGSRLYAQE QLRIALTSKI AESR |

The JAZ-interacting domain (JID) of the Solanum lycopersicum MYC-related protein sequence having SEQ ID NO:21 is shown below as SEQ ID NO:22.

| | |
|---|---|
| 81 | FAS QTVLGWGDGY YKGEEDKNKR RGSSSSAANF |
| 121 | VAEQEHRKKV LRELNSLISG V |

In some cases, the Solanum lycopersicum MYC-related protein with SEQ ID NO:21 can have one or more mutations within the JID, and/or one or more mutations outside of the JID region. For example, such Solanum lycopersicum MYC-related proteins can have mutations at positions 98, 161, 165, 168, or combinations thereof. In some cases, the positions of mutations can be at one position on either side of positions 98, 161, 165, or 168. Examples of mutations in Solanum lycopersicum MYC2-like proteins include mutations such as D98N, E161K, M168A, L165A, and combinations thereof. Thus modified MYC proteins described herein have less than 100% sequence identity to SEQ ID NO:21 and/or to SEQ ID NO:22.

An example of another MYC-related protein is a Solanum tuberosum MYC protein, shown below as SEQ ID NO:23, where the JAZ-interacting domain (JID) is shown in bold and with underlining.

| | |
|---|---|
| 1 | MTEYSLPTMN LWNNSTSDDN VSMMEAFMSS DLSFWATTNS |
| 41 | TTTNSASAAV VGVNSNLLHT NNNNPSVFPL SSSTSVSAAA |
| 81 | AVDATKSMPF FNQETLQQRL QALIDGARET WTYAIFWQSS |
| 121 | VVDFSSPSVL GWGDGYYKGE EDKAKRKLAV SSPAYIAEQE |
| 161 | HRKKVLRELN SLISGAPAGT DDAVDEEVTD TEWFFLISMT |
| 201 | QSFVNGSGLP GQALYSSSPI WVAGTEKLAA SHCERVRQAQ |
| 241 | GFGLQTIVCI PSANGVVELG STELIVESSD LMNKVRVLFN |
| 281 | FSNDLGSGSW AVQPESDPSA LWLTEPSSSG MEVRESLNTV |
| 321 | QTNSVPSSNS NKQIAYANEN NHQSGNGQSC YNLQQQQNNP |
| 361 | PQQQTQGFFT RELNFSEFGF DGSSNRNGNA SLSCKPESGE |
| 401 | ILNFGDSTKK SASSANVNLF TGQSQFGAVE ENNNNKNKKR |
| 441 | SATSRGSNEE GMLSFVSGTV LPSSGMKSGG GGGEDSEHSD |
| 481 | LEASVVKEAD SSRVVEPEKR PRKRGRKPAN GREEPLNHVE |
| 521 | AERQRREKLN QRFYALRAVV PNVSKMDKAS LLGDAISYIN |
| 561 | ELKSKLQNTE SDKEDLKSQI EDLKKESRRP GPPPPNQDLK |
| 601 | IGGKIVDVDI DVKIIGWDAM IGIQCNKKNH PAARLMAALM |
| 641 | ELDLDVHHAS VSVVNDLMIQ QATVKMGSRH YTEEQLRVAL |
| 681 | KSKIAETPLE SR |

The JAZ-interacting domain (JID) of the Solanum tuberosum MYC protein sequence having SEQ ID NO:23 is shown below as SEQ ID NO:24.

| | |
|---|---|
| 121 | FSSPSVL GWGDGYYKGE EDKAKRKLAV SSPAYIAEQE |
| 161 | HRKKVLRELN SLISGA |

In some cases, the Solanum tuberosum MYC protein with SEQ ID NO:23 can have one or more mutations within the JID, and/or one or more mutations outside of the JID region. Thus modified MYC proteins described herein have less than 100% sequence identity to SEQ ID NO:23 and/or to SEQ ID NO:24.

For example, such a Solanum tuberosum MYC protein with SEQ ID NO:23 can have modifications at positions corresponding to positions 94, 97, 102, 105, 107, 125, 128, 129, 132, 148, 152, 155, 163, 165, 167, 169, 170, and/or 172 from Arabidopsis thaliana MYC2 protein (highlighted in bold and with underlining in the comparison above). In some cases the amino acid positions in the endogenous Solanum tuberosum MYC protein can vary from those in the corresponding Arabidopsis thaliana MYC2 protein by 1-10 positions.

An example of another MYC-related protein is a Solanum tuberosum MYC protein, shown below as SEQ ID NO:25, where the JAZ-interacting domain (JID) is shown in bold and with underlining.

| | |
|---|---|
| 1 | MTDYRLWSNS NTTNTSDDNM MMDAFLSSDP SSFWPNRTSI |
| 41 | SPTPVNGGVG ETMPFFNQES LQQRLQALID GARESWAYAI |
| 81 | FWQSSSTSDF ATPSVLGWGD GYYKGEENKN KRRASSSSTN |
| 121 | FVAEQEHRKK VLRELNSLIS GVQATGAGSG GDDAVDEEVT |
| 161 | DTEWFFLISM TQSFANGNGL PGLAMYSSSP IWVTGTEKLA |
| 201 | GSQCERARQA QGFGLQTIVC IPSANGVVEL GSTELIFESS |
| 241 | DLMNKVKYLF NFNIDMGSVT GSGSGSCAVH PEPDPSALWL |
| 281 | TDPSSSVVEA KDSLINSSSR DVQLVFGNEN SENGTQNQQH |
| 321 | SQQTQGFFTK ELNFSGYGFD GSSTRNKNGN SSISCKPETR |
| 361 | EILNFGDSSK KSGSLFSGQS QFGPGTGLGL MEENKNNNKK |
| 401 | RSLASRGNNE KGMLSFVSGV ILPTSTMGKS GGGGNFDHSD |
| 441 | LEASVVKEAI VEPERKPRKR GRKPANGREE PLNHVEAERQ |
| 481 | RREKLNQRFY ALRAVVPNVS KMDKASLLGD AIAYINELKS |
| 521 | KVQNSDLDKE ELRSQIESLR KELANKGSSN YSSSPPSNQD |
| 561 | LKIVDMDIDV KVIGWDAMIR IQCSKKNHPA ARLMAALKDL |
| 601 | DLDVHHASVS VVNDLMIQQA TVKMGSRLYA QEQLTIALTS |
| 641 | KFAESR |

The JAZ-interacting domain (JID) of the Solanum tuberosum MYC protein sequence having SEQ ID NO:25 is shown below as SEQ ID NO:26.

| | |
|---|---|
| 81 | F ATPSVLGWGD GYYKGEENKN KRRASSSSTN |
| 121 | FVAEQEHRKK VLRELNSLIS GV |

In some cases, the Solanum tuberosum MYC protein with SEQ ID NO:25 can have one or more mutations within the JID, and/or one or more mutations outside of the JID region. Thus modified MYC proteins described herein have less than 100% sequence identity to SEQ ID NO:25 and/or to SEQ ID NO:26.

An example of another MYC-related protein is a Catharanthus roseus MYC2 protein, shown below as SEQ ID NO:27, where the JAZ-interacting domain (JID) is shown in bold and with underlining.

| | |
|---|---|
| 1 | MTDYRLQPKM NLWGTTTNTA ASPIITSDDN SSMMEAFMTS |
| 41 | SDPISLWPPS MSVNHHHPPT PTSSAVTTAV DSAKSMPAQP |
| 81 | AFFNQENLQQ RLQTLIDGAR ESWTYAIFWQ SSVVEFAGPS |
| 121 | VLGWGDGYYK GEEDKGKRKN SSSASSFAEQ EHRKKVLREL |
| 161 | NSLIAGPQGT ADDAVDEEVT DTEWFFLISM TQSFVSGSGL |
| 201 | PGQALYNSNP VWVTGAGRLA VSHCDRARQA QSFGLQTLVC |
| 241 | IPSANGVVEL GSTELIFQSS DLMNKVRILF NFNNIDLGSS |
| 281 | SGPWPENDPS SLWLTDPSPS GVGVKEGVNT NNNTSVQGNS |
| 321 | IPSGNKQQLV FGNNDNHPTT STLTDHPGAG AVNSYNNSSQ |
| 361 | NAQQPQGSFF TRELNFSEYG FERSSVKNGN CKPESGEILN |
| 401 | FGGESVTKKN SVSGNGNLFS VQSQFGAGEE NKNKKRPSPV |
| 441 | SRGSNDEGML SFTSGVVLPS TGVVKSSGGG GGGDSDHSDL |
| 481 | EASVVKEAES SRVVDPEKRP RKRGRKPANG REEPLNHVEA |
| 521 | ERQRREKLNQ RFYALRAVVP NVSKMDKASL LGDAISYINE |
| 561 | LKAKLQTTET DKDELKNQLD SLKKELASKE SRLLSSPDQD |
| 601 | LKSSNKQSVG NLDMDIDVKI IGREAMIRVQ SSKNNHPAAR |
| 641 | VMGALKDLDL ELLHASVSVV NDLMIQQNTV RMGSRFYTQE |
| 681 | QLRIALTSRI AGNSMRLLV |

A comparison of the Catharanthus roseus MYC2 protein sequence having SEQ ID NO:27 with the MYC2 protein sequence having SEQ ID NO:1 is shown below.

| 54.1% identity in 693 residues overlap; Score: 1363.0; Gap frequency: 10.7% | | |
|---|---|---|
| UserSeq1 | 1 | MTDYRLQPTMNLW------------TTDDNASMMEAFMSSSDISTLWPRAST-------T |
| UserSeq27 | 1 | MTDYRLQPKMNLWGTTTNTAASPITTSDDNSSMMEAFMTSSDPISLWPPSMSVNHHHPPT |
| | | ******** **** * *** ******* *** **** * |
| UserSeq1 | 42 | TTTATTETTPTPAMEIPAQ-AGFNQETLQQRLQALIEGTHEGWTYAIFWQPSY-DFSGAS |
| UserSeq27 | 61 | PTSSAVTTAVDSAKSMPAQPAFFNQENLQQRLQTLIDGARESWTYAIFWQSSVVEFAGPS |
| | | * * * *** * **** ****** ** * * ******** * * * * |
| UserSeq1 | 100 | VLGWGDGYYKGEEDKANPRRRSSSPPFSTPADQEYRKKVLRELNSLISGGVAPSDDAVDE |
| UserSeq27 | 121 | VLGWGDGYYKGEEDKGKRKNSSSASSF---AEQEHRKKVLRELNSLIAGPQGTADDAVDE |
| | | *************** ** * * ** ************ * ****** |
| UserSeq1 | 160 | EVTDTEWFFLVSMTQSFACGAGLAGKAFATGNAVWVSGSDQLSGSGCERAKQGGVFGMHT |
| UserSeq27 | 178 | EVTDTEWFFLISMTQSFVSGSGLPGQALYNSNPVWVTGAGRLAVSHCDRARQAQSFGLQT |
| | | ********** ****** * ** * * * *** * * * * ** * ** * |
| UserSeq1 | 220 | IACIPSANGVVEVGSTEPIRQSSDLINKVRILFNFDG-GAGDLSGLNWNLDPDQ-GENDP |
| UserSeq27 | 238 | LVCIPSANGVVELGSTELIFQSSDLMNKVRILFNFNNIDLGSSSGPWPENDPSSLWLTDP |
| | | ********** **** * ***** ********* * ** ** ** |
| UserSeq1 | 278 | S---MWINDPIGTPGSNE-PGNGAPSSSSQLFSKSIQFENGSSSTITENPNLDPTPSPVH |
| UserSeq27 | 298 | SPSGVGVKEGVNTNNNTSVQGNSIPSGNKQQLVFGNNDNHPTTSTLTDHPGAGAVNSYNN |
| | | * * ** ** * ** * * * |
| UserSeq1 | 334 | S-QTQNPKFNNTFSRELNES-------TSSSTLVKPRSGEILNFGDEG--KRSSGNPDPS |
| UserSeq27 | 358 | SSQNAQQPQGSFFTRELNFSEYGFERSSVKNGNCKPESGEILNFGGESVTKKNSVSGNGN |
| | | * * * ****** ** ******** * * * |
| UserSeq1 | 384 | SYSGQTQE----ENKRKR-----SMVLNEDKVLSF---------------GDKTAGESDH |
| UserSeq27 | 418 | LFSVQSQFGAGEENKNKKRPSPVSRGSNDEGMLSFTSGVVLPSTGVVKSSGGGGGGDSDH |
| | | * * ** *** * * * *** * * *** |
| UserSeq1 | 420 | SDLEASVVKE------VAVEKRPKKRGRKPANGREEPLNHVEAERQRREKLNQRFYALRA |
| UserSeq27 | 478 | SDLEASVVKEAESSRVVDPEKRPRKRGRKPANGREEPLNHVEAERQRREKLNQRFYALRA |
| | | ********** * **** ************************************ |
| UserSeq1 | 474 | VVPNVSKMDKASLLGDAIAYINELKSKVVKTESEKLQIKNQLEEVKLELAGRKA---SAS |
| UserSeq27 | 538 | VVPNVSKMDKASLLGDAISYINELKAKLQTTETDKDELKNQLDSLKKELASKESRLLSSP |
| | | ****************** ****** * ** * **** * *** * |
| UserSeq1 | 531 | GGDM-SSSCSSIKPVGMEIEVKIIGWDAMIRVESSKRNHPAARLMSALMDLELEVNHASM |
| UserSeq27 | 598 | DQDLKSSNKQSVGNLDMDIDVKIIGREAMIRVQSSKNNHPAARVMGALKDLDLELLHASV |
| | | * ** * * * ***** ***** *** ****** * ** ** ** *** |
| UserSeq1 | 590 | SVVNDLMIQQATVKMGFRIYTQEQLRASLISKI |
| UserSeq27 | 658 | SVVNDLMIQQNTVRMGSRFYTQEQLRIALTSRI |
| | | ********** ** ** * ******* * * * |

The JAZ-interacting domain (JID) of the Catharanthus roseus MYC2 protein sequence having SEQ ID NO:27 is shown below as SEQ ID NO:28.

| | |
|---|---|
| 81 | FAGPS |
| 121 | VLGWGDGYYK GEEDKGKRKN SSSASSFAEQ EHRKKVLREL |
| 161 | NSLIAG |

In some cases, the Catharanthus roseus MYC protein with SEQ ID NO:27 can have one or more mutations within the JID, and/or one or more mutations outside of the JID region. Thus modified MYC proteins described herein have less than 100% sequence identity to SEQ ID NO:27 and/or to SEQ ID NO:28.

For example, such a Catharanthus roseus MYC protein with SEQ ID NO:27 can have modifications at positions corresponding to positions 94, 97, 102, 105, 107, 125, 128, 129, 132, 148, 152, 155, 163, 165, 167, 169, 170, and/or 172 from Arabidopsis thaliana MYC2 protein (highlighted in bold and with underlining in the comparison above). In some cases the amino acid positions in the endogenous Catharanthus roseus MYC protein can vary from those in the corresponding Arabidopsis thaliana MYC2 protein by 1-10 positions.

### PhyB and Modifications of PhyB to Enhance Growth

Although jasmonate and MYC transcription factors are potent activators of defense responses, the jasmonate hormone is also a potent inhibitor of plant growth. Plants cells in which the jasmonate pathway and MYC transcription factors are activated exhibit slow growth and low yield. For example, treatment of taxus cells with exogenous jasmonate is currently used to boost taxol production, but the downside of such treatment is that such cells stop dividing and growing. Also, plants having the dominant atr2D mutant of MYC3 (a D94N missense mutation in the JAZ-interacting domain (JID)) grow very slowly. Hence even if expression of such MYC3 proteins can relieve repression by JAZ proteins and improve the environmental stress resistance of plants, use of such an atr2D mutation by itself may not be particularly useful.

The PhyB gene encodes a regulatory photoreceptor protein (Phytochrome B (PHYB)) that exists in two forms. The two forms are reversibly interconvertible by light, where a Pr form absorbs maximally in the red region of the spectrum and where a Pfr form absorbs maximally in the far-red region. As described herein phyB gene mutations improve plant growth, for example, in myc mutant plants that have increased resistance to environmental challenges such as insects. Plant cells, plants, and seeds from selected plant species can be modified to have loss-of-function phyB mutations to improve cell growth.

One example of an Arabidopsis thaliana phytochrome B (PHYB) protein sequence is shown below (SEQ ID NO:30). The codon encoding the arginine (R) at position 322 that is highlighted (in bold and with underlining) is replaced by a termination signal (TGA) in some of the mutant phyB plant lines described herein that have improved plant growth. This genetic mutation in such a mutant phyB is a C→T substitution, causing the arginine codon (CGA) to become a termination codon (TGA). Hence, mutant phyB plant lines with phyB loss of function mutations (e.g., deletions) exhibit improved plant growth, for example, when present in a mutant jaz genetic background.

| | |
|---|---|
| 1 | MVSGVGGSGG GRGGGRGGEE EPSSSHTPNN RRGGEQAQSS |
| 41 | GTKSLRPRSN TESMSKAIQQ YTVDARLHAV FEQSGESGKS |
| 81 | FDYSQSLKTT TYGSSVPEQQ ITAYLSRIQR GGYIQPFGCM |
| 121 | IAVDESSFRI IGYSENAREM LGIMPQSVPT LEKPEILAMG |
| 161 | TDVRSLFTSS SSILLERAFV AREITLLNPV WIHSKNTGKP |
| 201 | FYAILHRIDV GVVIDLEPAR TEDPALSIAG AVQSQKLAVR |
| 241 | AISQLQALPG GDIKLLCDTV VESVRDLTGY DRVMVYKFHE |
| 281 | DEHGEVVAES KRDDLEPYIG LHYPATDIPQ ASRFLFKQNR |
| 321 | VRMIVDCNAT PVLVVQDDRL TQSMCLVGST LRAPHGCHSQ |
| 361 | YMANMGSIAS LAMAVIINGN EDDGSNVASG RSSMRLWGLV |
| 401 | VCHHTSSRCI PFPLRYACEF LMQAFGLQLN MELQLALQMS |
| 441 | EKRVLRTQTL LCDMLLRDSP AGIVTQSPSI MDLVKCDGAA |
| 481 | FLYHGKYYPL GVAPSEVQIK DVVEWLLANH ADSTGLSTDS |
| 521 | LGDAGYPGAA ALGDAVCGMA VAYITKRDFL FWFRSHTAKE |
| 561 | IKWGGAKHHP EDKDDGQRMH PRSSFQAFLE VVKSRSQPWE |

| | |
|---|---|
| 601 | TAEMDAIHSL QLILRDSFKE SEAAMNSKVV DGVVQPCRDM |
| 641 | AGEQGIDELG AVAREMVPLI ETATVPIFAV DAGGCINGWN |
| 681 | AKIAELTGLS VEEAMGKSLV SDLIYKENEA TVNKLLSRAL |
| 721 | RGDEEKNVEV KLKTFSPELQ GKAVFVVVNA CSSKDYLNNI |
| 761 | VGVCFVGQDV TSQKIVMDKF INIQGDYKAI VHSPNPLIPP |
| 801 | IFAADENTCC LEWNMAMEKL TGWSRSEVIG KMIVGEVFGS |
| 841 | CCMLKGPDAL TKFMIVLHNA IGGQDTDKFP FPFFDRNGKF |
| 881 | VQALLTANKR VSLEGKVIGA FCFLQIPSPE LQQALAVQRR |
| 921 | QDTECFTKAK ELAYICQVIK NPLSGMRFAN SLLEATDLNE |
| 961 | DQKQLLETSV SCEKQISRIV GDMDLESIED GSFVLKREEF |
| 1001 | FLGSVINAIV SQAMFLLRDR GLQLIRDIPE EIKSIEVFGD |
| 1041 | QIRIQQLLAE FLLSIIRYAP SQEWVEIHLS QLSKQMADGF |
| 1081 | AAIRTEFRMA CPGEGLPPEL VRDMFHSSRW TSPEGLGLSV |
| 1121 | CRKILKLMNG EVQYIRESER SYFLIILELP VPRKRPLSTA |
| 1151 | SGSGDMMLMM PY |

A chromosomal DNA sequence for the Arabidopsis thaliana phytochrome B (PHYB) protein with SEQ ID NO:30 is shown below as SEQ ID NO:31.

| | |
|---|---|
| 1 | CTTCAATTTA TTTTATTGGT TTCTCCACTT ATCTCCGATC |
| 41 | TCAATTCTCC CCATTTTCTT CTTCCTCAAG TTCAAAATTC |
| 81 | TTGAGAATTT AGCTCTAGCA GAATTCGTCT CCGATAACTA |
| 121 | GTGGATGATG ATTCACCCTA AATCCTTCCT TGTCTCAAGG |
| 161 | TAATTCTGAG AAATTTCTCA AATTCAAAAT CAAACGGCAT |
| 201 | GGTTTCCGGA GTCGGGGGTA GTGGCGGTGG CCGTGGCGGT |
| 241 | GGCCGTGGCG GAGAAGAAGA ACCGTCGTCA AGTCACACTC |
| 281 | CTAATAACCG AAGAGGAGGA GAACAAGCTC AATCGTCGGG |
| 321 | AACGAAATCT CTCAGACCAA GAAGCAACAC TGAATCAATG |
| 361 | AGCAAAGCAA TTCAACAGTA CACCGTCGAC GCAAGACTCC |
| 401 | ACGCCGTTTT CGAACAATCC GGCGAATCAG GGAAATCATT |
| 441 | CGACTACTCA CAATCACTCA AAACGACGAC GTACGGTTCC |
| 481 | TCTGTACCTG AGCAACAGAT CACAGCTTAT CTCTCTCGAA |
| 521 | TCCAGCGAGG TGGTTACATT CAGCCTTTCG GATGTATGAT |
| 561 | CGCCGTCGAT GAATCCAGTT TCCGGATCAT CGGTTACAGT |

| | |
|---|---|
| 601 | GAAAACGCCA GAGAAATGTT AGGGATTATG CCTCAATCTG |
| 641 | TTCCTACTCT TGAGAAACCT GAGATTCTAG CTATGGGAAC |
| 681 | TGATGTGAGA TCTTTGTTCA CTTCTTCGAG CTCGATTCTA |
| 721 | CTCGAGCGTG CTTTCGTTGC TCGAGAGATT ACCTTGTTAA |
| 761 | ATCCGGTTTG GATCCATTCC AAGAATACTG GTAAACCGTT |
| 801 | TTACGCCATT CTTCATAGGA TTGATGTTGG TGTTGTTATT |
| 841 | GATTTAGAGC CAGCTAGAAC TGAAGATCCT GCGCTTTCTA |
| 881 | TTGCTGGTGC TGTTCAATCG CAGAAACTCG CGGTTCGTGC |
| 921 | GATTTCTCAG TTACAGGCTC TTCCTGGTGG AGATATTAAG |
| 961 | CTTTTGTGTG ACACTGTCGT GGAAAGTGTG AGGGACTTGA |
| 1001 | CTGGTTATGA TCGTGTTATG GTTTATAAGT TTCATGAAGA |
| 1041 | TGAGCATGGA GAAGTTGTAG CTGAGAGTAA ACGAGATGAT |
| 1081 | TTAGAGCCTT ATATTGGACT GCATTATCCT GCTACTGATA |
| 1121 | TTCCTCAAGC GTCAAGGTTC TTGTTTAAGC AGAACCGTGT |
| 1161 | CCGAATGATA GTAGATTGCA ATGCCACACC TGTTCTTGTG |
| 1201 | GTCCAGGACG ATAGGCTAAC TCAGTCTATG TGCTTGGTTG |
| 1241 | GTTCTACTCT TAGGGCTCCT CATGGTTGTC ACTCTCAGTA |
| 1281 | TATGGCTAAC ATGGGATCTA TTGCGTCTTT AGCAATGGCG |
| 1321 | GTTATAATCA ATGGAAATGA AGATGATGGG AGCAATGTAG |
| 1361 | CTAGTGGAAG AAGCTCGATG AGGCTTTGGG GTTTGGTTGT |
| 1401 | TTGCCATCAC ACTTCTTCTC GCTGCATACC GTTTCCGCTA |
| 1441 | AGGTATGCTT GTGAGTTTTT GATGCAGGCT TTCGGTTTAC |
| 1481 | AGTTAAACAT GGAATTGCAG TTAGCTTTGC AAATGTCAGA |
| 1521 | GAAACGCGTT TTGAGAACGC AGACACTGTT ATGTGATATG |
| 1561 | CTTCTGCGTG ACTCGCCTGC TGGAATTGTT ACACAGAGTC |
| 1601 | CCAGTATCAT GGACTTAGTG AAATGTGACG GTGCAGCATT |
| 1641 | TCTTTACCAC GGGAAGTATT ACCCGTTGGG TGTTGCTCCT |
| 1681 | AGTGAAGTTC AGATAAAAGA TGTTGTGGAG TGGTTGCTTG |
| 1721 | CGAATCATGC GGATTCAACC GGATTAAGCA CTGATAGTTT |
| 1761 | AGGCGATGCG GGGTATCCCG GTGCAGCTGC GTTAGGGGAT |
| 1801 | GCTGTGTGCG GTATGGCAGT TGCATATATC ACAAAAAGAG |
| 1841 | ACTTTCTTTT TTGGTTTCGA TCTCACACTG CGAAAGAAAT |
| 1881 | CAAATGGGGA GGCGCTAAGC ATCATCCGGA GGATAAAGAT |
| 1921 | GATGGGCAAC GAATGCATCC TCGTTCGTCC TTTCAGGCTT |
| 1961 | TTCTTGAAGT TGTTAAGAGC CGGAGTCAGC CATGGGAAAC |
| 2001 | TGCGGAAATG GATGCGATTC ACTCGCTCCA GCTTATTCTG |
| 2041 | AGAGACTCTT TTAAAGAATC TGAGGCGGCT ATGAACTCTA |
| 2081 | AAGTTGTGGA TGGTGTGGTT CAGCCATGTA GGGATATGGC |
| 2121 | GGGGGAACAG GGGATTGATG AGTTAGGTGC AGTTGCAAGA |
| 2161 | GAGATGGTTA GGCTCATTGA GACTGCAACT GTTCCTATAT |
| 2201 | TCGCTGTGGA TGCCGGAGGC TGCATCAATG GATGGAACGC |
| 2241 | TAAGATTGCA GAGTTGACAG GTCTCTCAGT TGAAGAAGCT |
| 2281 | ATGGGGAAGT CTCTGGTTTC TGATTTAATA TACAAAGAGA |
| 2321 | ATGAAGCAAC TGTCAATAAG CTTCTTTCTC GTGCTTTGAG |
| 2361 | AGGTATATTC AGTTCTTCAG CTATGTTGTA TCTGCGGTGT |
| 2401 | ATATACCAAT TCGCGGGTAT TTGATTATTT TGTTGCATTT |
| 2441 | GGCAATGCAG GGGACGAGGA AAAGAATGTG GAGGTTAAGC |
| 2481 | TGAAAACTTT CAGCCCCGAA CTACAAGGGA AAGCAGTTTT |
| 2521 | TGTGGTTGTG AATGCTTGTT CCAGCAAGGA CTACTTGAAC |
| 2561 | AACATTGTCG GCGTTTGTTT TGTTGGACAA GACGTTACTA |
| 2601 | GTCAGAAAAT CGTAATGGAT AAGTTCATCA ACATACAAGG |
| 2641 | AGATTACAAG GCTATTGTAC ATAGCCCAAA CCCTCTAATC |
| 2681 | CCGCCAATTT TTGCTGCTGA CGAGAACACG TGCTGCCTGG |
| 2721 | AATGGAACAT GGCGATGGAA AAGCTTACGG GTTGGTCTCG |
| 2761 | CAGTGAAGTG ATTGGGAAAA TGATTGTCGG GGAAGTGTTT |
| 2801 | GGGAGCTGTT GCATGCTAAA GGGTCCTGAT GCTTTAACCA |
| 2841 | AGTTCATGAT TGTATTGCAT AATGCGATTG GTGGCCAAGA |
| 2881 | TACGGATAAG TTCCCTTTCC CATTCTTTGA CCGCAATGGG |
| 2921 | AAGTTTGTTC AGGCTCTATT GACTGCAAAC AAGCGGGTTA |
| 2961 | GCCTCGAGGG AAAGGTTATT GGGGCTTTCT GTTTCTTGCA |
| 3001 | AATCCCGAGC CCTGAGCTGC AGCAAGCTTT AGCAGTCCAA |
| 3041 | CGGAGGCAGG ACACAGAGTG TTTCACGAAG GCAAAAGAGT |
| 3081 | TGGCTTATAT TTGTCAGGTG ATAAAGAATC CTTTGAGCGG |
| 3121 | TATGCGTTTC GCAAACTCAT TGTTGGAGGC CACAGACTTG |
| 3161 | AACGAGGACC AGAAGCAGTT ACTTGAAACA AGTGTTTCTT |
| 3201 | GCGAGAAACA GATCTCAAGG ATCGTCGGGG ACATGGATCT |
| 3241 | TGAAAGCATT GAAGACGGGT GAGTATAGTT AGAATTTATC |
| 3281 | TAGAAGCTAG TTTTGCTTAC TTCACAAAAT GTGACCAAAT |
| 3321 | CCCAAATTTT GTTTTTTTCA TTGATCAGTT CATTTGTGCT |
| 3361 | AAAGAGGGAA GAGTTTTTCC TTGGAAGTGT CATAAACGCG |
| 3401 | ATTGTAAGTC AAGCGATGTT CTTATTAAGG GACAGAGGTC |
| 3441 | TTCAGCTGAT CCGTGACATT CCCGAAGAGA TCAAATCAAT |
| 3481 | AGAGGTTTTT GGAGACCAGA TAAGGATTCA ACAGCTCCTG |
| 3521 | GCTGAGTTTC TGCTGAGTAT AATCCGGTAT GCACCATCTC |
| 3561 | AAGAGTGGGT GGAGATCCAT TTAAGCCAAC TTTCAAAGCA |
| 3601 | AATGGCTGAT GGATTCGCCG CCATCCGCAC AGAATTCAGG |
| 3641 | TACATTTCAT TGTTCCCGCT GTTGTCTCCA CATATCCATA |
| 3681 | ACCAAAATTA TGCAATCCGG TTTTTTTGGT TCCTTATTTT |
| 3721 | GTACATAAAG AAAATGAATT TGGTTTGGTT AATTACGAAT |
| 3761 | TTGATTTAGG CGTTTAAAGA ATTTGAGGTT TTAACCAATT |
| 3801 | CACTATTTGT TTTGGTTATT GTTTAGTTGG AACCTAGATT |
| 3841 | AGTTTGATTT TTGTATTCGG TTTAGTCGAC TTGGGAACTT |
| 3881 | TTAGACACAT CCATAGGCCT AGAATTAGCA GTCAAGGAAT |
| 3921 | GTAATGTTTT CAAATTGATG AAAACCAGCT CAAAAGTGTA |
| 3961 | AAACTTGGGT TTCATGTGTT GGTGTCTTTG TTATGTCTTT |
| 4001 | ATTCGTTGTT TGCAGAATGG CGTGTCCAGG TGAAGGTCTG |
| 4041 | CCTCCAGAGC TAGTCCGAGA CATGTTCCAT AGCAGCAGGT |
| 4081 | GGACAAGCCC TGAAGGTTTA GGTCTAAGCG TATGTCGAAA |
| 4121 | GATTTTAAAG CTAATGAACG GTGAGGTTCA ATACATCCGA |
| 4161 | GAATCAGAAC GGTCCTATTT CCTCATCATT CTGGAACTCC |
| 4201 | CTGTACCTCG AAAGCGACCA TTGTCAACTG CTAGTGGAAG |
| 4241 | TGGTGACATG ATGCTGATGA TGCCATATTA GTCACACTTC |
| 4281 | AGTTGGTATG AGAGTTTGTA TCATTGTATG AGTGTTTGTG |
| 4321 | TGTCTAACGA CGTCGGAGGA GGATAGAAAG TTTTTTTTTT |
| 4361 | GTTTCCGGTG AGATTAGTAG AGAAGAGGGA GATTATTTGC |
| 4401 | GTTCAGCTCA GCTCGCCGGA AAAAAAACGT AACAGTAGTT |
| 4441 | GTAGAGAATT TCAAGACTTT TGTTTGTGCT GTGTAAATTG |
| 4481 | ACAACTCCGA GAGAAACAAA ACAATGAGAT AAGAAGAGAG |
| 4081 | CATATTAATC GATGACCAAT CCTTTTAATT |

Chromosomal sequences that encode phytochrome B and/or phytochrome B-related polypeptides from many plant types and species can be modified to reduce or eliminate the expression and/or function of the encoded polypeptide. For example, chromosomal sequences encoding phytochrome B and/or phytochrome B-related polypeptides from agriculturally important plants such as alfalfa (e.g., forage legume alfalfa), algae, avocado, barley, broccoli, Brussels sprouts, cabbage, canola, cassava, cauliflower, cole vegetables, collards, corn, crucifers, grain legumes, grasses (e.g., forage grasses), jatropa, kale, kohlrabi, maize, miscanthus, mustards, nut sedge, oats, oil firewood trees, oilseeds, potato, radish, rape, rapeseed, rice, rutabaga, sorghum, soybean, sugar beets, sugarcane, sunflower, switchgrass, tobacco, tomato, turnips, and/or wheat can be modified reduce or eliminate the expression and/or function of one or more encoded phytochrome B and/or phytochrome B-related polypeptides.

In some cases, more than one gene or chromosomal segment encoding a phytochrome B and/or phytochrome B-related polypeptide can be modified to reduce or eliminate the expression and/or function of the encoded protein(s).

The following are examples of “phytochrome B-related” proteins and nucleic acids that can be modified to reduce or eliminate the expression and/or function thereof, and thereby generate plants with improved growth.

An uncharacterized Zea mays protein referred to as LOC100383702 (NCBI accession no. NP_001169810.1 (GI:293335473) has significant sequence identity to the Arabidopsis thaliana PHYB protein with SEQ ID NO:30, as illustrated by the sequence comparison shown below. Domains of sequence homology are identified by asterisks below the sequence comparisons.

| 73.1% identity in 1139 residues overlap; Score: 4271.0; Gap frequency: 0.9% | | |
|---|---|---|
| Seq30 | 22 | PSSSHTPNNRRGGEQAQSSGTKSLRPRSNTESMSKAIQQYTVDARLHAVFEQSGESGKSF |
| Seq32 | 18 | PEAPRHAHHHHHSQSSGGSTSRAGGGAAATESVSKAVAQYTLDARLHAVFEQSGASGRSF |
| | | * * *** *** *** ************ ** ** |
| Seq30 | 82 | DYSQSLKTTTYGSSVPEQQITAYLSRIQRGGYIQPFGCMIAV-DESSFRIIGYSENAPEM |
| Seq32 | 78 | DYSQSLRAPPTPSS--EQQIAAYLSRIQRGGHIQPFGCTLAVADDSSFRLLAFSENSPDL |
| | | ****** ** **** ********** ****** ** * **** *** |
| Seq30 | 141 | LGIMPQ-SVPTLEK--PEILAMGTDVRSLFTSSSSILLERAFVAREITLLNPVWIHSKNT |
| Seq32 | 136 | LDLSPHHSVPSLDSSAPPHVSLGADARLLFSPSSAVLLERAFAAREISLLNPIWIHSRVS |
| | | * * *** * * * * * ** ** ******* **** **** **** |
| Seq30 | 198 | GKPFYAILHRIDVGVVIDLEPARTEDPALSIAGAVQSQKLAVRAISQLQALPGGDIKLLC |
| Seq32 | 196 | SKPFYAILHRIDVGVVIDLEPARTEDPALSIAGAVQSQKLAVRAISRLQALPGGDVKLLC |
| | | ********************************************* ******** **** |
| Seq30 | 258 | DTVVESVRDLTGYDRVMVYKFHEDEHGEVVAESKRDDLEPYIGLHYPATDIPQASRFLFK |
| Seq32 | 256 | DTVVEHVRELTGYDRVMVYRFHEDEHGEVVAESRRDNLEPYLGLHYPATDIPQASRFLFR |
| | | ***** ** ********** ************* ** **** ***************** |
| Seq30 | 318 | QNRVRMIVDCNATPVLVVQDDRLTQSMCLVGSTLRAPHGCHSQYMANMGSIASLAMAVII |
| Seq32 | 316 | QNRVRMIADCHATPVRVIQDPGLSQPLCLVGSTLRAPHGCHAQYMANMGSIASLVMAVII |
| | | ******* ** **** * ** * * ************** ************ ***** |
| Seq30 | 378 | NGNEDDGSNVASGRSS-MRLWGLVVCHHTSSRCIPFPLRYACEFLMQAFGLQLNMELQLA |
| Seq32 | 376 | SSGGDDEQTGRGGISSAMKLWGLVVCHHTSPRCIPFPLRYACEFLMQAFGLQLNMELQLA |
| | | ** * ** * *********** ***************************** |
| Seq30 | 437 | LQMSEKRVLRTQTLLCDMLLRDSPAGIVTQSPSIMDLVKCDGAAFLYHGKYYPLGVAPSE |
| Seq32 | 436 | HQLSEKHILRTQTLLCDMLLRDSPTGIVTQSPSIMDLVKCDGAALYYHGKYYPLGVTPTE |
| | | * *** **************** ******************* ********** * * |
| Seq30 | 497 | VQIKDVVEWLLANHADSTGLSTDSLGDAGYPGAAALGDAVCGMAVAYITKRDFLFWFRSH |
| Seq32 | 496 | SQIKDIIEWLTVFHGDSTGLSTDSLADAGYLGAAALGEAVCGMAVAYITPSDYLFWFRSH |
| | | **** *** * ********** **** ****** *********** * ******* |
| Seq30 | 557 | TAKEIKWGGAKHHPEDKDDGQRMHPRSSFQAFLEVVKSRSQPWETAEMDAIHSLQLILRD |
| Seq32 | 556 | TAKEIKWGGAKHHPEDKDDGQRMHPRSSFKAFLEVVKSRSLPWENAEMDAIHSLQLILRD |
| | | ***************************** ********** *** *************** |
| Seq30 | 617 | SFKES-EAAMNSKVVDGVVQPCRDMAGEQGIDELGAVAREMVRLIETATVPIFAVDAGGC |
| Seq32 | 616 | SFRDAAEGTNNSKAIVNGQVQLRELE-LRGINELSSVAREMVRLIETATVPIFAVDTDGC |
| | | ** * *** * ** ** ******************** ** |
| Seq30 | 676 | INGWNAKIAELTGLSVEEAMGKSLVSDLIYKENEATVNKLLSRALRGDEEKNVEVKLKTF |
| Seq32 | 675 | INGWNAKIAELTGLSVEEAMGKSLVNDLIFKESEATVEKLLSRALRGEEDKNVEIKLKTF |
| | | ************************* *** ** **** ********* * **** ***** |
| Seq30 | 736 | SPELQGKAVFVVVNACSSKDYLNNIVGVCFVGQDVTSQKIVMDKFINIQGDYKAIVHSPN |
| Seq32 | 735 | GSEQSKGPIFVVVNACSSRDYTQNIVGVCFVGQDVTGQKVVMDKFVNIQGDYKAIVHNPN |
| | | * ********* ** ************* ** ***** *********** ** |
| Seq30 | 796 | PLIPPIFAADENTCCLEWNMAMEKLTGWSRSEVIGKMIVGEVFGSCCMLKGPDALTKFMI |
| Seq32 | 795 | PLIPPIFASDENTSCSEWNTAMEKLTGWSRGEVVGKFLIGEVFGNCCRLKGPDALTKFMV |
| | | ******** **** * *** ********** ** ** ***** ** *********** |
| Seq30 | 856 | VLHNAIGGQDTDKFPFPFFDRNGKFVQALLTANKRVSLEGKVIGAFCFLQIPSPELQQAL |
| Seq32 | 855 | IIHNAIGGQDYEKFPFSFFDKNGKYVQALLTANTRSKMDGKSIGAFCFLQIASTEIQQAF |
| | | ******** **** *** *** ******** * ** ********* * * *** |
| Seq30 | 916 | AVQRRQDTECFTKAKELAYICQVIKNPLSGMRFANSLLEATDLNEDQKQLLETSVSCEKQ |
| Seq32 | 915 | EIQRQQEKKCYARMKELAYICQEIKNPLSGIRFTNSLLQMTDLNDDQRQFLETSSACEKQ |
| | | ** * * ******** ******* ** **** **** ** * **** **** |
| Seq30 | 976 | ISRIVGDMDLESIEDGSFVLKREEFFLGSVINAIVSQAMFLLRDRGLQLIRDIPEEIKSI |
| Seq32 | 975 | MSKIVKDASLQSIEDGSLVLEQSEFSLGDVMNAVVSQAMLLLRERDLQLIRDIPDEIKDA |
| | | * ** * * ****** ** ** ** * ** ***** *** * ******** *** |
| Seq30 | 1036 | EVFGDQIRIQQLLAEFLLSIIRYAPSQE-WVEIHLSQLSKQMADGFAAIRTEFRMACPGE |
| Seq32 | 1035 | SAYGDQCRIQQVLADFLLSMVRSAPSENGWVEIQVRPNVKQNSDGTNTELFIFRFACPGE |
| | | *** **** ** **** * *** **** ** ** ** ***** |
| Seq30 | 1095 | GLPPELVRDMFHSSRWTSPEGLGLSVCRKILKLMNGEVQIRESERSYFLIILELPVPR |
| Seq32 | 1095 | GLPADVVQDMFSNSWSTQEGVGLSTCRKILKLMGGEVQYIRESERSFFLIVLEQPQPR |
| | | *** * *** * * ** *** ******** ************ *** ** *** |

This PHYB-related Zea mays protein referred to as LOC100383702 (NCBI accession no. NP_001169810.1 (GI:293335473) has the following sequence (SEQ ID NO:32).

| | |
|---|---|
| 1 | MASGSRATPT RSPSSARPEA PRHAHHHHHS QSSGGSTSRA |
| 41 | GGGAAATESV SKAVAQYTLD ARLHAVFEQS GASGRSFDYS |
| 81 | QSLRAPPTPS SEQQIAAYLS RIQRGGHIQP FGCTLAVADD |
| 121 | SSFRLLAFSE NSPDLLDLSP HHSVPSLDSS APPHVSLGAD |
| 161 | ARLLFSPSSA VLLERAFAAR EISLLNPIWI HSRVSSKPFY |
| 201 | AILHRIDVGV VIDLEPARTE DPALSIAGAV QSQKLAVRAI |
| 241 | SRLQALPGGD VKLLCDTVVE HVRELTGYDR VMVYRFHEDE |
| 281 | HGEVVAESRR DNLEPYLGLH YPATDIPQAS RFLFRQNRVR |
| 321 | MIADCHATPV RVIQDPGLSQ PLCLVGSTLR APHGCHAQYM |
| 361 | ANMGSIASLV MAVIISSGGD DEQTGRGGIS SAMKLWGLVV |
| 401 | CHHTSPRCIP FPLRYACEFL MQAFGLQLNM ELQLAHQLSE |
| 441 | KHILRTQTLL CDMLLRDSPT GIVTQSPSIM DLVKCDGAAL |
| 481 | YYHGKYYPLG VTPTESQIKD IIEWLTVFHG DSTGLSTDSL |
| 521 | ADAGYLGAAA LGEAVCGMAV AYITPSDYLF WFRSHTAKEI |
| 561 | KWGGAKHHPE DKDDGQRMHP RSSFKAFLEV VKSRSLPWEN |
| 601 | AEMDAIHSLQ LILRDSFRDA AEGTNNSKAI VNGQVQLREL |
| 641 | ELRGINELSS VAREMVRLIE TATVPIFAVD TDGCINGWNA |
| 681 | KIAELTGLSV EEAMGKSLVN DLIFKESEAT VEKLLSRALR |
| 721 | GEEDKNVEIK LKTFGSEQSK GPIFVVVNAC SSRDYTQNIV |
| 761 | GVCFVGQDVT GQKVVMDKFV NIQGDYKAIV HNPNPLIPPI |
| 801 | FASDENTSCS EWNTAMEKLT GWSRGEVVGK FLIGEVFGNC |
| 841 | CRLKGPDALT KFMVIIHNAI GGQDYEKFPF SFFDKNGKYV |
| 881 | QALLTANTRS KMDGKSIGAF CFLQIASTEI QQAFEIQRQQ |
| 921 | EKKCYARMKE LAYICQEIKN PLSGIRFTNS LLQMTDLNDD |
| 961 | QRQFLETSSA CEKQMSKIVK DASLQSIEDG SLVLEQSEFS |
| 1001 | LGDVMNAVVS QAMLLLRERD LQLIRDIPDE IKDASAYGDQ |
| 1041 | CRIQQVLADF LLSMVRSAPS ENGWVEIQVR PNVKQNSDGT |
| 1081 | NTELFIFRFA CPGEGLPADV VQDMFSNSQW STQEGVGLST |
| 1121 | CRKILKLMGG EVQYIRESER SFFLIVLEQP QPRPAAGREI |
| 1161 | V |

A codon encoding the arginine at position 320 of the SEQ ID NO:32 protein is equivalent to the codon encoding the arginine at position 322 of the SEQ ID NO:30 protein; a mutant phyB with a sequence encoding the first 319 amino acids of SEQ ID NO:32 can have a C→T substitution, causing the arginine codon (CGA) at position 320 to become a termination codon (TGA).

A cDNA encoding the SEQ ID NO:32 protein is available as NCBI accession number NM_001176339.1 (GI:293335472), and a chromosomal segment encoding the SEQ ID NO:32 protein is on Zea mays chromosome 1 at NC_024459.1 (50023180 . . . 50034310), sequence available as NCBI accession number NC_024459.1 (GI:662250330).

A Zea mays protein referred to as phytochromeB1 (NCBI accession no. DAA45039.1 (GI:414866482) has significant sequence identity to the Arabidopsis thaliana PHYB protein with SEQ ID NO:30, as illustrated by the sequence comparison shown below. Domains of sequence homology are identified by asterisks below the sequence comparison.

| 73.1% identity in 1139 residues overlap; Score: 4270.0; Gap frequency: 0.9% | | |
|---|---|---|
| Seq30 | 22 | PSSSHTPNNRRGGEQAQSSGTKSLRPRSNTESMSKAIQQYTVDARLHAVFEQSGESGKSF |
| Seq33 | 18 | PEAPRHAHHHHHSQSSGGSTSRAGGGAAATESVSKAVAQYTLDARLHAVFEQSGASGRSF |
| | | * * *** *** *** ************ ** ** |
| Seq30 | 82 | DYSQSLKTTTYGSSVPEQQITAYLSRIQRGGYIQPFGCMIAV-DESSFRIIGYSENAPEM |
| Seq33 | 78 | DYSQSLRAPPTPSS--EQQIAAYLSRIQRGGHIQPFGCTLAVADDSSFRLLAFSENSPDL |
| | | ****** ** **** ********** ****** * * **** *** |
| Seq30 | 141 | LGIMPQ-SVPTLEK--PEILAMGTDVRSLFTSSSSILLERAFVAREITLLNPVWIHSKNT |
| Seq33 | 136 | LDLSPHHSVPSLDSSAPPHVSLGALARLLFSPSSAVLLERAFAAREISLLNPIWIHSRVS |
| | | * * *** * * * * * ** ** ****** **** **** **** |
| Seq30 | 198 | GKPFYAILHRIDVGVVIDLEPARTEDPALSIAGAVQSQKLAVRAISQLQALPGGDIKLLC |
| Seq33 | 196 | SKPFYAILHRIDVGVVIDLEPARTEDPALSIAGAVQSQKLAVRAISRLQALPGGDVKLLC |
| | | ********************************************* ******** **** |
| Seq30 | 258 | DTVVESVRDLTGYDRVMVYKFHEDEHGEVVAESKRDDLEPYIGLHYPATDIPQASRFLFK |
| Seq33 | 256 | DTVVEHVRELTGYDRVMVYRFHEDEHGEVVAESRRDNLEPYLGLHYPATDIPQASRFLFR |
| | | ***** ** ********** ************* ** **** ***************** |
| Seq30 | 318 | QNRVRMIVDCNATPVLVVQDDRLTQSMCLVGSTLRAPHGCHSQYMANMGSIASLAMAVII |
| Seq33 | 316 | QNPVRMIADCHATPVRVIQDPGLSQPLCLVGSTLRAPHGCHAQYMANMGSIASLVMAVII |
| | | ******* ** **** * ** * * ************** ************ ***** |
| Seq30 | 378 | NGNEDDGSNVASGRSS-MRLWGLVVCHHTSSRCIPFPLRYACEFLMQAFGLQLNMELQLA |
| Seq33 | 376 | SSGGDDEQTGRGGISSAMKLWGLVVCHHTSPRCIPFPLPYACEFLMQAFGLQLNMELQLA |
| | | ** * ** * *********** ***************************** |
| Seq30 | 437 | LQMSEKRVLRTQTLLCDMLLRDSPAGIVTQSPSIMDLVKCDGAAFLYHGKYYPLGVAPSE |
| Seq33 | 436 | HQLSEKHILRTQTLLCDMLLRDSPTGIVTQSPSIMDLVKCDGAALYYHGKYYPLGVTPTE |
| | | * *** **************** ******************* ********** ** |
| Seq30 | 497 | VQIKDVVEWLLANHADSTGLSTDSLGDAGYPGAAALGDAVCGMAVAYITKRDFLFWFRSH |
| Seq33 | 496 | SQIKDIIEWLTVFHGDSTGLSTDSLADAGYLGAAALGEAVCGMAVAYITPSDYIFWFRSH |
| | | **** *** * ********** **** ****** *********** * ******* |
| Seq30 | 557 | TAKEIKWGGAKHHPEDKDDGQRMHPRSSFQAFLEVVKSRSQPWETAEMDAIHSLQLILRD |
| Seq33 | 556 | TAKEIKWGGAKHHPEDKDDGQRMHPRSSFKAFLEVVKSRSLPWENAEMDAIHSLQLILRD |
| | | ***************************** ********** *** *************** |
| Seq30 | 617 | SFKES-EAAMNSKVVDGVVQPCRDMAGEQGIDELGAVAREMVRLIETATVPIFAVDAGGC |
| Seq33 | 616 | SFRDAAEGTNNSKAIVNGQVQLRELE-LRGINELSSVAREMVRLIETATVPIFAVDTDGC |
| | | ** * *** * ** ** ******************** ** |
| Seq30 | 676 | INGWNAKIAELTGLSVEEAMGKSLVSDLIYKENEATVNKLLSRALRGDEEKNVEVKLKTF |
| Seq33 | 675 | INGWNAKIAELTGLSVEEAMGKSLVNDLIFKESEATVEKLLSRALRGEEDKNVEIKLKTF |
| | | ************************* *** ** **** ********* * **** ***** |
| Seq30 | 736 | SPELQGKAVFVVVNACSSKDYLNNIVGVCFVDQDVTSQKIVMDKFINIQGDYKAIVHSPN |
| Seq33 | 735 | GSEQYKGPIFVVVNACSSRDYTQNIVGVCFVGQDVTGQKVVMDKFVNIQGDYKAIVHNPN |
| | | * ********* ** ************* ** ***** *********** ** |
| Seq30 | 796 | PLIPPIFAADENTCCLEWNMAMEKLTGWSRSEVIGKMIVGEVFGSCCMLKGPDALTKFMI |
| Seq33 | 795 | PLIPPIFASDENTSCSEWNTAMEKLTGWSRGEVVGKFLIGEVFGNCCRLKGPDALTKFMV |
| | | ******** **** * *** ********** ** ** ***** ** *********** |
| Seq30 | 856 | VLHNAIGGQDTDKFPFPFFDRNGKFVQALLTANKRVSLEGKVIGAFCFLQIPSPELQQAL |
| Seq33 | 855 | IIHNAIGGQDYEKFPFSFFDKNGKYVQALLTANTRSKMDGKSIGAFCFLQTASTEIQQAF |
| | | ******** **** *** *** ******** * ** ********* * * *** |
| Seq30 | 916 | AVQRRQDTECFTKAKELAYICQVIKNPLSGMRFANSLLEATDLNEDQKQLLETSVSCEKQ |
| Seq33 | 915 | EIQRQQEKKCYARMKELAYICQEIKNPLSGIRFTNSLLQMTDLNDDQRQFLETSSACEKQ |
| | | ** * * ******** ******* ** **** **** ** * **** **** |
| Seq30 | 976 | ISRIVGDMDLESIEDGSFVLKREEFFLGSVINAIVSQAMFLLPDRGLQLIRDIPEEIKSI |
| Seq33 | 975 | MSKIVKDASLQSIEDGSLVLEQSEFSLGDVMNAVVSQAMLLLRERDLQLIRDIPDEIKDA |
| | | * ** * * ****** ** ** ** * ** ***** *** * ******** *** |
| Seq30 | 1036 | EVFGDQIRIQQLLAEFLLSIIRYAPSQE-WVEIHLSQLSKQMADGFAAIRTEFRMACPGE |
| Seq33 | 1035 | SAYGDQCRIQQVIADFLLSMVRSAPSENGWVEIQVRPNVYQNSDGTNTELFIFRFACPGE |
| | | *** **** ** **** * *** **** ** ** ** ***** |
| Seq30 | 1095 | GLPPELVRDMFHSSRWTSPEGLGLSVCRKILKLMNGEVQYIRESERSYFLIILELPVPR |
| Seq33 | 1095 | GLPADVVQDMFSNSQWSTQEGVGLSTCRKILKLMGGEVQYIRESERSFFLIVLEQPQPR |
| | | *** * *** * * ** *** ******** ************ *** ** * ** |

This PHYB-related Zea mays protein referred to as phytochromeB1 (NCBI accession no. DAA45039.1 (GI:414866482) has the following sequence (SEQ ID NO:33).

| | |
|---|---|
| 1 | MASGSRATPT RSPSSARPEA PRHAHHHHHS QSSGGSTSRA |
| 41 | GGGAAATESV SKAVAQYTLD ARLHAVFEQS GASGRSFDYS |
| 81 | QSLRAPPTPS SEQQIAAYLS RIQRGGHIQP FGCTLAVADD |
| 121 | SSFRLLAFSE NSPDLLDLSP HESVPSLDSS APPHVSLGAD |
| 161 | ARLLFSPSSA VLLERAFAAR EISLLNPIWI HSRVSSKPFY |
| 201 | AILHRIDVGV VIDLEPARTE DPALSIAGAV QSQKLAVRAI |
| 241 | SRLQALPGGD VKLLCDTVVE AVRELTGYDR VMVYRFHEDE |
| 281 | HGEVVAESRR DNLEPYLGLH YPATDIPQAS RFLFRQNRVR |
| 321 | MIADCHATPV RVIQDPGLSQ PLCLVGSTLR APHGCHAQYM |
| 361 | ANMGSIASLV MAVIISSGGD DEQTGRGGIS SAMKLWGLVV |
| 401 | CHHTSPRCIP FPLRYACEFL MQAFGLQLNM ELQLAHQLSE |
| 441 | KHILRTQTLL CDMLLRDSPT GIVTQSPSIM DLVKCDGAAL |
| 481 | YYHGKYYPLG VTPTESQIKD IIEWLTVFHG DSTGLSTDSL |
| 521 | ADAGYLGAAA LGEAVCGMAV AYITPSDYLF WFRSHTAKEI |
| 561 | KWGGAKHHPE DKDDGQRMHP RSSFKAFLEV VKSRSLPWEN |
| 601 | AEMDAIHSLQ LILRDSFRDA AEGTNNSKAI VNGQVQLREL |
| 641 | ELRGINELSS VAREMVRLIE TATVPIFAVD TDGCINGWNA |
| 681 | KIAELTGLSV EEAMGKSLVN DLIFKESEAT VEKLLSRALR |
| 721 | GEEDKNVEIK LKTFGSEQYK GPIFVVVNAC SSRDYTQNIV |
| 761 | GVCFVGQDVT GQKVVMDKFV NIQGDYKALV HNPNPLIPPI |
| 801 | FASDENTSCS EWNTAMEKLT GWSRGEVVGK FLIGEVFGNC |
| 841 | CRLKGPDALT KFMVIIHNAI GGQDYEKFPF SFFDKNGKYV |
| 881 | QALLTANTRS KMDGKSIGAF CFLQIASTEI QQAFEIQRQQ |
| 921 | EKKCYARMKE LAYICQEIKN PLSGIRFTNS LLQMTDLNDD |
| 961 | QRQFLETSSA CERQMSKIVK DASLQSIEDG SLVLEQSEFS |
| 1001 | LGDVMNAVVS QAMLLLRERD LQLIRDIPDE IKDASAYGDQ |
| 1041 | CRIQQVLADF LLSMVRSAPS ENGWVEIQVR PNVKQNSDGT |
| 1081 | NTELFIFRFA CPGEGLPADV VQDMFSNSQW STQEGVGLST |
| 1121 | CRKILKLMGG EVQYIRESER SFFLIVLEQP QPRPAAGREI |
| 1161 | V |

A codon encoding the arginine at position 320 of the SEQ ID NO:33 protein is equivalent to the codon encoding the arginine at position 322 of the SEQ ID NO:30 protein. A codon encoding the arginine at position 320 of the SEQ ID NO:33 protein can be changed to a termination codon. Hence, the PHYB-related Zea mays protein referred to as phytochromeB1 (NCBI accession no. DAA45039.1 (GI:414866482) with sequence SEQ ID NO:33) can be inactivated in a manner similar to the PhyB loci described above.

A chromosomal segment encoding the SEQ ID NO:33 protein is on Zea mays chromosome 1 at NC_024459.1 (50023180 . . . 50034310), sequence available as NCBI accession number NC_024459.1 (GI:662250330).

A Zea mays protein referred to as phytochromeB2 (NCBI accession no. NP_001168077.1 (GI:293336623) has significant sequence identity to the Arabidopsis thaliana PHYB protein with SEQ ID NO:30, as illustrated by the sequence comparison shown below. Domains of sequence homology are identified with asterisks below the sequence comparison.

| 72.3% identity in 1135 residues overlap; Score: 4203.0; Gap frequency: 0.9% | | |
|---|---|---|
| Seq30 | 26 | HTPNNRRGGEQAQSSGTKSLRPRSNTESMSKAIQQYTVDARLHAVFEQSGESGKSFDYSQ |
| Seq34 | 24 | HHHSQSSGGSTSRAGAGGGGGGAAATESVSKAVAQYNLDARLHAVFEQSGASGRSFDYSQ |
| | | * ** *** *** ** ************ ** ****** |
| Seq30 | 86 | SLKTTTYGSSVPEQQITAYLSRIQRGGYIQPFGCMIAV-DESSFRIIGYSENAREMLGIM |
| Seq34 | 84 | SLRAPPTPSS--EQQIAAYLSRIQRGGHIQPLGCTLAVADDSSFRLLAFSENAADLLDLS |
| | | ** ** **** ********** *** ** ** * **** **** * |
| Seq30 | 145 | PQ-SVPTLEKPEI--LAMGTDVRSLFTSSSSILLERAFVAREITLLNPVWIHSKNTGKPF |
| Seq34 | 142 | PHHSVPSLDSVALPPVSLGADARLYFSPSSAVLLERAFAAREISLLNPLWIHSRASSKPF |
| | | * *** * * * * * ** ****** **** **** **** *** |
| Seq30 | 202 | YAILHRIDVGVVIDLEPARTEDPALSIAGAVQSQKLAVRAISQLQALPGGDIKLLCDTVV |
| Seq34 | 202 | YAILHRIDVGVVIDLEPARTEDPALSIAGAVQSQKLAVPAISRLQALPGGDVKLLCDTVV |
| | | ****************************************** ******** ******** |
| Seq30 | 262 | ESVRDLTGYDRVMVYKFHEDEHGEVVAESKRDDLEPYIGLHYPATDIPQASRFLFKQNRV |
| Seq34 | 262 | EHVRELTGYDRVMVYKFHEDEHGEVVAESRRDNLEPYLGLHYPATDIPQASRFLFQQNRV |
| | | * ** ************************ ** **** ***************** **** |
| Seq30 | 322 | RMIVDCNATPVLVVQDDRLTQSMCLVGSTLRAPHGCHSQYMANMGSIASLAMAVIIN-GN |
| Seq34 | 322 | RMIADCHAIPVRVIQDPGLSQQLCLVGSTLRAPHGCHAQYMANMGSIASLVMAVIISSGG |
| | | *** ** * ** * ** * * ************** ************ ***** * |
| Seq30 | 381 | EDDGSNVASGRSSMRLWGLVVCHHTSSRCIPFPLRYACEFLMQAFGLQLNMELQLALQMS |
| Seq34 | 382 | DDERTGRGAISSSMKLWGLVVCHHTSPRCIPFPLRYACEFLMQAFGLQLNMELQLAHQLS |
| | | * *** *********** ***************************** ** |
| Seq30 | 441 | EKRVLRTQTLLCDMLLRDSPAGIVTQSPSIMDLVKCDGAAFLYHGKYYPLGVAPSEVQIK |
| Seq34 | 442 | EKHILRTQTLLCDMLLRDSPAGIITQSPSVMDLVKCDGAALYYRGKYYPLGVTPTESQIK |
| | | ** ******************* ***** ********** * ******** * * *** |
| Seq30 | 501 | DVVEWLLANHADSTGLSTDSLGDAGYPGAAALGDAVCGMAVAYITKRDELFWFRSHTAKE |
| Seq34 | 502 | DIIEWLTVCHGDSTGLSTDSLADAGYLGAVALGDAVCGMAVAYITPSDYLFWFRSHTAKE |
| | | * *** * ********** **** ** *************** * *********** |
| Seq30 | 561 | IKWGGAKHHPEDKDDGQRMHPRSSFQAFLEVVKSRSQPWETAEMDAIHSLQLILRDSFKE |
| Seq34 | 562 | IKWGGAKHHPEDKDDGQRMHPRSSFKAFLEVVKSRSLSWENAEMDAIHSLQLILRDSFRD |
| | | ************************* ********** ** ***************** |
| Seq30 | 621 | S-EAAMNSKVVDGVVQPCRDMAGEQGIDELGAVAREMVRLIETATVPIFAVDAGGCINGW |
| Seq34 | 622 | AAEGTSNSKAIVNGQRQLGELE-LRGINELSSVAREMVRLIETATVPIFAVDTDGCINGW |
| | | * *** ** ** ******************** ****** |
| Seq30 | 680 | NAKIAELTGLSVEEAMGKSLVSDLIYKENEATVNKLLSRALRGDEEKNVEVKLKTFSPEL |
| Seq34 | 681 | NAKIAELTGLSVEEAMGKSLVNDLIFKECDDIVEKLLSRALRGEEDKNVEIKLKTFGSEQ |
| | | ******************** *** ** ********* * **** ***** * |
| Seq30 | 740 | QGKAVFVVVNACSSKDYLNNIVGVCFVGQDVTSQKIVMDKFINIQGDYKAIVHSPNPLIP |
| Seq34 | 741 | SKGAIFVIVNACSSRDYTQNIVGVCFVGQDVTGQKVVMDKFINIQGDYKAIVHNPNPLLP |
| | | * ** ****** ** ************* ** ***************** ***** |
| Seq30 | 800 | PIFAADENTCCLEWNMAMEKLTGWSRSEVIGKMIVGEVFGSCCMLKGPDALTKFMIVLHN |
| Seq34 | 801 | PIFASDENTSCSEWNTAMEKLTGWSREEVVGKFLIGEVFGNCCRLKGPDALTKFMVVIHN |
| | | **** **** * *** ********** ** ** ***** ** *********** * ** |
| Seq30 | 860 | AIGGQDTDKFPFPFFDRNGKFVQALLTANKRVSLEGKVIGAFCFLQIPSPELQQALAVQR |
| Seq34 | 861 | AIEGHDSEKFPFSFFDKNGKYVQALLTANTRSKMDGKSIGAFCFLQIASAEIQQAFEIQR |
| | | ** * * **** *** *** ******** * ** ********* * * *** ** |
| Seq30 | 920 | RQDTECFTKAKELAYICQVIKNPLSGMRFANSLLEATDLNEDQKQLLETSVSCEKQISRI |
| Seq34 | 921 | QQEKKCYARMKELAYICQEIKNPLSGIRFTNSLLQMTDLNDDQRQFLETSSACEKQMSKI |
| | | * * ******** ******* ** **** **** ** * **** **** ** |
| Seq30 | 980 | VGDMDLESIEDGSFVLKREEFFLGSVINAIVSQAMFLLRDRGLQLIRDIPEEIKSIEVFG |
| Seq34 | 981 | VKDASLKSIEDGSLVLEKSEFSLGDVMNAVVSQTMSLLRERDLQLIRDIPDEIKDASAYG |
| | | * * * ****** ** ** ** * ** *** * *** * ******** **** |
| Seq30 | 1040 | DQIRIQQLLAEFLLSIIPYAPSQE-WVEIHLSQLSKQMADGFAAIRTEFRMACPGEGLPP |
| Seq34 | 1041 | DQFRIQQVLADFLLSMAQSAPSENGWVEIQVRPNVKQNYDGTDTELFIFRFACPGEGLPA |
| | | ** **** ** **** *** **** ** ** ** ******** |
| Seq30 | 1099 | ELVRDMFHSSRWTSPEGLGLSVCRKILKLMNGEVQYIRESERSYFLIILELPVPR |
| Seq34 | 1101 | DIVQDMFSNSQWSTQEGVGLSTCRKILKLMGGEVQYIRESERSFFLIVLELPQPR |
| | | * *** * * ** *** ******** ************ *** **** ** |

This PHYB-related Zea mays protein referred to as phytochromeB2 (NCBI accession no. NP_001168077.1 (GI:293336623) has the following sequence (SEQ ID NO:34).

| | |
|---|---|
| 1 | MASDSRPPKR SPSARRVAPR AAEEHHSQSS GGSTSRAGAG |
| 41 | GGGGGAAATE SVSKAVAQYN LDARLHAVFE QSGASGRSFD |
| 81 | YSQSLRAPPT PSSEQQIAAY LSRIQRGGHI QPLGCTLAVA |
| 121 | DDSSFRLLAF SENAADLLDL SPHHSVPSLD SVALPPVSLG |
| 161 | ADARLYFSPS SAVLLERAFA AREISLLNPL WIHSRASSKP |
| 201 | FYAILHRIDV GVVIDLEPAR TEDPALSIAG AVQSQKLAVR |
| 241 | AISRLQALPG GDVKLLCDTV VEHVRELTGY DRVMVYKFHE |
| 281 | DEHGEVVAES RRDNLEPYLG LHYPATDIPQ ASRFLFQQNR |
| 321 | VRMIADCHAI PVRVIQDPGL SQQLCLVGST LRAPHGCHAQ |
| 361 | YMANMGSIAS LVMAVIISSG GDDERTGRGA ISSSMKLWGL |
| 401 | VVCHHTSPRC IPFPLRYACE FLMQAFGLQL NMELQLAHQL |
| 441 | SEKHILRTQT LLCDMLLRDS PAGIITQSPS VMDLVKCDGA |
| 481 | ALYYRGKYYP LGVTPTESQI KDIIEWLTVC HGDSTGLSTD |
| 521 | SLADAGYLGA VALGDAVCGM AVAYITPSDY LFWFRSHTAK |
| 561 | EIKWGGAKHE PEDKDDGQRM HPRSSFKAFL EVVKSRSLSW |
| 601 | ENAEMDAIHS LQLILRDSFR DAAEGTSNSK AIVNGQRQLG |
| 641 | ELELRGINEL SSVAREMVRL IETATVPIFA VDTDGCINGW |
| 681 | NAKIAELTGL SVEEAMGKSL VNDLIFKECD DIVEKLLSRA |
| 721 | LRGEEDKNVE IKLKTFGSEQ SKGAIFVIVN ACSSRDYTQN |
| 761 | IVGVCFVGQD VTGQKVVMDK FINIQGDYKA IVHNPNPLLP |
| 801 | PIFASDENTS CSEWNTAMEK LTGWSREEVV GKFLIGEVFG |
| 841 | NCCRLKGPDA LTKFMVVIHN AIEGHDSEKF PFSFFDKNGK |
| 881 | YVQALLTANT RSKMDGKSIG AFCFLQIASA EIQQAFEIQR |
| 921 | QQEKKCYARM KELAYICQEI KNPLSGIRFT NSLLQMTDLN |
| 961 | DDQRQFLETS SACEKQMSKI VKDASLKSIE DGSLVLEKSE |
| 1001 | FSLGDVMNAV VSQTMSLLRE RDLQLIRDIP DEIKDASAYG |
| 1041 | DQFRIQQVLA DFLLSMAQSA PSENGWVEIQ VRPNVKQNYD |
| 1081 | GTDTELFIFR FACPGEGLPA DIVQDMFSNS QWSTQEGVGL |
| 1121 | STCRKILKLM GGEVQYIRES ERSFFLIVLE LPQPRLAAGR |
| 1161 | ENQLIC |

A codon encoding the arginine at position 322 of the SEQ ID NO:34 protein is equivalent to the codon encoding the arginine at position 322 of the SEQ ID NO:30 protein. A codon encoding the arginine at position 322 of the SEQ ID NO:34 protein can be changed to a termination codon. Hence, the PHYB-related Zea mays protein referred to as phytochromeB2 (NCBI accession no. NP_001168077.1 (GI:293336623) with sequence SEQ ID NO:34) can be inactivated in a manner similar to the PhyB loci described above.

A cDNA encoding the SEQ ID NO:34 protein is available as NCBI accession number NM_001174606.1 (GI:293336622), and a chromosomal segment encoding the SEQ ID NO:32 protein is on Zea mays chromosome 9 at NC_024467.1 (135245613 . . . 135251739, complement), sequence available as NCBI accession number NC_024467.1 (GI:662248440).

A Glycine max protein referred to as phytochrome B (NCBI accession no. NP_001240097 XP_003533157; NP_001240097.1 (GI:358248221)) has significant sequence identity to the Arabidopsis thaliana PHYB protein with SEQ ID NO:30, as illustrated by the sequence comparison shown below. Domains of sequence homology are identified with asterisks below the sequence comparison.

| 77.4% identity in 1109 residues overlap; Score: 4478.0; Gap frequency: 1.1% | | |
|---|---|---|
| Seq30 | 49 | SNTESMSKAIQQYTVDARLHAVFEQSGESGKSFDYSQSLKTTTYGSSVPEQQITAYLSRI |
| Seq35 | 33 | NNIDSMSKAIAQYTEDARLHAVFEQSGESGRSFNYSESIRIAS--ESVPEQQITAYLVKI |
| | | * ****** *** *************** ** ** * ************ |
| Seq30 | 109 | QRGGYIQPFGCMIAVDESSFRIIGYSENAREMLGIMPQSVPTLEKPE--ILAMGTDVRSL |
| Seq35 | 91 | QRGGFIQPFGSMIAVDEPSFRILGYSDNARDMLGITPQSVPSLDDKNDAAFALGTDVRAL |
| | | **** ***** ****** **** *** *** **** ***** * * ***** * |
| Seq30 | 167 | FTSSSSILLERAFVAREITLLNPVWIHSKNTGKPFYAILHRIDVGVVIDLEPARTEDPAL |
| Seq35 | 151 | FTHSSALLLEKAFSAREISLMNPIWIHSRTSGKPFYGILHRIDVGIVIDLEPARTEDPAL |
| | | ** ** *** ** **** * ** **** ***** ******** ************** |
| Seq30 | 227 | SIAGAVQSQKLAVRAISQLQALPGGDIKLLCDTVVESVRDLTGYDRVMVYKFHEDEHGEV |
| Seq35 | 211 | SIAGAVQSQKLAVRAISQLQSLPGGDVKLLCDTVVESVRELTGYDRVMVYKFHEDEHGEV |
| | | ******************** ***** ************ ******************** |
| Seq30 | 287 | VAESKRDDLEPYIGLHYPATDIPQASRFLFKQNRVRMIVDCNATPVLVVQDDRLTQSMCL |
| Seq35 | 271 | VSESKRPDLEPYIGLHYPATDIRQASRELFKQNRVRMIVDCHASAVRVVQDEALVQPLCL |
| | | * **** *********************************** * * **** * * ** |
| Seq30 | 347 | VGSTLRAPHGCHSQYMANMGSIASLAMAVIINGNEDDGSNVASGRSSMRLWGLVVCHHTS |
| Seq35 | 331 | VGSTLRAPHGCHAQYMANMGSIASLVMAVIINGNDEEG---VGGRSSMRLWGLVVCHHTS |
| | | ************ ************ ******** * ***************** |
| Seq30 | 407 | SRCIPFPLRYACEFLMQAFGLQLNMELQLALQMSEKRVLRTQTLLCDMLLRDSPAGIVTQ |
| Seq35 | 388 | ARCIPFPLRYACEFLMQAFGLQLNMELQLAAQSLEKRVLRTQTLLCDMLLRDSPTGIVTQ |
| | | ***************************** * ******************** ***** |
| Seq30 | 467 | SPSIMDLVKCDGAAFLYHGKYYPLGVAPSEVQIKDVVEWLLANHADSTGLSTDSLGDAGY |
| Seq35 | 448 | SPSIMDLVKCDGAALYFQGNYYPLGVTPTEAQIRDIIEWLLAFHGDSTGLSTDSLGDAGY |
| | | ************** * ****** * * ** * ***** * *************** |
| Seq30 | 527 | PGAAALGDAVCGMAVAYITKRDFLFWFRSHTAKEIKWGGAKHHPEDKDDGQRMHPRSSFQ |
| Seq35 | 508 | PGAASLGDAVCGMAVAYITEKDFLFWFRSHTAKEIKWGGAKHHPEDKDDGQRMHPRSSFK |
| | | **** ************** ************************************** |
| Seq30 | 587 | AFLEVVKSRSQPWETAEMDAIHSLQLILRDSFKESEAAMNSKVVDGVVQPCRDMAGEQGI |
| Seq35 | 568 | AFLEVVKSRSLPWENAEMDAIHSLQLILRDSFKDAEHRNSKAVVD----PHVSEQELQGV |
| | | ********** *** ****************** * *** * ** |
| Seq30 | 647 | DELGAVAREMVRLIETATVPIFAVDAGGCINGWNAKIAELTGLSVEEAMGKSLVSDLIYK |
| Seq35 | 624 | DELSSVAREMVRLIETATAPIFAVDVDGHVNGWNAKVSELTGLPVEEAMGKSLVHDLVFK |
| | | *** ************* ****** * ****** ***** ********** ** * |
| Seq30 | 707 | ENEATVNKLLSRALRGDEEKNVEVKLKTFSPELQGKAVFVVVNACSSKDYLNNIVGVCFV |
| Seq35 | 684 | ESEETMNKLLSRALKGEEDKNVEIKMRTFGPEHQNKAVFLVVNACSSKDFTNNVVGVCFV |
| | | * * * ******** * * **** * ** ** * **** ********* ** ****** |
| Seq30 | 767 | GQDVTSQKIVMDKFINIQGDYKAIVHSPNPLIPPIFAADENTCCLEWNMAMEKLTGWSRS |
| Seq35 | 744 | GQDVTGQKIVMDKFINIQGDYKAIVHSPNPLIPPIFASDDNTCCLEWNTAMEKLTGWGRV |
| | | ***** ******************************* * ******** ******** * |
| Seq30 | 827 | EVIGKMIVGEVFGSCCMLKGPDALTKFMIVLHNAIGGQDTDKFPFPFFDRNGKFVQALLT |
| Seq35 | 804 | DVIGKMLVGEVFGSCCQLKGSDSITKFMIVLHNALGGQDTDKFPFSFLDRHGKYVQTFLT |
| | | ***** ********* *** * ********** ********** * ** ** ** ** |
| Seq30 | 887 | ANKRVSLEGKVIGAFCFLQIPSPELQQALAVQRRQDTECFTKAKELAYICQVIKNPLSGM |
| Seq35 | 864 | ANKRVNMEGQIIGAFCFLQIMSPELQQALKAQRQQEKNSFGRMKELAYICQGVKNPLSGI |
| | | ***** ** ********* ******** ** * * ******** ****** |
| Seq30 | 947 | RFANSLLEATDLNEDQKQLLETSVSCEKQISRIVGDMDLESIEDGSFVLKREEFFLGSVI |
| Seq35 | 924 | RFTNSLLEATSLTNEQKQFLETSVACEKQMLKIIRDVDLESIEDGSLELEKGEFLLGNVI |
| | | ** ******* * *** ***** **** * * ********* * ** ** ** |
| Seq30 | 1007 | NAIVSQAMFLLRDRGLQLIRDIPEEIKSIEVFGDQIRIQQLLAEFLLSIIRYAPSQE-WV |
| Seq35 | 984 | NAVVSQVMLLLRERNLQLIRDIPEEIKTLAVYGDQLRIQQVLSDFLLNIVRYAPSPDGWV |
| | | ** *** * *** * ************ * *** **** * *** * ***** ** |
| Seq30 | 1066 | EIHLSQLSKQMADGFAAIRTEFRMACPGEGLPPELVRDMFHSSRWTSPEGLGLSVCRKIL |
| Seq35 | 1044 | EIHVRPRIKQISDGLTLLHAEFRMVCPGEGLPPELIQDMFNNSRWGTQEGLGLSMSRKIL |
| | | *** ** ** **** ********** *** *** ****** **** |
| Seq30 | 1126 | KLMNGEVQYIRESERSYFLIILELPVPRK |
| Seq35 | 1104 | KLMNGEVQYIREAERCYFYVLLELPVTRR |
| | | ************ ** ** ***** * |

This PHYB-related Glycine max protein referred to as phytochrome B (NCBI accession no. NP_001240097 XP_003533157; NP_001240097.1 (GI:358248221)) has the following sequence (SEQ ID NO:35).

| | |
|---|---|
| 1 | MASASGAANS SVPPPQIHTS RTKLSHHSSN NNNNIDSMSK |
| 41 | AIAQYTEDAR LHAVFEQSGE SGRSFNYSES IRIASESVPE |
| 81 | QQITAYLVKI QRGGFIQPFG SMIAVDEPSF RILGYSDNAR |
| 121 | DMLGITPQSV PSLDDKNDAA FALGTDVRAL FTHSSALLLE |
| 161 | KAFSAREISL MNPIWIHSRT SGKPFYGILH RIDVGIVIDL |
| 201 | EPARTEDPAL SIAGAVQSQK LAVRAISQLQ SLPGGDVKLL |
| 241 | CDTVVESVRE LTGYDRVMVY KFHEDEHGEV VSESKRPDLE |
| 281 | PYIGLHYPAT DIPQASRFLF KQNRVRMIVD CHASAVRVVQ |
| 321 | DEALVQPLCL VGSTLRAPHG CHAQYMANMG SIASLVMAVI |
| 361 | INGNDEEGVG GRSSMRLWGL VVCHHTSARC IPFPLRYACE |
| 401 | FLMQAFGLQL NMELQLAAQS LEKRVLRTQT LLCDMLLRDS |
| 441 | PTGIVTQSPS IMDLVKCDGA ALYFQGNYYP LGVTPTEAQI |
| 481 | RDIIEWLLAF HGDSTGLSTD SLGDAGYPGA ASLGDAVCGM |
| 521 | AVAYITEKDF LFWFRSHTAK EIKWGGAKHH PEDKDDGQRM |
| 561 | HPRSSFKAFL EVVKSRSLPW ENAEMDAIHS LQLILRDSFK |
| 601 | DAEHRNSKAV VDPHVSEQEL QGVDELSSVA REMVRLIETA |
| 641 | TAPIFAVDVD GHVNGWNAKV SELTGLPVEE AMGKSLVHDL |
| 681 | VFKESEETMN KLLSRALKGE EDKNVEIKMR TFGPEHQNKA |
| 721 | VFLVVNACSS KDFTNNVVGV CFVGQDVTGQ KIVMDKFINI |
| 761 | QGDYKAIVAS PNPLIPPIFA SDDNTCCLEW NTAMEKLTGW |
| 801 | GRVDVIGKML VGEVFGSCCQ LKGSDSITKF MIVLHNALGG |
| 841 | QDTDKFPFSF LDRHGKYVQT FLTANKRVNM EGQIIGAFCF |
| 881 | LQIMSPELQQ ALKAQRQQEK NSFGRMKELA YICQGVKNPL |
| 921 | SGIRFTNSLL EATSLTNEQK QFLETSVACE KQMLKIIRDV |
| 961 | DLESIEDGSL ELEKGEFLLG NVINAVVSQV MLLLRERNLQ |
| 1001 | LIRDIPEEIK TLAVYGDQLR IQQVLSDFLL NIVRYAPSPD |
| 1041 | GWVEIHVRPR IKQISDGLTL LHAEFRMVCP GEGLPPELIQ |
| 1081 | DMFNNSRWGT QEGLGLSMSR KILKLMNGEV QYIREAERCY |
| 1121 | FYYLLELPVT RRSSKKC |

A codon encoding the arginine at position 306 of the SEQ ID NO:35 protein is equivalent to the codon encoding the arginine at position 322 of the SEQ ID NO:30 protein. A codon encoding the arginine at position 306 of the SEQ ID NO:35 protein can be changed to a termination codon. Hence, the PHYB-related Glycine max protein referred to as phytochrome B (NCBI accession no. NP_001240097 XP_003533157; NP_001240097.1 (GI:358248221)) with sequence SEQ ID NO:35) can be inactivated in a manner similar to the PhyB loci described above.

A cDNA encoding the SEQ ID NO:35 protein is available as NCBI accession number NM_001253168.1 (GI:358248220), and a chromosomal segment encoding the SEQ ID NO:35 protein is on Glycine max chromosome 9 at NC_016096.2 (2960478 . . . 2966704, complement), sequence available as NCBI accession number NC_016096.2 (GI:952545307).

Another Glycine max protein referred to as phytochrome B (NCBI accession no. ACJ61499.1 (GI:214011498)) also has significant sequence identity to the Arabidopsis thaliana PHYB protein with SEQ ID NO:30, as illustrated by the sequence comparison shown below. Domains of sequence homology are identified with asterisks below the sequence comparison.

| 77.5% identity in 1104 residues overlap; Score: 4466.0; Gap frequency: 1.1% | | |
|---|---|---|
| Seq30 | 54 | MSKAIQQYTVDARLHAVFEQSGESGKSFDYSQSLKTTTYGSSVPEQQITAYLSRIQRGGY |
| Seq36 | 1 | MSKAIAQYTEDARLHAVFEQSGESGRSFNYSESIRIAS--ESVPEQQITAYLVKIQRGGF |
| | | ***** *** *************** ** ** * *********** ***** |
| Seq30 | 114 | IQPFGCMIAVDESSFRIIGYSENAREMLGIMPQSVPTLEKPE--ILAMGTDVRSLFTSSS |
| Seq36 | 59 | IQPFGSMIAVDEPSFRILGYSDNARDMLGITPQSVPSLDDKNDAAFALGTDVRALFTHSS |
| | | ***** ****** **** *** *** **** ***** * * ***** *** ** |
| Seq30 | 172 | SILLERAFVAREITLLNPVWIHSKNTGKPFYAILHRIDVGVVIDLEPARTEDPALSIAGA |
| Seq36 | 119 | ALLLEKAFSAREISLMNPIWIHSRTSGKPFYGILHPIDVGIVIDLEPAPTEDPALSIAGA |
| | | *** ** **** * ** **** ***** ******** ******************* |
| Seq30 | 232 | VQSQKLAVRAISQLQALPGGDIKLLCDTVVESVRDLTGYDRVMVYKFHEDEHGEVVAESK |
| Seq36 | 179 | VQSQKLAVRAISQLQSLPGGDVKLLCDTVVESVRELTGYDRVMVYKFHEDEHGEVVSESK |
| | | *************** ***** ************ ********************* *** |
| Seq30 | 292 | RDDLEPYIGLHYPATDIPQASRFLFKQNRVRMIVDCNATPVLVVQDDRLTQSMCLVGSTL |
| Seq36 | 239 | RPDLEPYIGLHYPATDIPQASRFLEKQNRVRMIVDCHASAVRVVQDEALVQPLCLVGSTL |
| | | * ********************************** * * **** * * ******* |
| Seq30 | 352 | RAPHGCHSQYMANMGSIASLAMAVIINGNEDDGSNVASGRSSMRLWGLVVCHHTSSRCIP |
| Seq36 | 299 | RAPHGCHAQYMANMGSIASLVMAVIINGNDEEG---VGGRSSMRLWGLVVCHHTSARCIP |
| | | ******* ************ ******** * ***************** **** |
| Seq30 | 412 | FPLRYACEFLMQAFGLQLNMELQLALQMSEKRVLRTQTLLCDMLLRDSPAGIVTQSPSIM |
| Seq36 | 356 | FPLRYACEFLMQAFGLQLNMELQLAAQSLEKRVLRTQTLLCDMLLRDSPTGIVTQSPSIM |
| | | ************************* * ******************** ********** |
| Seq30 | 472 | DLNKCDGAAFLYHGKYYPLGVAPSEVQIKDVVEWLLANHADSTGLSTDSLGDAGYPGAAA |
| Seq36 | 416 | DLNKCDGAALYFQGNYYPLGVTPTEAQIRDIIEWLLAFHGDSTGLSTDSLGDAGYPGAAS |
| | | ********* * ****** * * ** * ***** * ******************* |
| Seq30 | 532 | LGDAVCGMAVAYITKRDFLEWFRSHTAKEIKWGGAKHHPEDKDDGQRMHPRSSFQAFLEV |
| Seq36 | 476 | LGDAVCGMAVAYITEKDFLFWFRSHTAKEIKWGGAKHHPEDKDDGQRMHPRSSFKAFLEV |
| | | ************** ************************************** ***** |
| Seq30 | 592 | VKSRSQPWETAEMDAIHSLQLILRDSFKESEAAMNSKVVDGVVQPCRDMAGEQGIDELGA |
| Seq36 | 536 | VKSRSLPWENAEMDAIHSLQLILRDSFKDAEHRNSKAVVD----PHVSEQELQGVDELSS |
| | | ***** *** ****************** * *** * ** *** |
| Seq30 | 652 | VAREMVRLIETATVPIFAVDAGGCINGWNAKIAELTGLSVEEAMGKSLVSDLIYKENEAT |
| Seq36 | 592 | VAREMVRLIETATAPIFAVDVDGHVNGWNAKVSELTGLPVEEAMGKSLVHDLVFKESEET |
| | | ************* ****** * ****** ***** ********** ** ** * * |
| Seq30 | 712 | VNKLLSRALRGDEEKNVEVKLKTFSPELQGKAVFVVVNACSSKDYLNNIVGVCFVGQDVT |
| Seq36 | 652 | MNKLLSRALKGEEDKNVEIKMRTFGPEHQNKAVFLVVNACSSKDFTNNVVGVCFVGQDVT |
| | | ******** * * **** * ** ** * **** ********* ** *********** |
| Seq30 | 772 | SQKIVMDKFINIQGDYKAIVHSPNPLIPPIFAADENTCCLEWNMAMEKLTGWSRSEVIGK |
| Seq36 | 712 | GQKIVMDKFINIQGDYKAIVHSPNPLIPPIFASDDNTCCLEWNTAMEKLTGWGRVDVIGK |
| | | ******************************* * ******** ******** * **** |
| Seq30 | 832 | MIVGEVFGSCCMLKGPDALTKFMIVLHNAIGGQDTDKFPFPFFDRNGKFVQALLTANKRV |
| Seq36 | 772 | MLVGEVFGSCCQLKGSDSITKFMIVLHNALGGQDTDKFPFSFLDRHGKYVQTFLTANKRV |
| | | * ********* *** * ********** ********** * ** ** ** ******* |
| Seq30 | 892 | SLEGKVIGAFCFLQIPSPELQQALAVQRRQDTECFTKAKELAYICQVIKNPLSGMRFANS |
| Seq36 | 832 | NMEGQIIGAFCFLQIMSPELQQALKKQRQQEKNSFGRMKELAYICQGVKNPLSGIRFTNS |
| | | ** ********* ******** ** * * ******** ****** ** ** |
| Seq30 | 952 | LLEATDLNEDQKQLLETSVSCEKQISRIVGDMDLESIEDGSFVLKREEFFLGSVINAIVS |
| Seq36 | 892 | LLEATSLTNEQKQFLETSVACEKQMLKIIRDVDLESIEDGSLELEKGEFLLGNVINAVVS |
| | | ***** * *** ***** **** * * ********* * ** ** **** ** |
| Seq30 | 1012 | QAMFLLRDRGLQLIRDIPEEIKSIEVFGDQIRIQQLLAEFLLSIIRYAPSQE-WVEIHLS |
| Seq36 | 952 | QVMLLLRERNLQLIRDIPEEIKTLAVYGDQLRIQQVLSDFLLNIVRYAPSPDGWVEIHVR |
| | | * * *** * ************ * *** **** * *** * ***** ***** |
| Seq30 | 1071 | QLSKQMADGFAAIRTEFRMACPGEGLPPELVRDMFHSSRWTSPEGLGLSVCRKILKLMNG |
| Seq36 | 1012 | PRIKQISDGLTLLHAEFRMVCPGEGLPPELIQDMFNNSRWGTQEGLGLSMSRKILKLMNG |
| | | ** ** **** ********** *** *** ****** ********* |
| Seq30 | 1131 | EVQYIRESERSYFLIILELPVPRK |
| Seq36 | 1072 | EVQYIRRAERCYFYVLLELPVTRR |
| | | ******* ** ** ***** * |

This PHYB-related Glycine max protein referred to as phytochrome B (NCBI accession no. ACJ61499.1 (GI:214011498)) has the following sequence (SEQ ID NO:36).

| | |
|---|---|
| 1 | MSKAIAQYTE DARLEAVFEQ SGESGRSFNY SESIRIASES |
| 41 | VPEQQITAYL VKIQRGGFIQ PFGSMIAVDE PSFRILGYSD |
| 81 | NARDMLGITP QSVPSLDDKN DAAHALGTDV RALFTHSSAL |
| 121 | LLEKAFSARE ISLMNPIWIH SRTSGKPFYG ILHRIDVGIV |
| 161 | IDLEPARTED PALSIAGAVQ SQKLAVRAIS QLQSLPGGDV |
| 201 | KLLCDTVVES VRELTGYDRV MVYKFHEDEH GEVVSESKRP |
| 241 | DLEPYIGLHY PATDIPQASR FLFKQNRVRM IVDCHASAVR |
| 281 | VVQDEALVQP LCLVGSTLRA PHGCHAQYMA NMGSIASLVM |
| 321 | AVIINGNDEE GVGGRSSMRL WGLVVCHHTS ARCIPFPLRY |
| 361 | ACEFLMQAFG LQLNMELQLA AQSLEKRVLR TQTLLCDMLL |
| 401 | RDSPTGIVTQ SPSIMDLVKC DGAALYFQGN YYPLGVTPTE |
| 441 | AQIRDIIEWL LAFHGDSTGL STDSLGDAGY PGAASLGDAV |
| 481 | CGMAVAYITE KDFLFWFRSH TAKEIKWGGA KHHPEDKDDG |
| 521 | QRMHPRSSFK AFLEVVKSRS LPWENAEMDA IHSLQLILRD |
| 561 | SFKDAEHRNS KAVVDPHVSE QELQGVDELS SVAREMVRLI |
| 601 | ETATAPIFAV DVDGHVNGWN AKVSELTGLP VEEAMGKSLV |
| 641 | HDLVFKESEE TMNKLLSRAL KGEEDKNVEI KMRTFGPEHQ |
| 681 | NKAVFLVVNA CSSKDFTNNV VGVCFVGQDV TGQKIVMDKF |
| 721 | INIQGDYKAI VHSPNPLIPP IFASDDNTCC LEWNTAMEKL |
| 761 | TGWGRVDVIG KMLVGEVFGS CCQLKGSDSI TKFMIVLHNA |
| 801 | LGGQDTDKFP FSFLDRHGKY VQTFLTANKR VNMEGQIIGA |
| 841 | FCFLQIMSPE LQQALKAQRQ QEKNSFGRMK ELAYICQGVK |
| 881 | NPLSGIRFTN SLLEATSLTN EQKQFLETSV ACEKQMLKII |
| 921 | RDVDLESIED GSLELEKGEF LLGNVINAVV SQVMLLLRER |
| 961 | NLQLIRDIPE EIKTLAVYGD QLRIQQVLSD FLLNIVRYAP |
| 1001 | SPDGWVEIHV RPRIKQISDG LTLLHAEFRM VCPGEGLPPE |
| 1041 | LIUMFMNSR WGTQEGLGLS MSRKILKLMN GEVQYIREAE |
| 1081 | RCYFYVLLEL PVTRRSSKKC |

A codon encoding the arginine at position 269 of the SEQ ID NO:36 protein is equivalent to the codon encoding the arginine at position 322 of the SEQ ID NO:30 protein. A codon encoding the arginine at position 269 of the SEQ ID NO:36 protein can be changed to a termination codon. Hence, the PHYB-related Glycine max protein referred to as phytochrome B (NCBI accession no. ACJ61499.1 (GI:214011498) with sequence SEQ ID NO:36) can be inactivated in a manner similar to the PhyB loci described above.

A chromosomal segment encoding the SEQ ID NO:36 protein is on Glycine max chromosome 9, and also at NC_016096.2 (2960478 . . . 2966704, complement), with a sequence available as NCBI accession number NC_016096.2 (GI:952545307).

An Oryza sativa protein referred to as phytochrome B (NCBI accession no. AFK31004.1 (GI:388458276)) has significant sequence identity to the Arabidopsis thaliana PHYB protein with SEQ ID NO:30, as illustrated by the sequence comparison shown below. Domains of sequence homology are identified with asterisks below the sequence comparison.

| 75.6% identity in 1113 residues overlap; Score: 4309.0; Gap frequency: 1.3% | | |
|---|---|---|
| Seq30 | 52 | ESMSKAIQQYTVDARLHAVFEQSGESGKSFDYSQSLKTTTYGSSVPEQQITAYLSRIQRG |
| Seq37 | 56 | ESVSKAVAQYTLDARLHAVFEQSGASGRSFDYTQSLRASPTPSS--EQQIAAYLSRIQRG |
| | | ** *** *** ************ ** **** *** ** **** ********* |
| Seq30 | 112 | GYIQPFGCMIAV-DESSFRIIGYSENAREMLGIMPQ-SVPTLEK---PEILAMGTDVRSL |
| Seq37 | 114 | GHIQPFGCTLAVADDSSFRLLAYSENTADLLDLSPHHSVPSLDSSAVPPPVSLGADARLL |
| | | * ****** ** * **** **** * * *** * * * * * * |
| Seq30 | 167 | FTSSSSILLERAFVAREITLLNPVWIHSKNTGKPFYAILHRIDVGVVIDLEPARTEDPAL |
| Seq37 | 174 | FAPSSAVLLERAFAAREISLLNPLWIHSRVSSKPFYAILHRIDVGVVIDLEPARTEDPAL |
| | | * ** ****** **** **** **** **************************** |
| Seq30 | 227 | SIAGAVQSQKLAVRAISQLQALPGGDIKLLCDTVVESVRDLTGYDRVMVYKFHEDEHGEV |
| Seq37 | 234 | SIAGAVQSQKLAVRAISRLQALPGGDVKLLCDTVVEHVRELTGYDRVMVYRFHEDEHGEV |
| | | ***************** ******** ********* ** ********** ********* |
| Seq30 | 287 | VAESKRDDLEPYIGLHYPATDIPQASRELFKQNRVRMIVDCNATPVLVVQDDRLTQSMCL |
| Seq37 | 294 | VAESRRNNLEPYIGLHYPATDIPQASRFLFRQNRVRMIADCEAAPVRVIQDPALTQPLCL |
| | | **** * ********************** ******* ** * ** * ** *** ** |
| Seq30 | 347 | VGSTLRAPHGCHSQYMANMGSIASLAMAVIINGNEDDGSNVASGR--SSMRLWGLVVCHH |
| Seq37 | 354 | VGSTLRSPHGCHAQYMANMGSIASLVMAVIISSGGDDDHNIARGSIPSAMKLWGLVVCHH |
| | | ****** ***** ************ ***** ** * * * * * ********* |
| Seq30 | 405 | TSSRCIPFPLRYACEFLMQAFGLQLNMELQLALQMSEKRVLRTQTLLCDMLLRDSPAGIV |
| Seq37 | 414 | TSPRCIPFPLRYACEFLMQAFGLQLNMELQLAHQLSEKHILRTQTLLCDMLLRDSPTGIV |
| | | ** ***************************** * *** **************** *** |
| Seq30 | 465 | TQSPSIMDLVKCDGAAFLYHGRYYPLGVAPSEVQIKDVVEWLLANHADSTGLSTDSLGDA |
| Seq37 | 474 | TQSPSIMDLVKCDGAALYYHGKYYPLGVTPTEVQIKDIIEWLTMCHGDSTGLSTDSLADA |
| | | **************** ********** * ****** *** * ********** ** |
| Seq30 | 525 | GYPGAAALGDAVCGMAVAYITKRDFLFWFRSHTAKEIKWGGAKHHPEDKDDGQRMHPRSS |
| Seq37 | 534 | GYPGAAALGDAVSGMAVAYITPSDYLFWFRSHTAKEIKWGGAKHHPEDKDDGQRMHPRSS |
| | | ************ ******** * *********************************** |
| Seq30 | 585 | FQAFLEVVKSRSQPWETAEMDAIHSLQLILRDSFKES-EAAMNSK-VVDGVVQPCRDMAG |
| Seq37 | 594 | FKAFLEVVKSRSLPWENAEMDAIHSLQLILRDSFRDSAEGTSNSKAIVNGQVQ--LGELE |
| | | * ********** *** ***************** * * *** * * ** |
| Seq30 | 643 | EQGIDELGAVAREMVRLIETATVPIFAVDAGGCINGWNAKIAELTGLSVEEAMGKSLVSD |
| Seq37 | 652 | LRGIDELSSVAREMVRLIETATVPIFAVDTDGCINGWNAKVAELTGLSVEEAMGKSLVND |
| | | ***** ******************** ********* ***************** * |
| Seq30 | 703 | LIYKENEATVNKLLSRALRGDEEKNVEVKLKTFSPELQGKAVFVVVNACSSKDYLNNIVG |
| Seq37 | 712 | LIFKESEETVNKLLSRALRGDEDKNVEIKLKTFGPEQSKGPIFVIVNACSSRDYTKNIVG |
| | | ** ** * ************** **** ***** ** ** ****** ** **** |
| Seq30 | 763 | VCFVGQDVTSQKIVMDKFINIQGDYKAIVHSPNPLIPPIFAADENTCCLEWNMAMEKLTG |
| Seq37 | 772 | VCFVGQDVTGQKVVMDKFINIQGDYKAIVHNPNPLIPPIFASDENTCCSEWNTAMEKLTG |
| | | ********* ** ***************** ********** ****** *** ******* |
| Seq30 | 823 | WSRSEVIGKMIVGEVFGSCCMLKGPDALTKFMIVLHNAIGGQDTDKFPFPFFDRNGKFVQ |
| Seq37 | 832 | WSRGEVVGKLLVGEVFGNCCRLKGPDALTKFMIVLHNAIGGQDCEKFPFSFFDKNGKYVQ |
| | | *** ** ** ****** ** ********************** **** *** *** ** |
| Seq30 | 883 | ALLTANKRVSLEGKVIGAFCFLQIPSPELQQALAVQRRQDTECFTKAKELAYICQVIKNP |
| Seq37 | 892 | ALLTANTRSRMDGEAIGAFCFLQIASPELQQAFEIQRHHEKKCYARMKELAYIYQEIKNP |
| | | ****** * * ********* ******* ** * ****** * **** |
| Seq30 | 943 | LSGMRFANSLLEATDLNEDQKQLLETSVSCEKQISRIVGDMDLESIEDGSFVLKREEFFL |
| Seq37 | 952 | LNGIRFTNSLLEMTDLKDDQRQFLETSTACEKQMSKIVKDASLQSIEDGSLVLEKGEFSL |
| | | * * ** ***** *** ** * **** **** * ** * * ****** ** ** * |
| Seq30 | 1003 | GSVINAIVSQAMFLLRDRGLQLIRDIPEEIKSIEVFGDQIRIQQLLAEFLLSIIRYAPSQ |
| Seq37 | 1012 | GSVMNAVVSQVMIQLRERDLQLIRDIPDEIKEASAYGDQYRIQQVLCDFLLSMVRFAPAE |
| | | *** ** *** * ** * ******** *** *** **** * **** * ** |
| Seq30 | 1063 | E-WVEIHLSQLSKQMADGFAAIRTEFRMACPGEGLPPELVRDMFHSSRWTSPEGLGLSVC |
| Seq37 | 1072 | NGWVEIQVRPNIKQNSDGTDTMLFLFRFACPGEGLPPEIVQDMFSNSRWTTQEGIGLSIC |
| | | **** ** ** ** ********** * *** **** ** *** * |
| Seq30 | 1122 | RKILKLMNGEVQYIRESERSYFLIILELPVPRK |
| Seq37 | 1132 | RKILKLMGGEVQYIRESERSFFHIVLELPQPQQ |
| | | ******* ************ * * **** * |

This PHYB-related Oryza sativa protein referred to as phytochrome B (NCBI accession no. AFK31004.1 (GI:388458276)) has the following sequence (SEQ ID NO:37).

| | |
|---|---|
| 1 | MASGSRATPT RSPSSARPAA PRAQHHHSQS SGGSTSRAGG |
| 41 | GGGGGGGGGG GAAAAESVSK AVAQYTLDAR LHAVFEQSGA |
| 81 | SGRSFDYTQS LRASPTPSSE QQIAAYLSRI QRGGHIQPFG |
| 121 | CTLAVADDSS FRLLAYSENT ADLLDLSPHH SVPSLDSSAV |
| 161 | PPPVSLGADA RLLFAPSSAV LLERAFAARE ISLLNPLWIH |
| 201 | SRVSSKPFYA ILHRIDVGVV IDLEPARTED PALSIAGAVQ |
| 241 | SQKLAVRAIS RLQALPGGDV KLLCDTVVEH VRELTGYDRV |
| 281 | MVYRFHEDEH GEVVAESRRN NLEPYIGLHY PATDIPQASR |
| 321 | FLFRQNRVRM IADCHAAPVR VIQDPALTQP LCLVGSTLRS |
| 361 | PHGCHAQYMA NMGSIASLVM AVIISSGGDD DHNIARGSIP |
| 401 | SAMKLWGLVV CHHTSPRCIP FPLRYACEFL MQAFGLQLNM |
| 441 | ELQLAHQLSE KHILRTQTLL CDMLLRDSPT GIVTQSPSIM |
| 481 | DLVKCDGAAL YYHGKYYPLG VTPTEVQIKD IIEWLTMCHG |
| 521 | DSTGLSTDSL ADAGYPGAAA LGDAVSGMAV AYITPSDYLF |
| 561 | WFRSHTAKEI KWGGAKHHPE DKDDGQRMHP RSSFKAFLEV |
| 601 | VKSRSLPWEN AEMDAIHSLQ LILRDSFRDS AEGTSNSKAI |
| 641 | VNGQVQLGEL ELRGIDELSS VAREMVRLIE TATVPIFAVD |
| 681 | TDGCINGWNA KVAELTGLSV EEAMGKSLVN DLIFKESEET |
| 721 | VNKLLSRALR GDEDKNVEIK LKTFGPEQSK GPIFVIVNAC |
| 761 | SSRDYTKNIV GVCFVGQDVT GQKVVMDKFI NIQGDYKAIV |
| 801 | HNPNPLIPPI FASDENTCCS EWNTAMEKLT GWSRGEVVGK |
| 841 | LLVGEVFGNC CRLKGPDALT KFMIVLHNAI GGQDCEKFPF |
| 881 | SFFDKNGKYV QALLTANTRSRMDGEAIGAF CFLQIASPEL |
| 921 | QQAFEIQRHH EKKCYARMKE LAYIYQEIKN PLNGIRFTNS |
| 961 | LLEMTDLKDD QRQFLETSTA CEKQMSKIVK DASLQSIEDG |
| 1001 | SLVLEKGEFS LGSVMNAVVS QVMIQLRERD LQLIRDIPDE |
| 1041 | IKEASAYGDQ YRIQQVLCDF LLSMYRFAPA ENGWVEIQVR |
| 1081 | PNIKQNSDGT DTMLFLFRFA CPGEGLPPEI VQDMFSNSRW |
| 1121 | TTQEGIGLSI CRKILKLMGG EVQYIRESER SFFHIVLELP |
| 1181 | QPQQAASRGT S |

A codon encoding the arginine at position 329 of the SEQ ID NO:37 protein is equivalent to the codon encoding the arginine at position 322 of the SEQ ID NO:30 protein. A codon encoding the arginine at position 329 of the SEQ ID NO:37 protein can be changed to a termination codon. Hence, PHYB-related Oryza sativa protein referred to as phytochrome B (NCBI accession no. AFK31004.1 (GI:388458276) with sequence SEQ ID NO:37) can be inactivated in a manner similar to the PhyB loci described above.

A chromosomal segment encoding the SEQ ID NO:37 protein is on Oryza sativa chromosome 3 at NC_029258.1 (11020017 . . . 11028186), sequence available as NCBI accession number NC_029258.1 (GI:996703430).

Chromosomal sites encoding any of the conserved amino acids and conserved domains illustrated by the sequence comparisons shown above can be deleted or mutated to reduce the activity of the proteins described herein.

For example, a wild type plant can express PHYB polypeptides or PHYB-related polypeptides with at least 70%, or at least 75%, or at least 80%, or at least 85%, or at least 90%, or at least 95%, or at least 97%, or at least 98%, or at least 99% sequence identity to any of SEQ ID NOs:30, 32, 33, 34, 35, 36, or 37.

However, the mutant phyB plant cells, plants, and/or seeds with reduced PHYB function and improved plant growth can have mutations that delete at least a portion of the phyB loci or that delete at least a portion of phyB-related loci (so that PHYB function is reduced or lost altogether). Mutant phyB plant cells, plants, and/or seeds with reduced PHYB function and improved plant growth can express mutant phyB and/or mutant phyB-related polypeptides that have reduced activity. Such PHYB and/or PHYB-related polypeptides that have reduced PHYB activity can have less than 99%, or less than 98%, or less than 95%, or less than 90%, or less than 85%, or less than 75%, or less than 60%, or less than 50%, or less than 40%, or less than 30%, or less than 20% sequence identity to any of SEQ ID NOs:30, 32, 33, 34, 35, 36, or 37. In other words, mutations can delete or modify chromosomal PhyB or PhyB-related chromosomal sites so that a truncated polypeptide, a highly mutated polypeptide, or no polypeptide is expressed.

The mutant PHYB and/or PHYB-related can, for example, have mutations in at least one conserved amino acid position, or at least two conserved amino acid positions, or at least three conserved amino acid positions, or at least five conserved amino acid positions, or at least seven conserved amino acid positions, or at least eight conserved amino acid positions, or at least ten conserved amino acid positions, or at least fifteen amino acid positions, or at least twenty conserved amino acid positions, or at least twenty-five amino acid positions. In some cases, an entire conserved PhyB and/or PhyB-related domain or the entire endogenous PHYB and/or PHYB-related gene, loci, or chromosomal segment is deleted or mutated.

The conserved amino acids and/or domains are in some cases mutated by deletion or replacement with amino acids that have dissimilar physical and/or chemical properties. Examples of amino acids with different physical and/or chemical properties that can be used are shown in Tables 1 and 2.

### Pif4

As described herein, overexpression of PIF4 can improve myc mutant plant growth without compromising defense against insects. Hence, the mutant plants described herein can also express PIF4, for example, by transformation of plants having a myc, myc-related, phyB, and/or phyB-related mutant genomic background with an expression cassette or expression vector that encodes PIF4.

One example of an Arabidopsis thaliana PIF4 protein sequence is shown below as SEQ ID NO:38.

| | |
|---|---|
| 1 | MEHQGWSFEE NYSLSTNRRS IRPQDELVEL LWRDGQVVLQ |
| 41 | SQTHREQTQT QKQDHHEEAL RSSTFLEDQE TVSWIQYPPD |
| 81 | EDPFEPDDFS SHFFSTMDPL QRPTSETVKP KSSPEPPQVM |
| 121 | VKPKACPDPP PQVMPPPKFR LTNSSSGIRE TEMEQYSVTT |
| 161 | VGPSHCGSNP SQNDLDVSMS HDRSKNIEEK LNPNASSSSG |
| 201 | GSSGCSFGKD IKEMASGRCI TTDRKRKRIN HTDESVSLSD |
| 241 | AIGNKSNQRS GSNRRSRAAE VHNLSERRRR DRINERMKAL |
| 281 | QELIPHCSKT DKASILDEAI DYLKSLQLQL QVMWMGSGMA |
| 321 | AAAASAPMMF PGVQPQQFIR QIQSPVQLPR FPVMDQSAIQ |
| 361 | NNPGLVCQNP VQNQIISDRF ARYIGGFPAM QAATQPMEML |
| 401 | RFSSPAGQQS QQPSSVPTKT TDGSRLDH |

A cDNA that encodes the SEQ ID NO:38 PIF4 protein can have nucleotide sequence SEQ ID NO:39, shown below.

| | |
|---|---|
| 1 | ACTTTCTGTC TGTACCCAAA AGAAGTAATG AACCTCTCTC |
| 41 | ATCTTCTTCT TCTCTGTTTC TTTCATGTTT TGTGAGTTGT |
| 81 | TTCTCAACAA TTTTCTGGTC TCTTAGAGTG AGAGGAGAGA |
| 121 | GATAGAGAGT TGTGTTGGGC GTGGAACTTG GACTAGTTCC |
| 161 | ACATATCAGG TTATATAGAT CTTCTCTTTC AACTTCTGAT |
| 201 | TCGTCCAGAA GCTTTCCTAA TCTGAGATCT GACATGGAAC |
| 241 | ACCAAGGTTG GAGTTTTGAG GAGAATTATA GTTTGTCCAC |
| 281 | TAATAGAAGA TCTATCAGGC CACAAGATGA ACTAGTGGAG |
| 321 | TTATTATGGC GAGATGGACA AGTGGTTCTG CAGAGCCAAA |
| 361 | CTCATAGAGA ACAAACCCAA ACCCAGAAAC AAGATCATCA |
| 401 | TGAAGAAGCC CTAAGATCCA GCACCTTTCT TGAAGATCAA |
| 441 | GAAACTGTCT CTTGGATCCA ATACCCTCCA GATGAAGACC |
| 481 | CATTCGAACC CGACGACTTC TCCTCCCACT TCTTCTCAAC |
| 521 | CATGGATCCC CTCCAGAGAC CAACCTCAGA GACGGTTAAG |
| 561 | CCTAAGTCCA GTCCTGAACC TCCTCAAGTC ATGGTTAAGC |
| 601 | CTAAGGCCTG TCCTGACCCT CCTCCTCAAG TCATGCCTCC |
| 641 | TCCAAAATTT AGGTTAACAA ATTCATCATC GGGGATTAGG |
| 681 | GAAACAGAAA TGGAACAGTA CTCGGTAACG ACCGTTGGAC |
| 721 | CTAGCCATTG CGGAAGCAAC CCATCACAGA ACGATCTCGA |
| 761 | TGTCTCAATG AGTCATGATC GAAGCAAAAA CATAGAAGAA |
| 801 | AAGCTTAATC CGAACGCAAG TTCCTCATCA GGTGGCTCCT |
| 841 | CTGGTTGCAG CTTTGGCAAA GATATCAAAG AAATGGCTAG |
| 881 | TGGAAGATGC ATCACAACCG ACCGTAAGAG AAAACGTATA |
| 921 | AATCACACTG ACGAATCTGT ATCTCTATCA GATGCAATCG |
| 961 | GTAACAAGTC GAACCAACGA TCAGGATCAA ACCGAAGGAG |
| 1001 | TCGAGCAGCT GAAGTTCATA ATCTCTCCGA AAGGAGGAGG |
| 1041 | AGAGATAGGA TCAATGAGAG AATGAAGGCT TTGCAAGAAC |
| 1081 | TAATACCTCA CTGCAGTAAA ACTGATAAAG CTTCGATTTT |
| 1121 | AGACGAAGCC ATAGATTATT TGAAATCACT TCAGTTACAG |
| 1161 | CTTCAAGTGA TGTGGATGGG GAGTGGAATG GCGGCGGCGG |
| 1201 | CGGCTTCGGC TCCGATGATG TTCCCCGGAG TTCAACCTCA |
| 1241 | GCAGTTCATA CGTCAGATAC AGAGCCCGGT ACAGTTACCT |
| 1281 | CGATTTCCGG TTATGGATCA GTCTGCAATT CAGAACAATC |
| 1321 | CCGGTTTAGT TTGCCAAAAC CCGGTACAAA ACCAGATCAT |
| 1361 | CTCCGACCGG TTTGCTAGAT ACATCGGTGG GTTCCCACAC |
| 1401 | ATGCAGGCCG CGACTCAGCC GATGGAGATG TTGAGATTTA |
| 1441 | GTTCACCGGC GGGACAGCAA AGTCAACAAC CGTCGTCTGT |
| 1481 | GCCGACGAAG ACCACCGACG GTTCTCGTTT GGACCACTAG |
| 1521 | GTTGGTGAGC CACTTTTTTA CTTCCTTATT TTTGGTATGT |
| 1561 | TTCTTTTTTA TATCTATCTT TCTGAACATA CTTAAAACGT |
| 1601 | TCAAGGATGT ATTATTATAG AGTAAACGTG CAACTTCATT |
| 1641 | ACGTTATTTT CTGTATATGT GAGTTTATGT ATGTCAAAAT |
| 1681 | GACATGATGA GATTTTTTGT AAACAACATC TTAAAAACAG |
| 1721 | GACATGTGAT TTTTGTAATC GTAAAAA |

Nucleic acids from a variety of plant types that encode PIF4 and/or PIF4-related polypeptides can be transformed into plants as transgenes. For example, such nucleic acids that encode PIF4 and/or PIF4-related polypeptides can be incorporated into expression cassettes or expression vectors that are introduced into selected plant cells, for example, plant cells with a mutant myc, JAZ, and/or phyB genetic background. Plant lines can be generated from the plant cells.

A PIF4-related protein from Zea mays referred as a putative HLH DNA-binding domain superfamily protein (NCBI accession no. NP_001146660.1 (GI:226502090)) has substantial homology to the Arabidopsis thaliana PIF4 SEQ ID NO:38 protein sequence, as illustrated below. Domains of sequence homology are identified with asterisks below the sequence comparison.

| 63.6% identity in 99 residues overlap; Score: 316.0; Gap frequency: 0.0% | | |
|---|---|---|
| Seq38 | 224 | RKRKRINHTDESVSLSDAIGNKSNQRSGSNRRSRAAEVHNLSERRRRDRINERMKALQEL |
| Seq40 | 218 | RRSGKRKHNDATDAEDVGLECEPAQRTTTAKRRRAAQVHNLSERRRRDRINEKMKKLQEL |
| | | * * * ** * *** *************** ******* |
| Seq38 | 234 | IPHCSKTDKASILDEAIDYLKSLQLQLQVMWMGSGMAAA |
| Seq40 | 278 | IPHCNKADKASMLDEAIEYLKSLQLQLQVVWMGGGIAAA |
| | | **** * **** ***** *********** *** * *** |

The Zea mays protein referred as a putative HLH DNA-binding domain superfamily protein (NCBI accession no. NP_001146660.1 (GI:226502090)) has the following sequence (SEQ ID NO:40).

| | |
|---|---|
| 1 | MQTAIEHACS VVECAATARA AMDMSHYIPD WSSSMGDTFA |
| 41 | PLGGEDDDGL IELMWRNGHV VMQAQAPRKP PRPDDDEAAA |
| 81 | AQAQAWFQYP VEERADLFSE LFGEAQAAVG GARGEAARQS |
| 121 | IRMMPPPPPP PRPAQAPREE KACPGDGGTA TATDGAGSSV |
| 161 | LTVVSSLCGS NGNHVQATAP GDVARARDVL MVTSSSTTRS |
| 201 | RSCTTKSEQP GPGPGAARRS GKRKHNDATD AEDVGLECEP |
| 241 | AQRTTTAKRR RAAQVHNLSE RRRRDRINEK MKALQELIPH |
| 281 | CNKADKASML DEAIEYLKSL QLQLQVVWMG GGIALAGVHQ |
| 321 | RTMVAAPGRP PHVASLPASA PDLYTRYLAV DHLPPPPLVP |
| 361 | PPRTAAAMGL YPRQNPVPAT SSPSFRTTEN TRKLWQA |

A cDNA encoding the Zea mays protein referred as a putative HLH DNA-binding domain superfamily protein (NCBI accession no. NP_001146660.1 (GI:226502090); SEQ ID NO:38) has the following nucleotide sequence (SEQ ID NO:41).

| | |
|---|---|
| 1 | CCTTGCCCTG CTGCAACTTG AACCTCCTGG CAGCTCCTGT |
| 41 | TTCAGGCAGG CAGCAAGTAG GGAAGAGGCT CTGCAGATCA |
| 81 | GTTCCATGCA GACAGCGATC GAGCACGCCT GCTCGGTGGT |
| 121 | GGAATGCGCT GCGACAGCCC GAGCCGCCAT GGACATGAGC |
| 161 | CACTACATCC CCGATTGGAG CAGCAGCATG GGAGACACCT |
| 201 | TCGCGCCACT GGGCGGCGAG GACGACGACG GGCTCATCGA |
| 241 | GCTCATGTGG CGCAACGGCC ACGTGGTCAT GCAGGCCCAG |
| 281 | GCGCCGCGGA AGCCGCCGAG ACCCGACGAC GACGAGGCGG |
| 321 | CGGCGGCGCA GGCGCAGGCG TGGTTCCAGT ACCCGGTGGA |
| 361 | GGAGAGGGCC GACCTCTTCT CGGAGCTCTT CGGGGAGGCG |
| 401 | CAGGCGGCCG TCGGCGGCGC GCGCGGGGAG GCCGCGCGCC |
| 441 | AGAGTATCCG GATGATGCCG CCGCCGCCGC CGCCGCCGAG |
| 481 | GCCCGCGCAA GCGCCGCGGG AGGAGAAGGC GTGCCCGGGA |
| 521 | GACGGCGGCA CGGCGACGGC GACGGACGGC GCCGGCTCGT |
| 561 | CCGTGCTCAC GGTCGTGTCC AGCCTCTGCG GGAGCAACGG |
| 601 | CAACCACGTG CAGGCGACGG CGCCGGGGGA CGTCGCCAGG |
| 641 | GCCCGCGACG TGCTGATGGT GACCTCGTCG TCGACGACGC |
| 681 | GTTCCAGGTC ATGCACCACC AAGAGCGAGC AGCCGGGTCC |
| 721 | CGGGCCCGGC GCTGCCCGCC GGAGCGGCAA GAGGAAGCAT |
| 761 | AACGACGCCA CCGATGCCGA GGACGTGGGG CTGGAGTGCG |
| 801 | AGCCGGCGCA GAGGACGACG ACTGCCAAGC GGCGCCGCGC |
| 841 | CGCGCAAGTC CACAACCTCT CGGAGCGGAG GAGACGGGAC |
| 881 | AGGATCAACG AGAAGATGAA GGCCCTGCAG GAACTCATAC |
| 921 | CCCACTGCAA CAAAGCGGAC AAGGCGTCGA TGCTGGACGA |
| 961 | GGCGATCGAG TACCTCAAGT CGCTGCAGCT CCAGCTGCAG |
| 1001 | GTGGTGTGGA TGGGCGGCGG CATCGCGGCG GCGGGGGTGC |
| 1041 | ACCAGCGGAC GATGGTGGCC GCGCCCGGGC GTCCTCCCCA |
| 1081 | CGTGGCTTCC CTGCCGGCGT CGGCGCCCGA CCTCTATACG |
| 1121 | CGCTACCTCG CCGTCGACCA CCTGCCGCCA CCGCCCTTGG |
| 1161 | TGCCACCGCC ACGCACGGCG GCGGCGATGG GCTTGTACCC |
| 1201 | GCGCCAGAAC CCCGTGCCGG CGACGTCGTC TCCTTCCTTC |
| 1241 | CGAACGACCG AAAATACGCG AAAACTATGG CAAGCCTGAG |
| 1281 | ATTCAGATCC GGGGTATGGT GACCAGCTGA TGGGTCATCT |
| 1321 | AGCTGCATGC ATGTGTGTAT GTGTTGGTAG TATGGTTAAG |
| 1361 | CCTTGACAGA GACTTGTGAT CGAGACCGAG ATCGACCGAT |
| 1401 | AGGCCGTCAC TTCTTTTTTC TTCCATCTTT CAGTTTTTGG |
| 1441 | TTGATAGGCC GGAGTGTAAT TTGACCAGTG GTCGAGATTT |
| 1481 | GTCAAGCGAC AC |

A PIF4-related protein from Glycine max referred as a transcription factor PIF4 (NCBI accession no. XP_006575634.1 (GI:571442111)) has substantial homology to the Arabidopsis thaliana PIF4 SEQ ID NO:38 protein sequence, as illustrated below. Domains of sequence homology are identified with asterisks below the sequence comparison.

| 41.7% identify in 240 residues overlap; Score: 357.0; Gap frequency: 7.5% | | |
|---|---|---|
| Seq38 | 139 | EKLNPNASSSSGGSSGCSFGKDIKEMASGRCITTD-RKRKRINHTDESVSLSDAIGNKSN |
| Seq42 | 291 | EMIELTVTSSSGGSGSTGIGRTCSLSTRDHGQKRKGTEEEALEEQSEDTELKSADGNKAS |
| | | * ****** * * * * *** |
| Seq38 | 248 | QRSGSNRRSRAAEVHNLSERRRRDRINERMKALQELIPHCSKTDKASILDEAIDYLKSLQ |
| Seq42 | 351 | QRTRSSRRNPAAEVHNQSERRRRDPINEKMRTLQQLIPNSNKTDKASMLEEAIEYLKSLQ |
| | | ** * ** ******* *********** * ** *** ****** * *** ****** |
| Seq38 | 308 | LQLQVMWMGSGMAAAAASAPMMFPGVQPQQF----IRQIQSPVQLPRFPVMDQSAIQN-- |
| Seq42 | 411 | FQLQVMWMGGGMTPVMFPGIQHYMSQMGMGMGAPSLPSIHNPMQLPKVPHDQAMSVLQIP |
| | | ******** ** * * *** * |
| Seq38 | 362 | NPGLVCQNPV----------QNQIISDRFARYIGGFPHMQAATQPMEMLRFSSPAGQQSQ |
| Seq42 | 471 | NQNLMCQNPVLGAFNYQNQMQNPCLPEQYARYMG-YHLMQNASQPMNVFRYGSQAVQHSQ |
| | | * * ***** ** *** * ** * *** * * * * ** |

The PIF4-related protein from Glycine max referred as a transcription factor PIF4 (NCBI accession no. XP_006575634.1 (GI:571442111)) has the following sequence (SEQ ID NO:42).

| | |
|---|---|
| 1 | MNNSIPGWDF ESDTCLTNQR KLIGPDQELV ELLWKNGQVV |
| 41 | MHNQTHRKTL GNSSNLRQVQ KSDQSVLRSS GPYGNSSNLD |
| 81 | QEDAAPWVQF PLEDPLEQDF CSNLLSELPT CEFESYKPIR |
| 121 | QLEEEKFAKF FASGTPHHPT TSSSQPLPPN MKPSCIQGLQ |
| 161 | GNPIPMPAPR FHGPDSSQKI HDFGASRKVL NFPQFSTPRN |
| 201 | NVPSAPGITQ FREKTTANMS QSEAREYSVI TVGSSHCGSN |
| 241 | HIPQEQDVSR ISSTGVWATT NNNTTLSAEP EAVRDYVQRP |
| 281 | ICPKSGQGKS EMIELTVTSS SGGSGSTGIG RTCSLSTRDH |
| 321 | GQKRKGTEEE ALEEQSEDTE LKSADGNKAS QRTRSSRRNR |
| 361 | AAEVHNQSER RRRDRINEKM RTLQQLIPMS NKTDKASMLE |
| 401 | EAIEYLKSLQ FQLQVMWMGG GMTPVMFPGI QHYMSQMGMG |
| 441 | MGAPSLPSIH NPMQLPKVPH DQAMSVLQIP NQNLMCQNPV |
| 481 | LGAFNYQNQM QNPCLPEQYA RYMGYELMQN ASQPMNVFRY |
| 521 | GSQAVQHSQT MIAPGNNSSG PMSGTANIDD ADSGKAGSST |
| 561 | FN |

A cDNA encoding the PIF4-related protein from Glycine max referred as a transcription factor PIF4 (NCBI accession no. XP_006575634.1 (GI:571442111); SEQ ID NO:42) is shown below as SEQ ID NO: 43.

| | |
|---|---|
| 1 | GACCCCGTTT TCAACTGGTC CCGTGTTCCT TCATTTGATG |
| 41 | CCACATGTGC AGCTAGCCAT GTTTTTCTCG CTGTTGACGA |
| 81 | GCACAATATA TAATAAATAC CATTTTTTTC ATGCCATATT |
| 121 | TGCTCTCTTC TCTCTTTGTA CTAATAACTT GGATCTATGC |
| 161 | CACTGTCCTT CTCCTTGTTA AAAACTGTGC CACACGTCTG |
| 201 | TCACCAAACT CCCTAAGCAG AAGAAGCACA TGTTCAGAGG |
| 241 | GAGTTTTGTT TCATCAGTCT CTAGCTAGCA TATATTTCTA |
| 281 | GCTTCTATTC AACAAGTTGC AAAAAACAGA CTTTGCCTTA |
| 321 | ACCAAAAGAA AATCTGTTTT TACCTTAACT CAGACAACTC |
| 361 | GTTTGGTGAA CCATGAACAA CAGTATTCCT GGTTGGGATT |
| 401 | TTGAGAGTGA TACATGTCTC ACCAACCAAA GAAAGCTCAT |
| 441 | AGGGCCGGAC CAAGAACTTG TAGAGCTCCT ATGGAAAAAT |
| 481 | GGGCAAGTAG TTATGCACAA CCAAACACAT AGGAAGACAC |
| 521 | TTGGGAATTC ATCTAACTTG AGACAGGTGC AGAAAAGTGA |
| 561 | TCAATCAGTA TTAAGGTCTA GCGGTCCCTA TGGAAACTCA |
| 601 | AGCAACTTGG ATCAAGAAGA TGCCGCCCCA TGGGTCCAAT |
| 641 | TCCCACTTGA GGACCCATTG GAACAAGATT TTTGTTCAAA |
| 681 | CCTTTTATCT GAACTACCAA CTTGTGAATT TGAATCTTAC |
| 721 | AAGCCAATCA GGCAATTGGA AGAGGAAAAG TTTGCCAAAT |
| 761 | TTTTTGCTTC CGGTACCCCC CATCATCCTA CAACTTCAAG |
| 801 | TTCACAACCA CTACCACCTA ACATGAAACC CTCATGTATT |
| 841 | CAGGGACTCC AAGGGAATCC TATTCCTATG CCAGCTCCAA |
| 881 | GATTTCATGG TCCTGATTCA TCTCAGAAAA TCCATGACTT |
| 921 | TGGAGCATCA CGAAAGGTTC TAAATTTTCC TCAGTTTTCA |
| 961 | ACACCCCGTA ATAATGTTCC ATCAGCACCT GGTATTACAC |
| 1001 | AGTTTAGAGA GAAAACTACT GCTAACATGT CACAAAGTGA |
| 1041 | GGCTAGAGAG TACTCAGTGA TCACAGTTGG TTCAAGTCAC |
| 1081 | TGTGGCAGCA ATCACATCCC TCAGGAGCAA GATGTAAGCA |
| 1121 | GGATTTCAAG CACTGGTGTT TGGGCCACTA CTAATAATAA |
| 1161 | TACTACTTTA TCTGCTGAGC CTGAAGCTGT CAGAGATTAT |
| 1201 | GTCCAAAGAC CGATTTGTCC TAAGAGTGGC CAAGGAAAAT |
| 1241 | CAGAGATGAT TGAACTAACT GTGACTTCAT CTTCCGGTGG |
| 1281 | CTCGGGAAGT ACTGGTATCG GAAGAACCTG TTCCCTATCA |
| 1321 | ACAAGAGATC ATGGCCAAAA GAGAAAAGGG ACAGAAGAAG |
| 1361 | AAGCGTTAGA GGAACAAAGT GAGGACACAG AACTTAAATC |
| 1401 | AGCTGATGGA AACAAGGCTT CTCAGCGGAC GAGGTCTTCC |
| 1441 | AGAAGGAACC GTGCAGCAGA AGTGCATAAT CAATCAGAAA |
| 1481 | GGAGAAGAAG AGATAGGATC AACGAGAAGA TGAGGACATT |
| 1521 | GCAGCAACTG ATACCTAATA GTAACAAGAC AGACAAAGCA |
| 1561 | TCAATGTTAG AAGAGGCAAT CGAATACTTG AAATCACTTC |
| 1601 | AGTTTCAGCT TCAGGTTATG TGGATGGGGG GTGGCATGAC |
| 1641 | ACCAGTGATG TTCCCAGGAA TTCAGCACTA TATGTCACAA |
| 1681 | ATGGGTATGG GAATGGGTGC ACCTTCTTTG CCTTCCATTC |
| 1721 | ACAACCCGAT GCAATTGCCA AAAGTGCCAC ATGATCAAGC |
| 1761 | CATGTCTGTG CTTCAGATAC CAAACCAGAA TTTAATGTGT |
| 1801 | CAAAATCCAG TTTTGGGTGC CTTTAACTAC CAAAACCAGA |
| 1841 | TGCAGAACCC GTGCCTTCCA GAACAATATG CACGTTACAT |
| 1881 | GGGTTACCAT CTTATGCAAA ATGCCTCTCA GCCTATGAAT |
| 1921 | GTGTTCAGAT ATGGTTCCCA AGCAGTGCAA CACAGTCAAA |
| 1961 | CGATGATTGC ACCAGGCAAT AATAGCAGCG GACCCATGAG |
| 2001 | TGGAACAGCT AATATTGATG ATGCTGACAG TGGCAAAGCG |
| 2041 | GGTTCTTCCA CCTTTAATTG AATAGTGAAT AGCAATACCT |
| 2081 | TAAAATTACT CAATTGGGGG AATTACCTAA TGGAGTACGT |
| 2121 | CAATCCTCAC AAGCACCAAT ATGTGCTCCA ATTTTATGTA |
| 2161 | G |

A PIF4-related protein from Oryza sativa referred as a transcription factor PIF4 isoform X3 (NCBI accession no. XP_015618080.1 (GI:1002309425)) has substantial homology to the Arabidopsis thaliana PIF4 SEQ ID NO:38 protein sequence, as illustrated below. Domains of sequence homology are identified with asterisks below the sequence comparison.

| 36.8% identity in 323 residues overlap; Score: 322.0; Gap frequency: 8.4% | | |
|---|---|---|
| Seq38 | 25 | DELVELLWRDGQVVLQSQTHREQTQTQKQDHHEEALRSSTFLEDQETVSWIQYPPDEDPF |
| Seq44 | 24 | DGLVELLWCNGHVVMQSQAPRKPPRPEKT-----TAAAAAAMAEDESASWFQYPVD-DVL |
| | | * ****** * ** *** * * * ** *** * * |
| Seq38 | 85 | EPDDFSSHFFSTMDPLQRPTSETVKPKSSPEPPQVMVKPKACPDPPPQVMPPPKFRLTNS |
| Seq44 | 78 | EKDLFTE-LFGEMTAAGGGGGDVRRAACKEERGAVAAFQSRMMPPPWPARGKAEFGDVDD |
| | | * * * * * * * ** * |
| Seq38 | 145 | SSGIRETEMEQY------------SVTTVGPSHCGSNPSQND-LDVSMSHDRSKNIEEKL |
| Seq44 | 137 | VCGVSEVVMAKMDGAAAAETVGESSMLTIGSSICGSNHVQTPPVGNGKAGAGTAGAARRA |
| | | * * * * * * * **** * |
| Seq38 | 192 | NPNAS--SSSGGSSGCSFGKDIKEMASGRCITTDRKR----KRINHTDESVSLSDAIGNK |
| Seq44 | 197 | HDTATVASSSMRSRSCTAKAEPRDVAAAGVGGKRKQRGGAAMESGSPSEDVEFESAAATC |
| | | * *** * * * * * * * |
| Seq38 | 246 | SN-QRSGSNRRSRAAEVHNLSERRRRDRINERMKALQELIPHCSKTDKASILDEAIDYLK |
| Seq44 | 257 | SPAQKTTTAKRRRAAEVHNLSERRRRDRINEKMKALQELIPHCNKTDKASMLDEAIEYLK |
| | | * * * ******************* *********** ****** ***** *** |
| Seq38 | 305 | SLQLQLQVMWMGSGMAAAAASAP |
| Seq44 | 317 | SLQLQLQMMWMGGGMAPPAVMFP |
| | | ******* **** *** * * |

This PIF4-related protein from Oryza sativa referred as a transcription factor PIF4 isoform X3 (NCBI accession no. XP_015618080.1 (GI:1002309425)) has the following sequence (SEQ ID NO:44).

| | |
|---|---|
| 1 | MNQFVPDWNT TSMGDGFAPL GEDDGLVELL WCNGHWMQS |
| 41 | QAPRKPPRPE KTTAAAAAAM AEDESASWFQ YPVDDVLEKD |
| 81 | LFTELFGEMT AAGGGGGDVR RAACKEERGA VAAFQSRMMP |
| 121 | PPWPARGKAE FGDVDDVCGV SEWMAKMDG AAAAETVGES |
| 161 | SMLTIGSSIC GSMHVQTPPV GNGKAGAGTA GAARRAHDTA |
| 201 | TVASSSMRSR SCTAKAEPRD VAAAGVGGKR KQRGGAAMES |
| 241 | GSPSEDVEFE SAAATCSPAQ KTTTAKRRRA AEVHNLSERR |
| 281 | RRDRINEKMK ALQELIPHCN KTDKASMLDE AIEYLKSLQL |
| 321 | QLQMMWMGGG MAPPAVMFPA AGVHQYMQRM GAVGMGPPHM |
| 361 | ASLPRMPPFM APPPAAYQSS PVYSMADPYA RCLAVDHLQP |
| 401 | PPPMFRREY |

A cDNA encoding the PIF4-related protein from Oryza sativa referred as a transcription factor PIF4 isoform X3 (NCBI accession no. XP_015618080.1 (GI:1002309425); SEQ ID NO:44) has the following sequence (SEQ ID NO:45).

| | |
|---|---|
| 1 | GCGAGTCCTC TTCCTGCCCT GCCCTGCCCT GCCCTGCATT |
| 41 | CTTTCTTTCT CCACCAGGGG AATCCAGTTC ACCCCCAGTG |
| 81 | CTGCTTCTGC TGCTGCTTCT GCATCATCTT GCCCTGTTAA |
| 121 | AAAGACACAG TGCCCTTGTT CTTTCGCAGT TGCAACTAGC |
| 161 | ATCTCCTCCT CTACTTGTAC TCACTTCACA CCTCAGCTCA |
| 201 | GCTCAGCTCA TCTCCTGTCA TCTCAGCTCA AAGAGAAAGA |
| 241 | GCTGAAGGTG TAAGCTGATC ACCAGGAAGC AGAGGCTTTT |
| 281 | TTTCAGATTA CAGTTATCTG AAACAACCAA CTTCAGAATC |
| 321 | AATCAGCAAA GGTAGAAACA AGACAGAGCT GCTGTGCTTC |
| 361 | TGTGATTAAT TAGGGTTGTT AATGCCATGA ACCAGTTCGT |
| 401 | CCCTGATTGG AACACCACCA GCATGGGCGA CGGCTTTGCG |
| 441 | CCATTAGGCG AAGACGACGG GCTCGTCGAG CTGCTATGGT |
| 481 | GCAATGGCCA CGTCGTCATG CAGAGCCAGG CGCCGCGGAA |
| 521 | GCCGCCGAGG CCGGAGAAGA CGACGGCGGC GGCGGCGGCG |
| 561 | GCGATGGCGG AGGATGAGTC GGCGTCGTGG TTTCAGTACC |
| 601 | CGGTCGACGA CGTGCTTGAG AAGGACCTGT TCACCGAGCT |
| 641 | GTTCGGCGAA ATGACGGCGG CCGGCGGCGG CGGCGGCGAC |
| 681 | GTCCGCAGGG CGGCGTGCAA GGAGGAGCGC GGCGCGGTCG |
| 721 | CCGCGTTCCA GAGCAGGATG ATGCCGCCGC CGTGGCCGGC |
| 761 | GAGGGGGAAG GCGGAGTTCG GTGACGTCGA CGACGTGTGC |
| 801 | GGCGTCTCGG AGGTCGTCAT GGCGAAGATG GACGGGGCGG |
| 841 | CGGCGGCGGA GACGGTCGGC GAGTCATCGA TGCTGACAAT |
| 881 | CGGGTCGAGC ATCTGCGGGA GCAACCACGT CCAGACGCCG |
| 921 | CCGGTGGGGA ACGGGAAGGC CGGCGCCGGC ACCGCCGGCG |
| 961 | CCGCCAGAAG GGCGCACGAC ACGGCGACGG TGGCGTCGTC |
| 1001 | GTCGATGAGG TCGAGGTCCT GCACCGCCAA GGCCGAGCCG |
| 1041 | CGCGACGTCG CAGCCGCCGG CGTCGGCGGC AAGCGGAAGC |
| 1081 | AGCGCGGCGG CGCCGCCATG GAGTCCGGGA GCCCCAGCGA |
| 1121 | GGACGTGGAG TTCGAGTCCG CCGCCGCAAC GTGCTCGCCG |
| 1161 | GCGCAGAAGA CGACGACGGC GAAGCGGCGG CGCGCCGCCG |
| 1201 | AGGTGCACAA CCTCTCCGAG AGGAGGAGAA GAGATAGGAT |
| 1241 | CAATGAGAAG ATGAAAGCAT TACAGGAGCT CATACCTCAC |
| 1281 | TGCAACAAAA CGGACAAAGC ATCGATGCTG GATGAAGCGA |
| 1321 | TCGAGTATCT CAAGTCACTG CAGCTCCAGC TACAGATGAT |
| 1361 | GTGGATGGGC GGCGGAATGG CGCCGCCGGC GGTGATGTTC |
| 1401 | CCGGCGGCGG GCGTGCACCA GTACATGCAG CGGATGGGCG |
| 1441 | CCGTCGGGAT GGGCCCACCA CACATGGCGT CCCTGCCGAG |
| 1481 | GATGCCGCCG TTCATGGCGC CGCCGCCGGC CGCCGTGCAG |
| 1521 | AGCTCGCCGG TGGTCAGCAT GGCCGACCCC TACGCCCGCT |
| 1561 | GCCTCGCCGT CGACCACCTC CAGCCACCGC CTCCGATGTT |
| 1601 | TCGACGCGAA TACTAGGGAA GGAACTAATA TCAAATAATA |
| 1641 | GAAGGGGTGA GCCTTCGAAT CGAGATCGTC TAGCCCACCA |
| 1681 | CCTTATAGAG CTAGCCGGAA GGCCCTCGAG CGTTTCTCAT |
| 1721 | ATTTTCAGTT TCCTAAGAGT TTTTTTTTT |

Expression cassettes and expression vectors can include a nucleic acid segment encoding a PIF4 protein where the nucleic acid segment is operably linked to a promoter. The nucleic acid segment can encode a PIF4 protein with at least 70% sequence identity, or at least 80% sequence identity, or at least 85% sequence identity, or at least 90% sequence identity, or at least 95% sequence identity, or at least 97% sequence identity, or at least 98% sequence identity, or at least 99% sequence identity to any of amino acid sequences identified as SEQ ID NO: 38, 40, 42, or 44. For example, the Pif4 nucleic acid segment can have at least 70% sequence identity, or at least 80% sequence identity, or at least 85% sequence identity, or at least 90% sequence identity, or at least 95% sequence identity, or at least 97% sequence identity, or at least 98% sequence identity, or at least 99% sequence identity to any of nucleic acid sequences identified as SEQ ID NO: 38, 40, 42, or 44.

As stated, expression cassettes and expression vectors can include a nucleic acid segment encoding a PIF4 protein where the nucleic acid segment is operably linked to a promoter. Promoters provide for expression of mRNA from the PIF4 nucleic acids. The promoter can be heterologous to the PIF4 nucleic acid segment. In other words, such a heterologous promoter is not naturally linked to such a PIF4 nucleic acid segment. Instead, some expression cassettes and expression vectors have been recombinantly engineered to include a PIF4 nucleic acid segment operably linked to a heterologous promoter. A PIF4 nucleic acid is operably linked to the promoter, for example, when it is located downstream from the promoter.

A variety of promoters can be included in the expression cassettes and/or expression vectors. Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences can also contain regulatory sequences such as enhancer sequences that can influence the level of gene expression. Some isolated promoter sequences can provide for gene expression of heterologous DNAs, that is a DNA different from the native or homologous DNA.

Promoters can be strong or weak, or inducible. A strong promoter provides for a high level of gene expression, whereas a weak promoter provides for a very low level of gene expression. An inducible promoter is a promoter that provides for the turning on and off of gene expression in response to an exogenously added agent, or to an environmental or developmental stimulus. For example, a bacterial promoter such as the P_{tac} promoter can be induced to vary levels of gene expression depending on the level of isothiopropylgalactoside added to the transformed cells. Promoters can also provide for tissue specific or developmental regulation. A strong promoter for heterologous DNAs can be advantageous because it provides for a sufficient level of gene expression for easy detection and selection of transformed cells and provides for a high level of gene expression when desired. In some cases, the promoter within such expression cassettes/vectors can be functional during plant development or growth.

Expression cassettes/vectors can include, but are not limited to, a plant promoter such as the CaMV 35S promoter (Odell et al., Nature. 313:810-812 (1985)), or others such as CaMV 19S (Lawton et al., Plant Molecular Biology. 9:315-324 (1987)), nos (Ebert et al., Proc. Natl. Acad. Sci. USA. 84:5745-5749 (1987)), Adh1 (Walker et al., Proc. Natl. Acad. Sci. USA. 84:6624-6628 (1987)), sucrose synthase (Yang et al., Proc. Natl. Acad. Sci. USA. 87:4144-4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)) or those associated with the R gene complex (Chandler et al., The Plant Cell. 1:1175-1183 (1989)). Further suitable promoters include the poplar xylem-specific secondary cell wall specific cellulose synthase 8 promoter, cauliflower mosaic virus promoter, the Z10 promoter from a gene encoding a 10 kD zein protein, a Z27 promoter from a gene encoding a 27 kD zein protein, inducible promoters, such as the light inducible promoter derived from the pea rbcS gene (Coruzzi et al., EMBO J. 3:1671 (1971)) and the actin promoter from rice (McElroy et al., The Plant Cell. 2:163-171 (1990)). Seed specific promoters, such as the phaseolin promoter from beans, may also be used (Sengupta-Gopalan, Proc. Natl. Acad. Sci. USA. 83:3320-3324 (1985). Other promoters useful in the practice of the invention are available to those of skill in the art.

Alternatively, novel tissue specific promoter sequences may be employed in the practice of the present invention. cDNA clones from a particular tissue are isolated and those clones which are expressed specifically in that tissue are identified, for example, using Northern blotting. Preferably, the gene isolated is not present in a high copy number, but is relatively abundant in specific tissues. The promoter and control elements of corresponding genomic clones can then be localized using techniques well known to those of skill in the art.

A PIF4 nucleic acid can be combined with the promoter by standard methods to yield an expression cassette, for example, as described in Sambrook et al. (MOLECULAR CLONING: A LABORATORY MANUAL. Second Edition (Cold Spring Harbor, NY: Cold Spring Harbor Press (1989); MOLECULAR CLONING: A LABORATORY MANUAL. Third Edition (Cold Spring Harbor, NY: Cold Spring Harbor Press (2000)). Briefly, a plasmid containing a promoter such as the 35S CaMV promoter can be constructed as described in Jefferson (Plant Molecular Biology Reporter 5:387-405 (1987)) or obtained from Clontech Lab in Palo Alto, California (e.g., pBI121 or pBI221). Typically, these plasmids are constructed to have multiple cloning sites having specificity for different restriction enzymes downstream from the promoter. The PIF4 nucleic acids can be subcloned downstream from the promoter using restriction enzymes and positioned to ensure that the DNA is inserted in proper orientation with respect to the promoter so that the DNA can be expressed as sense or antisense RNA. Once the PIF4 nucleic acid is operably linked to a promoter, the expression cassette so formed can be subcloned into a plasmid or other vector (e.g., an expression vector).

In some embodiments, a cDNA clone encoding a PIF4 protein is isolated or obtained from a selected plant type. In other embodiments, cDNA clones from other species (that encode a PIF4 protein) are isolated from selected plant tissues. For example, the nucleic acid encoding a PIF4 protein can be any nucleic acid with a coding region that hybridizes to SEQ ID NO:39 and that has PIF4 activity. In another example, the PIF4 nucleic acid can encode a PIF4 protein with at least 90% sequence identity to SEQ ID NO:38. Using restriction endonucleases, the entire coding sequence for the PIF4 nucleic acid is subcloned downstream of the promoter in a 5′ to 3′ sense orientation.

jazQ Mutations

A quintet of JAZ transcriptional repressor genes can be modified to improve insect resistance in plants. The quintet of JAZ transcriptional repressor genes can encode JAZ1, JAZ3, JAZ4, JAZ9, JAZ10, and/or related proteins. Reduction or deletion of genes that encode JAZ1, JAZ3, JAZ4, JAZ9, JAZ10, and/or related proteins can provide insect resistance to plants.

JAZ1 proteins are repressors of the jasmonic acid signaling pathway. One example, of an Arabidopsis thaliana jasmonate-zim-domain protein 1 (JAZ1) protein sequence is shown below (SEQ ID NO:48).

| | |
|---|---|
| 1 | MSLFPCEASN MDSMVQDVKP TNLFPRQPSF SSSSSSLPKE |
| 41 | DVLKMTQTTR SVKPESQTAP LTIFYAGQVI VFNDFSAEKA |
| 81 | KEVINLASKG TANSLAKNQT DIRSNIATIA NQVPHPRKTT |
| 121 | TQEPIQSSPT PLTELPIARR ASLHRFLEKR KDRVTSKAPY |
| 161 | QLCDPAKASS NPQTTGNMSW LGLAAEI |

A chromosomal DNA sequence for the Arabidopsis thaliana jasmonate-zim-domain protein 1 (JAZ1) protein with SEQ ID NO:48 is shown below as SEQ ID NO:49.

| | |
|---|---|
| 1 | ATATTGGAGG TAGGAAGAAG AACTCTGCAA CCAAACCAAC |
| 41 | CAACCCCAAA GCCAAACAAA GTTTTATAGA GACCTTCCAT |
| 121 | TTCTCCCTCT CGTGAGAAAC GCAATTTGCA GAGAAGCAAC |
| 201 | AGCAACAACA AGAAGAAGAA GAAAAAGATT TGAGATTACT |
| 241 | TTGTATCGAT TTAGCTATTC GAGAAACTCT TGCCGTTTGA |
| 281 | AAGTTTTAAT TGTTAAAGAT GTCGAGTTCT ATGGAATGTT |
| 321 | CTGAGTTCGT CGGTAGCCGG AGATTTACTG GGAAGAAGCC |
| 361 | TAGCTTCTCA CAGACGTGTA GTCGATTGAG TCAGTATCTA |
| 401 | AAAGAGAACG GTAGCTTTGG AGATCTGAGC TTAGGAATGG |
| 441 | CATGCAAGCC TGATGTCAAT GGTAAGAAAC CTTCTCTTTC |
| 481 | TCCTAGATCC ACTTCTTTTT TCGTTTTCTC TGTTTTTTAT |
| 521 | TTCTTGAATC TTGATCTTGA AAACTTTTCA AGAAAATTTT |
| 561 | GAATCGATTT CAAAGAAATT AGGGAGAGTT AGTTTGCTAA |
| 601 | ATTTTGACAT AGAAAATGAT TGGAGAGAGT TCTAACTTTT |
| 641 | GGATCATATA TATTTGCAGG AACTTTAGGC AACTCACGTC |
| 681 | AGCCGACAAC AACCATGAGT TTATTCCCTT GTGAAGCTTC |
| 721 | TAACATGGAT TCCATGGTTC AAGATGTTAA ACCGACGAAT |
| 761 | CTGTTTCCTA GGCAACCAAG CTTTTCTTCC TCATCTTCCT |
| 801 | CTCTTCCAAA GGAAGATGTT TTGAAAATGA CACAGACTAC |
| 841 | CAGATCTGTG AAACCAGAGT CTCAAACTGC ACCATTGACT |
| 881 | ATATTCTACG CCGGGCAAGT GATTGTATTC AATGACTTTT |
| 921 | CTGCTGAGAA AGCCAAAGAA GTGATCAACT TGGCGAGCAA |
| 961 | AGGCACCGCT AATAGCTTAG CCAAGAATCA AACCGATATC |
| 1001 | AGAAGCAACA TCGCTACTAT CGCAAACCAA GTTCCTCATC |
| 1041 | CAAGAAAAAC CACAACACAA GAGCCAATCC AATCCTCCCC |
| 1081 | AACACCATTG ACAGAACTTC CTATTGCTAG AAGAGCTTCA |
| 1121 | CTTCACCGGT TCTTGGAGAA GAGAAAGGAC AGAGTTACGT |
| 1161 | CAAAGGCACC ATACCAATTA TGCGATCCAG CCAAAGCGTC |
| 1201 | TTCAAACCCT CAAACCACAG GCAACATGTC GTGGCTCGGT |
| 1241 | TTAGCAGCTG AAATATGAAT GCTAACCACC CTCAAGCCGT |
| 1281 | ACCAAGAAAT TCTTTTGACG ACGTTGCTTC AAGACAAGAT |
| 1321 | ATAAAAGCTC CTATCTTCAT GCTTTTTGAT TTAAGATACA |
| 1361 | AACTACTCAA TGATTAGGAA ACTTCATATA TTTGTATGTA |
| 1401 | TTGATTAGTG ATCAATTATT GTTAGTATTC GTTATAGTCT |
| 1441 | GTTTTTCTAC TAGTTATTGT CGCCTGTCTA AATCCCCTTG |
| 1481 | CTATGGGTTA TCTCAAAATT AGTTTCGTAT GTAACTAATT |
| 1521 | TTGTAAGAAC AATAATTTTT GTTGACGAAC CATACTATCA |
| 1561 | AATACTCTAA ATTATATCTT GATAAATCTA CCTATCAGGT |
| 1601 | AAGTAGG |

JAZ3 is also a repressor of jasmonate responses, and it is targeted by the SCF(COI1) complex for proteasome degradation in response to jasmonate. One example, of an Arabidopsis thaliana jasmonate-zim-domain protein 3 (JAZ3) protein sequence is shown below (SEQ ID NO:50).

| | |
|---|---|
| 1 | MERDFLGLGS KNSPITVKEE TSESSRDSAP NRGMNWSFSN |
| 41 | KVSASSSQFL SFRPTQEDRH RKSGNYHLPH SGSFMPSSVA |
| 81 | DVYDSTRKAP YSSVQGVRMF PNSNQHEETN AVSMSMPGFQ |
| 121 | SHHYAPGGRS FMNNNNNSQP LVGVPIMAPP ISILPPPGSI |
| 161 | VGTTDIRSSS KPIGSPAQLT IFYAGSVCVY DDISPEKAKA |
| 201 | IMLLAGNGSS MPQVFSPPQT HQQVVHHTRA SVDSSAMPPS |
| 241 | FMPTISYLSP EAGSSTNGLG ATKATRGLTS TYHNNQANGS |
| 281 | NINCPVPVSC STNVMAPTVA LPLARKASLA RFLEKRKERV |
| 321 | TSVSPYCLDK KSSTDCRRSM SECISSSLSS AT |

A chromosomal DNA sequence for the Arabidopsis thaliana jasmonate-zim-domain protein 3 (JAZ3) protein with SEQ ID NO:50 is shown below as SEQ ID NO:51.

| | |
|---|---|
| 1 | GCGATTTGTT AATAAAACTA GAAATTGCGG TGAATTAACT |
| 41 | TCATTCCACG TTTTTTCATT TTCTCCCTCA AAAGTCTCTG |
| 81 | TTTTTTTTCC TTTTTCCGGC GAAGCTCTAT TTAGCTTGAT |
| 121 | TCCGGCGTTT AACACGCGTT TTAATCGAAA CAGACATTTG |
| 161 | AGATCGAATT AATTTTGTAG CGGGCTGTGT CTTTATTATA |
| 201 | GATGGAGAGA GATTTTCTCG GGTTGGGTTC GAAAAATTCT |
| 241 | CCGATCACTG TCAAGGAGGA AACCAGCGAA AGCTCTAGAG |
| 281 | ATTCAGGTTA TTTATTACTC TTCTCAATTT TTCTGATTCT |
| 321 | GATTGTTTTT AAATCGTAGA TTTGTTTGAT TGATTAGGAG |
| 361 | TTATTAGGAC TACTTGTAGT ATGGAATTTG TTTTTGGATA |
| 401 | GCTGATTTTA TGGCTTGCTC GGGAACTGGA ATTGTCAGTT |
| 441 | TGTTGCTTGG AGCAGAACAT TGTCCTTTGC TTTTCTCGGG |
| 481 | AGATGTAGAA TTTGGATTTG GAAAAACTAG TGTTCTTTTC |
| 521 | CAAAGCCTTG TCTTAAACAT GCTTTCGGTC GGAGAAATTA |
| 561 | ACGAGAACTA ATCTCAAGCT TCTAACATAA TTAAACTCGG |
| 601 | TAAACTTTTT TTTACTAGAG TAAATTTTTT TGTTTTGTTT |
| 641 | GAAGAGTCTT ATAATTGAGA AATACTTTAT TAGTTTATAC |
| 681 | TAAAAAAAAA ACGAATACGT AAAATGTTGG AAAAGAGGGG |
| 721 | ATGTATAGAG ACTGATACAA AAATGATAAA ATAGAGACGG |
| 761 | TTGGTAGTAG GTAGAAAGAT TAAATATACT CAAAAGAGTG |
| 801 | AGTTGGATTA GTTTATAAGA TGATTAACTT CTTGATTGTG |
| 841 | TGAGTTGGAT TAGTTTATGA GATTATTAAA ATATTGATTG |
| 881 | TGTATTTGTG TTGTGTGTTG ATTAAGCGGA ACTTGCGTTA |
| 921 | GAATATTGTT CAAGGTACAA TGTGGAAATA ATAGTTTTCT |
| 961 | CACCACGAGG AATATAATTA TTTCAACTTT GTTTTCTTAT |
| 1001 | CAGCCAAAAC GTGCCACACC ATAAAAGTAG TGCATCAACA |
| 1041 | TGTGGTGTGG TGTGGTGGGG TTAAAGTTTG AATCTCTCTT |
| 1081 | TAATTTAAAC TATTAAAACA AACTTAAATT ATTGGAGTTT |
| 1121 | CGTACAATGA CTTTCAATCA AATGTTTTAG AATTAGACAC |
| 1161 | GGTTTTCGAA AGTGGTTTTC CCTCGTTGAA TTTGTCAACA |
| 1201 | GTATCAGATT CTACATTGTT GGTTACTAAT CTTTTCCTTG |
| 1241 | AAGTAGGTGT TGAATTAATC CTCTGTTGTT TATGTAAGGA |
| 1281 | GATCTCGAGA CATTTATGGT TAACAGTTAA CACTAGATGT |
| 1321 | TTGACTTTAA ACTGATTATC TTTTATTCTT TTTCTTTTGT |
| 1361 | AGCTCCCAAC AGAGGAATGA ACTGGTCTTT CTCAAACAAA |
| 1401 | GTATCAGCTT CTTCTTCTCA GTTTCTATCC TTCAGGCCAA |
| 1441 | CTCAAGAAGA TAGACATAGA AAGTCTGGAA ATTATCATCT |
| 1481 | TCCTCACTCT GGTTCCTTCA TGCCATCATC AGTAGCTGAT |
| 1521 | GTTTATGATT CAACCCGCAA AGCTCCTTAC AGTTCTGTAC |
| 1561 | AGGTATTTGT CATCAAAACC TATGTTAACC AAGACCCTTG |
| 1601 | TGTTTTTTTT ATCCTTCGCA AGATAGCTTT AAAAGTGAGC |
| 1641 | CCTGTTTTAT GAGCATATAG TAATTGGTTT TGAGTCTAGT |
| 1681 | TTAGCACAAG TTCATGGCAA TTAGTTTGTG GATCTAATCT |
| 1721 | TGGTTTAATA CTGATTCATT TTAAGTGTAA GCTAAGCTTC |
| 1761 | TCATTTTTGA TAAGTTAGTT CATACAATGC CTCACACCTA |
| 1801 | CTTTATGGCT TGTTACTCTC AGGGAGTGAG GATGTTCCCT |
| 1841 | AATTCCAATC AACACGAAGA AACTAACGCA GTTTCCATGT |
| 1881 | CGATGCCGGG TTTCCAGTCT CATCATTATG CACCAGGAGG |
| 1921 | AAGAAGCTTC ATGAACAATA ACAATAACTC ACAACCTTTG |
| 1961 | GTAGGAGTTC CTATCATGGC ACCTCCAATT TCAATCCTTC |
| 2001 | CTCCTCCAGG TTCCATTGTA GGGACTACTG ATATTAGGTA |
| 2041 | CCCACTAGTC ATCATATCAT ACAGAAACTC TTTCTACATT |
| 2081 | TTCATAGTTG ACTAAAGACT TATTTTTGTC AGATCTTCTT |
| 2121 | CCAAGCCAAT AGGTTCACCT GCGCAGTTGA CGATCTTTTA |
| 2161 | TGCCGGTTCA GTTTGTGTTT ACGATGACAT ATCTCCTGAA |
| 2201 | AAGGTATCTC AATCATTTTC TTCCATATAT GCATCTCTTT |
| 2241 | TACTCGTAAG GTATGGTACT CATTTGCTTT CTTTCATTTC |
| 2281 | TCAGGCAAAG GCGATAATGT TGCTAGCTGG GAACGGTTCC |
| 2321 | TCTATGCCTC AAGTCTTTTC GCCGCCTCAA ACTCATCAAC |
| 2361 | AAGTGGTCCA TCATACTCGT GCCTCTGTCG ATTCTTCAGC |
| 2401 | TATGCCTCCT AGCTTCATGC CTACAATATC TTATCTTAGC |
| 2441 | CCTGAAGCTG GAAGTAGCAC AAACGGACTC GGAGCCACAA |
| 2481 | AAGCGACAAG AGGCTTGACG TCAACATATC ACAACAACCA |
| 2521 | AGCTAATGGA TCCAATATTA ACTGCCCAGT ACCAGTTTCT |
| 2561 | TGTTCTACCA ATGTAATGGC TCCAACAGGT AAAAAACAAA |
| 2601 | GTCAGAGACC TGATACTACA TTCGCCATCT AACTTACTAG |
| 2641 | TATTTTCATG GATGTAACTT CATTCTCGTT CTGTTTCTTA |
| 2681 | TGCAGTGGCA TTACCTCTGG CTCGCAAAGC ATCCCTGGCT |
| 2721 | AGGTTTTTAG AGAAACGCAA AGAAAGGTAC GCAACACTTC |
| 2761 | TTTAGAATAC ACCATTCAAT AGTTTCTTGG GCTAACTCTC |
| 2801 | TTTCTCGCTG TGGGTTTCTC AGGGTCACGA GCGTATCCCC |
| 2841 | ATATTGCTTA GACAAGAAGT CATCGACAGA TTGTCGCAGA |
| 2881 | TCAATGTCTG AATGCATTAG TTCTTCTCTC AGCTCTGCAA |
| 2921 | CCTAATTTCA TCTACAGTAA GAAGGTTGCT TTAGACCACT |
| 2961 | CCACATCCAT ATTTGCATTT CAATGGCGGT CTTTTCAATG |
| 3001 | TCTCAGTTAA TTTTTCCTCA CTCGCCACAC TGAGTTTCTC |
| 3041 | CTTAGCTTTA TATATACGAT AGTGTATACT TTGTTTACAT |
| 3081 | GTTTTTTGGT GGAATGGAAC TTATGAGAGC ATATCAGATA |
| 3121 | TGTACTTGGG AAAATTAGTA GAAACTGTTT GTTTCTTTTT |
| 3161 | TTTTAACTCT GTTCTTTTGT ATATATCACT GAAGCTCGCA |
| 3201 | TATGTATAAT TCATGTAATG GAATTGCATC GCTTCTGTTT |
| 3241 | CCCTAAGTTA TTT |

JAZ4 is also a repressor of jasmonate responses. One example of an Arabidopsis thaliana jasmonate-zim-domain protein 4 (JAZ4) protein sequence is shown below (SEQ ID NO:52).

| | |
|---|---|
| 1 | MERDFLGLGS KLSPITVKEE TNEDSAPSRG MMDWSFSSKV |
| 41 | GSGPQFLSFG TSQQETRVNT VNDHLLSSAA MDQNQRTYFS |
| 81 | SLQEDRVFPG SSQQDQTTIT VSMSEPNYIN SFINHQHLGG |
| 121 | SPIMAPPVSV FPAPTTIRSS SKPLPPQLTI FYAGSVLVYQ |
| 161 | DIAPEKAQAI MLLAGNGPHA KPVSQPKPQK LVHHSLPTTD |
| 201 | PPTMPPSFLP SISYIVSETR SSGSNGVTGL GPTKTKASLA |
| 241 | STRNNQTAAF SMAPTVGLPQ TRKASLARFL EKRKERVINV |
| 281 | SPYYVDNKSS IDCRTLMSEC VSCPPAHHLH |

A chromosomal DNA sequence for the Arabidopsis thaliana jasmonate-zim-domain protein 4 (JAZ4) protein with SEQ ID NO:52 is shown below as SEQ ID NO:53.

| | |
|---|---|
| 1 | ATTAGAGGAA TCATAAATCG GCGGTGTGTG TAACTTCAAC |
| 41 | TCACGTTTTT CATTTCTCTC CAAAGTCCTT CAATTGTTAC |
| 81 | TAATTCTCTC TGATCTCTCA TTTCTTCTCT TCTCCGGTGA |
| 121 | CATTTTTTTT CTCCCCCGCG AAAGCTAAAC CGTTTTTGTA |
| 161 | TTCTCAACGA TTGATAAGCC TGATGGAGAG AGATTTTCTC |
| 201 | GGGCTGGGAT CAAAGTTATC TCCGATAACT GTGAAGGAGG |
| 241 | AAACTAACGA AGATTCAGGT AATTCATCTT CAACATCTTC |
| 281 | CATTATGATC TGATGATTGT GTTTTTCATC TCACTTTTTT |
| 321 | TTGTTTCTAT TTTTGTAATC TCTTTTTTTG TTTATTGTTC |
| 361 | AAGTACATAT ATATTGTTTT TCTAGCTTGA TTGGGAGTCC |
| 401 | TACTGTCTGG TTTTTTCTTG AACAAGAAAT TTTTTCTTCG |
| 441 | TTTTCTCGGG AAGAGAAAAA ATAAATTAGG GTTTCTTTTT |
| 481 | TCTTGATATA TATTTAAGAA ATTAGGTTTT AGTACTATAG |
| 521 | ACAGAAATTT AGCTACTCGA ATTTGTTTGA CGTAGCCGAT |
| 561 | GAAAAAACAC GTTTTGGGAC TCGATAGTTA GAAAATTCAT |
| 601 | ACGTTCACGA TCTACTTTTG AAGTTTTTTT CATTAAATAT |
| 641 | TTTTTGCAAA CTACAAATGT ACAAGTATAC AACTATACAA |
| 681 | GCAAACACCA AACTTGTTGA CGTTAGTAAT TTAACAAGTG |
| 721 | TTAGTATTAT CTTTGAAAAA TAATATTCAG AGAACAAACT |
| 761 | TGATTTTCTA GGTGACTAGG TGATGCATGT TTCTAAAGCT |
| 801 | GTTGGTAATG TTGAGTGTTT TCAAAATAAT TTCGTTTTTT |
| 841 | TCTTCAAACA GCCGACACCG ACAGAACAAA AATGCTATAT |
| 881 | TTTTTTTGTT GCTTACAAAA TTGATCAATT GGTTTCAATA |
| 921 | CAATAGTATC TTCTTTAGAA AAGATTGTTT TTTTCAAAGC |
| 1001 | CGGATTGAAT ATTGAGAATT AGAACATTGG CTGGTTATTC |
| 1041 | TTTTTGAAAA GTTTATGCCA TTTTTTAAGG TTTATTAAGC |
| 1081 | AACTTGAATT CTATCAGTAT TATTTAAAAA CGAAGACGTG |
| 1121 | AAATGTTGGG AAAAGAATGC GTTATATAGC GACCGGCTGA |
| 1161 | CGATTAGAGA TTTAACAACA AATGCAAGTT GAATTATATA |
| 1201 | AAAGCAAGAT TGATTGTGAC TTGATTAAGT TTTATTTCTA |
| 1241 | TCCAAGTAGA CTCATTGATT AAGTTAGGAT CATGTTGGGT |
| 1281 | ATTAAATTTA GATCAAGTTA CAATTTGGAT GAATAATTTA |
| 1321 | CTTACCCACG AGGAATTTAA TAGTTAGTTC TTGTCTTTTT |
| 1361 | ATATTCCGAA ACGTGCCATT TCTTGAAAGT ATTTGTATGA |
| 1401 | TCACTATTTT CCCCAGTGTG TTTGGCTTTA TGCAGATTTG |
| 1441 | TTCATTGTTG ATGAATCTAA TGTTAAGAGT CGTCCACTTT |
| 1481 | AGCATAGCTA GATCTGAGTG TTTCCTAGTT TGATAAAATC |
| 1521 | TAAAGACATT TGCTCATGTT TCAGCCCCAA GTAGAGGTAT |
| 1561 | GATGGATTGG TCATTCTCAA GCAAAGTCGG TTCTGGTCCT |
| 1601 | CAGTTTCTTT CTTTTGGGAC ATCCCAACAA GAAACGCGTG |
| 1641 | TAAACACAGT CAATGATCAT TTGCTTTCTT CTGCTGCAAT |
| 1681 | GGATCAAAAC CAGAGAACTT ACTTCAGCTC ACTACAGGTT |
| 1721 | AGGCTATTTC TTGAAAAGAA AAAAAGTAGT GATAAAGTGT |
| 1761 | GATTTAGTGA CCTTGTAAGA AAGCTTGGCA ATTGGTTTAG |
| 1801 | TTTCTTCTGG TCTCAAAATT GATACAAAAT GATCTCAGGA |
| 1841 | AGACAGAGTG TTCCCAGGTT CCAGTCAGCA AGACCAAACA |
| 1881 | ACCATCACAG TCTCCATGTC CGAACCAAAC TACATCAACA |
| 1921 | GTTTCATAAA CCACCAACAT TTAGGAGGAT CTCCTATCAT |
| 1961 | GGCACCTCCA GTTTCAGTAT TTCCTGCTCC AACCACTATT |
| 2001 | AGGCATGCAC TGCATTCTAT CTTCTTCTGT TTAACATCAG |
| 2041 | ATACAGAACC TCTTTACTTC TATAGTTGAC TCGAGCTCCT |
| 2081 | TTATGTTCAT CTCCAGATCT TCTTCAAAAC CACTTCCCCC |
| 2121 | TCAGTTGACA ATCTTTTATG CCGGTTCAGT ATTAGTTTAC |
| 2161 | CAAGACATAG CTCCTGAAAA GGTAACCAAA TTTCCTTCAA |
| 2221 | TATGTGTTAC ATTACAGTCC AAGCTATCCA CTGACTAAGT |
| 2241 | ATTCAATCAA AGAAATAAGT TTCACGTATA GACATGCTGA |
| 2281 | AGTTATAGAA AGTTACTAAC CTGGTTTCAA CATACAGTAT |
| 2321 | GTTAATGATT CATAGATATG ATAAATCTTT GTCCTTACTT |
| 2361 | CTTCATTTAT TTTGTATTCA TAGGCCCAAG CTATCATGTT |
| 2401 | GCTAGCCGGA AATGGACCTC ATGCTAAACC GGTTTCACAA |
| 2441 | CCTAAACCTC AAAAACTGGT TCATCACTCT CTTCCAACCA |
| 2481 | CTGATCCTCC AACTATGCCT CCTAGTTTCC TGCCTTCCAT |
| 2521 | CTCTTACATT GTCTCTGAAA CCAGAAGTAG TGGATCCAAC |
| 2561 | GGGGTTACTG GACTTGGACC AACAAAAACA AAGGCGAGTT |
| 2601 | TAGCATCCAC GCGCAACAAC CAAACTGCTG CCTTCTCTAT |
| 2641 | GGCTCCAACA GGTTATAAAT GAAGTCTTAA CTCCTATTAA |
| 2681 | TGTTTTGTCA TCAAACTTCT ATCTTAGGTT TAGTTTGTTA |
| 2721 | TAACCAAAAA ATCTTGCTAT GATTTAATAC AGTGGGTTTA |
| 2761 | CCACAAACAC GCAAAGCATC CTTGGCTCGG TTCTTAGAGA |
| 2801 | AACGCAAAGA AAGGTACTGA GCTACAAGAT TATTCACTTA |
| 2841 | TTCACAATAT CAAAACACAG GTTTGCTGTA TATTGGCTTC |
| 2881 | GTTTTCTTGC AGGGTCATTA ACGTATCACC TTATTACGTA |
| 2921 | GACAACAAGT CATCAATAGA CTGTAGAACA CTGATGTCTG |
| 2961 | AATGTGTAAG CTGTCCTCCA GCTCATCATC TGCACTAAAA |
| 3041 | CCAATTTAGA CCCCTCATTG TTCTAAAGGC TTTTTCTTTT |
| 3081 | TTCTCTGGCT CTGTATCCTA TAGACTATAG TATAGTTGTT |
| 3121 | ATAGCTTTTG TTTATTCAGA TTTTAGTACA CTGGGCTTGT |
| 3161 | AAAAGCAAGT TATTTATATA TATCCTATAA ATTTAATTTG |
| 3201 | GATACTGTAT GTTTTGTCTT TACTCTTGCA TGTGTATAAA |
| 3241 | AAACATAAAA GTAAGACTAT TCAAGCT |

JAZ9 is also a repressor of jasmonate responses. One example of an Arabidopsis thaliana jasmonate-zim-domain protein 9 (JAZ9) protein sequence is shown below (SEQ ID NO:54).

| | |
|---|---|
| 1 | MERDFLGLSD KQYLSNNVKH EVNDDAVEER GLSTKAAREW |
| 41 | GKSKVFATSS EMPSSDFQEA KAFPGAYQWG SVSAANVFRR |
| 81 | CQFGGAFQNA TPLLLGGSVP LPTHPSLVPR VASSGSSPQL |
| 121 | TIFYGGTISV FNDISPDKAQ AIMLCAGNGL KGETGDSKPV |
| 161 | REAERMYGKQ IHNTAATSSS SATHIDNFSR CRDTPVAATN |
| 201 | AMSMIESFNA APRNMIPSVP QARKASLARE LEKRKERLMS |
| 241 | AMPYKKMLLD LSTGESSGMN YSSTSPT |

A chromosomal DNA sequence for the Arabidopsis thaliana jasmonate-zim-domain protein 9 (JAZ9) protein with SEQ ID NO:54 is shown below as SEQ ID NO:55.

| | |
|---|---|
| 1 | GCAAAGAGTT AAATAAGCCT CTCCAAAAGT GTGTCTGTAA |
| 41 | CATTACCAAA ACGAAACCTT CCTTGTGGAT TCCCACTTCT |
| 81 | TTCTTCTGTT TTCTTCTTCC TCTTCTTTAA ATTGGATGTT |
| 121 | TTGGGCAAGA AACAGAGAGA AACACGTTAA TTTGAGAGTT |
| 161 | TGTCATTGAA TATTTGGTTT GCAATGGAAA GAGATTTTCT |
| 201 | GGGTTTGAGC GACAAGCAGT ATCTAAGTAA TAACGTTAAG |
| 241 | CATGAGGTTA ACGATGATGC TGTCGAAGAA CGAGGTTTGT |
| 281 | GTTCTTGTCT CGAGAATCTT TTATTTTAAT GTTTGAAGAA |
| 321 | GAGATCAGTT TTCACTTTTA ACATAGCCGT ATAAAGTTGT |
| 361 | TTATTTAAAT ATAATTTTTC AGATTCCAAA ACTTGAAAAA |
| 401 | AAAAAGATTC CATTAAATCT TTTATAAAAA TGAGATTGGA |
| 441 | TAGATTAGTC AAATTGACGA CCATAAAAAA TGATACTTAT |
| 481 | AGGGTTAAGT ACGAAGGCAG CTAGAGAATG GGGGAAGTCA |
| 521 | AAGGTTTTTG CTACTTCAAG TTTCATGCCT TCTTCAGATT |
| 561 | TCCAGGTTGG TTCATCTTAA AATTTAACTT ACTCTGTATC |
| 601 | AGTTTCAGAT GTTATGGCTA ATCTAATGGT TCTATAAGCT |
| 641 | ACCGCATAAT CATGGTCGTC TTTTAGCATG TGCAAGAGGA |
| 681 | GTACTCAATT ATGGTCTTGA TTAAAAAGAA GAATTTACTT |
| 721 | TCAAATTATG TTAAACACAT CAATCACATA TTTATGAGAA |
| 761 | AAGTTGTTTT CGTAAGAGAT AGCCACCGGA AAATGGTCGG |
| 801 | ATAAATGGCC GAACTTTATC ATTTTTGTGT ATGTGGCCAA |
| 841 | TCATTAACCA GGGAAAAAAA ATTGTTGGAT AAGTGCTAGT |
| 881 | TAAGAGCTGG TAGGGTCGGT CGTCTGCCAG CCGCAAAGTT |
| 921 | AGGGAAAAAA TAATTTAATA TTTTGTGGCG TTTGGTGTTT |
| 961 | GGCGTTTGGA TCACGTTTAT TTCTTGGCAT TTTTCTAAAT |
| 1001 | TTAGAATGTA CAAAAAATTT AAAGACGTTG ACGATTAAAA |
| 1041 | TTTGAATTTA ACAAATTAGG AGGCTAAGGC GTTTCCGGGT |
| 1081 | GCATACCAGT GGGGATCAGT TTCTGCGGCC AATGTTTTCC |
| 1121 | GCAGATGCCA ATTTGGTGGT GCGTTTCAAA ACGCGACGCC |
| 1161 | GCTTTTACTA GGCGGTTCAG TTCCTTTACC AACTCATCCT |
| 1201 | TCTCTTGTTC CACGGTAATT TCCATATTAT GATGCAAAAA |
| 1241 | CATTCAACAA TTTTTTTGCT CTTTTCATAT TTTGATTTGG |
| 1281 | TTATGTGGGT TTGTGGAAAC AGAGTGGCTT CCTCCGGATC |
| 1321 | ATCTCCTCAG CTCACAATCT TTTATGGCGG AACTATAAGC |
| 1361 | GTCTTTAATG ACATATCTCC CGATAAGGTA TATATAATCA |
| 1401 | AGATTCATAC AAATAACATT TACATAACAT TTACATGTTC |
| 1441 | TAAAACGGAC TATTCATGAT ATGTGAGTAG GCTCAAGCCA |
| 1481 | TCATGTTATG CGCCGGGAAC GGTTTGAAAG GTGAAACTGG |
| 1521 | AGATAGCAAA CCGGTTCGAG AAGCTGAAAG AATGTATGGA |
| 1561 | AAACAAATCC ATAACACTGC TGCTACCTCA TCAAGCTCTG |
| 1601 | CCACTCACAC TGATAATTTC TCAAGGTGTA GGGACACACC |
| 1641 | CGTTGCTGCG ACTAATGCAA TGAGCATGAT CGAATCATTC |
| 1681 | AATGCAGCTC CTCGTAACAT GATTCCTTCA GGTATGTGTG |
| 1721 | TCTAATATCA ACATCAAAAC AAAATATAAT CAAGATTTTT |
| 1761 | GCTTCCTCAA ATCATATGTC TAAACTCGAA AATTGCTTTT |
| 1801 | TTCCAGTCCC TCAAGCTCGG AAAGCATCCT TGGCTCGGTT |
| 1841 | CTTGGAGAAG CGCAAAGAGA GGTTTGATTT TGTATTTTTT |
| 1881 | TTCTTTATAG AAAATTTTGA GGTTTTTCAA TTGAATCTAA |
| 1921 | AAGAATTGAT GTTGTTGGTG CAGGCTTATG AGTGCAATGC |
| 1961 | CATACAAGAA GATGCTTCTT GATTTGTCGA CCGGAGAATC |
| 2001 | CAGTGGAATG AATTACTCTT CTACTTCTCC TACATAAAAC |
| 2041 | CTACACTTTT TTTTTTTTTT TTTACAATGG TAATTTGTAA |
| 2081 | TTGTAATCAT TAGATTATGA TTATATAGTT ACCATTTATA |
| 2121 | TTCTTACGAG CAGGAGAAGA CGTTAGGGCG TCTCTGTATT |
| 2161 | TGATCATTGT TTGTAATGCT TTGGTCTGTT TATTGTAGGA |
| 2201 | TTACATTATA ACTTTAAGAA CTAACAGATA TATGTTTGTC |
| 2241 | ATGGACTCAT GTCTGTCAAG AATTTAATAT CAAATAAAAT |
| 2281 | TCACTATAAT TTTTTTT |

JAZ10 is also a repressor of jasmonate responses. One example of an Arabidopsis thaliana jasmonate-zim-domain protein 10 (JAZ10) protein sequence is shown below (SEQ ID NO:56).

| | |
|---|---|
| 1 | MSKATIELDF LGLEKKQTNN APKPKFQKFL DRRRSFRDIQ |
| 41 | GAISKIDPEI IKSLLASTGN NSDSSAKSRS VPSTPREDQP |
| 81 | QIPISPVHAS LARSSTELVS GTVFMTIFYN GSVSVFQVSR |
| 121 | NKAGEIMKVA NEAASKKDES SMETDLSVIL PTTLRPKLFG |
| 161 | QNLEGDLPIA RRKSLQRFLE KRKERLVSTS PYYPTSA |

A chromosomal DNA sequence for the Arabidopsis thaliana jasmonate-zim-domain protein 10 (JAZ10) protein with SEQ ID NO:56 is shown below as SEQ ID NO:57.

| | |
|---|---|
| 1 | AAAAACTCTC ACATGAGAAA TCAGAATCCG TTATTATTCC |
| 41 | TCCATTTATT CATCTCAAAA CCCATATCTC TCTGTCTTGA |
| 81 | TCTCTCTCTC ACTTTCTAAT AAGATCAAAG AAGATGTCGA |
| 121 | AAGCTACCAT AGAACTCGAT TTCCTCGGAC TTGAGAAGAA |
| 161 | AGAAACCAAC AACGCTCCTA AGCCTAAGTT CCAGAAATTT |
| 201 | CTCGATCGCC GTCGTAGTTT CCGAGGTTCG TTTGGTTTTT |
| 241 | AGTCGCTCTC TCTTTTTTTT TTCTTGCGAT AAATCGAATT |
| 281 | TATTCATATG GAACTCCTGC AGATATTCAA GGTGCGATTT |
| 321 | CGAAAATCGA TCCGGAGATT ATGAAATCGC TGTTAGCTTC |
| 361 | CACTGGAAAC AATTCCGATT CATCGGCTAA ATCTCGTTCG |
| 401 | GTTCCGTCTA CTCCGAGGGA AGATCAGCCT CAGATCCCGA |
| 441 | TTTCTCCGGT CCACGCGTCT CTCGCCAGGT ATTTTTGTCT |
| 481 | TTCCGGTAAA GTTTTTTTTT TCTTTCTAAC TTTTTTGGCG |
| 521 | CTACCAGAAA AGACGAAAAA ATTTGAAATT CAAATTTTCA |
| 561 | AAACATTCAT TTTCCTCAGG TCTAGTACCG AACTCGTTTC |
| 601 | GGGAACTGTT CCTATGACGA TTTTCTACAA TGGAAGTGTT |
| 641 | TCAGTTTTCC AAGTGTCTCG TAACAAAGCT GGTGAAATTA |
| 681 | TGAAGGTCGC TAATGAAGCA GCATCTAAGA AAGACGAGTC |
| 721 | GTCGATGGAG ACAGATCTTT CGGTAATTCT TCCGACCACT |
| 761 | CTAAGACCAA AGCTCTTTGG CCAGAATCTA GAAGGAGGTT |
| 801 | AGTATAATAA AAATAAAAAT CACTTAGTGC TGGATTCTTC |
| 841 | TAGAATTTTA GTTACATATT ATTGCATGTA GAGATCTAAG |
| 881 | AAGAGTTTGT TGTTAGAGAG GAATTGGTTG CTAATTAGTT |
| 921 | TGGAATTAGA TATCAAAGAG TTAAAGACTA TAGTTTATGT |
| 961 | CTATACGTAT TAATATACGT TATTAATAAA AGTATAAACA |
| 1001 | TGTTGTTTAA TTTCTGATAA GAAACTGGTT TATGCGTGTG |
| 1041 | TATGCAGATC TTCCCATCGC AAGGAGAAAG TCACTGCAAC |
| 1081 | GTTTTCTCGA GAAGCGGAAG GAGAGGTAAT GATTCTTCAA |
| 1121 | CAATCCAAGG ATTTTTACCC CGAAATAATT AAAGAAAGGT |
| 1161 | TTTTATTTTT CTCTCTCTCG ACCTTTTTTT TACTATAAGT |
| 1201 | TATTTAAGAT AGTAATTATG GGTCCTGCCT CTTTTACTCT |
| 1241 | CACATACAAC TTAAGATTCA ACTAGTTTTG TTCAAGAACG |
| 1281 | CACATGCTTA TACGTAGATA GATAATGGAG ATCAGTAGTA |
| 1321 | ATATCGGTAT ACGTAGGTTA CTATTGTAAT GGAACTTTTA |
| 1361 | AAAAGCGCGT TGACTTTGAG TCTTTGACTC TAGTTCTGTT |
| 1401 | TGCTACACCG ACAAGTTATA TTTTTCAAAA TGATGAGAAA |
| 1441 | ACGAGGAGAA ACACCGGAAA AAAATTTGAA CTTTTACTTT |
| 1481 | TATCAGACCA TACGGCCAAA GAAAGATCTG TATATTATAT |
| 1521 | AAGTTATCAC AAAACGCGGT TTCACATTTT CTTTTTCGTC |
| 1561 | TTGTTGTGTT TGCAGATTAG TATCAACATC TCCTTACTAT |
| 1601 | CCGACATCGG CCTAAACGAT CTCTTTTTAG ATTGGGACAT |
| 1641 | GGACCAAATT TGTCTTTTTC AATCGGAAGA CATCCATGTT |
| 1681 | CGTTTTTGGA TTTGGCTTAT TTCCAATCTT CTTTTGAAGC |
| 1721 | CTTCTTCGTC GTTGCTAAAT CGTATACTAT TCACGACAAA |
| 1761 | CGTTTTTAGG AGATTACGTT ACCTACTAAG ATTATATATA |
| 1801 | TTGGTTTGTT TTTAAAAATG TCTATTATCT TTATTGTCAT |
| 1841 | TGATAGCTTG ATTTAAGAAG CTCTCTCTTA TCCCGTGACC |
| 1881 | TTCTACTTTT GTTTTATTTT TTAGTATATG GTAAAGAAAA |
| 1921 | TTATAAC |

Chromosomal sequences that encode repressors of jasmonic acid responses from many plant types and species can be modified to reduce or eliminate the expression and/or function of the encoded protein. For example, chromosomal sequences encoding jasmonic acid repressor genes from agriculturally important plants such as alfalfa (e.g., forage legume alfalfa), algae, avocado, barley, broccoli, Brussels sprouts, cabbage, canola, cassava, cauliflower, cole vegetables, collards, corn, crucifers, grain legumes, grasses (e.g., forage grasses), jatropa, kale, kohlrabi, maize, miscanthus, mustards, nut sedge, oats, oil firewood trees, oilseeds, potato, radish, rape, rapeseed, rice, rutabaga, sorghum, soybean, sugar beets, sugarcane, sunflower, switchgrass, tobacco, tomato, turnips, and/or wheat can be modified reduce or eliminate the expression and/or function of one or more encoded jasmonic acid regulatory proteins.

In some cases, more than one gene or chromosomal segment encoding a jasmonic acid regulatory protein can be modified to reduce or eliminate the expression and/or function of the encoded protein(s). In some cases, more than two genes or chromosomal segments encoding jasmonic acid regulatory proteins can be modified to reduce or eliminate the expression and/or function of the encoded proteins. In some cases, more than three genes or chromosomal segments encoding jasmonic acid regulatory proteins can be modified to reduce or eliminate the expression and/or function of the encoded proteins. In some cases, more than four genes or chromosomal segments encoding jasmonic acid regulatory proteins can be modified to reduce or eliminate the expression and/or function of the encoded proteins.

The following are examples of “JAZ-related” proteins and nucleic acids that can be modified to reduce or eliminate the expression and/or function thereof, and thereby generate plants with improved resistance to insects.

One example of a Brassica rapa protein called TIFY 10A-like (NCBI accession no. XP_009117562.1; GI:685367109) has significant sequence identity to the Arabidopsis thaliana JAZ1 protein with SEQ ID NO:48, as illustrated by the sequence comparison shown below. Domains of sequence homology are identified by asterisks below the sequence comparison.

| 73.0% identity in 211 residues overlap; Score: 634.0; Gap frequency: 11.4% | | |
|---|---|---|
| Seq 48 | 1 | MSLFPCEASNMDSMVQDVKPTNLFPRQPSFSSSSSSLPKEDVLKMTQ---TTRSVKPESQ |
| Seq 58 | 63 | MSLFPCEASNMEPIGQDVKPKNLFPRQPSFSSSSSSLPKEDILKMTQATSSTRSVKPEPQ |
| | | *********** ***** ******************** ***** ******* * |
| Seq 48 | 58 | TAPLTIFYAGQVIVFNDFSAEKAKEVINLASKGTANS-------------------LAKN |
| Seq 58 | 123 | TAPLTIFYGGQVIVFNDFSAEKAKEVMDLASKGTANTFTGFTSNVNNNIQSVYTTNLANN |
| | | ******** ***************** ******** ** * |
| Seq 48 | 99 | QTDIRSNIATIANQVPHPRKTTTQEPIQSSPTPLT-ELPIARRASLHRFLEKRKDRVTSK |
| Seq 58 | 183 | QTEMRSNIAPIPNQLPHLMKTTTQNPVQSSSTAMACELPIARRASLHRFLAKRKDRVTSK |
| | | ** ***** * ** ** ***** * *** * ************** ********* |
| Seq 48 | 158 | APYQLCDPAKASSNPQTTGNM-SWLGLAAEI |
| Seq 58 | 243 | APYQLNDPAKASSKPQTGDNTTSWLGLAAEM |
| | | ***** ******* *** * ******** |

This JAZ-related Brassica rapa protein, called TIFY 10A-like (NCBI accession no. XP_009117562.1; GI:685367109), has the following sequence (SEQ ID NO:58).

| | |
|---|---|
| 1 | MSSPMESSDF AATRRFSRKP SFSQTCSRLS QYLKENGSFG |
| 41 | DLSLGMACKP EVNGISRQPT TTMSLFPCEA SNMEPIGQDV |
| 81 | KPKNLFPRQP SFSSSSSSLP KEDILKMTQA TSSTRSVKPE |
| 121 | PQTAPLTIFY GGQVIVFNDF SAEKAKEVMD LASKGTANTF |
| 161 | TGFTSNVNNN IQSVYTTNLA NNQTEMRSNI APIPNQLPHL |
| 201 | MKTTTQNPVQ SSSTAMACEL PIARRASLHR FLAKRKDRVT |
| 241 | SKAPYQLNDP AKASSKPQTG DNTTSWLGLA AEM |

A cDNA encoding the SEQ ID NO:58 protein is available as NCBI accession number XM_009119314.1 (GI:685367108), and a chromosomal segment encoding the SEQ ID NO:58 protein is available as NCBI accession number AENI01008623.1 (GI:339949964).

One example of a Brassica oleracea protein, also referred to as protein TIFY 10A-like (NCBI accession no. XP_013583936.1; GI:922487335), has significant sequence identity to the Arabidopsis thaliana JAZ1 protein with SEQ ID NO:48, as illustrated by the sequence comparison shown below. Domains of sequence homology are identified by asterisks below the sequence comparison.

| 72.9% identity in 192 residues overlap; Score: 633.0; Gap frequency: 2.6% | | |
|---|---|---|
| Seq 48 | 1 | MSLFPCEASNMDSMV--QDVKPTNLFPRQPSFSSSSSSLPKEDVLKMTQTT-RSVKPESQ |
| Seq 59 | 61 | MSLFPCEASNVGSMAAAQDVKPKNLFPRQPSFSSSSSSIPKEDVPKMTQTTTRSLKPEPQ |
| | | ********** ** ***** *************** ***** ****** ** *** * |
| Seq 48 | 58 | TAPLTIFYAGQVIVFNDFSAEKAKEVINLASKGTANSLAKNQTDIRSNIATIANQVPHPR |
| Seq 59 | 121 | TAPLTIFYGGQVIVFNDFSAEKAKEVMNLANKGTANTFTGFTSTLNNNIAPTPNQVPHLM |
| | | ******** ***************** *** ***** *** ***** |
| Seq 48 | 118 | KTTTQEPIQSSPTPLT-ELPIARRASLHRFLEKRKDRVTSKAPYQLCDPAKASSNPQTTG |
| Seq 59 | 181 | KAATQDPKQTSSAAMACELPIARRASLHRFLAKRKDRVTSKAPYQLNDPAKAYSKPQTGN |
| | | * ** * * * ************** ************** ***** * *** |
| Seq 48 | 177 | NM-SWLGLAAEI |
| Seq 59 | 241 | TTTSWLGLAADM |
| | | ******* |

This JAZ-related Brassica oleracea protein referred to as protein TIFY 10A-like (NCBI accession no. XP_013583936.1; GI:922487335) has the following sequence (SEQ ID NO:59).

| | |
|---|---|
| 1 | MSSSMECSTT RRSSSGKPSF SLTCSRLSQY LKENGSFGDL |
| 41 | SLGMSCKPDT NGMSRKPTTT MSLFPCEASN VGSMAAAQDV |
| 81 | KPKNLFPRQP SFSSSSSSIP KEDVPKMTQT TTRSLKPEPQ |
| 121 | TAPLTIFYGG QVIVFNDFSA EKAKEVMNLA NKGTANTFTG |
| 161 | FTSTLNNNIA PTPNQVPHLM KAATQDPKQT SSAAMACELP |
| 201 | IARRASLHRF LAKRKDRVTS KAPYQLNDPA KAYSKPQTGN |
| 241 | TTTSWLGLAA DM |

A cDNA encoding the SEQ ID NO:59 protein is available as NCBI accession number XM_013728482.1 (GI:922487334), and a chromosomal segment encoding the SEQ ID NO:59 protein is available as NCBI accession number NC_027752.1 (GI:919506312).

An uncharacterized Zea mays protein referred to as LOC100276383 (NCBI accession no. NP_001308779.1 (GI:1013071036) has significant sequence identity to the Arabidopsis thaliana JAZ1 protein with SEQ ID NO:48, as illustrated by the sequence comparison shown below. Domains of sequence homology are identified by asterisks below the sequence comparison.

| 39.0% identity in 123 residues overlap; Score: 201.0; Gap frequency: 0.8% | | |
|---|---|---|
| Seq 48 | 61 | LTIFYAGQVIVFNDFSAEKAKEVINLASKGTANSLAKNQTDIRSNIATIANQVPHPRKTT |
| Seq 60 | 100 | LTIFYGGKVLVFDDFPADKAKDLMQLASKGSPVVQNVALPQPSAAAAVTTDKAVLDPVIS |
| | | ***** * * ** ** * *** ***** * |
| Seq 48 | 121 | TQEPIQSSPTPLTELPIARRASLHRFLEKRKDRVTSKAPYQLCDPAKASSNPQTTGNMSW |
| Seq 60 | 160 | LAAAKKPARTNASDMPIMRKASLHRFLEKRKDRLNAKTPYQTA-PSDAAPVKKEPESQPW |
| | | * ** * ************* * *** * * * |
| Seq 48 | 181 | LGL |
| Seq 60 | 219 | LGL |
| | | *** |

This JAZ-related uncharacterized Zea mays protein referred to as LOC100276383 (NCBI accession no. NP_001308779.1 (GI:1013071036) has the following sequence (SEQ ID NO:60).

| | |
|---|---|
| 1 | MAASARPGER ATSFAVACSL LSRFVRQNGV AAADLGLRIK |
| 41 | GEVEQQRTPA TTNSLPGAEG EEVERRKETM ELFPQSVGFS |
| 81 | IKDAAAPREE QGDKEKPKQL TIFYGGKVLV FDDFPADKAK |
| 121 | DLMQLASKGS PVVQNVALPQ PSAAAAVTTD KAVLDPVISL |
| 161 | AAAKKPARTN ASDMPIMRKA SLHRFLEKRK DRLNAKTPYQ |
| 201 | TAPSDAAPVK KEPESQPWLG LGPNAVDSSL NLS |

A cDNA encoding the SEQ ID NO:60 protein is available as NCBI accession number NM_001321850.1 (GI:1013071035), and a chromosomal segment encoding the SEQ ID NO:60 protein is on Zea mays chromosome 7 at NC_024465.1 (165496371 . . . 165497455), sequence available as NCBI accession number NC_024465.1 (GI:662248746).

A Glycine max protein referred to as protein TIFY 10A-like (NCBI accession no. NP_001276307.1 (GI:574584782)) has significant sequence identity to the Arabidopsis thaliana JAZ1 protein with SEQ ID NO:48, as illustrated by the sequence comparison shown below. Domains of sequence homology are identified by asterisks below the sequence comparison.

| 45.5% identity in 145 residues overlap; Score: 271.0; Gap frequency: 4.8% | | |
|---|---|---|
| Seq 48 | 42 | VLKMTQTTRSVKPESQTAPLTIFYAGQVIVFNDFSAEKAKEVINLASKGTANSLAKNQTD |
| Seq 61 | 101 | IMVKSSAFKSMEKEPKAAQLTIFYAGQVVVFDDFPAEKLEEITSLAGKGISQS-----QN |
| | | * * * ********* ** ** *** * ** ** * |
| Seq 48 | 102 | IRSNIATIANQVPHPRKTTTQEPIQSSPTPLTELPIARRASLHRFLEKRKDRVTSKAPYQ |
| Seq 61 | 156 | TSAYAHTHNQQVNHPSFVPNISPQAPSRPLVCDLPIARKASLHRFLSKRKDRIAAKAPYQ |
| | | * ** ** * * ***** ******* ***** ***** |
| Seq 48 | 162 | LCDPAKASSNPQTTGNMSWLGLAAE |
| Seq 61 | 216 | INNPNSASSKPAE--SMSWLGLGAQ |
| | | * *** * ****** * |

This JAZ-related Glycine max protein referred to as protein TIFY 10A-like (NCBI accession no. NP_001276307.1 (GI:574584782) has the following sequence (SEQ ID NO:61).

| | |
|---|---|
| 1 | MSSSSEYLVF SSHHPANSPA EKSTFSQTCS LLSQYIKEKG |
| 41 | TFGDLTLGMT CTAETNGSPE TSCHSATTME LFPTIITQRN |
| 61 | PTTVDFLSPQ TAYPHHSEVP IMVKSSAFKS MEKEPKAAQL |
| 121 | TIFYAGQVVV FDDFPAEKLE EITSLAGKGI SQSQNTSAYA |
| 161 | HTHNQQVNHP SFVPNISPQA PSRPLVCDLP IARKASLHRF |
| 201 | LSKRKDRIAA KAPYQINNPN SASSKPAESM SWLGLGAQST |

A cDNA encoding the SEQ ID NO:61 protein is available as NCBI accession number NM_001289378.1 (GI:574584781), and a chromosomal segment encoding the SEQ ID NO:61 protein is on Glycine max chromosome 13 at NC_016100.2 (22541885 . . . 22544240), sequence available as NCBI accession number NC_016100.2 (GI:952545303).

An Oryza sativa protein referred to as protein TIFY 10b (Japonica Group; NCBI accession no. XP_015647536.1 (GI:1002286463) has significant sequence identity to the Arabidopsis thaliana JAZ1 protein with SEQ ID NO:48, as illustrated by the sequence comparison shown below. Domains of sequence homology are identified by asterisks below the sequence comparison.

| 38.5% identity in 156 residues overlap; Score: 213.0; Gap frequency: 4.5% | | |
|---|---|---|
| Seq 48 | 34 | SSSLPKEDVLKMTQTTRSVKPESQTAPLTIFYAGQVIVFNDFSAEKAKEVINLASKGTA- |
| Seq 62 | 77 | SAGFGQQDAITADSAADAREQEPEKRQLTIFYGGKVLVFNDFPADKAKGLMQLASKGSPV |
| | | * * * ***** * * ***** * *** ***** |
| Seq 48 | 93 | ---NSLAKNQTDIRSNI-ATIANQVPHPRKTTTQEPIQS-SPTPLTELPIARRASLHPFL |
| Seq 62 | 137 | APQNAAAPAPAAVTDNTKAPMAVPAPVSSLPTAQADAQKPARANASDMPIARKASLHRFL |
| | | * * * * * * * * * ********* ** |
| Seq 48 | 148 | EKRKDRVTSKAPYQLCDPAKASSNPQTTGNMSWLGL |
| Seq 62 | 197 | EKRKDRLNAKTPYQ-ASPSDATPVKKEPESQPWLGL |
| | | ****** * *** * * **** |

This JAZ-related Oryza sativa protein referred to as protein TIFY 10b (Japonica Group; NCBI accession no. XP_015647536.1 (GI:1002286463) has the following sequence (SEQ ID NO:62).

| | |
|---|---|
| 1 | MAASARPVGV GGERATSFAM ACSLLSRYVR QNGAAAAELG |
| 41 | LGIRGEGEAP RAAPATMSLL PGEAERKKET MELFPQSAGF |
| 81 | GQQDAITADS AADAREQEPE KRQLTIFYGG KVLVFNDFPA |
| 121 | DKAKGLMQLA SKGSPVAPQN AAAPAPAAVT DNTKAPMAVP |
| 161 | APVSSLPTAQ ADAQKPARAN ASDMPIARKA SLHRFLEKRK |
| 201 | DRLNAKTPYQ ASPSDATPVK KEPESQPWLG LGPNAVVKPI |
| 241 | ERGQ |

A cDNA encoding the SEQ ID NO:62 protein is available as NCBI accession number XM_015792050.1 (GI:1002286462), and a chromosomal segment encoding the SEQ ID NO:62 protein is on Oryza sativa chromosome 7 at NC_029262.1 (25347990 . . . 25350243), sequence available as NCBI accession number NC_029262.1 (GI:996703426).

A Zea mays protein referred to as protein TIFY 6A-like (NCBI accession no. NP_001288506.1 (GI: 673921704) has significant sequence identity to the Arabidopsis thaliana JAZ3 protein with SEQ ID NO:50, as illustrated by the sequence comparison shown below. Domains of sequence homology are identified by asterisks below the sequence comparison.

| 36.6% identity in 161 residues overlap; Score: 165.0; Gap frequency: 6.8% | | |
|---|---|---|
| Seq 50 | 177 | AQLTIFYAGSVCVYDDISPEKAKAIMLLAGNGSSMPQVFSPPQTHQQVVHHTRASVDSSA |
| Seq 63 | 167 | AQLTIFYAGSVNVFNNVSAEKAQELMFLASRGSSAPVACKPEAPPTLAPAKVTAPEVLLP |
| | | *********** * * *** * ** *** * * * |
| Seq 50 | 237 | MPPSFMPTISYLSPEAGSSTNGLGATKATRGLTSTYH-NNQANGSNINCPVP-------- |
| Seq 63 | 227 | AKQMLFQKPQHLSPPPSSVPGILQSAALPRSASSSSNLDSPAPKSSVPLAVPPVSQAPPA |
| | | *** * * * * * * ** |
| Seq 50 | 288 | --VSCSTNVMAPTVALPLARKASLARFLEKRKERVTSVSPY |
| Seq 63 | 287 | TLIATTTAAAIMPRAVPQARKASLARFLEKRKERVTTAAPY |
| | | * * * ****************** ** |

This JAZ-related Zea mays protein referred to as protein TIFY 6A-like (NCBI accession no. NP_001288506.1 (GI: 673921704) has the following sequence (SEQ ID NO:63).

| | |
|---|---|
| 1 | MERDFLAAIG KEQQHPRKEK AGGGAEESAY FGAAAVPAMD |
| 41 | WSFASKPCAA PALMSFRSAA REEPSFPQFS ALDGTKNTAP |
| 81 | RMLTHQRSFG PDSTQYAALH RAQNGARVVP VSSPFSQSNP |
| 121 | MFRVQSSPSL PNSTAFKQPP FAISNAVASS TVGSYGGTRD |
| 161 | AVRPRTAQLT IFYAGSVNVF NNVSAEKAQE LMFLASRGSS |
| 201 | APVACKPEAP PTLAPAKVTA PEVLLPAKQM LFQKPQHLSP |
| 241 | PPSSVPGILQ SAALPRSASS SSNLDSPAPK SSVPLAVPPV |
| 281 | SQAPPATLIA TTTAAAIMPR AVPQARKASL ARFLEKRKER |
| 321 | VTTAAPYPSA KSPLESSDTF GSGSASANAN DKSSCTDIAL |
| 361 | SSNHEESLCL GGQPRSIISF SEESPSTKLQ I |

A cDNA encoding the SEQ ID NO:63 protein is available as NCBI accession number NM_001301577.1 (GI:673921703), and a chromosomal segment encoding the SEQ ID NO:63 protein is on Zea mays chromosome 2 at NC_024460.1 (180086924 . . . 180089758, complement), sequence available as NCBI accession number NC_024460.1 (GI:662249846).

A Glycine max protein referred to as protein TIFY 6B-like isoform X1 (NCBI accession no. XP_003534135.1 (GI:356531138) has significant sequence identity to the Arabidopsis thaliana JAZ3 protein with SEQ ID NO:50, as illustrated by the sequence comparison shown below. Domains of sequence homology are identified by asterisks below the sequence comparison.

| 38.9% identity in 378 residues overlap; Score: 417.0; Gap frequency: 8.5% | | |
|---|---|---|
| Seq 50 | 1 | MERDFLGLGSKNSP-ITVKEETSESSRDSAPNRGMNWSFSNKVSASSSQFLSFRPTQEDR |
| Seq 64 | 1 | MEREFFGLSSKNGAWTTMKDDAVNKSRDQVRSSGMQWSFPNKVSALP-QFLSFKTNQEDK |
| | | *** * ** *** * * *** ** *** ***** ***** *** |
| Seq 50 | 60 | HRKSGNYHLPHSGSFMPSSVADVYDSTRKA--------------PYSSVQGVRMFPNS-- |
| Seq 64 | 60 | PRKTILEPLASSG-YMAMSTQYAFDSNQKSFLGLTNRNLSISKHAAGNKQGMTVYPLQCC |
| | | ** * ** * * ** * ** * |
| Seq 50 | 104 | -NQHEETNAVSMSMPGFQ-----SHHYAPGGRSFMNNNNNSQPLVGVPIMAPPISILPPP |
| Seq 64 | 119 | DAQSEEARIFSVSNQSNQVSPVLQSNLASTGLNMVNSVIKPQPF-GSKSSGTPLSILPSI |
| | | * ** * * * * * * ** * * **** |
| Seq 50 | 158 | GSIVGTTDIRSSSKPIGSPAQLTIFYAGSVCVYDDISPEKAKAIMLLAGNGSSMPQVFSP |
| Seq 64 | 178 | GSIVGSTDLRNNSKSSTMPTQLTIFYAGSVCVYDDISPEKAKAIMLMAGNGYTPTEKMEL |
| | | ***** ** * ** * ************************** **** |
| Seq 50 | 218 | PQTHQQVVHHTRASVD----SSAMPPSFMPTISYLSPEAGSSTNGLGATKATRGLTSTYH |
| Seq 64 | 238 | PTVKLQPAISIPSKDDGFMISQSYPPSTFPTPLPLTSHVNSQPGGGSSSNKEISIIRQVG |
| | | * * * * *** ** * * * |
| Seq 50 | 274 | NNQANGSNINCPV--PVSCSTNVMAPTVALPLARKASLARFLEKRKERVTSVSPYCLDKK |
| Seq 64 | 298 | PSTAPTNHLESPIIGSIGSASKEKAQPVCLPQARKASLARFLEKRKGRMMRTSPYLYMSK |
| | | * * * * ** ************** * *** * |
| Seq 50 | 332 | SSTDCRRSMSECISSSLS |
| Seq 64 | 358 | KSPECSSSGSDSVSFSLN |
| | | * * * * * ** |

This JAZ-related Glycine max protein referred to as protein TIFY 6B-like isoform X1 (NCBI accession no. XP_003534135.1 (GI:356531138) has the following sequence (SEQ ID NO:64).

| | |
|---|---|
| 1 | MEREFFGLSS KNGAWTTMKD DAVNKSRDQV RSSGMQWSFP |
| 41 | NKVSALPQFL SFKTNQEDKP RKTILEPLAS SGYMAMSTQY |
| 81 | AFDSNQKSFL GLTNRNLSIS KHAAGNKQGM TVYPLQCCDA |
| 121 | QSEEARIFSV SNQSNQVSPV LQSNLASTGL NMVNSVIKPQ |
| 161 | PFGSKSSGTP LSILPSIGSI VGSTDLRNNS KSSTMPTQLT |
| 201 | IFYAGSVCVY DDISPEKAKA IMLMAGNGYT PTEKMELPTV |
| 241 | KLQPAISIPS KDDGFMISQS YPPSTFPTPL PLTSHVNSQP |
| 281 | GGGSSSNKEI SIIRQVGPST APTNHLESPI IGSIGSASKE |
| 321 | KAQPVCLPQA RKASLARFLE KRKGRMMRTS PYLYMSKKSP |
| 361 | ECSSSGSDSV SFSLNFSGSC SLPATN |

A cDNA encoding the SEQ ID NO:64 protein is available as NCBI accession number XM_003534087.3 (GI:955341633), and a chromosomal segment encoding the SEQ ID NO:64 protein is on Glycine max chromosome 9 at NC_016096.2 (39883473 . . . 39889992), sequence available as NCBI accession number NC_016096.2 (GI:952545307).

An Oryza sativa protein referred to as protein TIFY 6b (NCBI accession no. XP_015612402.1 (GI:1002297967) has significant sequence identity to the Arabidopsis thaliana JAZ3 protein with SEQ ID NO:50, as illustrated by the sequence comparison shown below. Domains of sequence homology are identified by asterisks below the sequence comparison.

| 37.3% identity in 177 residues overlap; Score: 142.0; Gap frequency: 10.2% | | |
|---|---|---|
| Seq 50 | 172 | PIGSPAQLTIFYAGSVCVYDDISPEKAKAIMLLAGNGS---------SMPQ--VFSPPQT |
| Seq 65 | 187 | PKAKAAQLTIFYAGSVNVFNNVSPEKAQELMFLASRGSLPSAPTTVARMPEAHVFPPAKV |
| | | * *********** * ***** * ** ** ** ** * |
| Seq 50 | 221 | HQQVVHHTRASV-DSSAMPPSFMPTISY---LSPEAGSSTNGLGATKATRGLTSTYHNNQ |
| Seq 65 | 247 | TVPEVSPTKPMMLQKPQLVSSPVPAISKPISVVSQATSLPRSASSSNVDSNVTKSSGPLV |
| | | * * * * ** * * * |
| Seq 50 | 277 | ANGSNINCPV-PVSCSTNVMAPTV--ALPLARKASLARFLEKRKERVTSVSPYCLDK |
| Seq 65 | 307 | VPPTSLPPPAQPETLATTTAAAIMPRAVPQARKASLARFLEKRKERVTTVAPYPLAK |
| | | * * * * * * ****************** * ** * * |

This JAZ-related Oryza sativa protein, referred to as protein TIFY 6b (NCBI accession no. XP_015612402.1 (GI:1002297967), has the following sequence (SEQ ID NO:65).

| | |
|---|---|
| 1 | MERDFLGAIG KDEEQRRHAE ERKESDYFGA GGGAAAAAMD |
| 41 | WSFASRAALM SFRSSSSAAA AAAREETREL AFPHFSALDG |
| 81 | AKMQQASHVL ARQKSFGAES HGIPQYALLA AVHGAHRGQP |
| 121 | PHVLNGARVI PASSPFNPNN PMFRVQSSPN LPNAVGAGGG |
| 161 | AFKQPPFAMG NAVAGSTVGV YGTRDMPKAK AAQLTIFYAG |
| 201 | SVNVFNNVSP EKAQELMFLA SRGSLPSAPT TVARMPEAHV |
| 241 | FPPAKVTVPE VSPTKPMMLQ KPQLVSSPVP AISKPISVVS |
| 281 | QATSLPRSAS SSNVDSNVTK SSGPLVVPPT SLPPPAQPET |
| 321 | LATTTAAAIM PRAVPQARKA SLARFLEKRK ERVTTVAPYP |
| 361 | LAKSPLESSD TMGSANDNKS SCTDIALSSN RDESLSLGQP |
| 401 | RTISFCEESP STKLQI |

A cDNA encoding the SEQ ID NO:65 protein is available as NCBI accession number XM_015756916.1 (GI:1002297966), and a chromosomal segment encoding the SEQ ID NO:65 protein is on Oryza sativa chromosome 9 at NC_029264.1 (14056084 . . . 14060320, complement), sequence available as NCBI accession number NC_029264.1 (GI:996703424).

An uncharacterized Zea mays protein referred to as LOC100273108 (NCBI accession no. NP_001141029.1 (GI:226500626) has significant sequence identity to the Arabidopsis thaliana JAZ4 protein with SEQ ID NO:52, as illustrated by the sequence comparison shown below. Domains of sequence homology are identified by asterisks below the sequence comparison.

55.0% identity in 40 residues overlap;
Score: 106.0; Gap frequency: 0.0%

| | | |
|---|---|---|
| Seq 52 | 138 | RSSSKPLPPQLTIFYAGSVLVYQDIAPEKAQAIMLLAGNG |
| Seq 66 | 172 | RDVVRPKTAQLTIFYAGSVNVFDNVSAEKAQELMLLASRG |
| | | * * ********** * **** **** * |

76.9% identity in 26 residues overlap;
Score: 102.0; Gap frequency: 0.0%

| | | |
|---|---|---|
| Seq 52 | 258 | LPQTRKASLARFLEKRKERVINVSPY |
| Seq 66 | 321 | VPQARKASLARFLEKRKERVTTAAPY |
| | | ** **************** ** |

48.0% identity in 25 residues overlap;
Score: 66.0; Gap frequency: 0.0%

| | | |
|---|---|---|
| Seq 52 | 25 | SAPSRGMMDWSFSSKVGSGPQFLSF |
| Seq 66 | 47 | AAAAAPAMDWSFASKPGAAPALMSF |
| | | * ***** ** * * ** |

38.5% identity in 26 residues overlap;
Score: 37.0; Gap frequency: 0.0%

| | | |
|---|---|---|
| Seq 52 | 1 | MERDFLGLGSKLSPITVKEETNEDSA |
| Seq 66 | 14 | MERDFLAAIGKEQQHPHKEEAGAEES |
| | | ****** * *** |

This JAZ-related uncharacterized Zea mays protein, referred to as LOC100273108 (NCBI accession no. NP_001141029.1 (GI:226500626), has the following sequence (SEQ ID NO:66).

| | |
|---|---|
| 1 | MAKSGASFPE SSWMERDFLA AIGKEQQHPH KEEAGAEESA |
| 41 | YTGGAGAAAA APAMDWSFAS KPGAAPALMS FRSASFPQFS |
| 81 | SFDGAKNPAP RILTHQRSFG PDSTHYAAAH RTQHALNGAR |
| 121 | VTPVSSPFNQ NSPMFRVQSS PSLPNGTAFK QPPFAINNNA |
| 161 | AASSTVGFYG TRDVVRPKTA QLTIFYAGSV NVFDNVSAEK |
| 201 | AQELMLLASR GSLPSSAPVA RKPEAPILAP AKVTAPEVLH |
| 241 | ATQMLFQKPQ HVSPPSSAIS KPIPGILQAA SLPRSASSSN |
| 281 | LDSPFPKSSV PFPVSPVSQA PRAQPATIAA TTAAAIMPRA |
| 321 | VPQARKASLA RFLEKRKERV TTAAPYPSAK SPMESSDTFG |
| 361 | SGSANDKSSC TDIALSSNHE ESLCLGQPRN ISFIQESPST |
| 401 | KLQI |

A cDNA encoding the SEQ ID NO:66 protein is available as NCBI accession number NM_001147557.1 (GI:226500625), and a chromosomal segment encoding the SEQ ID NO:66 protein is on Zea mays chromosome 7 at NC_024465.1 (108871356 . . . 108874213, complement), sequence available as NCBI accession number NC_024465.1 (GI:662248746).

A Glycine max protein, referred to as protein TIFY 6B isoform X5 (NCBI accession number XP_006580448.1 (GI:571456655), has significant sequence identity to the Arabidopsis thaliana JAZ4 protein with SEQ ID NO:52, as illustrated by the sequence comparison shown below. Domains of sequence homology are identified with asterisks below the sequence comparison.

| 37.0% identity in 322 residues overlap; Score: 273.0; Gap frequency: 8.7% | | |
|---|---|---|
| Seq 52 | 1 | MERDFLGLGSKLSPITVKEETNEDSAPSRG-----MMDWSFSSKVGSGPQFLSFGTSQQE |
| Seq 67 | 1 | MERDFMGLNLKEPLAVVKEEMNNDGCKNSGFKKGRIAQWPFSNKVSALPHLMSFKASQDD |
| | | ***** ** * **** * * * * ** ** * ** ** |
| Seq 52 | 56 | TRVNTVNDHLLSSAAMD-QNQRTYFSSLQEDRVFPGSSQQDQTTITVSMSEPNYINSFI- |
| Seq 67 | 61 | KTKNTVSDTLSSSGFMSILSQEAFDTSQKRSAGEPQMFSVPNQAISVSLGNPFLKNHFAA |
| | | *** * * ** * * * * * ** * * * |
| Seq 52 | 114 | --NHQHLGGSPIMAP----PVSVFPAPTTIRSSSKPLPPQLTIFYAGSVLVYQDIAPEKA |
| Seq 67 | 121 | AGQKPLLGGIPVTTSHSVLPSAVAVAGMTESCNSVKPSAQLTIFYAGTVNIFDDISAEKA |
| | | *** * * * * * * ******** * ** *** |
| Seq 52 | 168 | QAIMLLAGNG-PHAKPVSQPKPQKLVHHSLPTTDPPTMPPSFLPSISYIVSETRSSGSNG |
| Seq 67 | 181 | QAIMLLAGNSLSAASNMAQPNVQVPISKLGAGAGVPVSQPANTSPGSGLSSPLSVSSHTG |
| | | ********* * ** * * * * * * * |
| Seq 52 | 227 | V-TGLGPTKTKASLASTRNN--QTAAFSMAP----------TVGLPQTRKASLARFLEKR |
| Seq 67 | 241 | VQSGSGLTSTDEFLAAKTTGVPNTPICNVEPPKVVSATTMLTSAVPQARKASLARFLEKR |
| | | * * * * * ** * * * ** ************ |
| Seq 52 | 274 | KERVINVSPYYVDNKSSIDCRT |
| Seq 67 | 301 | KERVMSAAPYNL-NKKSEECAT |
| | | **** ** ** * * * |

This JAZ-related Glycine max protein, referred to as protein TIFY 6B isoform X5 (NCBI accession number XP_006580448.1 (GI:571456655), has the following sequence (SEQ ID NO:67).

| | |
|---|---|
| 1 | MERDFMGLNL KEPLAVVKEE MNNDGCKNSG FKKGRIAQWP |
| 41 | FSNKVSALPH LMSFKASQDD KTKNTVSDTL SSSGFMSILS |
| 61 | QEAFDTSQKR SAGEPQMFSV PNQAISVSLG NPFLKNHFAA |
| 121 | AGQKPLLGGI PVTTSHSVLP SAVAVAGMTE SCNSVKPSAQ |
| 161 | LTIFYAGTVN IFDDISAEKA QAIMLLAGNS LSAASNMAQP |
| 201 | NVQVPISKLG AGAGVPVSQP ANTSPGSGLS SPLSVSSHTG |
| 241 | VQSGSGLTST DEFLAAKTTG VPNTPICNVE PPKVVSATTM |
| 281 | LTSAVPQARK ASLARFLEKR KERVMSAAPY NLNKKSEECA |
| 321 | TAEYAGVNFS ATNTVLAKQG |

A cDNA encoding the SEQ ID NO:67 protein is available as NCBI accession number XM_006580385.2 (GI:955322108), and a chromosomal segment encoding the SEQ ID NO:67 protein is on Glycine max chromosome 5 at NC_016092.2 (41222014 . . . 41225906), sequence available as NCBI accession number NC_016092.2 (GI:952545311).

An Oryza sativa protein, referred to as protein TIFY 6a isoform X2 (NCBI accession number XP_015651050.1 (GI:1002293416), has significant sequence identity to the Arabidopsis thaliana JAZ4 protein with SEQ ID NO:52, as illustrated by the sequence comparison shown below. Domains of sequence homology are identified by asterisks below each sequence comparison.

| 80.8% identity in 26 residues overlap; Score: 106.0; Gap frequency: 0.0% | | |
|---|---|---|
| Seq 52 | 258 | LPQTRKASLARFLEKRKERVINVSPY |
| Seq 68 | 342 | VPQARKASLARFLEKRKERVSSVAPY |
| | | ** **************** * ** |
| 44.7% identity in 47 residues overlap; Score: 87.0; Gap frequency: 0.0% | | |
| Seq 52 | 138 | RSSSKPLPPQLTIFYAGSVLVYQDIAPEKAQAIMLLAGNGPHAKPVS |
| Seq 68 | 193 | RDLQNPKVTQMTIFYDGLVNVFDNIPVEKAQELMLLASRASIPSPPS |
| | | * * * **** * * * * **** **** * * |
| 41.7% identity in 24 residues overlap; Score: 39.0; Gap frequency: 0.0% | | |
| Seq 52 | 1 | MERDFLGLGSKLSPITVKEETNED |
| Seq 68 | 1 | MERDFLGAIWRKEEPAGKPEEHSD |
| | | ******* * * * |
| 19.0% identity in 63 residues overlap; Score: 36.0; Gap frequency: 0.0% | | |
| Seq 52 | 123 | IMAPPVSVFPAPTTIRSSSKPLPPQLTIFYAGSVLVYQDIAPEKAQAIMLLAGNGPHAKP |
| Seq 68 | 227 | LLASRASIPSPPSAARKSDSPISAAAKLTVPEALPARQIVVQKPEASVPLVSGVSNPITI |
| | | * * * * * * * * * |
| Seq 52 | 183 | VSQ |
| Seq 68 | 287 | VSQ |
| | | *** |

This JAZ-related Oryza sativa protein, referred to as protein TIFY 6a isoform X2 (NCBI accession number XP_015651050.1 (GI:1002293416), has the following sequence (SEQ ID NO:68).

| | |
|---|---|
| 1 | MERDFLGAIW RKEEAAGKPE EHSDYRGGGG GASAAMQWQF |
| 41 | PATKVGAASS AFMSFRSSAA AAREEDPKEA AVFDRFSLSG |
| 81 | FRPPPRPSPG DAFDGAAAMK QRQFGFNGRQ QYAAAAQHGH |
| 121 | REQGVDSYGV AAPHHFPSPS PSPRHPVPFG HANPMLRVHS |
| 161 | LPNVAGGSPY RNQSFSVGNS VAGSTVGVYG GPRDLQNPKV |
| 201 | TQMTIFYDGL VNVFDNIPVE KAQELMLLAS RASIPSPPSA |
| 241 | ARKSDSPISA AAKLTVPEAL PARQIVVQKP EASVPLVSGV |
| 281 | SNPITIVSQA VTLPKSFSSS NDSAGPKSGG LPLAVTPLSQ |
| 321 | ASPSQPIPVA TTNASAIMPR AVPQARKASL ARFLEKRKER |
| 361 | VSSVAPYPSS KSPLESSDTI GSPSTPSKSS CTDITPSTNN |
| 401 | CEDSLCLGQP RNISFSSQEP PSTKLQI |

A cDNA encoding the SEQ ID NO:68 protein is available as NCBI accession number XM_015795564.1 (GI:1002293415), and a chromosomal segment encoding the SEQ ID NO:68 protein is on Oryza sativa chromosome 8 at NC_029263.1 (20624989 . . . 20627964, complement), sequence available as NCBI accession number NC_029263.1 (GI:996703425).

A Zea mays protein referred to as putative tify domain/CCT motif transcription factor family protein (NCBI accession no. DAA40037.1 (GI:414589466)) has significant sequence identity to the Arabidopsis thaliana JAZ9 protein with SEQ ID NO:54, as illustrated by the sequence comparison shown below. Domains of sequence homology are identified by asterisks below each sequence comparison.

| 52.1% identity in 48 residues overlap; Score: 118.0; Gap frequency: | | |
|---|---|---|
| 0.0% | | |
| Seq54 | 218 | SVPQARKASLARFLEKRKERLMSAMPYKKMLLDLSTGESSGMNYSSTS |
| Seq69 | 263 | AVPQARKASLARFLEKRKERVTTAAPYPSAKSPLESSDTFGSGSASAN |
| | | ******************* * ** * * * |
| 54.8% dentity in 31 residues overlap; Score: 96.0; Gap frequency: | | |
| 0.0% | | |
| Seq54 | 119 | QLTIFYGGTISVFNDISPDKAQAIMLCAGNG |
| Seq69 | 130 | QLTIFYAGSVNVFNNVSAEKAQELMFLASRG |
| | | ****** * *** * *** * * * |
| 34.6% identity in 26 residues overlap; Score: 34.0; Gap frequency: | | |
| 0.0% | | |
| Seq54 | 110 | RVASSGSSPQLTIFYGGTISVFNDIS |
| Seq69 | 85 | RVQSSPSLPNSTAFKQPPFAISNAVA |
| | | ** ** * * * * * |

This JAZ-related uncharacterized Zea mays protein, referred to as putative tify domain/CCT motif transcription factor family protein (NCBI accession no. DAA40037.1 (GI:414589466)), has the following sequence (SEQ ID NO:69).

| | |
|---|---|
| 1 | MDWSFASKPC AAPALMSFRS AAREEPSFPQ FSALDGTKNT |
| 41 | APRMLTHQRS FGPDSTQYAA LHRAQNGARV VPVSSPFSQS |
| 81 | NPMFRVQSSP SLPNSTAFKQ PPFAISNAVA SSTVGSYGGT |
| 121 | RDAVRPRTAQ LTIFYAGSVN VFNNVSAEKA QELMFLASRG |
| 161 | SSAPVACKPE APPTLAPAKV TAPEVLLPAK QMLFQKPQHL |
| 201 | SPPPSSVPGI LQSAALPRSA SSSSNLDSPA PKSSVPLAVP |
| 241 | PVSQAPPATL IATTTAAAIM PRAMPQARRA SLARFLEKRK |
| 281 | ERVTTAAPYP SAKSPLESSD TFGSGSASAN ANDKSSCTDI |
| 321 | ALSSNHEESL CLGGQPRSII SFSEESPSTK LQI |

A chromosomal segment encoding the SEQ ID NO:69 protein is on Zea mays chromosome 2 at NC_024460.1 (180086924 . . . 180089758, complement), sequence available as NCBI accession number NC_024460.1 (GI:662249846).

A Glycine max protein referred to as protein TIFY 6A isoform X6 (NCBI accession no XP_006580449.1 (GI:571456657) has significant sequence identity to the Arabidopsis thaliana JAZ9 protein with SEQ ID NO:54, as illustrated by the sequence comparison shown below. Domains of sequence homology are identified by asterisks below each sequence comparison.

| 39.8% identity in 176 residues overlap; Score: 167.0; Gap frequency: 15.3% | | |
|---|---|---|
| Seq54 | 117 | SPQLTIFYGGTISVFNDISPDKAQAIMLCAGNGLKGETGDSKP----------------- |
| Seq70 | 156 | SAQLTIFYAGTVNIFDDISAEKAQAIMLLAGNSLSAASNMAQPNVQVPISKLGAGAGVPV |
| | | * ****** ** * *** ******* *** * * |
| Seq54 | 160 | VREAERMYGKQIHN------- |
| TAATSSSSATHTDNFSRCRDTPVAATNAMSMIESFNAAP | | |
| Seq70 | 216 | SQPANTSPGSGLSSPLSVSSHTGVQGSGLTSTDEFLAAKTTGVPNTPICNVEPPKVVSA |
| * * * * * * ** * * * * | | |
| Seq54 | 213 | RNMIPS-VPQARKASLARFLEKRKERLMSAMPYK--KMLLDLSTGESSGMNYSSTS |
| Seq70 | 276 | TTMLTSAVPQARKASLARFLEKRKERVMSAAPYNLNKKSEECATAEYAGVNFSATN |
| | | * * ******************* *** ** * * * * * * * |
| 40.0% identity in 40 residues overlap; Score: 66.0; Gap frequency: 2.5% | | |
| Seq54 | 1 | MERDFLGLSDKQYLSNNVKHEVNDDAVEERGLSTKAAREW |
| Seq70 | 1 | MERDFMGLNLKEPLAV-VKEEMNNDGCKNSGFKKGRIAQW |
| | | ***** ** * * ** * * * * * |

This JAZ-related Glycine max protein, referred to as protein TIFY 6A isoform X6 (NCBI accession no. XP_006580449.1 (GI:571456657)) has the following sequence (SEQ ID NO:70).

| | |
|---|---|
| 1 | MERDFMGLNL KEPLAVVKEE MNNDGCKNSG FKKGRIAQWP |
| 41 | FSNKVSALPH LMSFKASQDD KTKNTVSDTL SSSGFMSILS |
| 81 | QEAFDTSQKR SAGEPQMFSV PNQAISVSLG NPFLKNHFAA |
| 121 | AGQKPLLGGI PVTTSHSVLP SAVAVAGMTE SCVKPSAQLT |
| 161 | IFYAGTVNIF DDISAEKAQA IMLLAGNSLS AASNMAQPNV |
| 201 | QVPISKLGAG AGVPVSQPAN TSPGSGLSSP LSVSSHTGVQ |
| 241 | SGSGLTSTDE FLAAKTTGVP NTPICNVEPP KVVSATTMLT |
| 281 | SAVPQARKAS LARFLEKRKE RVMSAAPYNL NKKSEECATA |
| 321 | EYAGVNFSAT NTVLAKQG |

A cDNA encoding the SEQ ID NO:70 protein is available as NCBI accession number XM_006580386.2 (GI:955322109), and a chromosomal segment encoding the SEQ ID NO:70 protein is on Glycine max chromosome 5 at NC_016092.2 (41222014 . . . 41225906), sequence available as NCBI accession number NC_016092.2 (GI:952545311).

An unknown Oryza sativa protein with NCBI accession no. BAD28520.1 (GI:50251455) has significant sequence identity to the Arabidopsis thaliana JAZ9 protein with SEQ ID NO:54, as illustrated by the sequence comparison shown below. Domains of sequence homology are identified by asterisks below each sequence comparison.

| 40.9% identity in 66 residues overlap; Score: 116.0; Gap frequency: 1.5% | | |
|---|---|---|
| Seq54 | 84 | GGAFQNATPLLLGGSVPLPTHPSLVPRVASSGSSPQLTIFYGGTISVFNDISPDKAQAIM |
| Seq71 | 51 | GGAFKQP-PFAMGNAVAGSTVGVYGTRDMPKAKAAQLTIFYAGSVNVFNNVSPEKAQELM |
| | | **** * * * * * ****** * *** ** *** * |
| Seq54 | 144 | LCAGNG |
| Seq71 | 110 | FLASRG |
| | | * * |
| 56.1% identity in 41 residues overlap; Score: 110.0; Gap frequency: 0.0% | | |
| Seq54 | 218 | SVPQARKASLARFLEKRKERLMSAMPYKKMLLDLSTGESSG |
| Seq71 | 225 | AVPQARKASLARFLEKRKERVTTVAPYPLAKSPLESSDTMG |
| | | ******************* ** * * |

This JAZ-related Oryza sativa protein with NCBI accession no. BAD28520.1 (GI:50251455) has the following sequence (SEQ ID NO:71).

| | |
|---|---|
| 1 | MQQASHVLAR QPPHVLNGAR VIPASSPFNP NNPMFRVQSS |
| 41 | PNLPNAVGAG GGAFKQPPFA MGNAVAGSTV GVYGTRDMPK |
| 81 | AKAAQLTIFY AGSVNVFNNV SPEKAQELMF LASRGSLPSA |
| 121 | PTTVARMPEA HVFPPAKVTV PEVSPTKPMM LQKPQLVSSP |
| 161 | VPAISKPISV VSQATSLPRS ASSSNVDSNV TKSSGPLVVP |
| 201 | PTSLPPPAQP ETLATTTAAA IMPRAVPQAR KASLARFLEK |
| 241 | RKERVTTVAP YPLAKSPLES SDTMGSANDN KSSCTDIALS |
| 281 | SNRDESLSLG QPRTISFCEE SPSTKLQI |

A chromosomal segment encoding the SEQ ID NO:71 protein is on Oryza sativa chromosome 9 at NC_029264.1 (14056084 . . . 14060320, complement), sequence available as NCBI accession number NC_029264.1 (GI:996703424).

An uncharacterized Zea mays protein referred to as LOC100384222 (NCBI accession no. NP_001182812.1 (GI:308044557)) has significant sequence identity to the Arabidopsis thaliana JAZ10 protein with SEQ ID NO:56, as illustrated by the sequence comparison shown below. Domains of sequence homology are identified by asterisks below each sequence comparison.

| 36.2% identity in 94 residues overlap; Score: 126.0; Gap frequency: | | |
|---|---|---|
| 3.2% | | |
| Seq56 | 105 | MTIFYNGSVSVF- |
| QVSRNKAGEIMKVANEAASKKDESSMETDLSVILPTTLRPKLFGQL | | |
| Seq72 | 96 | LTIFYGGKVVVFDRFPSAKVKDLLQIVSPPGA-- |
| DAVVDGAGAGAAVPTQNLPRPSHDSL | | |
| **** * * ** * * ** * | | |
| * | | |
| Seq56 | 164 | EGDLPIARRKSLQRFLEKRKERLVSTSPYYPTSA |
| Seq72 | 154 | SADLPIARRNSLHRFLEKRKDRITAKAPYQVNSS |
| | | ******* ** ******* * ** * |

This JAZ-related uncharacterized Zea mays protein referred to as LOC100384222 (NCBI accession no. NP_001182812.1 (GI:308044557)) has the following sequence (SEQ ID NO:72).

| | |
|---|---|
| 1 | MAGHAPARDK TTTGFAATCS LLSQFLKEKK GGLQGLGGLA |
| 41 | MAPAPAAGAG AFRPPTTMNL LSALDAAKAT VGEPEGHGQR |
| 81 | TGGNPREAAG EEAQQLTIFY GGKVVVFDRF PSAKVKDLLQ |
| 121 | IVSPPGADAV VDGAGAGAAV PTQNLPRPSH DSLSADLPIA |
| 161 | RRNSLHRFLE KRKDRITAKA PYQVNSSVGA EASKAEKPWL |
| 201 | GLGQEQEGSD GRQAGEEM |

A cDNA encoding the SEQ ID NO:72 protein is available as NCBI accession number NM_001195883.1 (GI:308044556), and a chromosomal segment encoding the SEQ ID NO:72 protein is on Zea mays chromosome 7 at NC_024465.1 (121257106 . . . 121259180, complement), sequence available as NCBI accession number NC_024465.1 (GI:662248746).

An uncharacterized Glycine max protein referred to as LOC100306524 (NCBI accession number NP_001236269.1 (GI:351723837) has significant sequence identity to the Arabidopsis thaliana JAZ10 protein with SEQ ID NO:56, as illustrated by the sequence comparison shown below. Domains of sequence homology are identified by asterisks below each sequence comparison.

| 36.6% identity in 123 residues overlap; Score: 114.0; Gap frequency: 12.2% | | |
|---|---|---|
| Seq56 | 85 | SPVHASLARSSTELVSGTVPMTIFYNGSVSVFQ-VSRNKAGEIMKVANEAASKKDESSME |
| Seq73 | 38 | SPNKSVPASGLDAVIPSANQLTIFYNGSVCVYDGIPAEKVHEIMLIAAAAAKSTEMKKIG |
| | | ** * ******** * * *** * ** |
| Seq56 | 144 | TDLSVILPTTLRP--------------KLFGQNLEGDLPIARRKSLQRFLEKRKERLVST |
| Seq73 | 98 | TQTTLISPAPSRPSSPHGITNNIGSSQKSSICRLQAEFPIARRHSLQRFLEKRRDRLGSK |
| | | * * * ** * * ***** ********* ** * |
| Seq56 | 190 | SPY |
| Seq73 | 158 | TPY |
| | | ** |

This JAZ-related uncharacterized Glycine max protein referred to as LOC100306524 (NCBI accession number NP_001236269.1 (GI:351723837) has the following sequence (SEQ ID NO:73).

| | |
|---|---|
| 1 | MAAGVTVKSE VLESSPPEGV CSNTVENALV QTNLSDGSPN |
| 41 | KSVPASGLDA VIPSANQLTI FYNGSVCVYD GIPAEKVHEI |
| 81 | MLIAAAAAKS TEMKKIGTQT TLISPAPSRP SSPHGITNNI |
| 121 | GSSQKSSICR LQAEFPIARR HSLQRFLEKR RDRLGSKTPY |
| 161 | PSSPTTKVAD NIENNTCADN APELISLNRS EEEFQPTVSA |
| 201 | S |

A cDNA encoding the SEQ ID NO:73 protein is available as NCBI accession number NM_001249340.2 (GI:402766138), and a chromosomal segment encoding the SEQ ID NO:73 protein is on Glycine max chromosome 15 at NC_016102.2 (18552881 . . . 18556339), sequence available as NCBI accession number NC_016102.2 (GI:952545301).

An Oryza sativa protein referred to as protein TIFY 9 with NCBI accession no. XP_015634258.1 (GI:1002259863) has significant sequence identity to the Arabidopsis thaliana JAZ10 protein with SEQ ID NO:56, as illustrated by the sequence comparison shown below. Domains of sequence homology are identified by asterisks below each sequence comparison.

| 40.0% identity in 110 residues overlap; Score: 119.0; Gap frequency: | | |
|---|---|---|
| 13.6% | | |
| Seq56 | 83 | PISPVHASLARSSTELVSGTVPMTIFYNGSVSVFQVSRNKAGEIMKVANEAASKKDESSM |
| Seq74 | 65 | PPPPSTAPVPEEMPGAAAAAAPMTLFYNGSVAVFDVSHDKAEAIMRMATEATKAKGLA-- |
| | | * * * *** ****** ** ** ** ** * ** * |
| Seq56 | 143 | ETDLSVILPTTLRPKLFGQNLEGDLPIARRKSLQRFLEKRKERLVSTSPY |
| Seq74 | 123 | ------------RGNAIVGNFAKE-PLTRTKSLQRFLSKRKERLTSLGPY |
| | | * * * * ******* ****** * ** |
| 66.7% identity in 12 residues overlap; Score: 44.0; Gap frequency: | | |
| 0.0% | | |
| Seq56 | 2 | SKATIELDFLGL |
| Seq74 | 3 | TRAPVELDFLGL |
| | | * ******* |

This JAZ-related Oryza sativa protein referred to as protein TIFY 9 with NCBI accession no. XP_015634258.1 (GI:1002259863) has the following sequence (SEQ ID NO:74).

| | |
|---|---|
| 1 | MSTRAPVELD FLGLRAAAAD ADDRHAKSGG SSASSSSSIR |
| 41 | GMETSAIARI GPHLLRRVIA AAGPPPPPST APVPEEMPGA |
| 81 | AAAAAPMTLF YNGSVAVFDV SHDKAEAIMR MATEATKAKG |
| 121 | LARGNAIVGN FAKEPLTRTK SLQRFLSKRK ERLTSLGPYQ |
| 161 | VGGPAAVGAT TSTTTKSFLA KEEEHTAS |

A cDNA encoding the SEQ ID NO:74 protein is available as NCBI accession number XM_015778772.1 (GI:1002259862), and a chromosomal segment encoding the SEQ ID NO:74 protein is on Oryza sativa chromosome 4 at NC_029259.1 (19492605 . . . 19497181), sequence available as NCBI accession number NC_029259.1 (GI:996703429).

Chromosomal sites encoding any of the conserved amino acids and conserved domains illustrated by the sequence comparisons shown above can be deleted or mutated to reduce the activity of the proteins described herein.

For example, a wild type plant can express JAZ polypeptides or JAZ-related polypeptides with at least 70%, or at least 75%, or at least 80%, or at least 85%, or at least 90%, or at least 95%, or at least 97%, or at least 98%, or at least 99% sequence identity to any of SEQ ID NOs:48, 50, 52, 54, 56, 58-73, or 74.

However, the mutant jazQ plant cells, plants, and/or seeds with increased jasmonic acid responses and improved insect resistance can express mutant JAZ and/or JAZ-related polypeptides that have reduced activity. Such JAZ and/or JAZ-related polypeptides that have reduced JAZ activity can have less than 99%, or less than 98%, or less than 95%, or less than 90%, or less than 85%, or less than 75%, or less than 60%, or less than 50%, or less than 40%, or less than 30%, or less than 20% sequence identity to any of SEQ ID NOs:48, 50, 52, 54, 56, 58-73, or 74.

The mutant JAZ and/or JAZ-related polypeptides can, for example, have mutations in at least one conserved amino acid position, or at least two conserved amino acid positions, or at least three conserved amino acid positions, or at least five conserved amino acid positions, or at least seven conserved amino acid positions, or at least eight conserved amino acid positions, or at least ten conserved amino acid positions, or at least fifteen amino acid positions, or at least twenty conserved amino acid positions, or at least twenty-five amino acid positions. In some cases, an entire conserved JAZ and/or JAZ-related domain or the entire endogenous JAZ and/or JAZ-related gene or chromosomal segment is deleted or mutated.

The conserved amino acids and/or domains are in some cases mutated by deletion or replacement with amino acids that have dissimilar physical and/or chemical properties. Examples of amino acids with different physical and/or chemical properties that can be employed are shown in Tables 1 and 2.

### Transformation of Plant Cells

Mutations can be introduced into any of the MYC, MYC-related, JAZ, JAZ-related, PHYB or PHYB-related plant genomes by introducing targeting vectors, T-DNA, transposons, nucleic acids encoding TALENS, CRISPR, or ZFN nucleases, and combinations thereof into a recipient plant cell to create a transformed cell. In addition plant cells can be transformed to include a PIF4 transgene, for example, by transformation of the plant cells with a PIF4 expression cassette or expression vector.

The frequency of occurrence of cells taking up exogenous (foreign) DNA can sometimes be low. However, certain cells from virtually any dicot or monocot species can be stably transformed, and these cells can be regenerated into transgenic plants, through the application of the techniques disclosed herein. The plant cells, plants, and seeds can therefore be monocotyledons or dicotyledons.

The cell(s) that undergo transformation may be in a suspension cell culture or may be in an intact plant part, such as an immature embryo, or in a specialized plant tissue, such as callus, such as Type I or Type II callus.

Transformation of the cells of the plant tissue source can be conducted by any one of a number of methods available to those of skill in the art. Examples include: Transformation by direct DNA transfer into plant cells by electroporation (U.S. Pat. Nos. 5,384,253 and 5,472,869, Dekeyser et al., The Plant Cell. 2:591 602 (1990)); direct DNA transfer to plant cells by PEG precipitation (Hayashimoto et al., Plant Physiol. 93:857 863 (1990)); direct DNA transfer to plant cells by microprojectile bombardment (McCabe et al., Bio/Technology. 6:923 926 (1988); Gordon Kamm et al., The Plant Cell. 2:603 618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf disk protocol (Horsch et al., Science 227:1229 1231 (1985). Monocots such as Zea mays can be transformed via microprojectile bombardment of embryogenic callus tissue or immature embryos, or by electroporation following partial enzymatic degradation of the cell wall with a pectinase containing enzyme (U.S. Pat. Nos. 5,384,253; and 5,472,869). For example, embryogenic cell lines derived from immature Zea mays embryos can be transformed by accelerated particle treatment as described by Gordon Kamm et al. (The Plant Cell. 2:603 618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. application Ser. No. 08/112,245 and PCT publication WO 95/06128. Furthermore, methods for transformation of monocotyledonous plants utilizing Agrobacterium tumefaciens have been described by Hiei et al. (European Patent 0 604 662, 1994) and Saito et al. (European Patent 0 672 752, 1995).

Methods such as microprojectile bombardment or electroporation can be carried out with “naked” DNA where the expression cassette may be simply carried, for example, on any E. coli derived plasmid cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are exemplary Zea mays tissue sources. Selection of tissue sources for transformation of monocots is described in detail in U.S. application Ser. No. 08/112,245 and PCT publication WO 95/06128.

The transformation is carried out under conditions directed to the plant tissue of choice. The plant cells or tissue are exposed to the DNA or RNA carrying the targeting vector and/or other nucleic acids for an effective period of time. This may range from a less than one second pulse of electricity for electroporation to a 2-3 day co cultivation in the presence of plasmid bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

Where one wishes to introduce DNA by means of electroporation, it is contemplated that the method of Krzyzek et al. (U.S. Pat. No. 5,384,253) may be advantageous. In this method, certain cell wall degrading enzymes, such as pectin degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic cells were bombarded with intact cells of the bacteria E. coli or Agrobacterium tumefaciens containing plasmids with either the O-glucouronidase or bar gene engineered for expression in maize. Bacteria were inactivated by ethanol dehydration prior to bombardment. A low level of transient expression of the O-glucouronidase gene was observed 24-48 hours following DNA delivery. In addition, stable transformants containing the bar gene can be recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, is that the isolation of protoplasts (Christou et al., PNAS. 84:3962 3966 (1987)), the formation of partially degraded cells, or the susceptibility to Agrobacterium infection is not required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon Kamm et al., The Plant Cell. 2:603 618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing damage inflicted on the recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Through the use of techniques set forth here in one may obtain up to 1000 or more foci of cells transiently expressing a marker gene. The number of cells in a focus which express the exogenous gene product 48 hours post bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macroprojectiles or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

Examples of plants and/or plant cells that can be modified as described herein include alfalfa (e.g., forage legume alfalfa), algae, avocado, barley, broccoli, Brussels sprouts, cabbage, canola, cassava, cauliflower, cole vegetables, collards, corn, crucifers, grain legumes, grasses (e.g., forage grasses), jatropa, kale, kohlrabi, maize, miscanthus, mustards, nut sedge, oats, oil firewood trees, oilseeds, potato, radish, rape, rapeseed, rice, rutabaga, sorghum, soybean, sugar beets, sugarcane, sunflower, switchgrass, tobacco, tomato, turnips, and wheat. In some embodiments, the plant is a Brassicaceae or other Solanaceae species. In some embodiments, the plant or cell can be a maize plant or cell. In some embodiments, the plant is not a species of Arabidopsis, for example, in some embodiments, the plant is not Arabidopsis thaliana.

An exemplary embodiment of methods for identifying transformed cells involves exposing the bombarded cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. In an illustrative embodiment embryogenic Type II callus of Zea mays L. can be selected with sub-lethal levels of bialaphos. Slowly growing tissue was subsequently screened for expression of the luciferase gene and transformants can be identified.

### Regeneration and Seed Production

Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to have the mutations. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants in order to introgress the mutations into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced myc, JAZ, or phyB mutations or PIF4 expression cassette, the plant is self-pollinated at least once in order to produce a homozygous backcross converted inbred containing the mutations. Progeny of these plants are true breeding.

Alternatively, seed from transformed mutant plant lines regenerated from transformed tissue cultures is grown in the field and self-pollinated to generate true breeding plants.

Seed from the fertile transgenic plants can then be evaluated for the presence of the desired MYC, JAZ, or PhyB mutation, the desired PIF4 expression cassette, and/or the expression of the desired mutant protein. Transgenic plant and/or seed tissue can be analyzed using standard methods such as SDS polyacrylamide gel electrophoresis, liquid chromatography (e.g., HPLC) or other means of detecting a mutation.

Once a transgenic plant with a mutant sequence and having improved growth and insect resistance is identified, seeds from such plants can be used to develop true breeding plants. The true breeding plants are used to develop a line of plants with an increase insect resistance relative to wild type, and acceptable growth characteristics while still maintaining other desirable functional agronomic traits. Adding the mutation to other plants can be accomplished by back-crossing with this trait and with plants that do not exhibit this trait and studying the pattern of inheritance in segregating generations. Those plants expressing the target trait (insect resistance, good growth) in a dominant fashion are preferably selected. Back-crossing is carried out by crossing the original fertile transgenic plants with a plant from an inbred line exhibiting desirable functional agronomic characteristics while not necessarily expressing the trait of an increased insect resistance and good plant growth. The resulting progeny are then crossed back to the parent that expresses the increased insect resistance and good plant growth. The progeny from this cross will also segregate so that some of the progeny carry the trait and some do not. This back-crossing is repeated until an inbred line with the desirable functional agronomic traits, and with expression of the trait involving an increase in insect resistance and good plant growth. Such insect resistance and good plant growth can be expressed in a dominant fashion.

The new transgenic plants can also be evaluated for a battery of functional agronomic characteristics such as growth, lodging, kernel hardness, yield, resistance to disease and insect pests, drought resistance, and/or herbicide resistance.

Plants that may be improved by these methods include but are not limited to agricultural plants of all types, oil and/or starch plants (canola, potatoes, lupins, sunflower and cottonseed), forage plants (alfalfa, clover and fescue), grains (maize, wheat, barley, oats, rice, sorghum, millet and rye), grasses (switchgrass, prairie grass, wheat grass, sudangrass, sorghum, straw-producing plants), softwood, hardwood and other woody plants (e.g., those used for paper production such as poplar species, pine species, and eucalyptus). In some embodiments the plant is a gymnosperm. Examples of plants useful for pulp and paper production include most pine species such as loblolly pine, Jack pine, Southern pine, Radiata pine, spruce, Douglas fir and others. Hardwoods that can be modified as described herein include aspen, poplar, eucalyptus, and others. Plants useful for making biofuels and ethanol include corn, grasses (e.g., miscanthus, switchgrass, and the like), as well as trees such as poplar, aspen, willow, and the like. Plants useful for generating dairy forage include legumes such as alfalfa, as well as forage grasses such as bromegrass, and bluestem.

### Determination of Stably Transformed Plant Tissues

To confirm the presence of MYC, JAZ, and/or PHYB mutations and/or a PIF4 expression cassette in the regenerating plants, or seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced MYC, JAZ, or PhyB mutations or of RNA expressed from an introduced PIF4 expression cassette. For example, PCR also be used to reverse transcribe RNA into DNA, using enzymes such as reverse transcriptase, and then this DNA can be amplified through the use of conventional PCR techniques.

For example, if no amplification of PHYB mRNAs is observed, then a deletion mutation has successfully been introduced.

Information about mutations can also be obtained by primer extension or single nucleotide polymorphism (SNP) analysis.

Further information about the nature of the RNA product may be obtained by Northern blotting. This technique will demonstrate the presence of an RNA species and give information about the integrity of that RNA. The presence of some mutations can be detected by Northern blotting. The presence or absence of an RNA species (e.g., PIF4 RNA) can also be determined using dot or slot blot Northern hybridizations. These techniques are modifications of Northern blotting and also demonstrate the presence or absence of an RNA species.

While Southern blotting and PCR may be used to detect the presence of MYC, JAZ, and/or PHYB mutations or the presence of a PIF4 expression cassette, they do not provide information as to whether the preselected DNA segment is being expressed. Expression may be evaluated by specifically identifying the protein products of the introduced PIF4 expression cassette or the introduced mutations, by detecting that no PHYB proteins are expressed, or evaluating the phenotypic changes brought about by such mutation.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products, or the absence thereof, that have been separated by electrophoretic techniques. Additional techniques may be employed to absolutely confirm the identity of a mutation such as evaluation by screening for reduced transcription (or no transcription) of MYC, JAZ, and/or PHYB mRNAs, by screening for PIF4 expression, or by amino acid sequencing following purification. The Examples of this application also provide assay procedures for detecting and quantifying insect resistance and plant growth. Other procedures may be additionally used.

The expression of a gene product can also be determined by evaluating the phenotypic results of its expression. These assays also may take many forms including but not limited to analyzing changes in the insect resistance, growth characteristics, or other physiological properties of the plant. Expression of selected DNA segments encoding different amino acids or having different sequences and may be detected by amino acid analysis or sequencing.

The following Examples describe some of the experiments performed in the development of the invention.

### Example 1: Materials and Methods

This Example describes some of the material and methods employed in the development of the invention.

Plant material and growth conditions. Arabidopsis thaliana Columbia ecotype (Col-0) was used as a wild-type (WT) parent for all experiments. Soil-grown plants were maintained at 20° C. (+1° C.) with 16 h light at a light intensity of 120 μE m⁻² s⁻¹ and 8 h dark unless otherwise noted. For the first 10 days after seed sowing, trays containing potted plants were covered with a transparent plastic dome to increase humidity. For experiments involving growth of seedlings on agar plates, seeds were surface sterilized for 15 min in a solution containing 50% (v/v) bleach and 0.1% (v/v) Triton X-100, washed 10 times with sterile water and then stratified in dark at 4° C. for 2 days. Seeds were then sown on 0.7% (w/v) agar media containing half-strength Murashige and Skoog (MS; Caisson Labs) salts supplemented with 0.8% (w/v) sucrose.

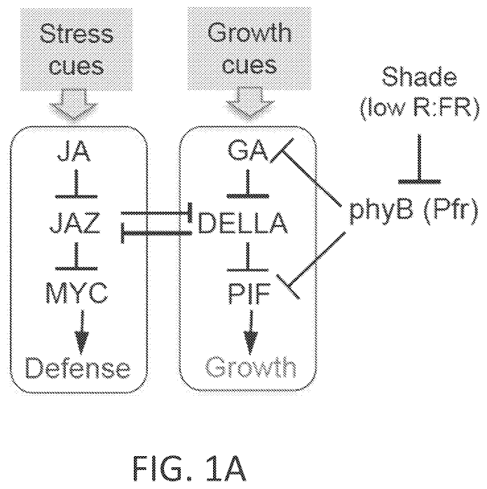

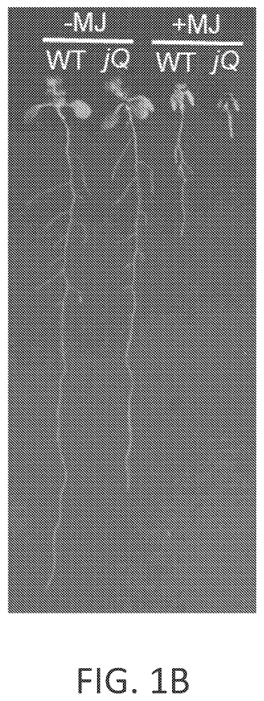

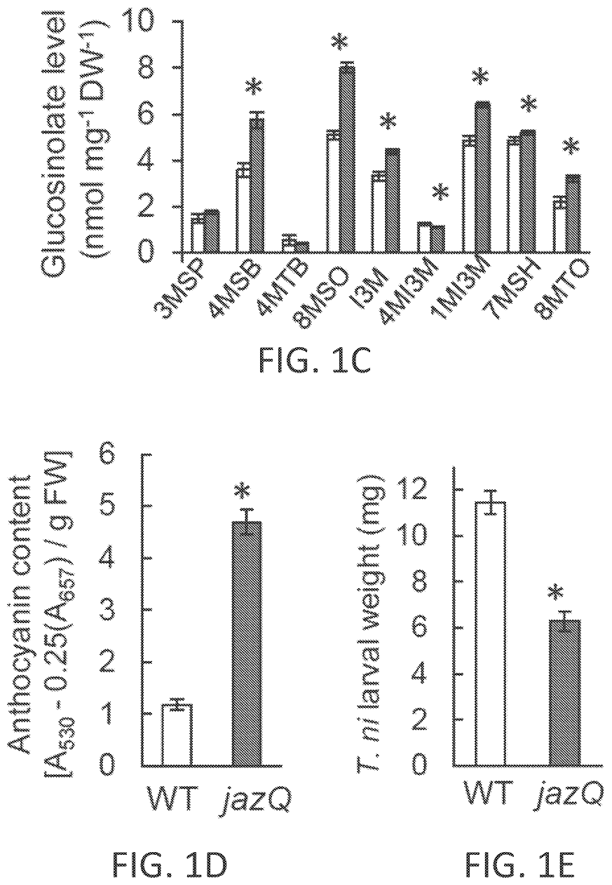

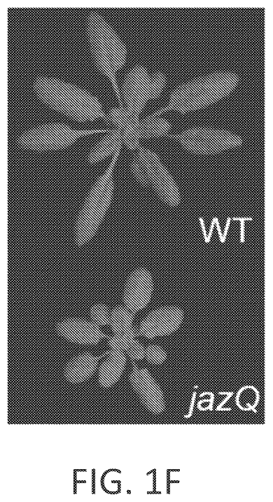

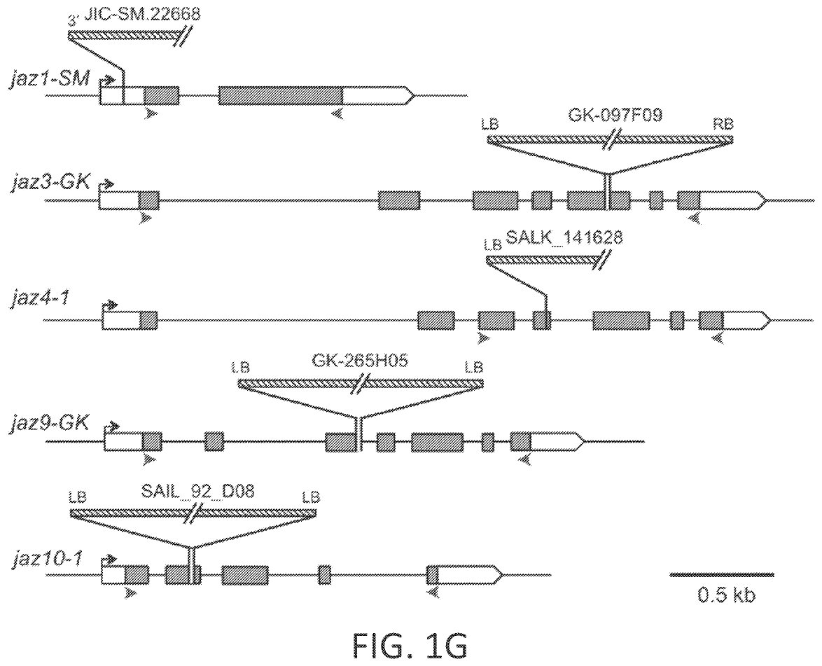

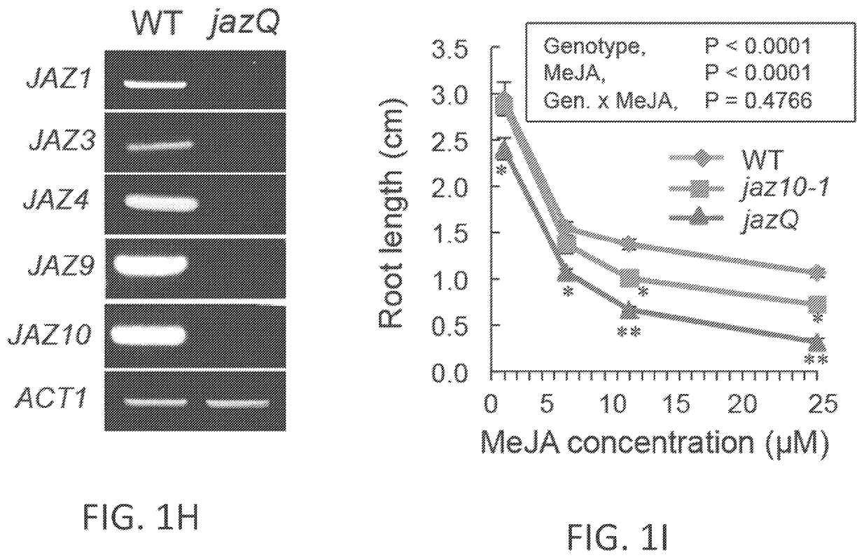

Transfer DNA (T-DNA) insertion mutants used for construction of jazQ were obtained from the Arabidopsis Biological Research Center (ABRC; The Ohio State University) and named as follows: jaz1-SM (jaz1, JIC-SM.22668), jaz3-GK (jaz3, GK-097F09), jaz4-1 (jaz4, SALK_141628), jaz9-GK (jaz9, GK-265H05) and jaz10-1 (jaz10, SAIL_92_D08). See FIG. 1G.

The jaz3-GK and jaz9-GK lines were backcrossed to Col-0 to remove unlinked T-DNA insertions. The jaz10-1 was backcrossed to Col-0 to remove a qrt1-2 mutation present in the SAIL lines (McElver et al., Genetics 159, 1751-1763 (2001)). The jaz4-1 and jaz10-1 mutants are described by Jiang et al. (Plant Cell 26, 230-245 (2014)), and Sehr et al. (Plant J 63, 811-822 (2010)). The jazQ phyB sextuple mutant was obtained from a genetic cross between mutant jazQ and the mutant phyB reference allele phyB-9 (Reed et al., Plant Cell 5, 147-157 (1993)). The higher-order pifq (pif1-1/pif3-3/pif4-2/pif5-3) and dellaQ (gai-t6/rgat2/rgl1-1/rgl2-1/rgl3-1) mutants are discussed by Feng et al. (Nature 451, 475-479 (2008)) and Leivar et al., (Curr Biol 18, 1815-1823 (2008)).

PCR analysis. PCR-based genotyping of jazQ and lower-order mutants relied on primer sets flanking T-DNA insertion sites, together with a third primer recognizing the border of the inserted T-DNA. The forward, reverse, and border primers used were the following:

JAZ1 (At1g19180):
(SEQ ID NO: 75)
5′-ACCGAGACACATTCCCGATT-3′,
(SEQ ID NO: 76)
5′-CATCAGGCTTGCATGCCATT-3′,
and
(SEQ ID NO: 77)
5′-ACGAATAAGAGCGTCCATTTTAGAG-3′;
JAZ3 (At3g17860):
5′-ACGGTTCCTCTATGCCTCAAGTC-3′,
(SEQ ID NO: 78)
5′-GTGGAGTGGTCTAAAGCAACCTTC-3′,
and
(SEQ ID NO: 79)
5′-ATAACGCTGCGGACATCTACATT-3′;
JAZ4 (At1g48500):
(SEQ ID NO: 80)
5′-TCAGGAAGACAGAGTGTTCCC-3′,
(SEQ ID NO: 81)
5′-TGCGTTTCTCTAAGAACCGAG-3′,
and
(SEQ ID NO: 83)
5′-TTGGGTGATGGTTCACGTAG-3′;
JAZ9 (At1g70700):
(SEQ ID NO: 84)
5′-TACCGCATAATCATGGTCGTC-3′,
(SEQ ID NO: 85)
5′-TCATGCTCATTGCATTAGTCG-3′,
and
(SEQ ID NO: 86)
5′-CTTTGAAGACGTGGTTGGAACG-3′;
JAZ10 (At5g13220):
(SEQ ID NO: 87)
5′-ATTTCTCGATCGCCGTCGTAGT-3′,
(SEQ ID NO: 88)
5′-GCCAAAGAGCTTTGGTCTTAGAGTG-3′,
and
(SEQ ID NO: 89)
5′-GTCTAAGCGTCAATTTGTTTACACC-3′.

Reverse transcription-PCR (RT-PCR) was used to confirm the presence or absence of JAZ transcripts in wild type (WT) and mutant jazQ plants. For this purpose, RNA was extracted from eight-day-old seedlings grown on MS plates containing 20 M MeJA. Frozen tissue was homogenized with a mortar and pestle and RNA was extracted using an RNeasy kit (Qiagen) with on-column DNase (Qiagen) treatment. cDNA was reverse transcribed from one μg total RNA with a High Capacity cDNA Reverse Transcription kit (Applied Biosystems, ABI). RT-PCR was performed using primer sets designed to amplify the five JAZ genes and the internal control ACTIN1 (At2g37620). The forward and reverse primer sets used were as follows:

JAZ1:
(SEQ ID NO: 90)
5′-ATGTCGAGTTCTATGGAATGTTCTG-3′,
and
(SEQ ID NO: 91)
5′-TCATATTTCAGCTGCTAAACCGAGCC-3′;
JAZ3:
(SEQ ID NO: 92)
5′-ATGGAGAGAGATTTTCTCGGG-3′,
and
(SEQ ID NO: 93)
5′-TTAGGTTGCAGAGCTGAGAGAAG-3′;
JAZ4:
(SEQ ID NO: 94)
5′-ATGGAGAGAGATTTTCTCGG-3′,
and
(SEQ ID NO: 95)
5′-CAGATGATGAGCTGGAGGAC-3;
JAZ9:
(SEQ ID NO: 96)
5′-ATGGAAAGAGATTTTCTGGGTTTG-3′,
and
(SEQ ID NO: 97)
5′-TTATGTAGGAGAAGTAGAAGAGTAATTCA-3′;
JAZ10:
(SEQ ID NO: 98)
5′-ATGTCGAAAGCTACCATAGAAC-3′,
and
(SEQ ID NO: 99)
5′-GATAGTAAGGAGATGTTGATACTAATCTCT-3′;
and
ACTIN1:
(SEQ ID NO: 100)
5′-ATGGCTGATGGTGAAGACATTCAA-3′,
and
(SEQ ID NO: 101)
5′-TCAGAAGCACTTCCTGTGAACAAT-3′.

RT-PCR reactions were performed with the following conditions: 94° C. for 5 min, followed by 30 cycles of denaturation (45 sec at 94° C.), annealing (30 sec at 52° C.), and elongation (1.5 min at 72° C.). Final elongation step was performed at 72° C. for 10 min and completed reactions were maintained at 12° C. Forty elongation cycles were used to detect the JAZ4 transcripts, which accumulate at low levels in WT plants (Chung et al., Plant Physiol 146, 952-964 (2008)).

Root growth assays. The effect of exogenous JA on seedling root growth inhibition has been described by Shyu et al. (Plant Cell 24, 536-550 (2012)). Seedlings were grown on square Petri plates (Fisher) containing MS medium supplemented with the indicated concentration of methyl-jasmonic acid (MeJA; Sigma-Aldrich). Plates were incubated vertically in a growth chamber maintained at 21° C. under continuous light for 8 days. Primary root length was measured using the ImageJ software (see website at imagej.nih.gov/ij/). WT and mutant lines were grown on the same plate to control for plate-to-plate variation.

Quantification of secondary metabolites. Anthocyanins were quantified as described by Kang et al. (Plant Physiol 164 (2014)), with minor modifications. Petioles were excised from 4-week-old plants and extracted in 1 ml methanol (MeOH) containing 1% (v/v) HCl. Samples were incubated overnight at 4° C. with constant agitation. Anthocyanin pigments in the resulting extract were measured spectrophotometrically and calculated as A₅₃₀−0.25(A₆₅₇) g⁻¹ fresh weight. Glucosinolates were quantified as described by Barth & Jander (Plant J 46, 549-562 (2006)) with minor modifications. Eight-day-old seedlings grown on solid MS medium were collected into two-mL tubes (approximately 50 seedlings per tube) and immediately frozen in liquid nitrogen. WT and mutant lines were grown on the same plate to avoid plate-to-plate variation. Frozen tissue was lyophilized, ground to a fine powder and extracted with 1 ml 80% MeOH containing an internal standard (25 nmol sinigrin, Sigma-Aldrich). Samples were briefly vortexed, incubated at 75° C. for 15 min, and then centrifuged at 23° C. at 10,000×g for 10 min. Resulting supernatants were applied to Sephadex A-25 columns (Amersham). Glucosinolates were released from the columns as desulfoglucosinolates with a solution containing L of aryl sulfatase (3.0 mg ml⁻¹; Sigma) and 70 μL water (HPLC-grade). Following an overnight incubation in the dark at 23° C., samples were eluted from the columns with 200 μL 80% MeOH and 200 μL water. Samples were then lyophilized to complete dryness and re-dissolved in 100 μL water.

Desulfoglucosinoaltes were detected by HPLC and quantified as described by Barth & Jander (Plant J 46, 549-562 (2006)). Compound abbreviations in FIG. 1C correspond to the following: 3MSP, 3-methylsulfinylpropylglucosinolate; 4MSB, 4-methylsulfinylbutylglucosinolate; 5MSP, 5-methylsulfinylpentylyglucosinolate; 40H13M, 4-hydroxyindol-3-ylmethylglucosinolate; 7MSH, 7-methylsulfinylheptyl-glucosinolate; 4MTB, 4-methylthiobutylglucosinolate; 8MSO, 8-methylsulfinyloctyl-glucosinolate; I3M, indol-3-ylmethylglucosinolate; 4MI3M, 4-methoxyindol-3-ylmethylglucosinolate; 1MI3M, 1-methoxyindol-3-ylmethylglucosinolate; 7MTH, 7-methylthioheptylglucosinolate; and 8MTO, 8-methylthiooctylglucosinolate.

Insect feeding assays. Insect feeding assays were performed with soil-grown plants maintained in a growth chamber at 19° C. and a photoperiod of 8 h light (120 μE m⁻² s⁻¹) and 16 h dark. Neonate Trichoplusia ni larvae (Benzon Research) were transferred to the center of fully expanded rosette leaves of 6-week-old plants, as previously described by Herde et al. (Methods Mol Biol 1011, 51-61 (2013)). Four larvae were reared on each of 12 plants per genotype. Plants were then covered with a transparent dome and returned to the chamber for 10 d, after which larval weights were measured.

Growth and flowering time measurements. Three-to-four week-old soil-grown plants were used for all measurements (10 plants per measurement), unless indicated otherwise. Petiole length of the third true leaf was measured with a caliper after leaf excision. Bolting time was measured in a separate set of plants by counting the number of true leaves on the main stem and the number of days from sowing until bolting (i.e., floral buds visible in the center of the rosette). The same set of plants was subsequently used to assess the length of time to opening of the first flower. Rosette diameter and leaf area were determined by photographing rosettes from the top with a Nikon D80 camera. The resulting images were used to calculate Feret diameter using ImageJ analysis. Total leaf area was determined with GIMP software (see website at gimp.org). Leaf dry weight was determined by weighing excised rosettes (without roots) after freeze drying for two days in a lyophilizer.

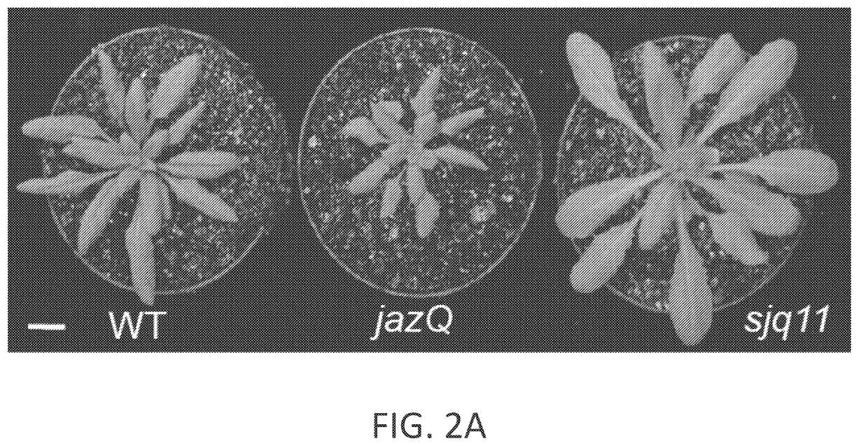

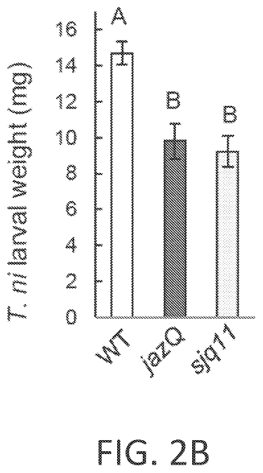

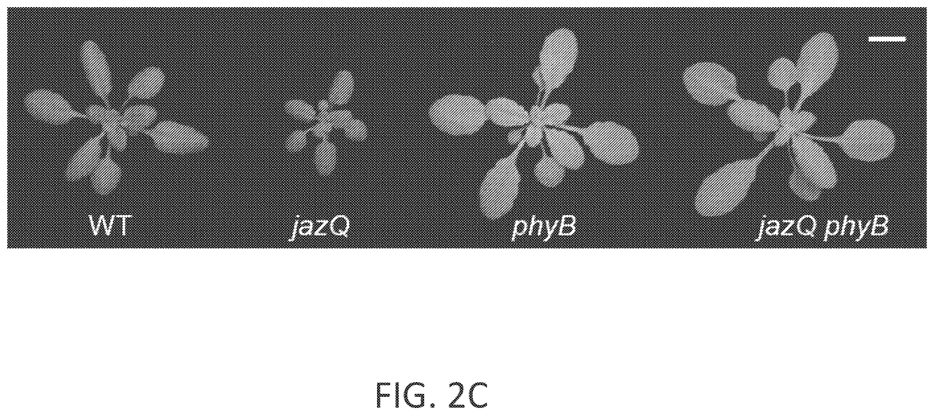

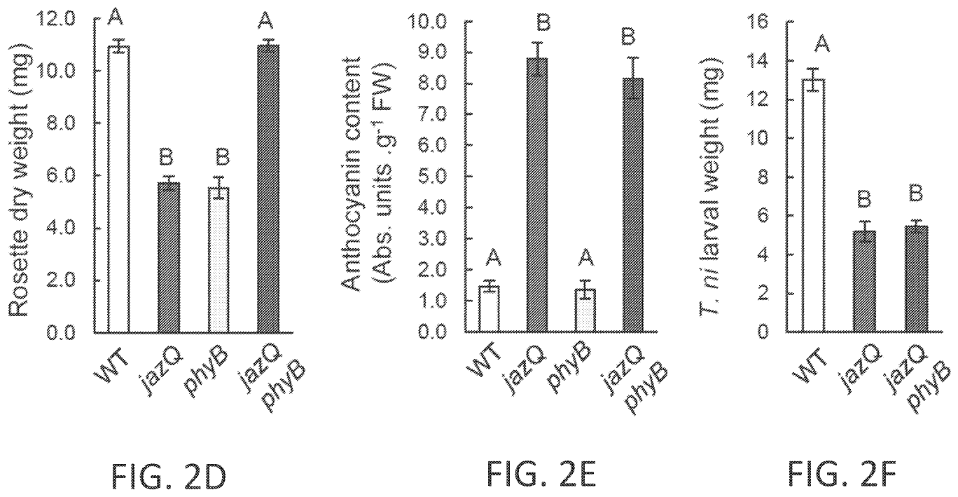

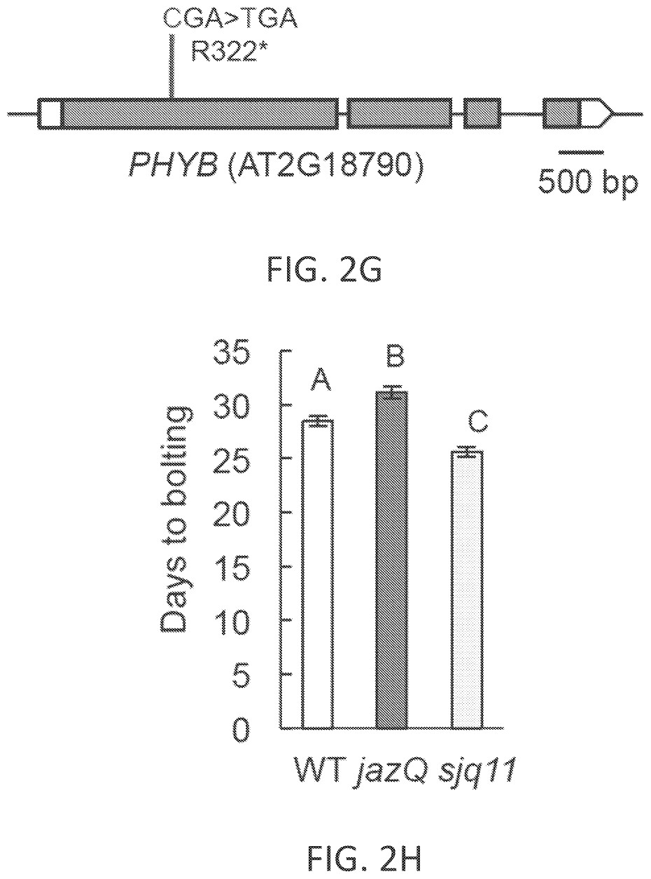

jazQ suppressor screen and identification of sjg11. Approximately 50,000 jazQ mutant seeds were further mutagenized by immersion in a solution of 0.1% or 0.2% (v/v) ethyl methanesulfonate (EMS, Sigma-Aldrich) for 16 hours at room temperature, with constant agitation. Seeds (M₁ generation) were thoroughly washed with water, stratified in the dark at 4° C. for two days and then immediately sown on soil. M2 seed was collected from 16 pools of self-pollinated M₁ plants (approximately 1,000 M₁ plants/pool). Soil-grown M2 plants (˜2000 plants/pool) were visually screened for individuals having a larger rosette size than jazQ mutant seedlings. Putative sjq (suppressors of the jazQ) mutants were rescreened in the M3 generation to confirm heritability of phenotypes. Insight into the causal mutation in sjg11 came from the observation that sjq11 seedlings grown on MS medium in continuous white light for 3 days have elongated hypocotyls. Subsequent hypocotyl growth assays in monochromatic red light (Wamasooriya & Montgomery, Plant Physiol 149, 424-433 (2009)) confirmed a defect in red light signaling. Briefly, sjg11 (M₃ generation) and control seeds were plated on MS medium lacking sucrose and stratified at 4° C. in the dark for two days. Mutant and control lines were grown on the same plate to control for plate-to-plate variation. A 3 hours a pulse of white light was then administered to improve synchronous seed germination. Plates were then returned to darkness for one day at 21° C. and then transferred to a monochromatic LED chamber outfitted to emit red light (670±20 nm; 25 μE μE m⁻² s⁻¹). As a control, a set of plates containing each genotype was maintained in darkness. Following three days of growth, seedling hypocotyls were measured by ImageJ software analysis of scanned images. Allelism tests performed with F1 seedlings (obtained from the cross between sjg11 and phyB-9) revealed a lack of genetic complementation. Sequencing of the PHYB gene (AT2G18790) in sjg11 revealed a C→T transition that introduces a stop codon in a region of the gene that encodes the chromophore-binding domain of PHYB (see FIG. 2G).

Gene expression profiling. Global gene expression profiling in 8-day-old whole seedlings (Col-0 WT, mutant jazQ, mutant phyB-9, mutant jazQ phyB-9) was assessed by mRNA sequencing (RNA-seq) performed on the Ilumina HiSeq 2000 platform. Seedlings were grown in continuous light on solid MS medium supplemented with sucrose. For each replicate sample, approximately 200 seedlings were harvested for RNA extraction. WT and mutant seedlings were grown on the same plate to minimize plate-to-plate variation.

Three independent RNA samples (biological replicates) were sequenced per genotype. Total RNA was isolated as described above and RNA integrity was assessed with a 2100 Bioanalyzer (Agilent Technologies). All samples utilized had an integrity score of at least 7.0. Single-end (50 bp) sequencing was performed at the Michigan State University Research Technologies Service Facility (see website at rtsfnatsci.msu.edu). Barcoded sequencing libraries were constructed using the Illumina RNAseq kit according to the manufacturer's instructions and were multiplexed in six libraries per lane. The average number of sequencing reads was 18.4±4.3 million per sample. Raw sequencing reads were assessed with Illumina quality control tools filters and FASTX toolkit (see website at hannonlab.cshl.edu/fastx_toolkit/). Reads were mapped to gene models in TAIR10 with the program RSEM (version 1.2.11) set for default parameters (Li & Dewey, BMC Bioinformatics 12, 323, (2011)). Data was expressed as transcripts per million (TPM), and the average TPM±standard error for Arabidopsis genes.

DESeq (version 1.18.0; see Anders & Huber, Genome Biol 11, R106 (2010)) was used to normalize expected counts from RSEM and to assess differential gene expression by comparing normalized counts in WT to those in a particular mutant. Gene ontology (GO) analysis of enriched functional categories was performed using BiNGO (version 2.44; Maere et al., Bioinformatics 21, 3448-3449 (2005)). The hypergeometric test with Benjamini & Hochberg's FDR correction was used to calculate over-represented and under-represented GO categories among differentially expressed genes, using a P value <0.05.

For wounding experiments, three-week old soil-grown seedlings were wounded twice across the midvein of four leaves (leaves 3-6, counted from first rosette leaf). After 1 h, the wounded leaves of two plants were pooled and immediately frozen in liquid nitrogen. Equivalent leaves of two unwounded plants were pooled and collected as controls. The experiment was independently replicated twice, with each experiment consisting of 3-4 biological replicates. Frozen tissue was homogenized with a TissueLyser II (Qiagen) and RNA was extracted using an RNeasy kit (Qiagen) with on-column DNase (Qiagen) treatment, as per the manufacturer's instructions. RNA quality was assessed by A₂₆₀/A₂₈₀ ratios using a ND-1000 UV Nanodrop spectrophotometer (Thermo Scientific). cDNA was reverse transcribed using a High-Capacity cDNA Reverse Transcription kit (Applied Biosystems, ABI), as per the manufacturer's instructions, and cDNA was diluted to 0.5 ng/L with nuclease-free water. qRT-PCR was performed as described by Attaran et al. (Plant Physiol 165, 1302-1314 (2014)), with minor modifications. Briefly, reactions were performed on an ABI 7500 Fast qPCR instrument, and consisted of 5 L of 2× Power SYBR Green (ABI) master mix, 2 uL diluted cDNA template (1 ng total), 1 μL 5 uM forward and reverse primers, and nuclease-free water for 10 μL total reaction volume. The forward and reverse primers used were the following:

PP2A:
(SEQ ID NO: 102)
5′-AAGCAGCGTAATCGGTAGG-3′
and
(SEQ ID NO: 103)
5′-GCACAGCAATCGGGTATAAAG-3′;
AOS:
(SEQ ID NO: 104)
5′-GGAGAACTCACGATGGGAGCGATT-3′
and
(SEQ ID NO: 105)
5′-GCGTCGTGGCTTTCGATAACCAGA-3′;
LOX3:
(SEQ ID NO: 106)
5′-GCTGGCGGTTCGACATG-3′
and
(SEQ ID NO: 107)
5′-GCCATTCCTCTGCGAATTAGA-3′;
and
MYC2:
(SEQ ID NO: 108)
5′-AGAAACTCCAAATCAAGAACCAGCTC-3′
and
(SEQ ID NO: 109)
5′-CCGGTTTAATCGAAGAACACGAAGAC-3′.

Reactions were run with the following conditions: 95° C. for 10 min, then 40 cycles of 15 s at 95° C. for denaturation and 60 s at 60° C. for annealing and polymerization. A dissociation curve was performed at the end of each reaction to confirm primer specificity using default parameters (15 s at 95° C., 60 s at 60° C.-95° C. in 1° C. increments, and 15 s at 95° C.). Target gene expression was normalized to the expression of PP2a, which is stable under JA-inducing conditions. The normalization incorporated primer efficiencies determined for each primer pair using LinRegPCR v2012.0⁴⁶ from the log-linear phase of each amplification plot.

Overexpression of PIF4 in the mutant jazQ background. The 35S::PIF4-TAP overexpression construct (see Lee & Thomashow, Proc Natl Acad Sci USA 109, 15054-15059 (2012)). Transformation of mutant jazQ plants with Agrobacterium tumefaciens (strain C58C1) was performed using the flower dip method (Clough & Bent, Plant J 16, 735-743 (1998)). Multiple independent transformed lines (T1 generation) were selected on MS plates containing gentamycin and transferred to soil for subsequent analysis. Homozygous lines were selected by testing the T3 progeny for gentamycin resistance.

Photosynthesis measurements. Gas exchange measurements were obtained as described by Attaran et al. (Plant Physiol 165, 1302-1314 (2014)), and Li et al. (Photosynth Res 112, 49-61 (2012)). Plants were grown in plastic containers (“Cone-tainers”, Steuwe and Sons, Tangent, OR, USA) on an 8 h light (19° C.)/16 h dark (16° C.) photoperiod and 120 μmol m⁻² s⁻¹ photosynthetic photon flux density (PPFD). Single mature rosette leaves (attached) from 8- to 10-week-old plants were used to obtain CO₂ response curves on a LI-6400XT system (LI-COR Biosciences, Lincoln, NE, USA) outfitted with a standard leaf chamber (chamber area=6 cm²). Leaves were supplied with an artificial air mixture consisting of 20% O₂, 80% N₂, and 400 ppm CO₂ at intensity of light 500 μmol m⁻² s⁻¹. Leaf temperature was maintained at ˜20° C. (block temperature set to 18° C.). Leaves were acclimated under this condition for at least 30 min before the start of each experiment. Assimilation rates were normalized to projected leaf area as measured by image analysis with the GIMP software. Area-based and whole plant-based photosynthesis and respiration was determined at four time points of the Arabidopsis growth cycle as described by Weraduwage et al. (Front Plant Sci 6, 167 (2015)), in plants grown under short-day conditions.

In situ chlorophyll a fluorescence measurements were performed in a Percival AR41L2 (Geneva Scientific, see website at geneva-scientific.com) refitted as a Dynamic Environment Photosynthesis imager (DEPI), as described by Attaran et al. (Plant Physiol 165, 1302-1314 (2014)), Dutta et al. (Plant J 84, 428-442 (2015)), and Kramer et al. (WO 2013181433 A2 (2013)). Images were processed using visual phenomics software (Tessmer et al., BMC Syst Biol 7, (Suppl 6) S17 (2013)). The quantum yield of PSII (ΦII) was calculated as (F′_{M}−F_{S})/F′_{M}, where F_{S} is the steady-state fluorescence and F′_{M} is the fluorescence maximum at steady state (Baker, Annu Rev Plant Biol 59, 89-113 (2008)).

Leaf thickness measurements. Leaf cross sections obtained from the 5^{th} leaf of 22-day old rosette leaves were examined under an Olympus FluoView FV1000, Confocal Laser Scanning Microscope (Olympus, NJ, USA) in the Center for Advanced Microscopy, Michigan State University. Leaf thickness was measured as the distance between the abaxial and adaxial surfaces of the leaf as described by Weraduwage et al. (Front Plant Sci 6, 167 (2015)).

Measurement of total chlorophyll and Rubisco concentration in leaves. Extraction and quantification of chlorophyll was carried out using a protocol modified from Lichtenthaler & Wellburn (Biochem Soc Trans 11, 591-592 (1983)). Total chlorophyll was extracted from 54-d old Arabidopsis rosette leaves with 96% ethanol. Absorbance of the extracted chlorophyll was measured spectrophotometrically at 665 nm and 649 nm and the total chlorophyll was calculated using the following equation:

Chlₐ+Chl_{b}=(13.95A₆₆₅−6.88A₆₄₉)+(24.96A₆₄₉−7.32A₆₆₅).

Total protein was extracted from 54-d old Arabidopsis rosette leaves using a Plant Total Protein Extraction Kit (Sigma-Aldrich, MO, USA). A modified Lowry Assay was performed to measure the total protein concentration in the extract and the purity and quality of the extracted protein were determined by denaturing polyacrylamide gel electrophoresis. Equal amounts of total protein were loaded onto an automated capillary-based size western blotting system (ProteinSimple Wes System, San Jose CA, USA). All procedures were performed with manufacturer's reagents according to their user manual. Protein separation and immunodetection were performed automatically on the individual capillaries using the default settings.

Antibodies raised against the large subunit of Rubisco (rabbit antibodies, AS03 037; Agrisera, Sweden; dilution used 1:650) were used to detect Rubisco in each protein sample. For quantification, all subsequent data generated was analyzed with the ‘Compass Software’ provided by manufacturer (ProteinSimple, San Jose CA). Peak heights of the fluorescence signals were used to calculate relative differences of Rubisco concentration between samples. Rubisco concentration per unit leaf area was calculated based on the total protein concentration and measurements of leaf area per unit mass.

### Example 2: Mutants with Enhanced Jasmonate (JA)-Regulated Defense Against Insects

A genetic screen was performed to identify mutants of Arabidopsis that display enhanced jasmonate (JA)-regulated defense against insect herbivory without an associated reduction in leaf growth. This screen leveraged a signaling model predicting that removal of JAZ repressor proteins would constitutively activate defense and inhibit growth (FIG. 1A). A mutant plant line (jaz quintuple or jazQ) was developed with T-DNA insertion mutations in five (JAZ1/3/4/9/10) of the 13 Arabidopsis JAZ genes (FIG. 1G). These JAZs were selected on the basis of their phylogenetic relationship, their demonstrated role in inhibiting MYC transcription factors, and their capacity to interact with DELLA proteins that antagonistically link JA signaling to gibberellic acid (GA)-mediated growth responses (FIG. 1A).

Root growth assays showed that mutant jazQ seedlings have both an increased sensitivity to exogenous JA and a constitutive short-root phenotype (FIGS. 1B and 1I). The short root phenotype is consistent with recent genetic analysis of JA signaling in roots (Gasperini et al., PLoS Genet 11, e1005300 (2015)). Glucosinolates and anthocyanins, whose biosynthesis in Arabidopsis is positively regulated by jasmonic acid, accumulated to higher levels in jazQ mutant seedlings than in wild type (WT) seedlings (FIG. 1C-1D). Soil-grown jazQ mutant plants had remarkably heightened resistance to attack by the generalist herbivore Trichoplusia ni (FIG. 1E). In contrast to these elevated defense traits, leaf area, petiole length, and rosette dry weight were all reduced in jazQ mutants relative to WT (FIG. 1F). The jazQ mutations also delayed the time to bolting but did not affect the number of leaves at the time of bolting. These results demonstrate that jazQ mutant plants exhibit constitutive growth-defense antagonism (i.e., reduced growth with enhanced defense) and thus provide a unique genetic model with which to interrogate how JA-triggered immunity inhibits growth.

### Example 3: Screen for Mutants with Enhanced Jasmonate (JA)-Regulated Defense Against Insects without Reduced Leaf Growth

An ethyl methanesulfonate (EMS)-mutagenized population of jazQ was visually screened for mutants with increased rosette size and persistence of elevated leaf anthocyanin content. Among several suppressor of jazQ (sjq) mutants identified, one line (sjg11) showed a particularly striking leaf growth pattern that was heritable in the M3 generation (FIG. 2A). Importantly, bioassays performed with T. ni larvae showed that sjg11 plants also maintained heightened defenses (FIG. 2B). Characterization of sjg11 plants revealed phenotypes similar to those described for phytochrome B (phyB) photoreceptor mutants, including early flowering time, as well as elongated hypocotyls and petioles under continuous white light. Genetic allelism tests and DNA sequencing confirmed that sjg11 harbors a null mutation in the PHYB gene (FIG. 2G). To eliminate the possibility that additional EMS mutations contribute to the sjg11 phenotype, further studies were performed with a jazQ phyB sextuple mutant obtained by crossing the reference phyB-9 null allele into the jazQ mutant background.

### Example 4: Analysis of Growth and Defense Traits in jazQ phyB Plants

Analysis of growth and defense traits in jazQ phyB mutant plants showed that the jazQ and phyB “single” mutant phenotypes were largely additive and often tissue specific. Mutant jazQ phyB seedlings, for example, retained the JA-hypersensitive root growth inhibition and red-light insensitive hypocotyl elongation phenotypes of jazQ and phyB, respectively. Adult jazQ phyB mutant plants grown in soil resembled phyB in having elongated petioles, flat rosette leaves, and early flowering time (FIG. 2). The phyB mutation is thus epistatic to jazQ for these traits. The rosette diameter, projected leaf area, and dry mass of jazQ phyB rosette leaves exceeded that of the jazQ and phyB parents, indicating that the combination of jazQ and phyB has transgressive effects on leaf growth (FIG. 2D).

Despite its robust vegetative growth, jazQ phyB mutant plants maintained the heightened anti-insect defense and anthocyanin content of jazQ (FIG. 2E-2F). The effect of combining jazQ and phyB mutations on resistance to T. ni feeding was particularly striking because phyB mutations alone cause high susceptibility to this herbivore. The jazQ phenotype is therefore epistatic to phyB with respect to leaf defense traits. These data demonstrate that phyB mutations fully suppresses the slow growth of mutant jazQ rosette leaves without compromising heightened resistance to T. ni feeding.

The JA and PHYB signaling pathways interact to mediate growth-defense balance during the shade avoidance response. Within this signaling network, GA stimulates cell extension growth by promoting the degradation of DELLA proteins that repress PIF transcription factors (FIG. 1A; see also de Lucas et al. Nature 451, 480-484 (2008)). Reciprocal antagonism between the JA and GA pathways involves JAZ-DELLA interactions that prevent these repressors from inhibiting their cognate transcription factors (Hou et al., Dev Cell 19, 884-894 (2010); Yang et al., Proc Natl Acad Sci USA 109, 1192-1200 (2012)). JA-GA crosstalk is integrated with the shade avoidance response through PHYB-mediated perception of changes in the ratio of red to far red (R:FR) light. Low R:FR ratios indicative of leaf shading reduce PHYB activity to relieve repression on PIFs, thereby promoting rapid growth through the concerted action of auxin and brassinosteroids (FIG. 1A). Concurrent with this growth response to plant competitors, inactivation of PHYB by low R:FR (or phyB mutation) also leads to depletion of DELLA proteins, increased JAZ stability, accelerated turnover of MYCs, and suppression of JA-triggered immune responses.

### Example 5: Expression Patterns of Wild Type, jazQ, phyB, and jazQ phyB Seedlings

This Example describes transcript profiles obtained by mRNA sequencing of WT, mutant jazQ, mutant phyB, and mutant jazQ phyB seedling tissues to ascertain how the combination of phyB and jazQ mutations affects gene expression.

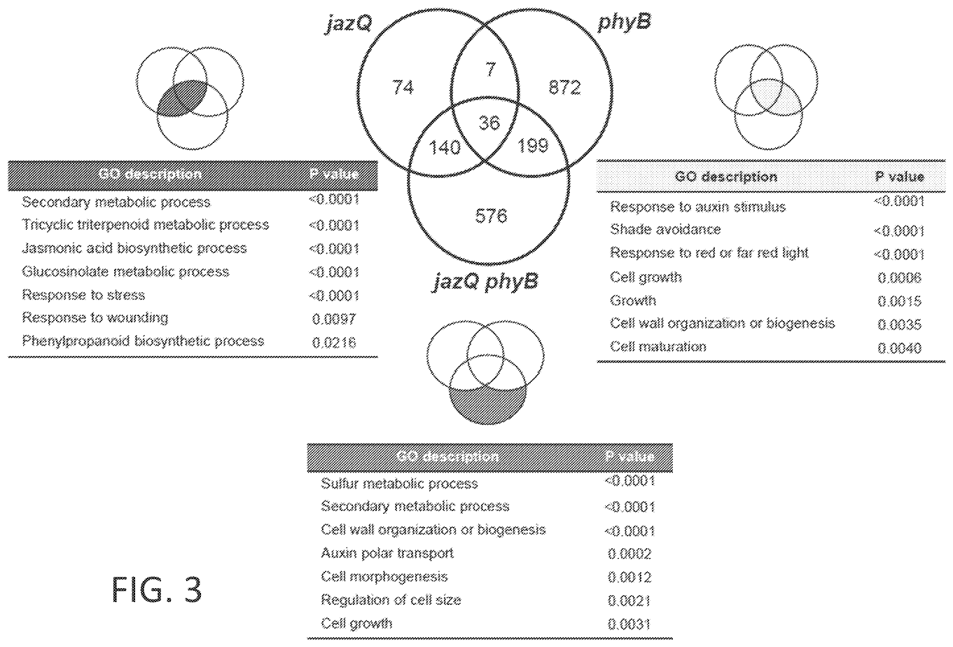

“Secondary metabolism” and “response to stress” and were among the biological processes most significantly overrepresented in ontologies of 257 genes expressed to higher levels in jazQ than in WT (FIG. 3). This gene set included glucosinolate biosynthesis genes that are direct targets of MYC2, as well as genes involved in the synthesis of triterpenoids, jasmonates, and various defense proteins.

Consistent with their enhanced defense stature, jazQ phyB plants maintained increased expression of the majority (68%) of genes that are upregulated in jazQ (FIG. 3). By comparison, analysis of growth-related genes revealed that the set of 235 genes upregulated in both phyB and jazQ phyB genetic backgrounds is enriched for functional classes involved in responses to auxin, shade avoidance, cell wall organization, and light stimulus (FIG. 3). Several genes within this group have been shown to be direct targets for PIF transcription factor binding (Oh et al., Nat Cell Biol 14, 802-809 (2012); Hornitschek et al., Plant J 71, 699-711 (2012); and Zhang et al., PLoS Genet 9, e1003244 (2013)). These data indicate that the combination of jazQ and phyB mutations promotes simultaneous expression of defense and growth-related genes that are controlled, at least in part, by the MYC and PIF transcriptional modules, respectively.

Among the 576 transcripts whose abundance was significantly increased in jazQ phyB mutants but not jazQ or phyB mutants, there was a strong over-representation of GO terms related to secondary metabolism, cell wall organization, growth, and auxin transport (FIG. 3). These data indicate that the combination of jazQ and phyB mutations leads to increased expression of certain growth and defense responses in jazQ phyB mutant plants. Quantitative PCR analysis showed that wound-induced expression of select JA-response genes was significantly higher in jazQ phyB mutant leaves than WT leaves, which may also contribute to the heightened defense of jazQ phyB mutant plants relative to WT. The synergistic effects of jazQ and phyB mutations on gene expression may thus result from functional interaction between MYCs and PIFs at the level of protein-protein interaction or altered binding to common cis-regulatory elements in target genes.

### Example 6: Photosynthetic Efficiency

This Example describes investigations of whether jazQ and phyB mutations interact to modulate leaf photosynthetic efficiency.

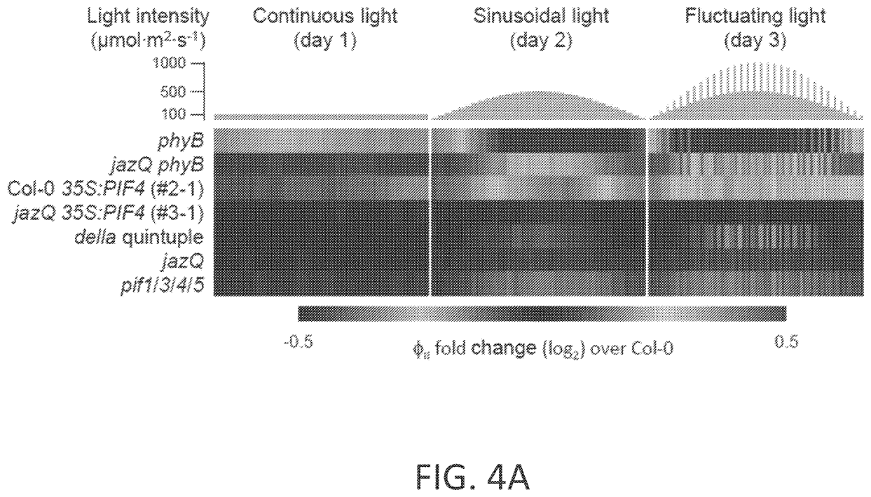

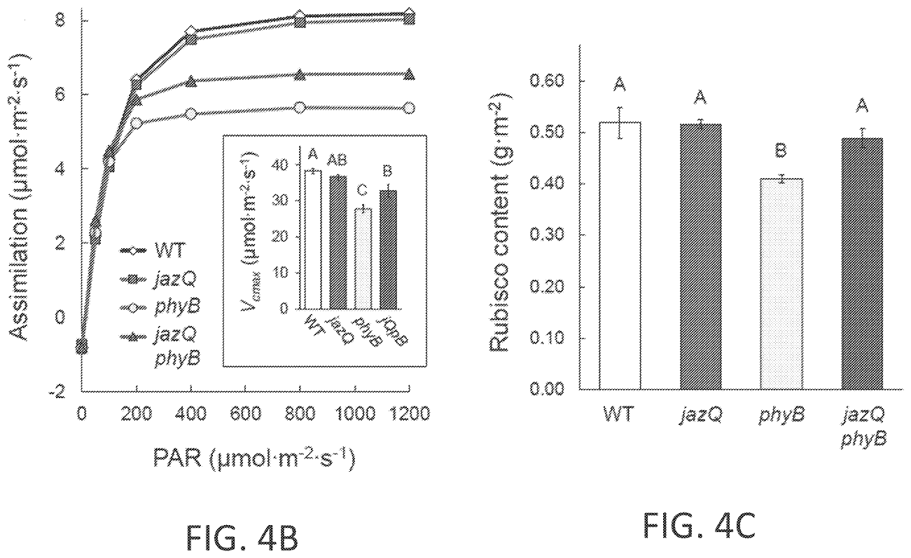

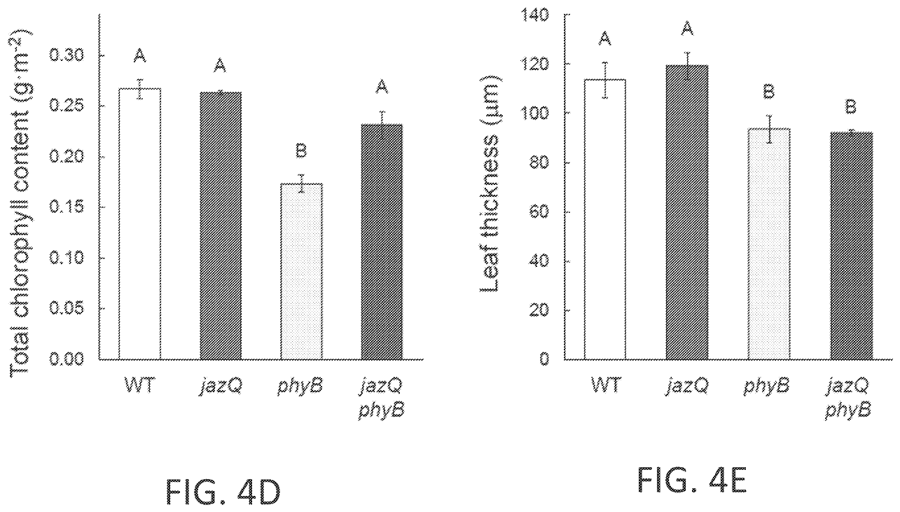

PIF activity can repress chloroplast development and photosynthetic competency, and the inventors have observed that “photosynthesis” was a characteristic most significantly overrepresented among genes that are repressed in both phyB mutant and jazQ phyB mutant seedlings. Non-invasive, whole-plant chlorophyll fluorescence imaging (Attaran et al., Plant Physiol 165, 1302-1314 (2014)) was used to determine how genetic perturbations within the PHYB-GA-JA signaling network affect photosystem II efficiency (Φ_{II}) under various light regimes, including those designed to simulate natural environments (see FIG. 4A).

Mutant phyB plants had reduced Φ_{II} under continuous low light intensity and this effect was exacerbated under the sinusoidal and fluctuating light regimes. A similar decrease in (DII was observed in Col-0 transgenic plants (35S:PIF4) that overexpressed PIF4. Interestingly, the negative effect of phyB mutations and 35S:PIF4 on Φ_{II} was rescued by jazQ mutations, which alone had little (or very weak positive) effect on Φ_{II} (FIG. 4A). Consistent with the role of PIFs in repressing photosynthesis, apif1/3/4/5 quadruple mutant (pifq) showed increased (DII under fluctuating light conditions, whereas loss of DELLAs in the della quintuple mutant (dellaQ) reduced (DII. That (DII was lower in phyB leaves than in dellaQ leaves suggests that phyB has a predominate role in repressing PIF activity in leaves under these growth conditions.

To obtain additional insight into physiological processes that underlie growth-defense vigor of jazQ phyB mutant plants, the relationship between photosynthesis and leaf growth was investigated to obtain an estimate of leaf construction costs. Gas exchange experiments showed that phyB mutant eaves have significantly lower photosynthetic rate per unit leaf area whereas photosynthetic capacity of jazQ mutant plants relating to leaf area or dry weight basis was comparable to WT (FIG. 4B), consistent with our chlorophyll fluorescence measurements. Mutant phyB leaves also contained less area-based chlorophyll and Rubisco (D-ribulose-1,5-bisphosphate carboxylase/oxygenase) than WT.

Modeling of photosynthetic parameters showed that the reduced photosynthetic capacity of mutant phyB at high light results in part from a limitation in Rubisco activity. Mutations in jazQ partially rescued the low photosynthetic capacity of phyB leaves, as well as the low area-based Rubisco and chlorophyll content of phyB mutations (FIG. 4C-4D). In addition, mutant phyB leaves were thinner than WT and mutant jazQ leaves; this trait was retained in jazQ phyB mutant plants (FIG. 4D).

Because of the greater projected leaf area available to intercept light (due to longer petioles and flatter, thinner leaves), the whole plant photosynthetic rate in jazQ phyB mutant plants was similar to WT. Thus, costs associated with jazQ phyB mutations one leaf structure may be lowered through increased partitioning of carbon to leaf area at the expense of leaf thickness. These data suggest that changes in leaf architecture rather than increased efficiency of the photosynthetic apparatus may contribute to the growth-defense vigor of jazQ phyB mutant plants relative to WT plants.

In summary, an unbiased genetic approach was employed to show that JA-mediated growth-defense antagonism can be effectively eliminated through genetic removal of JAZ and phyB repressors that respectively restrain the MYC and PIF transcription modules in WT plants. In highlighting a general role for transcriptional repressors in tuning photoassimilate use efficiency in mature plants, the results described herein provide new insight into how JA and light signaling pathways are integrated to control the balance between growth and immune responses. Note also that the expression of genes involved in abiotic stress response is repressed in jazQ phyB mutant plants.

The ability of jazQ phyB mutant leaves to grow and at the same time defend against insects and other threats indicates that JA signaling does not simply divert photoassimilates or other metabolic resources to defense processes at the expense of growth. The data described herein support a conclusion that the linkage between JA-triggered immunity and growth inhibition is more accurately viewed as a phenotypic consequence of interconnected transcriptional networks that evolved to optimize fitness in the face of episodic, unpredictable encounters with plant consumers and competitors.

Genetic removal of transcriptional repressors that coordinate growth and immune responses provides an approach to combine desirable traits in new ways, and increase the productivity of densely planted crops with less dependence on pesticides.

### Example 7: Overexpression of PIF4 in the jazQ Background Leads to Partial Rescue of Growth without Compromising Defense

The phyB-Jasmonic acid crosstalk led the inventors to test the hypothesis that the combination of jazQ and phyB mutations causes concomitant de-repression of the MYC and PIF transcriptional programs to drive growth and defense simultaneously (FIG. 1A). The inventors then tested what effect overexpression of PIF4 would have in the jazQ mutant background.

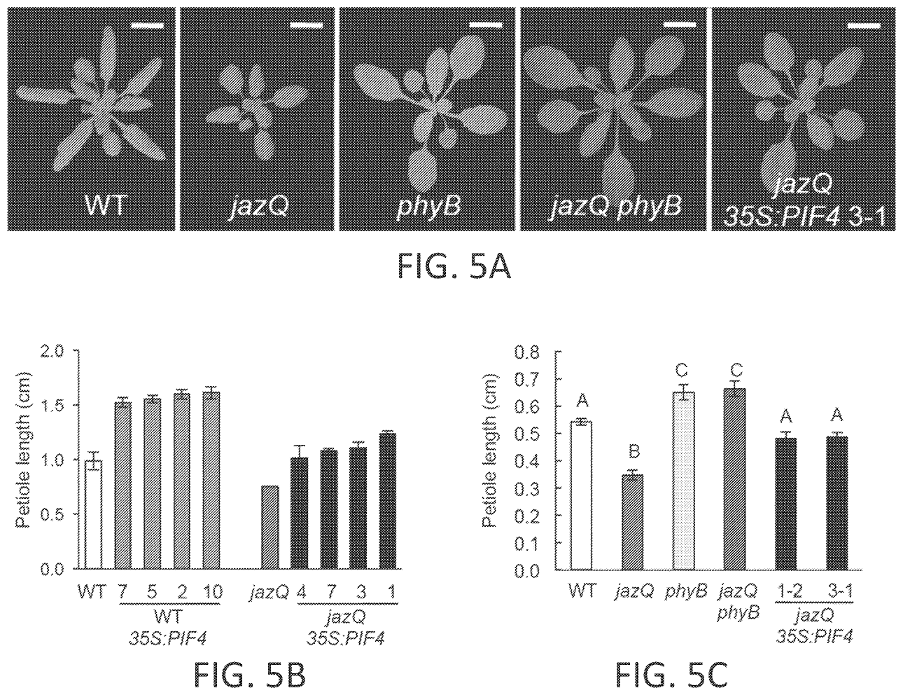

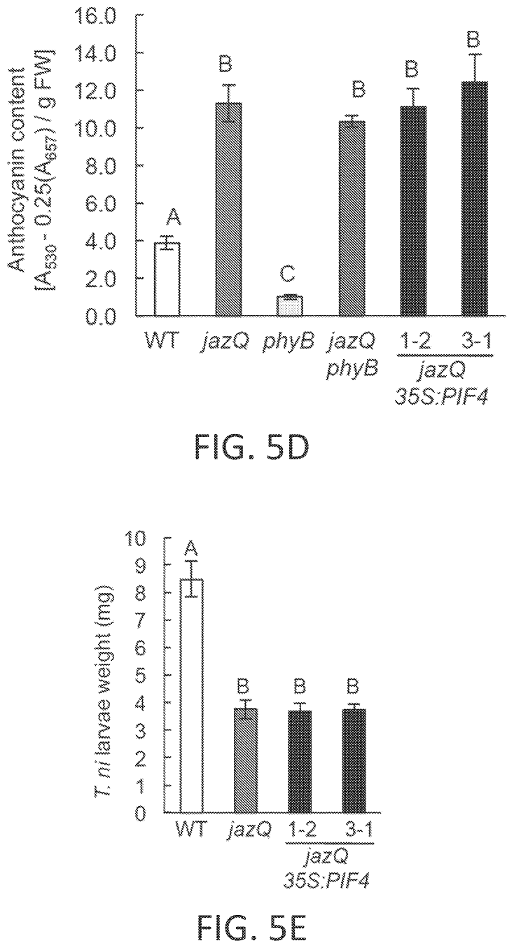

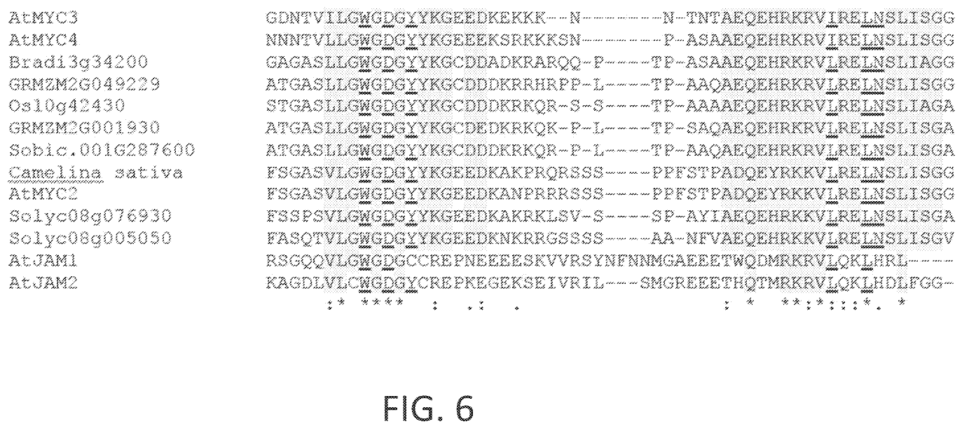

As shown in FIG. 5A-5E, overexpression of PIF4 partially rescued the small rosette size and short petiole length of jazQ mutations without affecting anthocyanin accumulation and resistance to T. ni feeding. This finding indicates that increased PIF4-mediated growth does not attenuate the defense status of jazQ mutation on leaf structures. These findings also indicate that other PIFs may contribute to the growth vigor of jazQ phyB mutant plants.

### Example 8: MYC3^{L152A} and MYC3^{E148A/M155}A Mutants

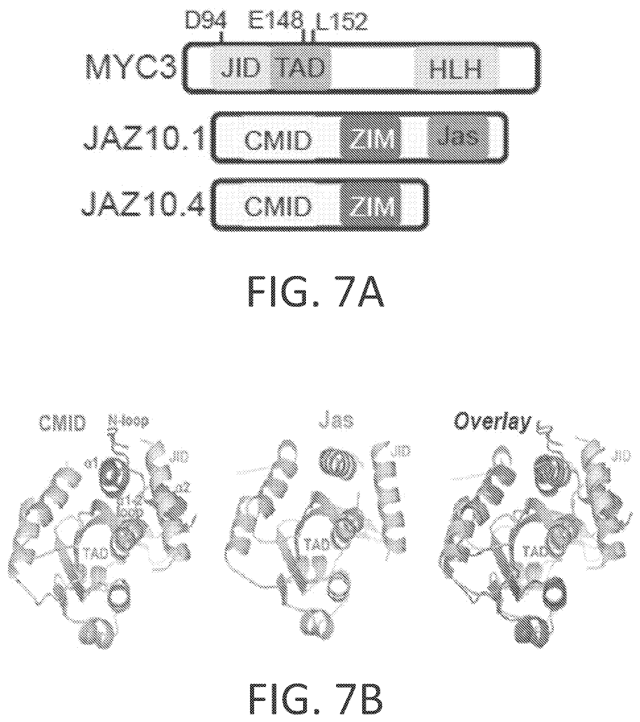

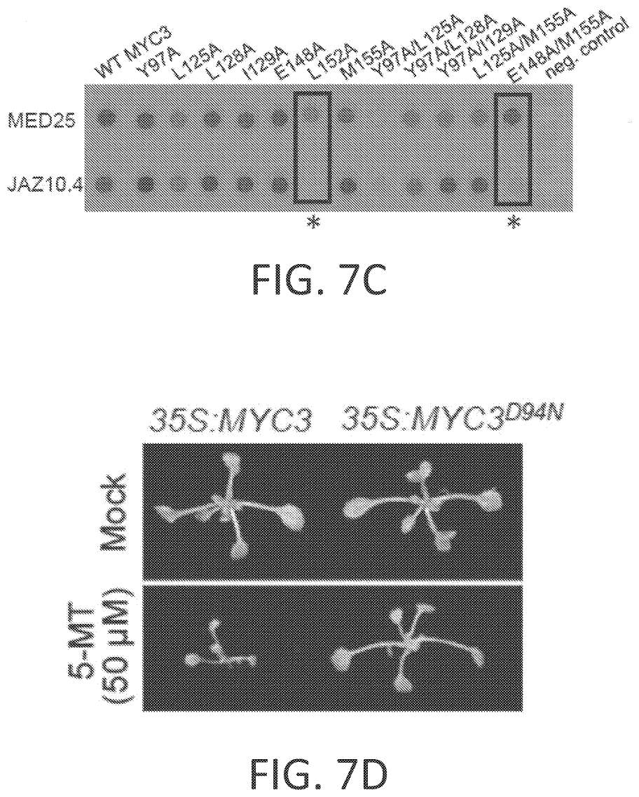

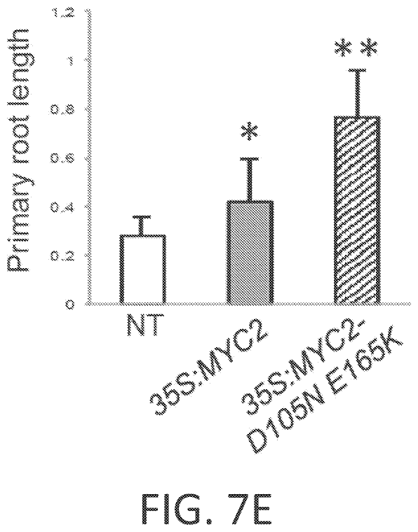

JAZ proteins contain two distinct structural motifs that bind to the JAZ-interacting domain (JID) of MYC (FIG. 7A-7B). All JAZ proteins contain a C-terminal Jas motif that, in the absence of jasmonic acid, forms an extended α-helix that binds JID (Zhang et al. 2015; Katsir et al. 2008; Melotto et al. 2008). A subset of JAZ proteins (e.g., JAZ10 and JAZ1) contain a cryptic MYC-interaction domain (CMID) near the N-terminus that also binds the JID (Moreno et al. 2013; Chung and Howe 2009; Goossens et al. 2015). Comparison of the x-ray crystal structures of the MYC3-JAZ10^{CMID} and MYC3-JAZ10^{Jas} complexes showed that whereas the Jas motif binds MYC as a single continuous α-helix, the CMID adopts a bipartite structure in which one helix occupies the Jas-binding groove of MYC and a second helix makes contact with the backside of this groove (FIG. 7B). This clamp-like action of the CMID engages MYCs with higher affinity than the Jas helix and also effectively masks the MED25 binding site of MYC (Zhang et al. 2017). Sequence alignments indicate that CMID-containing JAZs are present in diverse plant species but none have been characterized to date.

The inventors have used structural information for rational design of dominant MYC transcription factors (MYC^{D} TFs) that are insensitive to binding by both the Jas and CMID of JAZ. The inventors hypothesize that such dominant mutant MYC proteins can strongly activate defense gene expression in the absence of JA elicitation. Three AtMYC^{D} variants have been reported in the literature: MYC3^{D94N} (atr2D allele)(Smolen et al. 2002), the corresponding MYC2 mutant (MYC2^{D105N})(Goossens et al. 2015), and MYC2^{E165K} (myc2-322B) (Gasperini et al. 2015). These MYC variants exhibit weak constitutive JA responses as a consequence of losing interaction with the Jas motif of JAZ.

The inventors hypothesize that design of MYC^{D} TFs that are insensitive to both Jas and CMID binding will result in much stronger activation of JA-dependent defenses, and that the combination of these myc^{D} alleles with phyB will drive robust growth and defense simultaneously.

The structural information from the MYC3-CMID complex was used to generate site-directed mutants of MYC3 that have reduced interaction with the JAZ10.4 splice variant, which contains a CMID but not a Jas motif (Chung and Howe 2009). Several promising mutants were made.

Binding studies were performed to evaluate the mutant MYC proteins. AtMYC2 and its paralogs (MYC3, MYC4, and MYC5) within subclade IIIe of the bHLH superfamily bind to G-box motifs to promote the expression of a large portion of JA-responsive genes in cells containing elevated JA levels (Fernández-Calvo et al. 2011; Schweizer et al. 2013; Major et al. 2017). Low levels of JA stabilize JAZs to permit JAZ binding to the JAZ-interacting domain (JID) of MYCs. JAZ binding to the JID represses MYC activity by two distinct mechanisms.

First, JAZs use their EAR and ZIM motifs to recruit the TOPLESS (TPL) co-repressor and associated chromatin-modifying enzymes (Shyu et al. 2012; Pauwels et al. 2010). Second, JAZ binding to the JID competitively inhibits MYC interaction with the MED25 subunit of the Mediator of RNA polymerase II transcription complex, which promotes transcription by bridging DNA-bound transcription factors to RNA polymerase II (Zhang et al. 2015). Although the JID and adjacent transactivation domain (TAD) of MYC were initially mapped as discrete regions (Kazan & Manners 2013), recent structural analysis of JAZ9-MYC3 complexes revealed that the JID and TAD functionally overlap to form a continuous groove that binds both JAZ and the MED25 in a competitive manner (Zhang et al. 2015).

The interactions of mutants (MYC3^{L152A} and MYC3^{E148A/M155A}) with JAZ10.4 and MED25 was evaluated using yeast two-hybrid analyses to visualize MED25 and JAZ10.4 (bait) interactions with wild-type MYC3 and MYC3 point mutants (prey).

The MYC3^{L152A} and MYC3^{E148A/M155A} mutant protein exhibited strongly reduced JAZ10.4 interaction but still interacted with MED25 (FIG. 7C).

Functional analysis of these and other MYC^{D} transcription factors in planta was performed by overexpressing the transcription factors in Arabidopsis with subsequent testing of the resulting transgenic lines for increased resistance to 5-methyl tryptophan (5-MT), a toxic analog of the amino acid tryptophan.

For example, a dominant allele (atr2D allele) encoding MYC3^{D94N} was identified in a genetic screen for 5-MT-resistant plants (Smolen et al. 2002). Preliminary results show that overexpression from the 35S promoter of MYC3^{D94N} but not wild-type MYC3 confers 5-MT resistance (FIG. 7D).

Similarly, overexpression in Arabidopsis of a tomato (Solanum lycopersicum) MYC2 protein with SEQ ID NO:19 that was modified to have a D132N mutation confers resistance of the genetically modified Arabidopsis plants to 5-MT (data not shown).

### Example 9: Expression of Dominant Mutant Myc2 Reduces Primary Root Inhibition

This Example illustrates that overexpression of a dominant MYC2 mutant transcription factor (with D105N+E165K mutations) in Arabidopsis reduces primary root inhibition in response to treatment with 5-methyltryptophan.

### Methods

Primary root length in the presence of 15 μM 5-MT was evaluated for 14-day-old seedlings of non-transgenic (NT)phyB-9 mutant plants or phyB-9 mutant plants that overexpressed either wild-type (WT) MYC2 or a MYC2^{D105N/E165K} mutant allele that harbors two point mutations (D105N and E165K) that prevent JAZ binding. Primary root length was determined as the root length of a given genotype grown in the presence of 15 μM 5-MT normalized to the root length of the same genotype grown in the absence of 5-MT.

### Results

FIG. 7E graphically illustrates that the primary root length of 14-day-old seedlings of non-transgenic (NT)phyB-9 mutant plants (left bar) was less than observed for the phyB-9 mutant plants that overexpress either wild-type (WT) MYC2 (middle bar) or a MYC2 derivative (MYC2^{D105N/E165K}; right bar) harboring two point mutations (D105N and E165K) that prevent JAZ binding. Asterisks indicate significant differences in relative root length (*P=0.0017, **P=2E-08) in comparisons to phyB. Error bars represent SEM, n>20. Hence, expression of such dominant MYC transcription factors conferred resistance to 15 μM 5-MT and fostered growth of the plant as indicated by the significantly increased length of the plants' primary root.

These findings provide proof-of-concept that 5-MT resistance can be used to quantify MYC^{D} activity in planta for rationale design of dominant MYC TFs.

In summary, the inventors have developed a structure-guided pipeline (that incorporates site-directed mutagenesis, yeast two-hybrid analysis of JAZ repressor and MED25 co-activator interaction, and functional analysis of 5-MT resistance in Arabidopsis) to design and characterize MYC^{D} TFs, from any plant species. Plants engineered to express dominant MYC transcription factors in a phyB mutant background can exhibit enhanced growth and defense at the same time.

### REFERENCES

1. Stamp, N. Out of the quagmire of plant defense hypotheses. Q Rev Biol 78, 23-55 (2003).
2 Zust, T., Rasmann, S. & Agrawal, A. A. Growth-defense tradeoffs for two major anti-herbivore traits of the common milkweed Asclepias syriaca. Oikos 124, 1404-1415 (2015).
3 Herms, D. A. & Mattson, W. J. The dilemma of plants—To grow or defend. Q Rev Biol 67, 283-335 (1992).
4 Huot, B., Yao, J., Montgomery, B. L. & He, S. Y. Growth-defense tradeoffs in plants: a balancing act to optimize fitness. Mol Plant 7, 1267-1287 (2014).
5 Moreno, J. E., Tao, Y., Chory, J. & Ballare, C. L. Ecological modulation of plant defense via phytochrome control of jasmonate sensitivity. Proc Natl Acad Sci USA 106, 4935-4940 (2009).
6 Havko, N. E. et al. Control of carbon assimilation and partitioning by jasmonate: An accounting of growth-defense balance. Plants 5, 7 (2016).
7 Kliebenstein, D. J. False idolatry of the mythical growth versus immunity tradeoff in molecular systems plant pathology. Physiol Mol Plant Pathol, in press, doi:10.1016/j.pmpp.2016.02.004 (2016).
8 Hu, P. et al. JAV1 controls jasmonate-regulated plant defense. Mol Cell 50, 504-515 (2013).
9 Leone, M., Keller, M. M., Cerrudo, I. & Ballare, C. L. To grow or defend? Low red:far-red ratios reduce jasmonate sensitivity in Arabidopsis seedlings by promoting DELLA degradation and increasing JAZ10 stability. New Phytol 204, 355-367 (2014).
10 Thireault, C. et al. Repression of jasmonate signaling by a non-TIFY JAZ protein in Arabidopsis. Plant J 82, 669-679 (2015).
11 Thines, B. et al. JAZ repressor proteins are targets of the SCF^{COI1} complex during jasmonate signalling. Nature 448, 661-665 (2007).
12 Chini, A. et al. The JAZ family of repressors is the missing link in jasmonate signalling. Nature 448, 666-671 (2007).
13 Yan, Y. et al. A downstream mediator in the growth repression limb of the jasmonate pathway. Plant Cell 19, 2470-2483 (2007).
14 Hou, X., Lee, L. Y., Xia, K., Yan, Y. & Yu, H. DELLAs modulate jasmonate signaling via competitive binding to JAZs. Dev Cell 19, 884-894 (2010).
15 Yang, D. L. et al. Plant hormone jasmonate prioritizes defense over growth by interfering with gibberellin signaling cascade. Proc Natl Acad Sci USA 109, 1192-1200 (2012).
16 Femandez-Calvo, P. et al. The Arabidopsis bHLH Transcription Factors MYC3 and MYC4 are targets of JAZ repressors and act additively with MYC2 in the activation of jasmonate responses. Plant Cell 23, 701-715 (2011).
17 Schweizer, F. et al. Arabidopsis basic helix-loop-helix transcription factors MYC2, MYC3, and MYC4 regulate glucosinolate biosynthesis, insect performance, and feeding behavior. Plant Cell 25, 3117-3132 (2013).
18 Gasperini, D. et al. Multilayered organization of jasmonate signalling in the regulation of root growth. PLoS Genet 11, e1005300 (2015).
19 Qi, T. et al. The Jasmonate-ZIM-domain proteins interact with the WD-Repeat/bHLH/MYB complexes to regulate jasmonate-mediated anthocyanin accumulation and trichome initiation in Arabidopsis thaliana. Plant Cell 23, 1795-1814 (2011).
20 de Lucas, M. et al. A molecular framework for light and gibberellin control of cell elongation. Nature 451, 480-484 (2008).
21 Casal, J. J. Photoreceptor signaling networks in plant responses to shade. Annu Rev Plant Biol 64, 403-427 (2013).
22 Ballare, C. L. Light regulation of plant defense. Annu Rev Plant Biol 65, 335-363 (2014).
23 Oh, E., Zhu, J. Y. & Wang, Z. Y. Interaction between BZR1 and PIF4 integrates brassinosteroid and environmental responses. Nat Cell Biol 14, 802-809 (2012).
24 Chico, J. M. et al. Repression of jasmonate-dependent defenses by shade involves differential regulation of protein stability of MYC transcription factors and their JAZ repressors in Arabidopsis. Plant Cell 26, 1967-1980 (2014).
25 Hornitschek, P. et al. Phytochrome interacting factors 4 and 5 control seedling growth in changing light conditions by directly controlling auxin signaling. Plant J 71, 699-711 (2012).
26 Zhang, Y. et al. A quartet of PIF bHLH factors provides a transcriptionally centered signaling hub that regulates seedling morphogenesis through differential expression-patterning of shared target genes in Arabidopsis. PLoS Genet 9, e1003244 (2013).
27 Leivar, P. & Monte, E. PIFs: systems integrators in plant development. Plant Cell 26, 56-78 (2014).
28 Attaran, E. et al. Temporal dynamics of growth and photosynthesis suppression in response to jasmonate signaling. Plant Physiol 165, 1302-1314 (2014).
29 Boccalandro, H. E. et al. Phytochrome B enhances photosynthesis at the expense of water-use efficiency in Arabidopsis. Plant Physiol 150, 1083-1092 (2009).
30 Weraduwage, S. M. et al. The relationship between leaf area growth and biomass accumulation in Arabidopsis thaliana. Front Plant Sci 6, 167 (2015).
31 McElver, J. et al. Insertional mutagenesis of genes required for seed development in Arabidopsis thaliana. Genetics 159, 1751-1763 (2001).
32 Jiang, Y., Liang, G., Yang, S. & Yu, D. Arabidopsis WRKY57 functions as a node of convergence for jasmonic acid- and auxin-mediated signaling in jasmonic acid-induced leaf senescence. Plant Cell 26, 230-245 (2014).
33 Sehr, E. M. et al. Analysis of secondary growth in the Arabidopsis shoot reveals a positive role of jasmonate signalling in cambium formation. Plant J 63, 811-822 (2010).
34 Reed, J. W., Nagpal, P., Poole, D. S., Furuya, M. & Chory, J. Mutations in the gene for the red/far-red light receptor phytochrome B alter cell elongation and physiological responses throughout Arabidopsis development. Plant Cell 5, 147-157 (1993).
35 Feng, S. H. et al. Coordinated regulation of Arabidopsis thaliana development by light and gibberellins. Nature 451, 475-479 (2008).
36 Leivar, P. et al. Multiple phytochrome-interacting bHLH transcription factors repress premature seedling photomorphogenesis in darkness. Curr Biol 18, 1815-1823 (2008).
37 Chung, H. S. et al. Regulation and function of Arabidopsis JASMONATE ZIM-domain genes in response to wounding and herbivory Plant Physiol 146, 952-964 (2008).
38 Shyu, C. et al. JAZ8 lacks a canonical degron and has an EAR motif that mediates transcriptional repression of jasmonate responses in Arabidopsis. Plant Cell 24, 536-550 (2012).
39 Kang, J. H. et al. The flavonoid biosynthetic enzyme chalcone isomerase modulates terpenoid production in glandular trichomes of tomato. Plant Physiol 164 (2014).
40 Barth, C. & Jander, G. Arabidopsis myrosinases TGG1 and TGG2 have redundant function in glucosinolate breakdown and insect defense. Plant J 46, 549-562 (2006).
41 Herde, M., Koo, A. J. & Howe, G. A. Elicitation of jasmonate-mediated defense responses by mechanical wounding and insect herbivory. Methods Mol Biol 1011, 51-61 (2013).
42 Warnasooriya, S. N. & Montgomery, B. L. Detection of spatial-specific phytochrome responses using targeted expression of biliverdin reductase in Arabidopsis. Plant Physiol 149, 424-433 (2009).
43 Li, B. & Dewey, C. N. RSEM: accurate transcript quantification from RNA-Seq data with or without a reference genome. BMC Bioinformatics 12, 323, (2011).
44 Anders, S. & Huber, W. Differential expression analysis for sequence count data. Genome Biol 11, R106 (2010).
45 Maere, S., Heymans, K. & Kuiper, M. BiNGO: a Cytoscape plugin to assess overrepresentation of Gene Ontology categories in biological networks. Bioinformatics 21, 3448-3449 (2005).
46 Ruijter, J. M. et al. Amplification efficiency: linking baseline and bias in the analysis of quantitative PCR data. Nucleic Acids Res 37, e45 (2009).
47 Lee, C. M. & Thomashow, M. F. Photoperiodic regulation of the C-repeat binding factor (CBF) cold acclimation pathway and freezing tolerance in Arabidopsis thaliana. Proc Natl Acad Sci USA 109, 15054-15059 (2012).
48 Clough, S. J. & Bent, A. F. Floral Dip: A simplified method for Agrobacterium-mediated transformation of Arabidopsis thaliana. Plant J 16, 735-743 (1998).
49 Li, Z. R., Gao, J. P., Benning, C. & Sharkey, T. D. Characterization of photosynthesis in Arabidopsis ER-to-plastid lipid trafficking mutants. Photosynth Res 112, 49-61 (2012).
50 Dutta, S. et al. Non-invasive, whole-plant imaging of chloroplast movement and chlorophyll fluorescence reveals photosynthetic phenotypes independent of chloroplast photorelocation defects in chloroplast division mutants. Plant J 84, 428-442 (2015).
51 Kramer, D., Cruz, J., Hall, C., Kovac, W. K. & Zegarac, R. Plant phenometrics systems and methods and devices related thereto. United States Patent WO 2013181433 A2 (2013).
52 Tessmer, O. L., Jiao, Y., Cruz, J. A., Kramer, D. M. & Chen, J. Functional approach to high-throughput plant growth analysis. BMC Syst Biol 7, (Suppl 6) S17 (2013).
53 Baker, N. R. Chlorophyll fluorescence: a probe of photosynthesis in vivo. Annu Rev Plant Biol 59, 89-113 (2008).
54 Lichtenthaler, H. K. & Wellburn, A. R. Determinations of total carotenoids and chlorophylls a and b of leaf extracts in different solvents. Biochem Soc Trans 11, 591-592 (1983).
55 Sonderby, I. E., Geu-Flores, F. & Halkier, B. A. Biosynthesis of glucosinolates-gene discovery and beyond. Trends Plant Sci 15, 283-290 (2010).
56. Zhang et al., Structural basis of JAZ repression of MYC transcription factors in jasmonate signalling, Nature 525: 269-273 (2015).
57. Katsir et al., COI1 is a critical component of a receptor for jasmonate and the bacterial virulence factor coronatine, Proc. Natl. Acad. Sci. U.S.A. 105(19): 7100-7105 (2008).
58. Melotto et al., A critical role of two positively charged amino acids in the Jas motif of Arabidopsis JAZ proteins in mediating coronatine- and jasmonoyl isoleucine-dependent interactions with the COI1 F-box protein, The Plant journal: for cell and molecular biology 55(6): 979-988 (2008).
59. Smolen et al., Dominant alleles of the basic helix-loop-helix transcription factor ATR2 activate stress-responsive genes in Arabidopsis, Genetics 161(3): 1235-1246 (2002).
60. Moreno et al., Negative feedback control of jasmonate signaling by an alternative splice variant of JAZ10, Plant physiology 162(2): 1006-1017 (2013).
61. Chung & Howe, A critical role for the TIFY motif in repression of jasmonate signaling by a stabilized splice variant of the JASMONATE ZIM-domain protein JAZ10 in Arabidopsis, The Plant Cell 21(1): 131-145 (2009).
62. Goossens et al., Change of a conserved amino acid in the MYC2 and MYC3 transcription factors leads to release of JAZ repression and increased activity, The New Phytologist 206(4): 1229-1237 (2015).
63. Zhang et al., Structural insights into alternative splicing-mediated desensitization of jasmonate signaling, Proc. Natl. Acad. Sci. U.S.A. 114(7): 1720-1725 (2017).
64. Major et al., Regulation of growth-defense balance by the JASMONATE ZIM-DOMAIN (JAZ)-MYC transcriptional module, The New Phytologist (2017).
65. Pauwels et al., NINJA connects the co-repressor TOPLESS to jasmonate signalling, Nature 464(7289): 788-791 (2010).
66. Kazan & Manners, MYC2: the master in action, Molecular Plant 6(3): 686-703 (2013).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements

1. A plant, plant cell, or plant seed comprising at least one mutant MYC protein with at least one mutation within or outside of a JAZ-interacting domain (JID) of the MYC protein, and aphyB loss-of-function mutation.
2. The plant, plant cell, or plant seed of statement 1, wherein the mutation within or outside of the JAZ-interacting domain (JID) reduces binding of the MYC protein to a JAZ protein selected from a JAZ1 protein, JAZ2 protein, JAZ3 protein, JAZ4 protein, JAZ5 protein, JAZ6 protein, JAZ7 protein, JAZ8 protein, JAZ9 protein, JAZ10 protein, JAZ11 protein, JAZ12 protein, JAZ13 protein, or a combination thereof.
3. The plant, plant cell, or plant seed of statement 1 or 2, wherein the mutation within or outside of the JAZ-interacting domain (JID) reduces binding of the MYC protein to a JAZ protein selected from a JAZ1 protein, JAZ2 protein, JAZ3 protein, JAZ4 protein, JAZ5 protein, JAZ6 protein, JAZ7 protein, JAZ8 protein, JAZ9 protein, JAZ10 protein, JAZ11 protein, JAZ12 protein, JAZ13 protein, or a combination thereof by at least 20%, or at least 30%, or at least 40%, or at least 50%, or at least 60%, or at least 70%, or at least 80%, or at least 90%, or at least 95%, or at least 97%, or at least 99% compared to wild type plant cells, plants, and seeds of the same species (that do not have the MYC mutation(s)).
4. The plant, plant cell, or plant seed of statement 1, 2, or 3, wherein the JAZ-interacting domain (JID) has less than 100%, or less than 99.5%, or less than 99%, or less than 98%, or at less than 97%, or less than 96%, or less than 95%, or less than 94%, or less than 93%, or less than 92%, or less than 91%, or less than 90% sequence identity to any of SEQ ID NOs: 2, 4, 6, 8, 10, 12, 14, 16, 18, 20, 22, 24, 26, or 28.
5. The plant, plant cell, or plant seed of statement 1-3 or 4, wherein the mutant MYC protein has less than 100%, or less than 99.5%, or less than 99%, or less than 98%, or at less than 97%, or less than 96%, or less than 95%, or less than 94%, or less than 93%, or less than 92%, or less than 91%, or less than 90% sequence identity to any of SEQ ID NOs:1, 3, 5, 7, 9, 11, 13, 15, 17, 19, 21, 23, 25, or 27.
6. The plant, plant cell, or plant seed of statement 1-4 or 5, wherein the mutant MYC protein has at least 40%, or at least 50%, or at least 60%, or at least 70%, or at least 80%, or at least 90%, or at least 95%, or at least 97%, or at least 99% sequence identity to any of SEQ ID NOs:1, 3, 5, 7, 9, 11, 13, 15, 17, 19, 21, 23, 25, or 27.
7. The plant, plant cell, or plant seed of statement 1-5 or 6, wherein the mutant MYC protein is expressed from a dominant MYC genomic allele, from an expression cassette encoding the mutant MYC protein, or from an expression cassette encoding a dominant mutant MYC protein.
8. The plant, plant cell, or plant seed of statement 1-6 or 7, comprising a heterologous MYC transgene or MYC expression cassette that encodes the mutant MYC protein.
9. The plant, plant cell, or plant seed of statement 1-7 or 8, comprising a heterologous MYC transgene comprising a promoter operably linked to a nucleic acid segment encoding the mutant MYC protein.
10. The plant, plant cell, or plant seed of statement 1-8 or 9, comprising a heterologous MYC transgene comprising a promoter operably linked to a cDNA encoding the mutant MYC protein.
11. The plant, plant cell, or plant seed of statement 1-9 or 10, wherein the plant, plant cell, or plant seed (or plant grown from the plant seed) has reduced PHYB activity compared to a wild type plant without the phyB loss-of-function mutation.
12. The plant, plant cell, or plant seed of statement 1-10 or 11, wherein the plant, plant cell, or plant seed expresses a PHYB protein with less than 99%, or less than 98%, or less than 95%, or less than 90%, or less than 85%, or less than 75%, or less than 60%, or less than 50%, or less than 40%, or less than 30%, or less than 20% sequence identity to any of SEQ ID NOs:30, 32, 33, 34, 35, 36, or 37.
13. The plant, plant cell, or plant seed of statement 1-11 or 12, wherein the plant, plant cell, or plant seed comprises a deletion in a chromosomal PhyB or PhyB-related chromosomal site, a substitution within a chromosomal PhyB or PhyB-related chromosomal site, or an insertion into a chromosomal PhyB or PhyB-related chromosomal site.
14. The plant, plant cell, or plant seed of statement 1-12 or 13, wherein the plant, plant cell, or plant seed comprises a deletion, substitution, or insertion of a chromosomal PhyB or PhyB-related chromosomal site so that a truncated PHYB polypeptide, a mutant PHYB polypeptide, or no PHYB polypeptide is expressed.
15. The plant, plant cell, or plant seed of statement 1-13 or 14, comprising at least one loss-of-function mutation(s) in at least four or five genes encoding transcriptional repressors of jasmonic acid (JAZ) responses.
16. The plant, plant cell, or plant seed of statement 1-14 or 15, comprising at least one loss-of-function mutation(s) in at least four or five genes encoding transcriptional repressors of jasmonic acid (JAZ) responses that comprise one or more deletions, substitutions, or insertions into at least four or five genomic nucleic acids encoding transcriptional repressors of jasmonic acid response proteins with at least at least 70%, or at least 75%, or at least 80%, or at least 85%, or at least 90%, or at least 95%, or at least 97%, or at least 98%, or at least 99% sequence identity to any of amino acid sequence SEQ ID NO:48, 50, 52, 54, 56, 58-73, or 74.
17. The plant, plant cell, or plant seed of statement 1-15 or 16, wherein the plant or a plant grown from the seed has less leaf damage from insect feeding than a wild type plant of the same species (but without phyB, phyB-related, or MYC gene mutations) grown under the same environmental conditions.
18. The plant, plant cell, or plant seed of statement 1-16 or 17, wherein the plant or a plant grown from the seed has 5% less, or 10% less, or 20% less, or 30% less, or 40% less, or 50% less, or 60% less, or 70% less, or 80% less, or 90% less, or 100% less leaf damage from insect feeding than a wild type plant of the same species (but without PHYB, PHYB-related, or MYC gene mutations) grown under the same environmental conditions.
19. The plant, plant cell, or plant seed of statement 1-17 or 18, further comprising a heterologous PIF4 transgene or PIF4 expression cassette.
20. The plant, plant cell, or plant seed of statement 1-18 or 19, further comprising a heterologous PIF4 transgene comprising a promoter operably linked to a nucleic acid segment encoding a PIF4 polypeptide.
21. The plant, plant cell, or plant seed of statement 1-19 or 20, further comprising a heterologous PIF4 transgene encoding a PIF4 protein with at least 70% sequence identity, or at least 80% sequence identity, or at least 85% sequence identity, or at least 90% sequence identity, or at least 95% sequence identity, or at least 97% sequence identity, or at least 98% sequence identity, or at least 99% sequence identity to any of amino acid sequences identified as SEQ ID NO: 38, 40, 42, or 44.
22. The plant, plant cell, or plant seed of statement 1-20 or 21, further comprising a heterologous PIF4 transgene comprising a promoter operably linked to a cDNA encoding a PIF4 polypeptide.
23. The plant, plant cell, or plant seed of statement 1-21 or 22, further comprising a heterologous PIF4 transgene comprising a promoter operably linked to a cDNA encoding a PIF4 polypeptide, where the promoter functions (e.g., promotes transcription) during plant development or growth.
24. The plant, plant cell, or plant seed of statement 1-22 or 23, wherein the plant or a plant grown from the seed exhibits resistance to environmental stress compared to a wild type plant of the same species under the same environmental conditions.
25. The plant, plant cell, or plant seed of statement 1-23 or 24, wherein the plant or a plant grown from the seed has at least about 10%, or at least about 20%, or at least about 30%, or at least about 40%, or at least about 50%, or at least about 60%, or at least about 70%, or at least about 80%, or at least about 90% fewer insects or insect larvae than a wild type plant of the same species grown under the same environmental conditions.
26. The plant, plant cell, or plant seed of statement 1-24 or 25, wherein rosette weight of the plant or a plant grown from the seed is about 80% to about 120%, or about 90% to about 110% of the rosette dry weight of wild type plants grown for the same time and under the same conditions.
27. The plant, plant cell, or plant seed of statement 1-25 or 26, which is a food crop species (e.g., sugar beets, beets, tomatoes, lettuce, spinach, carrots, peppers, peas, broccoli, beans, asparagus), a legume species (e.g., peas, beans, lentils, peanuts), a fiber-containing plant species, a tree species, flax, a grain species (e.g., maize, wheat, barley, oats, rice, sorghum, millet, and rye), a grass species (e.g., switchgrass, prairie grass, wheat grass, sudangrass, sorghum, straw-producing plants), a woody plant species (e.g., a poplar species, pine species, or eucalyptus species), a softwood, a hardwood, an oil and/or starch producing plant species (e.g., canola, potatoes, lupins, sunflower and cottonseed), a forage plant species (e.g., alfalfa, clover, or fescue).
28. A method comprising cultivating the plant or plant seed of statement 1-26 or 27.
29. The method of statement 28, wherein the plant or plant grown from the plant seed has less insect damage than a plant or plant grown from a seed without the mutation(s) but cultivated under similar growing conditions.
30. The method of statements 28 or 29, wherein the plant or plant grown from the plant seed has less insect larval and/or adult insect feeding than a plant or plant grown from a seed without the mutation(s) but cultivated under similar growing conditions.
31. The method of statement 28, 29, or 30, further comprising harvesting the plant or harvesting seeds, grain, fruit, vegetables, or biomass of the plant.
32. A method comprising (a) introducing into one or more plant cell at least one chromosomal loss-of-function mutation in a PHYB or PHYB-related gene and introducing at least one mutation into an endogenous MYC gene; and (b) generating a plant from the one or more plant cell(s).
33. The method of statement 32, wherein the mutation in the MYC gene is within or outside of a MYC JAZ-interacting domain (JID) encoded by the MYC gene.
34. The method of statement 32 or 33, wherein the mutation in the MYC gene is a dominant mutation.
35. The method of statement 32, 33, or 34, wherein the mutation in the MYC gene reduces binding of an encoded MYC protein to a JAZ protein selected from a JAZ1 protein, JAZ2 protein, JAZ3 protein, JAZ4 protein, JAZ5 protein, JAZ6 protein, JAZ7 protein, JAZ8 protein, JAZ9 protein, JAZ10 protein, JAZ11 protein, JAZ12 protein, JAZ13 protein, or a combination thereof.
36. The method of statement 32-34 or 35, wherein the mutation in the MYC gene reduces binding of an encoded MYC protein to a JAZ protein selected from a JAZ1 protein, JAZ2 protein, JAZ3 protein, JAZ4 protein, JAZ5 protein, JAZ6 protein, JAZ7 protein, JAZ8 protein, JAZ9 protein, JAZ10 protein, JAZ11 protein, JAZ12 protein, JAZ13 protein, or a combination thereof by at least 20%, or at least 30%, or at least 40%, or at least 50%, or at least 60%, or at least 70%, or at least 80%, or at least 90%, or at least 95%, or at least 97%, or at least 99% compared to a wild type plant of the same species (that does not have the MYC mutation(s)).
37. The method of statement 32-35 or 36, wherein the MYC gene encodes a JAZ-interacting domain (JID) that has less than 100%, or less than 99.5%, or less than 99%, or less than 98%, or at less than 97%, or less than 96%, or less than 95%, or less than 94%, or less than 93%, or less than 92%, or less than 91%, or less than 90% sequence identity to any of SEQ ID NOs: 2, 4, 6, 8, 10, 12, 14, 16, 18, 20, 22, 24, 26, or 28.
38. The method of statement 32-36 or 37, wherein the MYC gene encodes a MYC protein less than 100%, or less than 99.5%, or less than 99%, or less than 98%, or at less than 97%, or less than 96%, or less than 95%, or less than 94%, or less than 93%, or less than 92%, or less than 91%, or less than 90% sequence identity to any of SEQ ID NOs:1, 3, 5, 7, 9, 11, 13, 15, 17, 19, 21, 23, 25, or 27.
39. The method of statement 32-37 or 38, wherein the MYC protein has at least 40%, or at least 50%, or at least 60%, or at least 70%, or at least 80%, or at least 90%, or at least 95%, or at least 97%, or at least 99% sequence identity to any of SEQ ID NOs:1, 3, 5, 7, 9, 11, 13, 15, 17, 19, 21, 23, 25, or 27.
40. The method of statement 32-38 or 39, wherein the plant has reduced PHYB activity compared to a wild type plant without the PhyB loss-of-function mutation.
41. The method of statement 32-39 or 40, wherein the plant expresses a PHYB protein with less than 99%, or less than 98%, or less than 95%, or less than 90%, or less than 85%, or less than 75%, or less than 60%, or less than 50%, or less than 40%, or less than 30%, or less than 20% sequence identity to any of SEQ ID NOs:30, 32, 33, 34, 35, 36, or 37.
42. The method of statement 32-40 or 41, wherein the plant comprises a deletion of a chromosomal PhyB or PhyB-related chromosomal site, a substitution within a chromosomal PhyB or PhyB-related chromosomal site, or an insertion into a chromosomal PhyB or PhyB-related chromosomal site.
43. The method of statement 32-41 or 42, wherein the plant comprises a deletion, substitution, or insertion of a chromosomal PhyB or PhyB-related chromosomal site so that a truncated PHYB polypeptide, a mutant PHYB polypeptide, or no PHYB polypeptide is expressed.
44. The method of statement 32-42 or 43, wherein the plant cell comprises at least one loss-of-function mutation(s) in at least four or five genes encoding transcriptional repressors of jasmonic acid (JAZ) responses.
45. The method of statement 32-43 or 44, further comprising introducing into one or more plant cell at least one chromosomal loss-of-function mutation in at least four or five genes encoding transcriptional repressors of jasmonic acid (JAZ) responses.
46. The method of statement 32-44 or 45, wherein the plant comprises one or more deletions, substitutions, or insertions into at least four or five genomic nucleic acids encoding transcriptional repressors of jasmonic acid response proteins with at least at least 70%, or at least 75%, or at least 80%, or at least 85%, or at least 90%, or at least 95%, or at least 97%, or at least 98%, or at least 99% sequence identity to any of amino acid sequence SEQ ID NO:48, 50, 52, 54, 56, 58-73, or 74.
47. The method of statement 32-45 or 46, wherein the plant exhibits resistance to environmental stress compared to a wild type plant of the same species under the same environmental conditions.
48. The method of statement 32-46 or 47, further comprising obtaining seeds from the plant.
49. The method of statement 32-47 or 48, further comprising harvesting the plant or harvesting grain, fruit, vegetables, or biomass of the plant.
50. A method comprising (a) introducing into one or more plant cell at least one transgene or expression cassette encoding a mutant MYC nucleic acid segment that encodes a mutant MYC protein; and (b) generating a plant from the one or more plant cell(s).
51. The method of statement 50, wherein the mutant MYC nucleic acid segment has at least one mutation within or outside of an encoded MYC JAZ-interacting domain (JID).
52. The method of statement 50 or 51, wherein the mutant MYC nucleic acid has a dominant mutation.
53. The method of statement 50, 51 or 52, wherein the mutant MYC protein has reduced binding to a JAZ protein selected from a JAZ1 protein, JAZ2 protein, JAZ3 protein, JAZ4 protein, JAZ5 protein, JAZ6 protein, JAZ7 protein, JAZ8 protein, JAZ9 protein, JAZ10 protein, JAZ11 protein, JAZ12 protein, JAZ13 protein, or a combination thereof.
54. The method of statement 50-52 or 53, wherein the mutant MYC protein has binding to a JAZ protein selected from a JAZ1 protein, JAZ2 protein, JAZ3 protein, JAZ4 protein, JAZ5 protein, JAZ6 protein, JAZ7 protein, JAZ8 protein, JAZ9 protein, JAZ10 protein, JAZ11 protein, JAZ12 protein, JAZ13 protein, or a combination thereof that is reduced by at least 20%, or at least 30%, or at least 40%, or at least 50%, or at least 60%, or at least 70%, or at least 80%, or at least 90%, or at least 95%, or at least 97%, or at least 99% compared to a wild type plant of the same species (that does not have the MYC mutation(s)).
55. The method of statement 50-53 or 54, wherein the mutant MYC protein has a JAZ-interacting domain (JID) that has less than 100%, or less than 99.5%, or less than 99%, or less than 98%, or at less than 97%, or less than 96%, or less than 95%, or less than 94%, or less than 93%, or less than 92%, or less than 91%, or less than 90% sequence identity to any of SEQ ID NOs: 2, 4, 6, 8, 10, 12, 14, 16, 18, 20, 22, 24, 26, or 28.
56. The method of statement 50-54 or 55, wherein the mutant MYC protein has less than 100%, or less than 99.5%, or less than 99%, or less than 98%, or at less than 97%, or less than 96%, or less than 95%, or less than 94%, or less than 93%, or less than 92%, or less than 91%, or less than 90% sequence identity to any of SEQ ID NOs:1, 3, 5, 7, 9, 11, 13, 15, 17, 19, 21, 23, 25, or 27.
57. The method of statement 50-55 or 56, wherein the mutant MYC protein has at least 40%, or at least 50%, or at least 60%, or at least 70%, or at least 80%, or at least 90%, or at least 95%, or at least 97%, or at least 99% sequence identity to any of SEQ ID NOs:1, 3, 5, 7, 9, 11, 13, 15, 17, 19, 21, 23, 25, or 27.
58. The method of statement 50-56 or 57, wherein the plant has reduced PHYB activity compared to a wild type plant without the PhyB loss-of-function mutation.
59. The method of statement 50-57 or 58, wherein the plant expresses a PHYB protein with less than 99%, or less than 98%, or less than 95%, or less than 90%, or less than 85%, or less than 75%, or less than 60%, or less than 50%, or less than 40%, or less than 30%, or less than 20% sequence identity to any of SEQ ID NOs:30, 32, 33, 34, 35, 36, or 37.
60. The method of statement 50-58 or 59, wherein the plant comprises a deletion of a chromosomal PhyB or PhyB-related chromosomal site, a substitution within a chromosomal PhyB or PhyB-related chromosomal site, or an insertion into a chromosomal PhyB or PhyB-related chromosomal site.
61. The method of statement 50-59 or 60, wherein the plant comprises a deletion, substitution, or insertion of a chromosomal PhyB or PhyB-related chromosomal site so that a truncated PHYB polypeptide, a mutant PHYB polypeptide, or no PHYB polypeptide is expressed.
62. The method of statement 50-60 or 61, wherein the plant cell comprises at least one loss-of-function mutation(s) in at least four or five genes encoding transcriptional repressors of jasmonic acid (JAZ) responses.
63. The method of statement 50-61 or 62, further comprising introducing into one or more plant cell at least one chromosomal loss-of-function mutation in one to five genes encoding transcriptional repressors of jasmonic acid (JAZ) responses.
64. The method of statement 50-62 or 63, wherein the plant comprises one or more deletions, substitutions, or insertions into one or five genomic nucleic acids encoding transcriptional repressors of jasmonic acid response proteins with at least at least 70%, or at least 75%, or at least 80%, or at least 85%, or at least 90%, or at least 95%, or at least 97%, or at least 98%, or at least 99% sequence identity to any of amino acid sequence SEQ ID NO:48, 50, 52, 54, 56, 58-73, or 74.
65. The method of statement 50-63 or 64, wherein the plant exhibits resistance to environmental stress compared to a wild type plant of the same species under the same environmental conditions.
66. The method of statement 50-64 or 65, further comprising obtaining seeds from the plant.
67. The method of statement 50-65 or 67, further comprising harvesting the plant or harvesting grain, fruit, vegetables, or biomass of the plant.
68. A plant, plant cell, or plant seed comprising at least one chromosomal loss-of-function mutation in a PHYB or PHYB-related gene and a loss-of-function mutation in at least one gene encoding a transcriptional repressor of jasmonic acid responses.
69. The plant, plant cell, or plant seed of statement 68, comprising at least one chromosomal loss-of-function mutation in a PHYB or PHYB-related gene and a loss-of-function mutation in at least two or at least three genes encoding transcriptional repressors of jasmonic acid responses.
70. The plant, plant cell, or plant seed of statement 68 or 69, comprising at least one loss-of-function mutation(s) in at least four or five genes encoding transcriptional repressors of jasmonic acid responses.
71. The plant, plant cell, or plant seed of statement 68, 69 or 70, wherein the loss-of-function mutation(s) comprise one or more deletions, substitutions, or insertions into at least four or five genomic nucleic acids encoding transcriptional repressors of jasmonic acid response (JAZ) proteins with at least at least 70%, or at least 75%, or at least 80%, or at least 85%, or at least 90%, or at least 95%, or at least 97%, or at least 98%, or at least 99% sequence identity to any of amino acid sequence SEQ ID NO:48, 50, 52, 54, 56, 58-73, or 74.
72. The plant, plant cell, or plant seed of statement 68-70 or 71, wherein the loss-of-function mutation(s) comprise one or more insertions into at least four or five genomic nucleic acids encoding transcriptional repressors of jasmonic acid response (JAZ) proteins with at least at least 70%, or at least 75%, or at least 80%, or at least 85%, or at least 90%, or at least 95%, or at least 97%, or at least 98%, or at least 99% sequence identity to any of amino acid sequence SEQ ID NO:48, 50, 52, 54, 56, 58-73, or 74.
73. The plant, plant cell, or plant seed of statement 68-71 or 72, wherein the loss-of-function mutation(s) comprise one or more deletions in genomic nucleic acids encoding transcriptional repressors of jasmonic acid response (JAZ) proteins with at least at least 70%, or at least 75%, or at least 80%, or at least 85%, or at least 90%, or at least 95%, or at least 97%, or at least 98%, or at least 99% sequence identity to any of amino acid sequence SEQ ID NO:48, 50, 52, 54, 56, 58-73, or 74.
74. The plant, plant cell, or plant seed of statement 68-72 or 74, wherein the loss-of-function mutation(s) comprise one or more nucleotide substitutions in genomic nucleic acids encoding transcriptional repressors of jasmonic acid response (JAZ) proteins with at least at least 70%, or at least 75%, or at least 80%, or at least 85%, or at least 90%, or at least 95%, or at least 97%, or at least 98%, or at least 99% sequence identity to any of SEQ ID NO:48, 50, 52, 54, 56, 58-73, or 74.
75. The plant, plant cell, or plant seed of statement 68-73 or 74, wherein the loss-of-function mutation(s)s reduce transcription of genomic nucleic acids encoding transcriptional repressors of jasmonic acid response (JAZ) proteins by at least 70%, or at least 75%, or at least 80%, or at least 85%, or at least 90%, or at least 95%, or at least 97%, or at least 98%, or at least 99% compared to a wild type plant of the same species (without the loss-of-function mutations) grown under the same conditions.
76. The plant, plant cell, or plant seed of statement 68-74 or 75, wherein the chromosomal loss-of-function mutation(s) comprise one or more deletions, substitutions, or insertions into one or more genomic nucleic acid that encodes a PHYB protein or a PHYB-related protein with at least at least 70%, or at least 75%, or at least 80%, or at least 85%, or at least 90%, or at least 95%, or at least 97%, or at least 98%, or at least 99% sequence identity to any of SEQ ID NOs: 30, 32, 33, 34, 35, 36, or 37.
77. The plant, plant cell, or plant seed of statement 68-75 or 76, wherein the loss-of-function mutations reduce transcription and/or translation of at least three genes encoding transcriptional repressors of jasmonic acid responses by at least 20%, or at least 30%, or at least 40%, or at least 50%, or at least 60%, or at least 70%, or at least 80%, or at least 90%, or at least 95%, or at least 97%, or at least 99% compared to wild type plant cells, plants, and seeds of the same species (that do not have mutations in genes encoding transcriptional repressors of jasmonic acid response).
78. The plant, plant cell, or plant seed of statement 68-76 or 77, wherein the loss-of-function mutations reduce transcription and/or translation of the phyB gene, or of the phyB-related gene by at least 20%, or at least 30%, or at least 40%, or at least 50%, or at least 60%, or at least 70%, or at least 80%, or at least 90%, or at least 95%, or at least 97%, or at least 99% compared to wild type plant cells, plants, and seeds of the same species (that do not have phyB, phyB-related, or transcriptional repressors of jasmonic acid response gene mutations).
79. The plant, plant cell, or plant seed of statement 68-77 or 78, wherein the plant or a plant grown from the seed has less leaf damage from insect feeding than a wild type plant of the same species (but without phyB, phyB-related, or transcriptional repressors of jasmonic acid response gene mutations) grown under the same conditions.
80. The plant, plant cell, or plant seed of statement 68-78 or 79, wherein the plant or a plant grown from the seed has 5% less, or 10% less, or 20% less, or 30% less, or 40% less, or 50% less, or 60% less, or 70% less, or 80% less, or 90% less, or 100% less leaf damage from insect feeding than a wild type plant of the same species (but without PHYB, PHYB-related, or transcriptional repressors of jasmonic acid response gene mutations) grown under the same conditions.
81. The plant, plant cell, or plant seed of statement 68-79 or 80, wherein the plant or a plant grown from the seed has at least about 10%, or at least about 20%, or at least about 30%, or at least about 40%, or at least about 50%, or at least about 60%, or at least about 70%, or at least about 80%, or at least about 90% fewer insects or insect larvae than a wild type plant of the same species grown under the same conditions.
82. The plant, plant cell, or plant seed of statement 68-80 or 81, wherein rosette weight of the plant or a plant grown from the seed is about 80% to about 120%, or about 90% to about 110% of the rosette dry weight of wild type plants grown for the same time and under the same conditions.
83. The plant, plant cell, or plant seed of statement 68-81 or 82, further comprising a heterologous PIF4 transgene or PIF4 expression cassette.
84. The plant, plant cell, or plant seed of statement 68-82 or 83, further comprising a heterologous PIF4 transgene comprising a promoter operably linked to a nucleic acid segment encoding a PIF4 polypeptide.
85. The plant, plant cell, or plant seed of statement 68-83 or 84, further comprising a heterologous PIF4 transgene encoding a PIF4 protein with at least 70% sequence identity, or at least 80% sequence identity, or at least 85% sequence identity, or at least 90% sequence identity, or at least 95% sequence identity, or at least 97% sequence identity, or at least 98% sequence identity, or at least 99% sequence identity to any of amino acid sequences identified as SEQ ID NO: 38, 40, 42 or 44.
86. The plant, plant cell, or plant seed of statement 68-84 or 85, further comprising a heterologous PIF4 transgene comprising a promoter operably linked to a cDNA encoding a PIF4 polypeptide.
87. The plant, plant cell, or plant seed of statement 68-85 or 86, further comprising a heterologous PIF4 transgene comprising a promoter operably linked to a cDNA encoding a PIF4 polypeptide, where the promoter functions (e.g., promotes transcription) during plant development or growth.
88. A method comprising cultivating the plant or plant seed of statement 50-86 or 87.
89. The method of statement 88, wherein less insecticide is needed or less insecticide is applied to a plant or plant grown from the seed than would be applied to a plant or plant grown from a seed without the mutation(s) but cultivated under similar growing conditions.
90. The method of statement 88 or 89, wherein the plant or plant grown from the plant seed has less insect damage than a plant or plant grown from a seed without the mutation(s) but cultivated under similar growing conditions.
91. The method of statement 88, 89, or 90, wherein the plant or plant grown from the plant seed has less insect larval and/or adult insect feeding than a plant or plant grown from a seed without the mutation(s) but cultivated under similar growing conditions.
92. A method comprising (a) introducing at least one chromosomal loss-of-function mutation in a PHYB or PHYB-related gene and introducing at least one loss-of-function mutation in at least one gene encoding a transcriptional repressor of jasmonic acid responses into one or more plant cells; and (b) generating a plant from the one or more plant cells.
93. A method comprising (a) introducing at least one chromosomal loss-of-function mutation in a PHYB or PHYB-related gene and introducing at least one loss-of-function mutation in at least three genes encoding transcriptional repressors of jasmonic acid responses into one or more plant cells; and (b) generating a plant from the one or more plant cells.
94. The method of statement 92 or 93, further comprising introducing a heterologous PIF4 transgene comprising a promoter operably linked to a cDNA encoding a PIF4 polypeptide into the one or more plant cells.
95. The method of statement 92, 93 or 94, further comprising introducing at least one mutation in at least one JAZ interacting domain (JID) of a MYC protein encoded by a MYC gene into the one or more plant cells.
96. The method of statement 92-94 or 95, further comprising obtaining seeds from the plant.
97. The method of statement 28-67, 88-95 or 96, wherein the plant has a primary root that 1.5-fold longer, of 2-fold longer, or 2.3-fold longer, or 2.5-fold longer, or 2.7-fold longer, or 3-fold longer than the average primary root length of wild type plants grown for the same time and under the same conditions.
98. The method of statement 97, wherein the conditions are environmental stress conditions.
99. The method of statement 97 or 98, wherein the conditions comprise growth in the presence of 5-methyl-tryptophan.
100. The plant, plant cell, or plant seed of statement 1-26, 68-86, or 87, wherein the plant has a primary root that 1.5-fold longer, of 2-fold longer, or 2.3-fold longer, or 2.5-fold longer, or 2.7-fold longer, or 3-fold longer than the average primary root length of wild type plants grown for the same time and under the same conditions.
101. The plant, plant cell, or plant seed of statement 100, wherein the conditions are environmental stress conditions.
102. The plant, plant cell, or plant seed of statement 100 or 101, wherein the conditions comprise growth in the presence of 5-methyl-tryptophan.

The specific compositions and methods described herein are representative, exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a plant” or “a seed” or “a cell” includes a plurality of such plants, seeds or cells, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

What is claimed:

1. A plant, plant cell, or plant seed comprising a PhyB loss-of-function mutation, and (a) a modified MYC nucleic acid encoding a mutant MYC protein comprising at least one mutation within or outside of a JAZ-interacting domain (JID) polypeptide region, (b) a loss-of-function mutation in at least one gene encoding a transcriptional repressor of jasmonic acid response (JAZ) protein; or (c) a combination of (a) and (b).


SEQUENCE LISTING (129 sequences)

SEQ ID NO: 1 - protein, 623 aa, Arabidopsis thaliana
MTDYRLQPTMNLWTTDDNASMMEAFMSSSDISTLWPPASTTTTTATTETTPTPAMEIPAQAGFNQETLQQRLQALIEGTHEGWTYAIFWQPSYDFSGASVLGWGDGYYKGEEDKANPRRRSSSPPFSTPADQEYRKKVLRELNSLISGGVAPSDDAVDEEVTDTEWFFLVSMTQSFACGAGLAGKAFATGNAVWVSGSDQLSGSGCERAKQGGVFGMHTIACIPSANGVVEVGSTEPIRQSSDLINKVRILFNFDGGAGDLSGLNWNLDPDQGENDPSMWINDPIGTPGSNEPGNGAPSSSSQLFSKSIQFENGSSSTITENPNLDPTPSPVHSQTQNPKFNNTFSRELNFSTSSSTLVKPRSGEILNFGDEGKRSSGNPDPSSYSGQTQFENKRKRSMVLNEDKVLSFGDKTAGESDHSDLEASVVKEVAVEKRPKKRGRKPANGREEPLNHVEAERQRREKLNQRFYALRAVVPNVSKMDKASLLGDAIAYINELKSKVVKTESEKLQIKNQLEEVKLELAGRKASASGGDMSSSCSSIKPVGMEIEVKIIGWDAMIRVESSKRNHPAARLMSALMDLELEVNHASMSVVNDLMIQQATVKMGFRIYTQEQLRASLISKIG

SEQ ID NO: 2 - protein, 61 aa, Arabidopsis thaliana
YDFSGASVLGWGDGYYKGEEDKANPRRRSSSPPFSTPADQEYRKKVLRELNSLISGGVAPS

SEQ ID NO: 3 - protein, 592 aa, Arabidopsis thaliana
MNGTTSSINFLTSDDDASAAAMEAFIGTNHHSSLFPPPPQQPPQPQFNEDTLQQRLQALIESAGENWTYAIFWQISHDFDSSTGDNTVILGWGDGYYKGEEDKEKKKNNTNTAEQEHRKRVIRELNSLISGGIGVSDESNDEEVTDTEWFFLVSMTQSFVNGVGLPGESFLNSRVIWLSGSGALTGSGCERAGQGQIYGLKTMVCIATQNGVVELGSSEVISQSSDLMHKVNNLFNFNNGGGNNGVEASSWGFNLNPDQGENDPALWISEPTNTGIESPARVNNGNNSNSNSKSDSHQISKLEKNDISSVENQNRQSSCLVEKDLTFQGGLLKSNETLSFCGNESSKKRTSVSKGSNNDEGMLSFSTVVRSAANDSDHSDLEASVVKEAIVVEPPEKKPRKRGRKPANGREEPLNHVEAERQRREKLNQRFYSLRAVVPNVSKMDKASLLGDAISYINELKSKLQQAESDKEEIQKKLDGMSKEGNNGKGCGSRAKERKSSNQDSTASSIEMEIDVKIIGWDVMIRVQCGKKDHPGARFMEALKELDLEVNHASLSVVNDLMIQQATVKMGSQFFNHDQLKVALMTKVGENY

SEQ ID NO: 4 - protein, 55 aa, Arabidopsis thaliana
STGDNTVILGWGDGYYKGEEDKEKKKNNTNTAEQEHRKRVIRELNSLISGGIGVS

SEQ ID NO: 5 - protein, 589 aa, Arabidopsis thaliana
MSPTNVQVTDYHLNQSKTDTTNLWSTDDDASVMEAFIGGGSDHSSLFPPLPPPPLPQVNEDNLQQRLQALIEGANENWTYAVFWQSSHGFAGEDNNNNNTVLLGWGDGYYKGEEEKSRKKKSNPASAAEQEHRKRVIRELNSLISGGVGGGDEAGDEEVTDTEWFFLVSMTQSFVKGTGLPGQAFSNSDTIWLSGSNALAGSSCERARQGQIYGLQTMVCVATENGVVELGSSEIIHQSSDLVDKVDTFFNFNNGGGEFGSWAFNLNPDQGENDPGLWISEPNGVDSGLVAAPVMNNGGNDSTSNSDSQPISKLCNGSSVENPNPKVLKSCEMVNFKNGIENGQEEDSSNKKRSPVSNNEEGMLSFTSVLPCDSNHSDLEASVAKEAESNRVVVEPEKKPRKRGRKPANGREEPLNHVEAERQRREKLNQRFYSLRAVVPNVSKMDKASLLGDAISYISELKSKLQKAESDKEELQKQIDVMNKEAGNAKSSVKDRKCLNQESSVLIEMEVDVKIIGWDAMIRIQCSKRNHPGAKFMEALKELDLEVNHASLSVVNDLMIQQATVKMGNQFFTQDQLKVALTEKVGECP

SEQ ID NO: 6 - protein, 57 aa, Arabidopsis thaliana
NNNNNTVLLGWGDGYYKGEEEKSRKKKSNPASAAEQEHRKRVIRELNSLISGGVGGG

SEQ ID NO: 7 - protein, 751 aa, Oryza sativa
MWVLLSPLLTTKNPFHPIPIPTFPLLLFSSSLVGVLFQIKSNLEEEEIEIKSMNLWTDDNASMMEAFMASADLPAFPWGAASTPPPPPPPPHHHHQQQQQQVLPPPAAAPAAAAFNQDTLQQRLQSIIEGSRETWTYAIFWQSSIDVSTGASLLGWGDGYYKGCDDDKRKQRSSTPAAAAEQEHRKRVLRELNSLIAGAGAAPDEAVEEEVTDTEWFFLVSMTQSFPNGLGLPGQALFAAQPTWIATGLSSAPCDRARQAYTFGLRTMVCLPLATGVLELGSTDVIFQTGDSIPRIRALFNLSAAAASSWPPHPDAASADPSVLWLADAPPMDMKDSISAADISVSKPPPPPPHQIQHFENGSTSTLTENPSPSVHAPTPSQPAAPPQRQQQQQQSSQAQQGPFRRELNFSDFASNGGAAAPPFFKPETGEILNFGNDSSSGRRNPSPAPPAATASLTTAPGSLFSQHTPTLTAAANDAKSNNQKRSMEATSRASNTNNHPAATANEGMLSFSSAPTTRPSTGTGAPAKSESDHSDLEASVREVESSRVVAPPPEAEKRPRKRGRKPANGREEPLNHVEAERQRREKLNQRFYALRAVVPNVSKMDKASLLGDAISYINELRGKLTALETDKETLQSQMESLKKERDARPPAPSGGGGDGGARCHAVEIEAKILGLEAMIRVQCHKRNHPAARLMTALRELDLDVYHASVSVVKDLMIQQVAVKMASRVYSQDQLNAALYTRIAEPGTAAR

SEQ ID NO: 8 - protein, 52 aa, Oryza sativa
STGASLLGWGDGYYKGCDDDKRKQRSSTPAAAAEQEHRKRVLRELNSLIAGA

SEQ ID NO: 9 - protein, 705 aa, Zea mays
MNLWTDDNASMMEAFMASADLPTFPWGAPAGGGNSSAAAASPPPPQMPAATAPGFNQDTLQQRLQAMIEGSRETWTYAIFWQSSLDSATGASLLGWGDGYYKGCDEDKRKQKPLTPSAQAEQEHRKRVLRELNSLISGAAAAPDEAVEEEVTDTEWFFLVSMTQSFLNGSGLPGQALFAGQPTWIASGLSSAPCERARQAYNFGLRTMVCFPVGTGVLELGSTDVVFKTAESMAKIRSLFGGGAGGGSWPPVQPQAPSSQQPAAGADHAETDPSMLWLADAPVMDIKDSLSHPSAEISVSKPPPHPPQIHFENGSTSTLTENPSPSVHAPPPPPAPAAPQQRQHQHQNQAHQGPFRRELNFSDFASTPSLAATPPFFKPESGEILSFGADSNARRNPSPVPPAATASLTTAPGSLFSQHTATMTAAAANDAKNNNKRSMEATSRASNTNHHPAATANEGMLSFSSAPTTRPSTGTGAPAKSESDHSDLDASVREVESSRVVAPPPEAEKRPRKRGRKPANGREEPLNHVEAERQRREKLNQRFYALRAVVPNVSKMDKASLLGDAISYINELRGKLTSLETDKETLQTQVEALKKERDARPPSHSAGLGGHDGGPRCHAVEIDAKILGLEAMIRVQCHKRNHPSARLMTALRELDLDVYHASVSVVKDLMIQQVAVKMASRVYTQDQLSAALYSRLAEPGSAMGR

SEQ ID NO: 10 - protein, 52 aa, Zea mays
ATGASLLGWGDGYYKGCDEDKRKQKPLTPSAQAEQEHRKRVLRELNSLISGA

SEQ ID NO: 11 - protein, 703 aa, Zea mays
MNLWTDDNASMMEAFMASADLPAYPWGAPAGGGNPPPPQMPPAMAMAPGFNQDTLQQRLQAMIEGSRETWTYAIFWQSSLDAATGASLLGWGDGYYKGCDDDKRRHRPPLTPAAQAEQEHRKRVLRELNSLISGGASAAPAPAPDEAVEEEVTDTEWFFLVSMTQSFLNGSGLPGQALFAGHHTWIAAGLSSAPCDRARQAYNFGLRTMVCFPVGTGVLELGSTDVVFQTAETMAKIRSLFGGGPGGGSWPPVQPQAAPQQQHAAEADQAAETDPSVLWLADAPVVDIKDSYSHPSAAEISVSKPPPPPPPPQIHFENGSTSTLTENPSPSVHAPPAPPAPPQRQQQNQGPFRRELNFSDFASNPSLAAAPPFFKPESGEILSFGVDSNAQRNPSPAPPASLTTAPGSLFSQSQHTATAAANDAKNNNNNNKRSMEATSLASNTNHHPAAAANEGMLSFSSAPTARPSAGTGAPAKSESDHSDLDASVREVESSRVVAPPPEAEKRPRKRGRKPANGREEPLNHVEAERQRREKLNQRFYALRAVVPNVSKMDKASLLGDAISYINELRGKLTSLESDRETLQAQVEALKKERDARPHPHPAAGLGGHDAGGPRCHAVEIDAKILGLEAMIRVQCHKRNHPSARLMTALRELDLDVYHASVSVVKDLMIQQVAVKMASRMYSQDQLSAALYSRLAEPGSVMGR

SEQ ID NO: 12 - protein, 53 aa, Zea mays
ATGASLLGWGDGYYKGCDDDKRRHRPPLTPAAQAEQEHRKRVLRELNSLISGG

SEQ ID NO: 13 - protein, 706 aa, Brachypodium distachyon
MNLWTDDNASMMEAFMASAADLPTFPWGAAAATPPPPAAVMPQQPAFNQDTLQQRLQAIIEGSRETWTYAIFWQSSTDAGAGASLLGWGDGYYKGCDDADKRARQQPTPASAAEQEHRKRVLRELNSLIAGGGAAAPDEAVEEEVTDTEWFFLVSMTQSFPNGMGLPGQALYTRQPTWIASGLASAPCERARQAYTFGLRTMVCIPVGTGVLELGATEVIFQTADSLGRIRSLFNLNGGGGGGGAGSSWPPVAPHQQHGGDQAETDPSVLWLTDAPVGDMKESPSVEISVSKPPPPPQIHHFENGSTSTLTENAGPSLHAHQQPATLAPAAPPRQNQHPHQLQLQHQQSQQQQQQQQGPFRRELNFSDFATNASVTVTPPFFKPESGEILNFGADSTSRRNPSPAPPAAAASLTTAPGSLFSQHTATVTAPTNEAKNNPKRSMEATSRASNTNHHPSATANEGMLSFSSAPTTRPSTGTGAPAKSESDHSDLEASVREVESSRVVPPPEEKRPRKRGRKPANGREEPLNHVEAERQRREKLNQRFYALRAVVPNVSKMDKASLLGDAISYINELRGKMTALESDKDTLHSQIEALKKERDARPVAPLSGVHDSGPRCHAVEIEAKILGLEAMIRVQCHKRNHPAAKLMTALRELDLDVYHASVSVVKDIMIQQVAVKMPNRVYSQDQLNAALYSRLAEPGAPVPIR

SEQ ID NO: 14 - protein, 52 aa, Brachypodium distachyon
AGASLLGWGDGYYKGCDDADKRARQQPTPASAAEQEHRKRVLRELNSLIAGG

SEQ ID NO: 15 - protein, 707 aa, Sorghum bicolor
MNLWTDDNASMMEAFMASADLPTFPWGATAGGGNSSAAAATPPPPPQMPAAAMAPGFNQDTLQQRLQAMIEGSSETWTYAIFWQSSLDAATGASLLGWGDGYYKGCDDDKRKQRPLTPAAQAEQEHRKRVLRELNSLISGAAAAPDEAVEEEVTDTEWFFLVSMTQSFLNGSGLPGQALFAGQPTWIASGLSSAPCERARQAYNFGLRTMVCFPVGTGVLELGSTDVVFQTAESMAKIRSLFGGGAGGGSWPPPQAPSHQQPAAGPDQAETDLWLADAPVMDIKDSMSHPSAEISVSKPPPPPPPPQIHFENASTSTLTENPSPSVHAAPPQPAPAAAPQRQHQHQNQAHQGPFRRELNFSDFASTNPSSLAATPPFFKPESGEILSFGADSNARRNPSPAPPAATASLTTAPGSLFSQHTATMTQAAAANDAKNNNKRSMEATSRASNTNHHPAATANEGMLSFSSAPTTRPSTGTGAPAKSESDHSDLDASVREVESSRVVAPPPEAEKRPRKRGRKPANGREEPLNHVEAERQRREKLNQRFYALRAVVPNVSKMDKASLLGDAISYINELRGKLTSLESDKDTLQAQIEALKKERDARPPAHAAGLGGHDGGPRCHAVEIDAKILGLEAMIRVQCHKRNHPSARLMTALRELDLDVYHASVSVVKDLMIQQVAVKMASRIYSQDQLNAALYSRLAEPGSAMGR

SEQ ID NO: 16 - protein, 51 aa, Sorghum bicolor
ATGASLLGWGDGYYKGCDDDKRKQRPLTPAAQAEQEHRKRVLRELNSLISG

SEQ ID NO: 17 - protein, 462 aa, Camelina sativa
  VARIANT 1..462: note = Xaa = Any Amino Acid
MTDYRLQPTMNLWTTDDNASMMEAFISSSDISTLWPMATTTTTTTTATTSAPATAMDIPAPAGFNQETLQQRLQALIEGTNEGWTYAIFWQPSYDFSGASVLGWGDGYYKGEEDKAKPRQRSSSPPFSTPADQEYRKKVLRELNSLISGGVAPSDDAVDEEVTDTEWFFLVSMTQSFACGAGLAGRAFSTGNAVWVSGSDQLSGSGCERAKQGGVFGMQTIACIPSANGVVEVGSTEQIRQSSDLINKVRVLFNLDGGAGDLSGLDWNLDPDQGENDPSMWINDPIGAPGSNEPGNGAPSSSSQLFSKSIQFENGSSSTITENPNPDPTPSPVHSQTQNPKFSNNFSRELNFSTSSSTLVKPRSGEILSFGDDGKRGSGNPDPSSYSGQTQFENKRKKSPNEDKVLSFGDKTTGESDHSDLEASVVKEVAVEKRPKKRGRKPANGREEPLNXMIYVIHSPNP

SEQ ID NO: 18 - protein, 80 aa, Camelina sativa
QRLQALIEGTNEGWTYAIFWQPSYDFSGASVLGWGDGYYKGEEDKAKPRQRSSSPPFSTPADQEYRKKVLRELNSLISGG

SEQ ID NO: 19 - protein, 689 aa, Solanum lycopersicum
MTEYSLPTMNLWNNSTSDDNVSMMEAFMSSDLSFWATNNSTSAAVVGVNSNLPHASSNTPSVFAPSSSTSASTLSAAATVDASKSMPFFNQETLQQRLQALIDGARETWTYAIFWQSSVVDFSSPSVLGWGDGYYKGEEDKAKRKLSVSSPAYIAEQEHRKKVLRELNSLISGAPPGTDDAVDEEVTDTEWFFLISMTQSFVNGSGLPGQALYSSSPIWVAGTEKLAASHCERVRQAQGFGLQTIVCIPSANGVVELGSTELIVQSSDLMNKVRVLFNFSNDLGSGSWAVQPESDPSALWLTDPSSSGMEVRESLNTVQTNSVPSSNSNKQIAYGNENNHPSGNGQSCYNQQQQKNPPQQQTQGFFTRELNFSEFGFDGSSNRNGNSSVSCKPESGEILNFGDSTKKSASSANVNLFTGQSQFGAGEENNNKNKKRSATSRGSNEEGMLSFVSGTVLPSSGMKSGGGGGEDSEHSDLEASVVKEADSSRVVEPEKRPRKRGRKPANGREEPLNHVEAERQRREKLNQRFYALRAVVPNVSKMDKASLLGDAISYINELKSKLQNTESDKEDLKSQIEDLKKESRRPGPPPPPNQDLKMSSHTGGKIVDVDIDVKIIGWDAMIRIQCNKKNHPAARLMAALMELDLDVHHASVSVVNDLMIQQATVKMGSRHYTEEQLRVALTSKIAETH

SEQ ID NO: 20 - protein, 53 aa, Solanum lycopersicumv
FSSPSVLGWGDGYYKGEEDKAKRKLSVSSPAYIAEQEHRKKVLRELNSLISGA

SEQ ID NO: 21 - protein, 464 aa, Solanum lycopersicum
MTDYRLWSNTNTTNTCDDTMMMDSFLSSDPSSFWPASTPNRPTPVNGVGETMPFFNQESLQQRLQALIDGARESWAYAIFWQSSVVDFASQTVLGWGDGYYKGEEDKNKRRGSSSSAANFVAEQEHRKKVLRELNSLISGVQASAGNGTDDAVDEEVTDTEWFFLISMTQSFVNGNGLPGLAMYSSSPIWVTGTEKLAASQCERARQAQGFGLQTIVCIPSPESREILNFGDSSKRFSGQSQLGPGPGLMEENKNKNKNKKRSLGSRGNNEEGMLSFVSGVILPTSTMGKSGDSDHSDLEASVVKEAVVEPEKKPRKRGRKPANGREEPLNHVEAERQRREKLNQRFYELRSQIECLRKELTNKGSSNYSASPPLNQDVKIVDMDIDVKVIGWDAMIRIQCSKKNHPAARLMAALKDLDLDVHHASVSVVNDLMIQQATVKMGSRLYAQEQLRIALTSKIAESR

SEQ ID NO: 22 - protein, 54 aa, Solanum lycopersicum
FASQTVLGWGDGYYKGEEDKNKRRGSSSSAANFVAEQEHRKKVLRELNSLISGV

SEQ ID NO: 23 - protein, 692 aa, Solanum tuberosum
MTEYSLPTMNLWNNSTSDDNVSMMEAFMSSDLSFWATTNSTTTNSASAAVVGVNSNLLHTNNNNPSVFPLSSSTSVSAAAAVDATKSMPFFNQETLQQRLQALIDGARETWTYAIFWQSSVVDFSSPSVLGWGDGYYKGEEDKAKRKLAVSSPAYIAEQEHRKKVLRELNSLISGAPAGTDDAVDEEVTDTEWFFLISMTQSFVNGSGLPGQALYSSSPIWVAGTEKLAASHCERVRQAQGFGLQTIVCIPSANGVVELGSTELIVESSDLMNKVRVLFNFSNDLGSGSWAVQPESDPSALWLTEPSSSGMEVRESLNTVQTNSVPSSNSNKQIAYANENNHQSGNGQSCYNLQQQQNNPPQQQTQGFFTRELNFSEFGFDGSSNRNGNASLSCKPESGEILNFGDSTKKSASSANVNLFTGQSQFGAVEENNNNKNKKRSATSRGSNEEGMLSFVSGTVLPSSGMKSGGGGGEDSEHSDLEASVVKEADSSRVVEPEKRPRKRGRKPANGREEPLNHVEAERQRREKLNQRFYALRAVVPNVSKMDKASLLGDAISYINELKSKLQNTESDKEDLKSQIEDLKKESRRPGPPPPNQDLKIGGKIVDVDIDVKIIGWDAMIGIQCNKKNHPAARLMAALMELDLDVHHASVSVVNDLMIQQATVKMGSRHYTEEQLRVALKSKIAETPLESR

SEQ ID NO: 24 - protein, 53 aa, Solanum tuberosum
FSSPSVLGWGDGYYKGEEDKAKRKLAVSSPAYIAEQEHRKKVLRELNSLISGA

SEQ ID NO: 25 - protein, 646 aa, Solanum tuberosum
MTDYRLWSNSNTTNTSDDNMMMDAFLSSDPSSFWPNRTSISPTPVNGGVGETMPFFNQESLQQRLQALIDGARESWAYAIFWQSSSTSDFATPSVLGWGDGYYKGEENKNKRRASSSSTNFVAEQEHRKKVLRELNSLISGVQATGAGSGGDDAVDEEVTDTEWFFLISMTQSFANGNGLPGLAMYSSSPIWVTGTEKLAGSQCERARQAQGFGLQTIVCIPSANGVVELGSTELIFESSDLMNKVKYLFNFNIDMGSVTGSGSGSCAVHPEPDPSALWLTDPSSSVVEAKDSLINSSSRDVQLVFGNENSENGTQNQQHSQQTQGFFTKELNFSGYGFDGSSTRNKNGNSSISCKPETREILNFGDSSKKSGSLFSGQSQFGPGTGLGLMEENKNNNKKRSLASRGNNEKGMLSFVSGVILPTSTMGKSGGGGNFDHSDLEASVVKEAIVEPERKPRKRGRKPANGREEPLNHVEAERQRREKLNQRFYALRAVVPNVSKMDKASLLGDAIAYINELKSKVQNSDLDKEELRSQIESLRKELANKGSSNYSSSPPSNQDLKIVDMDIDVKVIGWDAMIRIQCSKKNHPAARLMAALKDLDLDVHHASVSVVNDLMIQQATVKMGSRLYAQEQLTIALTSKFAESR

SEQ ID NO: 26 - protein, 53 aa, Solanum tuberosum
FATPSVLGWGDGYYKGEENKNKRRASSSSTNFVAEQEHRKKVLRELNSLISGV

SEQ ID NO: 27 - protein, 699 aa, Catharanthus roseus
MTDYRLQPKMNLWGTTTNTAASPIITSDDNSSMMEAFMTSSDPISLWPPSMSVNHHHPPTPTSSAVTTAVDSAKSMPAQPAFFNQENLQQRLQTLIDGARESWTYAIFWQSSVVEFAGPSVLGWGDGYYKGEEDKGKRKNSSSASSFAEQEHRKKVLRELNSLIAGPQGTADDAVDEEVTDTEWFFLISMTQSFVSGSGLPGQALYNSNPVWVTGAGRLAVSHCDRARQAQSFGLQTLVCIPSANGVVELGSTELIFQSSDLMNKVRILFNFNNIDLGSSSGPWPENDPSSLWLTDPSPSGVGVKEGVNTNNNTSVQGNSIPSGNKQQLVFGNNDNHPTTSTLTDHPGAGAVNSYNNSSQNAQQPQGSFFTRELNFSEYGFERSSVKNGNCKPESGEILNFGGESVTKKNSVSGNGNLFSVQSQFGAGEENKNKKRPSPVSRGSNDEGMLSFTSGVVLPSTGVVKSSGGGGGGDSDHSDLEASVVKEAESSRVVDPEKRPRKRGRKPANGREEPLNHVEAERQRREKLNQRFYALRAVVPNVSKMDKASLLGDAISYINELKAKLQTTETDKDELKNQLDSLKKELASKESRLLSSPDQDLKSSNKQSVGNLDMDIDVKIIGREAMIRVQSSKNNHPAARVMGALKDLDLELLHASVSVVNDLMIQQNTVRMGSRFYTQEQLRIALTSRIAGNSMRLLV

SEQ ID NO: 28 - protein, 51 aa, Catharanthus roseus
FAGPSVLGWGDGYYKGEEDKGKRKNSSSASSFAEQEHRKKVLRELNSLIAG

SEQ ID NO: 29 - skipped

SEQ ID NO: 30 - protein, 1172 aa, Arabidopsis thaliana
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVQRRQDTECFTKAKELAYICQVIKNPLSGMRFANSLLEATDLNEDQKQLLETSVSCEKQISRIVGDMDLESIEDGSFVLKREEFFLGSVINAIVSQAMFLLRDRGLQLIRDIPEEIKSIEVFGDQIRIQQLLAEFLLSIIRYAPSQEWVEIHLSQLSKQMADGFAAIRTEFRMACPGEGLPPELVRDMFHSSRWTSPEGLGLSVCRKILKLMNGEVQYIRESERSYFLIILELPVPRKRPLSTASGSGDMMLMMPY

SEQ ID NO: 31 - DNA, 4550 bp, Arabidopsis thaliana
  source 1..4550: mol_type = other DNA
cttcaatttattttattggtttctccacttatctccgatctcaattctccccattttcttcttcctcaagttcaaaattcttgagaatttagctctaccagaattcgtctccgataactagtggatgatgattcaccctaaatccttccttgtctcaaggtaattctgagaaatttctcaaattcaaaatcaaacggcatggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggtctctcagttgaagaagctatggggaagtctctggtttctgatttaatatacaaagagaatgaagcaactgtcaataagcttctttctcgtgctttgagaggtatattcagttcttcagctatgttgtatctgcggtgtatataccaattcgcgggtatttgattattttgttgcatttggcaatgcaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtctcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagccctgagctgcagcaagctttagcagtccaacggaggcaggacacagagtgtttcacgaaggcaaaagagttggcttatatttgtcaggtgataaagaatcctttgagcggtatgcgtttcgcaaactcattgttggaggccacagacttgaacgaggaccagaagcagttacttgaaacaagtgtttcttgcgagaaacagatctcaaggatcgtcggggacatggatcttgaaagcattgaagacgggtgagtatagttagaatttatctagaagctagttttgcttacttcacaaaatgtgaccaaatcccaaattttgtttttttcattgatcagttcatttgtgctaaagagggaagagtttttccttggaagtgtcataaacgcgattgtaagtcaagcgatgttcttattaagggacagaggtcttcagctgatccgtgacattcccgaagagatcaaatcaatagaggtttttggagaccagataaggattcaacagctcctggctgagtttctgctgagtataatccggtatgcaccatctcaagagtgggtggagatccatttaagccaactttcaaagcaaatggctgatggattcgccgccatccgcacagaattcaggtacatttcattgttcccgctgttgtctccacatatccataaccaaaattatgcaatccggtttttttggttccttattttgtacataaagaaaatgaatttggtttggttaattacgaatttgatttaggcgtttaaagaatttgaggttttaaccaattcactatttgttttggttattgtttagttggaacctagattagtttgatttttgtattcggtttagtcgacttgggaacttttagacacatccataggcctagaattagcagtcaaggaatgtaatgttttcaaattgatgaaaaccagctcaaaagtgtaaaacttgggtttcatgtgttggtgtctttgttatgtctttattcgttgtttgcagaatggcgtgtccaggtgaaggtctgcctccagagctagtccgagacatgttccatagcagcaggtggacaagccctgaaggtttaggtctaagcgtatgtcgaaagattttaaagctaatgaacggtgaggttcaatacatccgagaatcagaacggtcctatttcctcatcattctggaactccctgtacctcgaaagcgaccattgtcaactgctagtggaagtggtgacatgatgctgatgatgccatattagtcacacttcagttggtatgagagtttgtatcattgtatgagtgtttgtgtgtctaacgacgtcggaggaggatagaaagttttttttttgtttccggtgagattagtagagaagagggagattatttgcgttcagctcagctcgccggaaaaaaaacgtaacagtagttgtagagaatttcaagacttttgtttgtgctgtgtaaattgacaactccgagagaaacaaaacaatgagataagaagagagcatattaatcgatgaccaatccttttaatt

SEQ ID NO: 32 - protein, 1161 aa, Zea mays
MASGSRATPTRSPSSARPEAPRHAHHHHHSQSSGGSTSRAGGGAAATESVSKAVAQYTLDARLHAVFEQSGASGRSFDYSQSLRAPPTPSSEQQIAAYLSRIQRGGHIQPFGCTLAVADDSSFRLLAFSENSPDLLDLSPHHSVPSLDSSAPPHVSLGADARLLFSPSSAVLLERAFAAREISLLNPIWIHSRVSSKPFYAILHRIDVGVVIDLEPARTEDPALSIAGAVQSQKLAVRAISRLQALPGGDVKLLCDTVVEHVRELTGYDRVMVYRFHEDEHGEVVAESRRDNLEPYLGLHYPATDIPQASRFLFRQNRVRMIADCHATPVRVIQDPGLSQPLCLVGSTLRAPHGCHAQYMANMGSIASLVMAVIISSGGDDEQTGRGGISSAMKLWGLVVCHHTSPRCIPFPLRYACEFLMQAFGLQLNMELQLAHQLSEKHILRTQTLLCDMLLRDSPTGIVTQSPSIMDLVKCDGAALYYHGKYYPLGVTPTESQIKDIIEWLTVFHGDSTGLSTDSLADAGYLGAAALGEAVCGMAVAYITPSDYLFWFRSHTAKEIKWGGAKHHPEDKDDGQRMHPRSSFKAFLEVVKSRSLPWENAEMDAIHSLQLILRDSFRDAAEGTNNSKAIVNGQVQLRELELRGINELSSVAREMVRLIETATVPIFAVDTDGCINGWNAKIAELTGLSVEEAMGKSLVNDLIFKESEATVEKLLSRALRGEEDKNVEIKLKTFGSEQSKGPIFVVVNACSSRDYTQNIVGVCFVGQDVTGQKVVMDKFVNIQGDYKAIVHNPNPLIPPIFASDENTSCSEWNTAMEKLTGWSRGEVVGKFLIGEVFGNCCRLKGPDALTKFMVIIHNAIGGQDYEKFPFSFFDKNGKYVQALLTANTRSKMDGKSIGAFCFLQIASTEIQQAFEIQRQQEKKCYARMKELAYICQEIKNPLSGIRFTNSLLQMTDLNDDQRQFLETSSACEKQMSKIVKDASLQSIEDGSLVLEQSEFSLGDVMNAVVSQAMLLLRERDLQLIRDIPDEIKDASAYGDQCRIQQVLADFLLSMVRSAPSENGWVEIQVRPNVKQNSDGTNTELFIFRFACPGEGLPADVVQDMFSNSQWSTQEGVGLSTCRKILKLMGGEVQYIRESERSFFLIVLEQPQPRPAAGREIV

SEQ ID NO: 33 - protein, 1161 aa, Zea mays
MASGSRATPTRSPSSARPEAPRHAHHHHHSQSSGGSTSRAGGGAAATESVSKAVAQYTLDARLHAVFEQSGASGRSFDYSQSLRAPPTPSSEQQIAAYLSRIQRGGHIQPFGCTLAVADDSSFRLLAFSENSPDLLDLSPHHSVPSLDSSAPPHVSLGADARLLFSPSSAVLLERAFAAREISLLNPIWIHSRVSSKPFYAILHRIDVGVVIDLEPARTEDPALSIAGAVQSQKLAVRAISRLQALPGGDVKLLCDTVVEHVRELTGYDRVMVYRFHEDEHGEVVAESRRDNLEPYLGLHYPATDIPQASRFLFRQNRVRMIADCHATPVRVIQDPGLSQPLCLVGSTLRAPHGCHAQYMANMGSIASLVMAVIISSGGDDEQTGRGGISSAMKLWGLVVCHHTSPRCIPFPLRYACEFLMQAFGLQLNMELQLAHQLSEKHILRTQTLLCDMLLRDSPTGIVTQSPSIMDLVKCDGAALYYHGKYYPLGVTPTESQIKDIIEWLTVFHGDSTGLSTDSLADAGYLGAAALGEAVCGMAVAYITPSDYLFWFRSHTAKEIKWGGAKHHPEDKDDGQRMHPRSSFKAFLEVVKSRSLPWENAEMDAIHSLQLILRDSFRDAAEGTNNSKAIVNGQVQLRELELRGINELSSVAREMVRLIETATVPIFAVDTDGCINGWNAKIAELTGLSVEEAMGKSLVNDLIFKESEATVEKLLSRALRGEEDKNVEIKLKTFGSEQYKGPIFVVVNACSSRDYTQNIVGVCFVGQDVTGQKVVMDKFVNIQGDYKAIVHNPNPLIPPIFASDENTSCSEWNTAMEKLTGWSRGEVVGKFLIGEVFGNCCRLKGPDALTKFMVIIHNAIGGQDYEKFPFSFFDKNGKYVQALLTANTRSKMDGKSIGAFCFLQIASTEIQQAFEIQRQQEKKCYARMKELAYICQEIKNPLSGIRFTNSLLQMTDLNDDQRQFLETSSACEKQMSKIVKDASLQSIEDGSLVLEQSEFSLGDVMNAVVSQAMLLLRERDLQLIRDIPDEIKDASAYGDQCRIQQVLADFLLSMVRSAPSENGWVEIQVRPNVKQNSDGTNTELFIFRFACPGEGLPADVVQDMFSNSQWSTQEGVGLSTCRKILKLMGGEVQYIRESERSFFLIVLEQPQPRPAAGREIV

SEQ ID NO: 34 - protein, 1166 aa, Zea mays
MASDSRPPKRSPSARRVAPRHAHHHHSQSSGGSTSRAGAGGGGGGAAATESVSKAVAQYNLDARLHAVFEQSGASGRSFDYSQSLRAPPTPSSEQQIAAYLSRIQRGGHIQPLGCTLAVADDSSFRLLAFSENAADLLDLSPHHSVPSLDSVALPPVSLGADARLYFSPSSAVLLERAFAAREISLLNPLWIHSRASSKPFYAILHRIDVGVVIDLEPARTEDPALSIAGAVQSQKLAVRAISRLQALPGGDVKLLCDTVVEHVRELTGYDRVMVYKFHEDEHGEVVAESRRDNLEPYLGLHYPATDIPQASRFLFQQNRVRMIADCHAIPVRVIQDPGLSQQLCLVGSTLRAPHGCHAQYMANMGSIASLVMAVIISSGGDDERTGRGAISSSMKLWGLVVCHHTSPRCIPFPLRYACEFLMQAFGLQLNMELQLAHQLSEKHILRTQTLLCDMLLRDSPAGIITQSPSVMDLVKCDGAALYYRGKYYPLGVTPTESQIKDIIEWLTVCHGDSTGLSTDSLADAGYLGAVALGDAVCGMAVAYITPSDYLFWFRSHTAKEIKWGGAKHHPEDKDDGQRMHPRSSFKAFLEVVKSRSLSWENAEMDAIHSLQLILRDSFRDAAEGTSNSKAIVNGQRQLGELELRGINELSSVAREMVRLIETATVPIFAVDTDGCINGWNAKIAELTGLSVEEAMGKSLVNDLIFKECDDIVEKLLSRALRGEEDKNVEIKLKTFGSEQSKGAIFVIVNACSSRDYTQNIVGVCFVGQDVTGQKVVMDKFINIQGDYKAIVHNPNPLLPPIFASDENTSCSEWNTAMEKLTGWSREEVVGKFLIGEVFGNCCRLKGPDALTKFMVVIHNAIEGHDSEKFPFSFFDKNGKYVQALLTANTRSKMDGKSIGAFCFLQIASAEIQQAFEIQRQQEKKCYARMKELAYICQEIKNPLSGIRFTNSLLQMTDLNDDQRQFLETSSACEKQMSKIVKDASLKSIEDGSLVLEKSEFSLGDVMNAVVSQTMSLLRERDLQLIRDIPDEIKDASAYGDQFRIQQVLADFLLSMAQSAPSENGWVEIQVRPNVKQNYDGTDTELFIFRFACPGEGLPADIVQDMFSNSQWSTQEGVGLSTCRKILKLMGGEVQYIRESERSFFLIVLELPQPRLAAGRENQLIC

SEQ ID NO: 35 - protein, 1137 aa, Glycine max
MASASGAANSSVPPPQIHTSRTKLSHHSSNNNNNIDSMSKAIAQYTEDARLHAVFEQSGESGRSFNYSESIRIASESVPEQQITAYLVKIQRGGFIQPFGSMIAVDEPSFRILGYSDNARDMLGITPQSVPSLDDKNDAAFALGTDVRALFTHSSALLLEKAFSAREISLMNPIWIHSRTSGKPFYGILHRIDVGIVIDLEPARTEDPALSIAGAVQSQKLAVRAISQLQSLPGGDVKLLCDTVVESVRELTGYDRVMVYKFHEDEHGEVVSESKRPDLEPYIGLHYPATDIPQASRFLFKQNRVRMIVDCHASAVRVVQDEALVQPLCLVGSTLRAPHGCHAQYMANMGSIASLVMAVIINGNDEEGVGGRSSMRLWGLVVCHHTSARCIPFPLRYACEFLMQAFGLQLNMELQLAAQSLEKRVLRTQTLLCDMLLRDSPTGIVTQSPSIMDLVKCDGAALYFQGNYYPLGVTPTEAQIRDIIEWLLAFHGDSTGLSTDSLGDAGYPGAASLGDAVCGMAVAYITEKDFLFWFRSHTAKEIKWGGAKHHPEDKDDGQRMHPRSSFKAFLEVVKSRSLPWENAEMDAIHSLQLILRDSFKDAEHRNSKAVVDPHVSEQELQGVDELSSVAREMVRLIETATAPIFAVDVDGHVNGWNAKVSELTGLPVEEAMGKSLVHDLVFKESEETMNKLLSRALKGEEDKNVEIKMRTFGPEHQNKAVFLVVNACSSKDFTNNVVGVCFVGQDVTGQKIVMDKFINIQGDYKAIVHSPNPLIPPIFASDDNTCCLEWNTAMEKLTGWGRVDVIGKMLVGEVFGSCCQLKGSDSITKFMIVLHNALGGQDTDKFPFSFLDRHGKYVQTFLTANKRVNMEGQIIGAFCFLQIMSPELQQALKAQRQQEKNSFGRMKELAYICQGVKNPLSGIRFTNSLLEATSLTNEQKQFLETSVACEKQMLKIIRDVDLESIEDGSLELEKGEFLLGNVINAVVSQVMLLLRERNLQLIRDIPEEIKTLAVYGDQLRIQQVLSDFLLNIVRYAPSPDGWVEIHVRPRIKQISDGLTLLHAEFRMVCPGEGLPPELIQDMFNNSRWGTQEGLGLSMSRKILKLMNGEVQYIREAERCYFYVLLELPVTRRSSKKC

SEQ ID NO: 36 - protein, 1100 aa, Glycine max
MSKAIAQYTEDARLHAVFEQSGESGRSFNYSESIRIASESVPEQQITAYLVKIQRGGFIQPFGSMIAVDEPSFRILGYSDNARDMLGITPQSVPSLDDKNDAAFALGTDVRALFTHSSALLLEKAFSAREISLMNPIWIHSRTSGKPFYGILHRIDVGIVIDLEPARTEDPALSIAGAVQSQKLAVRAISQLQSLPGGDVKLLCDTVVESVRELTGYDRVMVYKFHEDEHGEVVSESKRPDLEPYIGLHYPATDIPQASRFLFKQNRVRMIVDCHASAVRVVQDEALVQPLCLVGSTLRAPHGCHAQYMANMGSIASLVMAVIINGNDEEGVGGRSSMRLWGLVVCHHTSARCIPFPLRYACEFLMQAFGLQLNMELQLAAQSLEKRVLRTQTLLCDMLLRDSPTGIVTQSPSIMDLVKCDGAALYFQGNYYPLGVTPTEAQIRDIIEWLLAFHGDSTGLSTDSLGDAGYPGAASLGDAVCGMAVAYITEKDFLFWFRSHTAKEIKWGGAKHHPEDKDDGQRMHPRSSFKAFLEVVKSRSLPWENAEMDAIHSLQLILRDSFKDAEHRNSKAVVDPHVSEQELQGVDELSSVAREMVRLIETATAPIFAVDVDGHVNGWNAKVSELTGLPVEEAMGKSLVHDLVFKESEETMNKLLSRALKGEEDKNVEIKMRTFGPEHQNKAVFLVVNACSSKDFTNNVVGVCFVGQDVTGQKIVMDKFINIQGDYKAIVHSPNPLIPPIFASDDNTCCLEWNTAMEKLTGWGRVDVIGKMLVGEVFGSCCQLKGSDSITKFMIVLHNALGGQDTDKFPFSFLDRHGKYVQTFLTANKRVNMEGQIIGAFCFLQIMSPELQQALKAQRQQEKNSFGRMKELAYICQGVKNPLSGIRFTNSLLEATSLTNEQKQFLETSVACEKQMLKIIRDVDLESIEDGSLELEKGEFLLGNVINAVVSQVMLLLRERNLQLIRDIPEEIKTLAVYGDQLRIQQVLSDFLLNIVRYAPSPDGWVEIHVRPRIKQISDGLTLLHAEFRMVCPGEGLPPELIQDMFNNSRWGTQEGLGLSMSRKILKLMNGEVQYIREAERCYFYVLLELPVTRRSSKKC

SEQ ID NO: 37 - protein, 1171 aa, Oryza sativa
MASGSRATPTRSPSSARPAAPRHQHHHSQSSGGSTSRAGGGGGGGGGGGGGAAAAESVSKAVAQYTLDARLHAVFEQSGASGRSFDYTQSLRASPTPSSEQQIAAYLSRIQRGGHIQPFGCTLAVADDSSFRLLAYSENTADLLDLSPHHSVPSLDSSAVPPPVSLGADARLLFAPSSAVLLERAFAAREISLLNPLWIHSRVSSKPFYAILHRIDVGVVIDLEPARTEDPALSIAGAVQSQKLAVRAISRLQALPGGDVKLLCDTVVEHVRELTGYDRVMVYRFHEDEHGEVVAESRRNNLEPYIGLHYPATDIPQASRFLFRQNRVRMIADCHAAPVRVIQDPALTQPLCLVGSTLRSPHGCHAQYMANMGSIASLVMAVIISSGGDDDHNIARGSIPSAMKLWGLVVCHHTSPRCIPFPLRYACEFLMQAFGLQLNMELQLAHQLSEKHILRTQTLLCDMLLRDSPTGIVTQSPSIMDLVKCDGAALYYHGKYYPLGVTPTEVQIKDIIEWLTMCHGDSTGLSTDSLADAGYPGAAALGDAVSGMAVAYITPSDYLFWFRSHTAKEIKWGGAKHHPEDKDDGQRMHPRSSFKAFLEVVKSRSLPWENAEMDAIHSLQLILRDSFRDSAEGTSNSKAIVNGQVQLGELELRGIDELSSVAREMVRLIETATVPIFAVDTDGCINGWNAKVAELTGLSVEEAMGKSLVNDLIFKESEETVNKLLSRALRGDEDKNVEIKLKTFGPEQSKGPIFVIVNACSSRDYTKNIVGVCFVGQDVTGQKVVMDKFINIQGDYKAIVHNPNPLIPPIFASDENTCCSEWNTAMEKLTGWSRGEVVGKLLVGEVFGNCCRLKGPDALTKFMIVLHNAIGGQDCEKFPFSFFDKNGKYVQALLTANTRSRMDGEAIGAFCFLQIASPELQQAFEIQRHHEKKCYARMKELAYIYQEIKNPLNGIRFTNSLLEMTDLKDDQRQFLETSTACEKQMSKIVKDASLQSIEDGSLVLEKGEFSLGSVMNAVVSQVMIQLRERDLQLIRDIPDEIKEASAYGDQYRIQQVLCDFLLSMVRFAPAENGWVEIQVRPNIKQNSDGTDTMLFLFRFACPGEGLPPEIVQDMFSNSRWTTQEGIGLSICRKILKLMGGEVQYIRESERSFFHIVLELPQPQQAASRGTS

SEQ ID NO: 38 - protein, 428 aa, Arabidopsis thaliana
MEHQGWSFEENYSLSTNRRSIRPQDELVELLWRDGQVVLQSQTHREQTQTQKQDHHEEALRSSTFLEDQETVSWIQYPPDEDPFEPDDFSSHFFSTMDPLQRPTSETVKPKSSPEPPQVMVKPKACPDPPPQVMPPPKFRLTNSSSGIRETEMEQYSVTTVGPSHCGSNPSQNDLDVSMSHDRSKNIEEKLNPNASSSSGGSSGCSFGKDIKEMASGRCITTDRKRKRINHTDESVSLSDAIGNKSNQRSGSNRRSRAAEVHNLSERRRRDRINERMKALQELIPHCSKTDKASILDEAIDYLKSLQLQLQVMWMGSGMAAAAASAPMMFPGVQPQQFIRQIQSPVQLPRFPVMDQSAIQNNPGLVCQNPVQNQIISDRFARYIGGFPHMQAATQPMEMLRFSSPAGQQSQQPSSVPTKTTDGSRLDH

SEQ ID NO: 39 - DNA, 1747 bp, Arabidopsis thaliana
  source 1..1747: mol_type = other DNA
actttctgtctgtacccaaaagaagtaatgaacctctctcatcttcttcttctctgtttctttcatgttttgtgagttgtttctcaacaattttctggtctcttagagtgagaggagagagatagagagttgtgttgggcgtggaacttggactagttccacatatcaggttatatagatcttctctttcaacttctgattcgtccagaagctttcctaatctgagatctgacatggaacaccaaggttggagttttgaggagaattatagtttgtccactaatagaagatctatcaggccacaagatgaactagtggagttattatggcgagatggacaagtggttctgcagagccaaactcatagagaacaaacccaaacccagaaacaagatcatcatgaagaagccctaagatccagcacctttcttgaagatcaagaaactgtctcttggatccaataccctccagatgaagacccattcgaacccgacgacttctcctcccacttcttctcaaccatggatcccctccagagaccaacctcagagacggttaagcctaagtccagtcctgaacctcctcaagtcatggttaagcctaaggcctgtcctgaccctcctcctcaagtcatgcctcctccaaaatttaggttaacaaattcatcatcggggattagggaaacagaaatggaacagtactcggtaacgaccgttggacctagccattgcggaagcaacccatcacagaacgatctcgatgtctcaatgagtcatgatcgaagcaaaaacatagaagaaaagcttaatccgaacgcaagttcctcatcaggtggctcctctggttgcagctttggcaaagatatcaaagaaatggctagtggaagatgcatcacaaccgaccgtaagagaaaacgtataaatcacactgacgaatctgtatctctatcagatgcaatcggtaacaagtcgaaccaacgatcaggatcaaaccgaaggagtcgagcagctgaagttcataatctctccgaaaggaggaggagagataggatcaatgagagaatgaaggctttgcaagaactaatacctcactgcagtaaaactgataaagcttcgattttagacgaagccatagattatttgaaatcacttcagttacagcttcaagtgatgtggatggggagtggaatggcggcggcggcggcttcggctccgatgatgttccccggagttcaacctcagcagttcatacgtcagatacagagcccggtacagttacctcgatttccggttatggatcagtctgcaattcagaacaatcccggtttagtttgccaaaacccggtacaaaaccagatcatctccgaccggtttgctagatacatcggtgggttcccacacatgcaggccgcgactcagccgatggagatgttgagatttagttcaccggcgggacagcaaagtcaacaaccgtcgtctgtgccgacgaagaccaccgacggttctcgtttggaccactaggttggtgagccacttttttacttccttatttttggtatgtttcttttttatatctatctttctgaacatacttaaaacgttcaaggatgtattattatagagtaaacgtgcaacttcattacgttattttctgtatatgtgagtttatgtatgtcaaaatgacatgatgagattttttgtaaacaacatcttaaaaacaggacatgtgatttttgtaatcgtaaaaa

SEQ ID NO: 40 - protein, 397 aa, Zea mays
MQTAIEHACSVVECAATARAAMDMSHYIPDWSSSMGDTFAPLGGEDDDGLIELMWRNGHVVMQAQAPRKPPRPDDDEAAAAQAQAWFQYPVEERADLFSELFGEAQAAVGGARGEAARQSIRMMPPPPPPPRPAQAPREEKACPGDGGTATATDGAGSSVLTVVSSLCGSNGNHVQATAPGDVARARDVLMVTSSSTTRSRSCTTKSEQPGPGPGAARRSGKRKHNDATDAEDVGLECEPAQRTTTAKRRRAAQVHNLSERRRRDRINEKMKALQELIPHCNKADKASMLDEAIEYLKSLQLQLQVVWMGGGIAAAGVHQRTMVAAPGRPPHVASLPASAPDLYTRYLAVDHLPPPPLVPPPRTAAAMGLYPRQNPVPATSSPSFRTTENTRKLWQA

SEQ ID NO: 41 - DNA, 1492 bp, Zea mays
  source 1..1492: mol_type = other DNA
ccttgccctgctgcaacttgaacctcctggcagctcctgtttcaggcaggcagcaagtagggaagaggctctgcagatcagttccatgcagacagcgatcgagcacgcctgctcggtggtggaatgcgctgcgacagcccgagccgccatggacatgagccactacatccccgattggagcagcagcatgggagacaccttcgcgccactgggcggcgaggacgacgacgggctcatcgagctcatgtggcgcaacggccacgtggtcatgcaggcccaggcgccgcggaagccgccgagacccgacgacgacgaggcggcggcggcgcaggcgcaggcgtggttccagtacccggtggaggagagggccgacctcttctcggagctcttcggggaggcgcaggcggccgtcggcggcgcgcgcggggaggccgcgcgccagagtatccggatgatgccgccgccgccgccgccgccgaggcccgcgcaagcgccgcgggaggagaaggcgtgcccgggagacggcggcacggcgacggcgacggacggcgccggctcgtccgtgctcacggtcgtgtccagcctctgcgggagcaacggcaaccacgtgcaggcgacggcgccgggggacgtcgccagggcccgcgacgtgctgatggtgacctcgtcgtcgacgacgcgttccaggtcatgcaccaccaagagcgagcagccgggtcccgggcccggcgctgcccgccggagcggcaagaggaagcataacgacgccaccgatgccgaggacgtggggctggagtgcgagccggcgcagaggacgacgactgccaagcggcgccgcgccgcgcaagtccacaacctctcggagcggaggagacgggacaggatcaacgagaagatgaaggccctgcaggaactcataccccactgcaacaaagcggacaaggcgtcgatgctggacgaggcgatcgagtacctcaagtcgctgcagctccagctgcaggtggtgtggatgggcggcggcatcgcggcggcgggggtgcaccagcggacgatggtggccgcgcccgggcgtcctccccacgtggcttccctgccggcgtcggcgcccgacctctatacgcgctacctcgccgtcgaccacctgccgccaccgcccttggtgccaccgccacgcacggcggcggcgatgggcttgtacccgcgccagaaccccgtgccggcgacgtcgtctccttccttccgaacgaccgaaaatacgcgaaaactatggcaagcctgagattcagatccggggtatggtgaccagctgatgggtcatctagctgcatgcatgtgtgtatgtgttggtagtatggttaagccttgacagagacttgtgatcgagaccgagatcgaccgataggccgtcacttcttttttcttccatctttcagtttttggttgataggccggagtgtaatttgaccagtggtcgagatttgtcaagcgacac

SEQ ID NO: 42 - protein, 562 aa, Glycine max
MNNSIPGWDFESDTCLTNQRKLIGPDQELVELLWKNGQVVMHNQTHRKTLGNSSNLRQVQKSDQSVLRSSGPYGNSSNLDQEDAAPWVQFPLEDPLEQDFCSNLLSELPTCEFESYKPIRQLEEEKFAKFFASGTPHHPTTSSSQPLPPNMKPSCIQGLQGNPIPMPAPRFHGPDSSQKIHDFGASRKVLNFPQFSTPRNNVPSAPGITQFREKTTANMSQSEAREYSVITVGSSHCGSNHIPQEQDVSRISSTGVWATTNNNTTLSAEPEAVRDYVQRPICPKSGQGKSEMIELTVTSSSGGSGSTGIGRTCSLSTRDHGQKRKGTEEEALEEQSEDTELKSADGNKASQRTRSSRRNRAAEVHNQSERRRRDRINEKMRTLQQLIPNSNKTDKASMLEEAIEYLKSLQFQLQVMWMGGGMTPVMFPGIQHYMSQMGMGMGAPSLPSIHNPMQLPKVPHDQAMSVLQIPNQNLMCQNPVLGAFNYQNQMQNPCLPEQYARYMGYHLMQNASQPMNVFRYGSQAVQHSQTMIAPGNNSSGPMSGTANIDDADSGKAGSSTFN

SEQ ID NO: 43 - DNA, 2161 bp, Glycine max
  source 1..2161: mol_type = other DNA
gaccccgttttcaactggtcccgtgttccttcatttgatgccacatgtgcagctacccatgtttttctcgctgttgacgagcacaatatataataaataccatttttttcatgccatatttgctctcttctctctttgtactaataacttggatctatgccactgtccttctccttgttaaaaactgtgccacacgtctgtcaccaaactccctaagcagaagaagcacatgttcagagggagttttgtttcatcagtctctagctagcatatatttctagcttctattcaacaagttgcaaaaaacagactttgccttaaccaaaagaaaatctgtttttaccttaactcagacaactcgtttggtgaaccatgaacaacagtattcctggttgggattttgagagtgatacatgtctcaccaaccaaagaaagctcatagggccggaccaagaacttgtagagctcctatggaaaaatgggcaagtagttatgcacaaccaaacacataggaagacacttgggaattcatctaacttgagacaggtgcagaaaagtgatcaatcagtattaaggtctagcggtccctatggaaactcaagcaacttggatcaagaagatgccgccccatgggtccaattcccacttgaggacccattggaacaagatttttgttcaaaccttttatctgaactaccaacttgtgaatttgaatcttacaagccaatcaggcaattggaagaggaaaagtttgccaaattttttgcttccggtaccccccatcatcctacaacttcaagttcacaaccactaccacctaacatgaaaccctcatgtattcagggactccaagggaatcctattcctatgccagctccaagatttcatggtcctgattcatctcagaaaatccatgactttggagcatcacgaaaggttctaaattttcctcagttttcaacaccccgtaataatgttccatcagcacctggtattacacagtttagagagaaaactactgctaacatgtcacaaagtgaggctagagagtactcagtgatcacagttggttcaagtcactgtggcagcaatcacatccctcaggagcaagatgtaagcaggatttcaagcactggtgtttgggccactactaataataatactactttatctgctgagcctgaagctgtcagagattatgtccaaagaccgatttgtcctaagagtggccaaggaaaatcagagatgattgaactaactgtgacttcatcttccggtggctcgggaagtactggtatcggaagaacctgttccctatcaacaagagatcatggccaaaagagaaaagggacagaagaagaagcgttagaggaacaaagtgaggacacagaacttaaatcagctgatggaaacaaggcttctcagcggacgaggtcttccagaaggaaccgtgcagcagaagtgcataatcaatcagaaaggagaagaagagataggatcaacgagaagatgaggacattgcagcaactgatacctaatagtaacaagacagacaaagcatcaatgttagaagaggcaatcgaatacttgaaatcacttcagtttcagcttcaggttatgtggatggggggtggcatgacaccagtgatgttcccaggaattcagcactatatgtcacaaatgggtatgggaatgggtgcaccttctttgccttccattcacaacccgatgcaattgccaaaagtgccacatgatcaagccatgtctgtgcttcagataccaaaccagaatttaatgtgtcaaaatccagttttgggtgcctttaactaccaaaaccagatgcagaacccgtgccttccagaacaatatgcacgttacatgggttaccatcttatgcaaaatgcctctcagcctatgaatgtgttcagatatggttcccaagcagtgcaacacagtcaaacgatgattgcaccaggcaataatagcagcggacccatgagtggaacagctaatattgatgatgctgacagtggcaaagcgggttcttccacctttaattgaatagtgaatagcaataccttaaaattactcaattgggggaattacctaatggagtacgtcaatcctcacaagcaccaatatgtgctccaattttatgtag

SEQ ID NO: 44 - protein, 409 aa, Oryza sativa
MNQFVPDWNTTSMGDGFAPLGEDDGLVELLWCNGHVVMQSQAPRKPPRPEKTTAAAAAAMAEDESASWFQYPVDDVLEKDLFTELFGEMTAAGGGGGDVRRAACKEERGAVAAFQSRMMPPPWPARGKAEFGDVDDVCGVSEVVMAKMDGAAAAETVGESSMLTIGSSICGSNHVQTPPVGNGKAGAGTAGAARRAHDTATVASSSMRSRSCTAKAEPRDVAAAGVGGKRKQRGGAAMESGSPSEDVEFESAAATCSPAQKTTTAKRRRAAEVHNLSERRRRDRINEKMKALQELIPHCNKTDKASMLDEAIEYLKSLQLQLQMMWMGGGMAPPAVMFPAAGVHQYMQRMGAVGMGPPHMASLPRMPPFMAPPPAAVQSSPVVSMADPYARCLAVDHLQPPPPMFRREY

SEQ ID NO: 45 - DNA, 1749 bp, Oryza sativa
  source 1..1749: mol_type = other DNA
gcgagtcctcttcctgccctgccctgccctgccctgcattctttctttctccaccaggggaatccagttcacccccagtgctgcttctgctgctgcttctgcatcatcttgccctgttaaaaagacacagtgcccttgttctttcgcagttgcaactagcatctcctcctctacttgtactcacttcacacctcagctcagctcagctcatctcctgtcatctcagctcaaagagaaagagctgaaggtgtaagctgatcaccaggaagcagaggctttttttcagattacagttatctgaaacaaccaacttcagaatcaatcagcaaaggtagaaacaagacagagctgctgtgcttctgtgattaattagggttgttaatgccatgaaccagttcgtccctgattggaacaccaccagcatgggcgacggctttgcgccattaggcgaagacgacgggctcgtcgagctgctatggtgcaatggccacgtcgtcatgcagagccaggcgccgcggaagccgccgaggccggagaagacgacggcggcggcggcggcggcgatggcggaggatgagtcggcgtcgtggtttcagtacccggtcgacgacgtgcttgagaaggacctgttcaccgagctgttcggcgaaatgacggcggccggcggcggcggcggcgacgtccgcagggcggcgtgcaaggaggagcgcggcgcggtcgccgcgttccagagcaggatgatgccgccgccgtggccggcgagggggaaggcggagttcggtgacgtcgacgacgtgtgcggcgtctcggaggtcgtcatggcgaagatggacggggcggcggcggcggagacggtcggcgagtcatcgatgctgacaatcgggtcgagcatctgcgggagcaaccacgtccagacgccgccggtggggaacgggaaggccggcgccggcaccgccggcgccgccagaagggcgcacgacacggcgacggtggcgtcgtcgtcgatgaggtcgaggtcctgcaccgccaaggccgagccgcgcgacgtcgcagccgccggcgtcggcggcaagcggaagcagcgcggcggcgccgccatggagtccgggagccccagcgaggacgtggagttcgagtccgccgccgcaacgtgctcgccggcgcagaagacgacgacggcgaagcggcggcgcgccgccgaggtgcacaacctctccgagaggaggagaagagataggatcaatgagaagatgaaagcattacaggagctcatacctcactgcaacaaaacggacaaagcatcgatgctggatgaagcgatcgagtatctcaagtcactgcagctccagctacagatgatgtggatgggcggcggaatggcgccgccggcggtgatgttcccggcggccggcgtgcaccagtacatgcagcggatgggcgccgtcgggatgggcccaccacacatggcgtccctgccgaggatgccgccgttcatggcgccgccgcccgccgccgtgcagagctcgccggtggtcagcatggccgacccctacgcccgctgcctcgccgtcgaccacctccagccaccgcctccgatgtttcgacgcgaatactagggaaggaactaatatcaaataatagaaggggtgagccttcgaatcgagatcgtctagcccaccaccttatagagctagccggaaggccctcgagcgtttctcatattttcagtttcctaagagtttttttttt

SEQ ID NO: 46-47 - skipped (2 entries)

SEQ ID NO: 48 - protein, 187 aa, Arabidopsis thaliana
MSLFPCEASNMDSMVQDVKPTNLFPRQPSFSSSSSSLPKEDVLKMTQTTRSVKPESQTAPLTIFYAGQVIVFNDFSAEKAKEVINLASKGTANSLAKNQTDIRSNIATIANQVPHPRKTTTQEPIQSSPTPLTELPIARRASLHRFLEKRKDRVTSKAPYQLCDPAKASSNPQTTGNMSWLGLAAEI

SEQ ID NO: 49 - DNA, 1527 bp, Arabidopsis thaliana
  source 1..1527: mol_type = other DNA
atattggaggtaggaagaagaactctgcaaccaaaccaaccaaccccaaagccaaacaaagttttatagagaccttccatttctccctctcgtgacaaacgcaatttgcagagaagcaacagcaacaacaagaagaagaagaaaaagatttgagattactttgtatcgatttagctattcgagaaactcttgccgtttgaaagttttaattgttaaagatgtcgagttctatggaatgttctgagttcgtcggtagccggagatttactgggaagaagcctagcttctcacagacgtgtagtcgattgagtcagtatctaaaagagaacggtagctttggagatctgagcttaggaatggcatgcaagcctgatgtcaatggtaagaaaccttctctttctcctagatccacttcttttttcgttttctctgttttttatttcttgaatcttgatcttgaaaacttttcaagaaaattttgaatcgatttcaaagaaattagggagagttagtttgctaaattttgacatagaaaatgattggagagagttctaacttttggatcatatatatttgcaggaactttaggcaactcacgtcagccgacaacaaccatgagtttattcccttgtgaagcttctaacatggattccatggttcaagatgttaaaccgacgaatctgtttcctaggcaaccaagcttttcttcctcatcttcctctcttccaaaggaagatgttttgaaaatgacacagactaccagatctgtgaaaccagagtctcaaactgcaccattgactatattctacgccgggcaagtgattgtattcaatgacttttctgctgagaaagccaaagaagtgatcaacttggcgagcaaaggcaccgctaatagcttagccaagaatcaaaccgatatcagaagcaacatcgctactatcgcaaaccaagttcctcatccaagaaaaaccacaacacaagagccaatccaatcctccccaacaccattgacagaacttcctattgctagaagagcttcacttcaccggttcttggagaagagaaaggacagagttacgtcaaaggcaccataccaattatgcgatccagccaaagcgtcttcaaaccctcaaaccacaggcaacatgtcgtggctcggtttagcagctgaaatatgaatgctaaccaccctcaagccgtaccaagaaattcttttgacgacgttgcttcaagacaagatataaaagctcctatcttcatgctttttgatttaagatacaaactactcaatgattaggaaacttcatatatttgtatgtattgattagtgatcaattattgttagtattcgttatagtctgtttttctactagttattgtcgcctgtctaaatccccttgctatgggttatctcaaaattagtttcgtatgtaactaattttgtaagaacaataatttttgttgacgaaccatactatcaaatactctaaattatatcttgataaatctacctatcaggtaagtagg

SEQ ID NO: 50 - protein, 352 aa, Arabidopsis thaliana
MERDFLGLGSKNSPITVKEETSESSRDSAPNRGMNWSFSNKVSASSSQFLSFRPTQEDRHRKSGNYHLPHSGSFMPSSVADVYDSTRKAPYSSVQGVRMFPNSNQHEETNAVSMSMPGFQSHHYAPGGRSFMNNNNNSQPLVGVPIMAPPISILPPPGSIVGTTDIRSSSKPIGSPAQLTIFYAGSVCVYDDISPEKAKAIMLLAGNGSSMPQVFSPPQTHQQVVHHTRASVDSSAMPPSFMPTISYLSPEAGSSTNGLGATKATRGLTSTYHNNQANGSNINCPVPVSCSTNVMAPTVALPLARKASLARFLEKRKERVTSVSPYCLDKKSSTDCRRSMSECISSSLSSAT

SEQ ID NO: 51 - DNA, 3253 bp, Arabidopsis thaliana
  source 1..3253: mol_type = other DNA
gcgatttgttaataaaactagaaattgcggtgaattaacttcattccacgttttttcattttctccctcaaaagtctctgttttttttcctttttccggcgaagctctatttagcttgattccggcgtttaacacgcgttttaatcgaaacagacatttgagatcgaattaattttgtagcgggctgtgtctttattatagatggagagagattttctcgggttgggttcgaaaaattctccgatcactgtcaaggaggaaaccagcgaaagctctagagattcaggttatttattactcttctcaatttttctgattctgattgtttttaaatcgtagatttgtttgattgattaggagttattaggactacttgtagtatggaatttgtttttggatagctgattttatggcttgctcgggaactggaattgtcagtttgttgcttggagcagaacattgtcctttgcttttctcgggagatgtagaatttggatttggaaaaactagtgttcttttccaaagccttgtcttaaacatgctttcggtcggagaaattaacgagaactaatctcaagcttctaacataattaaactcggtaaacttttttttactagagtaaatttttttgttttgtttgaagagtcttataattgagaaatactttattagtttatactaaaaaaaaaacgaatacgtaaaatgttggaaaagaggggatgtatagagactgatacaaaaatgataaaatagagacggttggtagtaggtagaaagattaaatatactcaaaagagtgagttggattagtttataagatgattaacttcttgattgtgtgagttggattagtttatgagattattaaaatattgattgtgtatttgtgttgtgtgttgattaagcggaacttgcgttagaatattgttcaaggtacaatgtggaaataatagttttctcaccacgaggaatataattatttcaactttgttttcttatcagccaaaacgtgccacaccataaaagtagtgcatcaacatgtggtgtggtgtggtggggttaaagtttgaatctctctttaatttaaactattaaaacaaacttaaattattggagtttcgtacaatgactttcaatcaaatgttttagaattagacacggttttcgaaagtggttttccctcgttgaatttgtcaacagtatcagattctacattgttggttactaatcttttccttgaagtaggtgttgaattaatcctctgttgtttatgtaaggagatctcgagacatttatggttaacagttaacactacatgtttgactttaaactgattatcttttattctttttcttttgtagctcccaacagaggaatgaactggtctttctcaaacaaagtatcagcttcttcttctcagtttctatccttcaggccaactcaagaagatagacatagaaagtctggaaattatcatcttcctcactctggttccttcatgccatcatcagtagctgatgtttatgattcaacccgcaaagctccttacagttctgtacaggtatttgtcatcaaaacctatgttaaccaagacccttgtgttttttttatccttcgcaagatagctttaaaagtgagccctgttttatgagcatatagtaattggttttgagtctagtttagcacaagttcatggcaattagtttgtggatctaatcttggtttaatactgattcattttaagtgtaagctaagcttctcatttttgataagttagttcatacaatgcctcacacctactttatggcttgttactctcagggagtgaggatgttccctaattccaatcaacacgaagaaactaacgcagtttccatgtcgatgccgggtttccagtctcatcattatgcaccaggaggaagaagcttcatgaacaataacaataactcacaacctttggtaggagttcctatcatggcacctccaatttcaatccttcctcctccaggttccattgtagggactactgatattaggtacccactagtcatcatatcatacagaaactctttctacattttcatagttgactaaagacttatttttgtcagatcttcttccaagccaataggttcacctgcgcagttgacgatcttttatgccggttcagtttgtgtttacgatgacatatctcctgaaaaggtatctcaatcattttcttccatatatgcatctcttttactcgtaaggtatggtactcatttgctttctttcatttctcaggcaaaggcgataatgttgctagctgggaacggttcctctatgcctcaagtcttttcgccgcctcaaactcatcaacaagtggtccatcatactcgtgcctctgtcgattcttcagctatgcctcctagcttcatgcctacaatatcttatcttagccctgaagctggaagtagcacaaacggactcggagccacaaaagcgacaagaggcttgacgtcaacatatcacaacaaccaagctaatggatccaatattaactgcccagtaccagtttcttgttctaccaatgtaatggctccaacaggtaaaaaacaaagtcagagacctgatactacattcgccatctaacttactagtattttcatggatgtaacttcattctcgttctgtttcttatgcagtggcattacctctggctcgcaaagcatccctggctaggtttttagagaaacgcaaagaaaggtacgcaacacttctttagaatacaccattcaatagtttcttgggctaactctctttctcgctgtgggtttctcagggtcacgagcgtatccccatattgcttagacaagaagtcatcgacagattgtcgcagatcaatgtctgaatgcattagttcttctctcagctctgcaacctaatttcatctacagtaagaaggttgctttagaccactccacatccatatttgcatttcaatggcggtcttttcaatgtctcagttaatttttcctcactcgccacactgagtttctccttagctttatatatacgatagtgtatactttgtttacatgttttttggtggaatggaacttatgagagcatatcagatatgtacttgggaaaattagtagaaactgtttgtttctttttttttaactctgttcttttgtatatatcactgaagctcgcatatgtataattcatgtaatggaattgcatcgcttctgtttccctaagttattt

SEQ ID NO: 52 - protein, 310 aa, Arabidopsis thaliana
MERDFLGLGSKLSPITVKEETNEDSAPSRGMMDWSFSSKVGSGPQFLSFGTSQQETRVNTVNDHLLSSAAMDQNQRTYFSSLQEDRVFPGSSQQDQTTITVSMSEPNYINSFINHQHLGGSPIMAPPVSVFPAPTTIRSSSKPLPPQLTIFYAGSVLVYQDIAPEKAQAIMLLAGNGPHAKPVSQPKPQKLVHHSLPTTDPPTMPPSFLPSISYIVSETRSSGSNGVTGLGPTKTKASLASTRNNQTAAFSMAPTVGLPQTRKASLARFLEKRKERVINVSPYYVDNKSSIDCRTLMSECVSCPPAHHLH

SEQ ID NO: 53 - DNA, 3187 bp, Arabidopsis thaliana
  source 1..3187: mol_type = other DNA
attagaggaatcataaatcggcggtgtgtgtaacttcaactcacgtttttcatttctctccaaagtccttcaattgttactaattctctctgatctctcatttcttctcttctccggtgacattttttttctcccccgcgaaagctaaaccgtttttgtattctcaacgattgataagcctgatggagagagattttctcgggctgggatcaaagttatctccgataactgtgaaggaggaaactaacgaagattcaggtaattcatcttcaacatcttccattatgatctgatgattgtgtttttcatctcacttttttttgtttctatttttgtaatctctttttttgtttattgttcaagtacatatatattgtttttctagcttgattgggagtcctactgtctggttttttcttgaacaagaaattttttcttcgttttctcgggaagagaaaaaataaattagggtttcttttttcttgatatatatttaagaaattaggttttagtactatagacagaaatttagctactcgaatttgtttgacgtagccgatgaaaaaacacgttttgggactcgatagttagaaaattcatacgttcacgatctacttttgaagtttttttcattaaatattttttgcaaactacaaatgtacaagtatacaactatacaagcaaacaccaaacttgttgacgttagtaatttaacaagtgttagtattatctttgaaaaataatattcagagaacaaacttgattttctaggtgactaggtgatgcatgtttctaaagctgttggtaatgttgagtgttttcaaaataatttcgtttttttcttcaaacagccgacaccgacagaacaaaaatgctatattttttttgttgcttacaaaattgatcaattggtttcaatacaatagtatcttctttagaaaagattgtttttttcaaagccggattgaatattgagaattagaacattggctggttattctttttgaaaagtttatgccattttttaaggtttattaagcaacttgaattctatcagtattatttaaaaacgaagacgtgaaatgttgggaaaagaatgcgttatatagcgaccggctgacgattagagatttaacaacaaatgcaagttgaattatataaaagcaagattgattgtgacttgattaagttttatttctatccaagtagactcattgattaagttaggatcatgttgggtattaaatttagatcaagttacaatttggatgaataatttacttacccacgaggaatttaatagttagttcttgtctttttatattccgaaacgtgccatttcttgaaagtatttgtatgatcactattttccccagtgtgtttggctttatgcagatttgttcattgttgatgaatctaatgttaagagtcgtccactttagcatagctagatctgagtgtttcctagtttgataaaatctaaagacatttgctcatgtttcagccccaagtagaggtatgatggattggtcattctcaagcaaagtcggttctggtcctcagtttctttcttttgggacatcccaacaagaaacgcgtgtaaacacagtcaatgatcatttgctttcttctgctgcaatggatcaaaaccagagaacttacttcagctcactacaggttaggctatttcttgaaaagaaaaaaagtagtgataaagtgtgatttagtgaccttgtaagaaagcttggcaattggtttagtttcttctggtctcaaaattgatacaaaatgatctcaggaagacagagtgttcccaggttccagtcagcaagaccaaacaaccatcacagtctccatgtccgaaccaaactacatcaacagtttcataaaccaccaacatttaggaggatctcctatcatggcacctccagtttcagtatttcctgctccaaccactattaggcatgcactgcattctatcttcttctgtttaacatcagatacagaacctctttacttctatagttgactcgagctcctttatgttcatctccagatcttcttcaaaaccacttccccctcagttgacaatcttttatgccggttcagtattagtttaccaagacatagctcctgaaaaggtaaccaaatttccttcaatatgtgttacattacagtccaagctatccactgactaagtattcaatcaaagaaataagtttcacgtatagacatgctgaagttatagaaagttactaacctggtttcaacatacagtatgttaatgattcatagatatgataaatctttgtccttacttcttcatttattttgtattcataggcccaagctatcatgttgctagccggaaatggacctcatgctaaaccggtttcacaacctaaacctcaaaaactggttcatcactctcttccaaccactgatcctccaactatgcctcctagtttcctgccttccatctcttacattgtctctgaaaccagaagtagtggatccaacggggttactggacttggaccaacaaaaacaaaggcgagtttagcatccacgcgcaacaaccaaactgctgccttctctatggctccaacaggttataaatgaagtcttaactcctattaatgttttgtcatcaaacttctatcttaggtttagtttgttataaccaaaaaatcttgctatgatttaatacagtgggtttaccacaaacacgcaaagcatccttggctcggttcttagagaaacgcaaagaaaggtactgagctacaagattattcacttattcacaatatcaaaacacaggtttgctgtatattggcttcgttttcttgcagggtcattaacgtatcaccttattacgtagacaacaagtcatcaatagactgtagaacactgatgtctgaatgtgtaagctgtcctccagctcatcatctgcactaaaaccaatttagacccctcattgttctaaaggctttttcttttttctctggctctgtatcctatagactatagtatagttgttatagcttttgtttattcagattttagtacactgggcttgtaaaagcaagttatttatatatatcctataaatttaatttggatactgtatgttttgtctttactcttgcatgtgtataaaaaacataaaagtaagactattcaagct

SEQ ID NO: 54 - protein, 267 aa, Arabidopsis thaliana
MERDFLGLSDKQYLSNNVKHEVNDDAVEERGLSTKAAREWGKSKVFATSSFMPSSDFQEAKAFPGAYQWGSVSAANVFRRCQFGGAFQNATPLLLGGSVPLPTHPSLVPRVASSGSSPQLTIFYGGTISVFNDISPDKAQAIMLCAGNGLKGETGDSKPVREAERMYGKQIHNTAATSSSSATHTDNFSRCRDTPVAATNAMSMIESFNAAPRNMIPSVPQARKASLARFLEKRKERLMSAMPYKKMLLDLSTGESSGMNYSSTSPT

SEQ ID NO: 55 - DNA, 2297 bp, Arabidopsis thaliana
  source 1..2297: mol_type = other DNA
gcaaagagttaaataagcctctccaaaagtgtgtctgtaacattaccaaaacgaaaccttccttgtggattcccacttctttcttctgttttcttcttcctcttctttaaattggatgttttgggcaagaaacagagagaaacacgttaatttgagagtttgtcattgaatatttggtttgcaatggaaagagattttctgggtttgagcgacaagcagtatctaagtaataacgttaagcatgaggttaacgatgatgctgtcgaagaacgaggtttgtgttcttgtctcgagaatcttttattttaatgtttcaagaagagatcagttttcacttttaacatagccgtataaagttgtttatttaaatataatttttcagattccaaaacttgaaaaaaaaaagattccattaaatcttttataaaaatgagattggatagattagtcaaattgacgaccataaaaaatgatacttatagggttaagtacgaaggcagctagagaatgggggaagtcaaaggtttttgctacttcaagtttcatgccttcttcagatttccaggttggttcatcttaaaatttaacttactctgtatcagtttcagatgttatggctaatctaatggttctataagctaccgcataatcatggtcgtcttttagcatgtgcaagaggagtactcaattatggtcttgattaaaaagaagaatttactttcaaattatgttaaacacatcaatcacatatttatgagaaaagttgttttcgtaagagatagccaccggaaaatggtcggataaatggccgaactttatcatttttgtgtatgtggccaatcattaaccagggaaaaaaaattgttggataagtgctagttaagagctggtagggtcggtcgtctgccagccgcaaagttagggaaaaaataatttaatattttgtggcgtttggtgtttggcgtttggatcacgtttatttcttggcatttttctaaatttagaatgtacaaaaaatttaaagacgttgacgattaaaatttgaatttaacaaattaggaggctaaggcgtttccgggtgcataccagtggggatcagtttctgcggccaatgttttccgcagatgccaatttggtggtgcgtttcaaaacgcgacgccgcttttactaggcggttcagttcctttaccaactcatccttctcttgttccacggtaatttccatattatgatgcaaaaacattcaacaatttttttgctcttttcatattttgatttggttatgtgggtttgtggaaacagagtggcttcctccggatcatctcctcagctcacaatcttttatggcggaactataagcgtctttaatgacatatctcccgataaggtatatataatcaagattcatacaaataacatttacataacatttacatgttctaaaacggactattcatgatatgtgagtaggctcaagccatcatgttatgcgccgggaacggtttgaaaggtgaaactggagatagcaaaccggttcgagaagctgaaagaatgtatggaaaacaaatccataacactgctgctacctcatcaagctctgccactcacactgataatttctcaaggtgtagggacacacccgttgctgcgactaatgcaatgagcatgatcgaatcattcaatgcagctcctcgtaacatgattccttcaggtatgtgtgtctaatatcaacatcaaaacaaaatataatcaagatttttgcttcctcaaatcatatgtctaaactcgaaaattgcttttttccagtccctcaagctcggaaagcatccttggctcggttcttggagaagcgcaaagagaggtttgattttgtattttttttctttatagaaaattttgaggtttttcaattgaatctaaaagaattgatgttgttggtgcaggcttatgagtgcaatgccatacaagaagatgcttcttgatttgtcgaccggagaatccagtggaatgaattactcttctacttctcctacataaaacctacactttttttttttttttttacaatggtaatttgtaattgtaatcattagattatgattatatagttaccatttatattcttacgagcaggagaagacgttagggcgtctctgtatttgatcattgtttgtaatgctttggtctgtttattgtaggattacattataactttaagaactaacagatatatgtttgtcatggactcatgtctgtcaagaatttaatatcaaataaaattcactataatttttttt

SEQ ID NO: 56 - protein, 197 aa, Arabidopsis thaliana
MSKATIELDFLGLEKKQTNNAPKPKFQKFLDRRRSFRDIQGAISKIDPEIIKSLLASTGNNSDSSAKSRSVPSTPREDQPQIPISPVHASLARSSTELVSGTVPMTIFYNGSVSVFQVSRNKAGEIMKVANEAASKKDESSMETDLSVILPTTLRPKLFGQNLEGDLPIARRKSLQRFLEKRKERLVSTSPYYPTSA

SEQ ID NO: 57 - DNA, 1927 bp, Arabidopsis thaliana
  source 1..1927: mol_type = other DNA
aaaaactctcacatgagaaatcagaatccgttattattcctccatttattcatctcaaaacccatatctctctgtcttgatctctctctcactttctaataagatcaaagaagatgtcgaaagctaccatagaactcgatttcctcggacttgagaagaaacaaaccaacaacgctcctaagcctaagttccagaaatttctcgatcgccgtcgtagtttccgaggttcgtttggtttttagtcgctctctcttttttttttcttgcgataaatcgaatttattcatatggaactcctgcagatattcaaggtgcgatttcgaaaatcgatccggagattatcaaatcgctgttagcttccactggaaacaattccgattcatcggctaaatctcgttcggttccgtctactccgagggaagatcagcctcagatcccgatttctccggtccacgcgtctctcgccaggtatttttgtctttccggtaaagttttttttttctttctaacttttttggcgctaccagaaaagacgaaaaaatttgaaattcaaattttcaaaacattcattttcctcaggtctagtaccgaactcgtttcgggaactgttcctatgacgattttctacaatggaagtgtttcagttttccaagtgtctcgtaacaaagctggtgaaattatgaaggtcgctaatgaagcagcatctaagaaagacgagtcgtcgatggagacagatctttcggtaattcttccgaccactctaagaccaaagctctttggccagaatctagaaggaggttagtataataaaaataaaaatcacttagtgctggattcttctagaattttagttacatattattgcatgtagagatctaagaagagtttgttgttagagaggaattggttgctaattagtttggaattagatatcaaagagttaaagactatagtttatgtctatacgtattaatatacgttattaataaaagtataaacatgttgtttaatttctgataagaaactggtttatgcgtgtgtatgcagatcttcccatcgcaaggagaaagtcactgcaacgttttctcgagaagcgcaaggagaggtaatgattcttcaacaatccaaggatttttacccccaaataattaaagaaaggtttttatttttctctctctcgaccttttttttactataagttatttaagatagtaattatgggtcctgcctcttttactctcacatacaacttaagattcaactagttttgttcaacaacgcacatgcttatacgtagatagataatggagatcagtagtaatatcggtatacgtaggttactattgtaatggaacttttaaaaagcgcgttgactttgagtctttgactctagttctgtttgctacaccgacaagttatatttttcaaaatgatgagaaaacgaggagaaacaccggaaaaaaatttgaacttttacttttatcagaccatacggccaaagaaagatctgtatattatataagttatcacaaaacgcggtttcacattttctttttcgtcttgttgtgtttgcagattagtatcaacatctccttactatccgacatcggcctaaacgatctctttttagattgggacatggaccaaatttgtctttttcaatcggaagacatccatgttcgtttttggatttggcttatttccaatcttcttttgaagccttcttcgtcgttgctaaatcgtatactattcacgacaaacgtttttaggagattacgttacctactaagattatatatattggtttgtttttaaaaatgtctattatctttattgtcattgatagcttgatttaagaagctctctcttatcccgtgaccttctacttttgttttattttttagtatatggtaaagaaaattataac

SEQ ID NO: 58 - protein, 273 aa, Brassica rapa
MSSPMESSDFAATRRFSRKPSFSQTCSRLSQYLKENGSFGDLSLGMACKPEVNGISRQPTTTMSLFPCEASNMEPIGQDVKPKNLFPRQPSFSSSSSSLPKEDILKMTQATSSTRSVKPEPQTAPLTIFYGGQVIVFNDFSAEKAKEVMDLASKGTANTFTGFTSNVNNNIQSVYTTNLANNQTEMRSNIAPIPNQLPHLMKTTTQNPVQSSSTAMACELPIARRASLHRFLAKRKDRVTSKAPYQLNDPAKASSKPQTGDNTTSWLGLAAEM

SEQ ID NO: 59 - protein, 252 aa, Brassica oleracea
MSSSMECSTTRRSSSGKPSFSLTCSRLSQYLKENGSFGDLSLGMSCKPDTNGMSRKPTTTMSLFPCEASNVGSMAAAQDVKPKNLFPRQPSFSSSSSSIPKEDVPKMTQTTTRSLKPEPQTAPLTIFYGGQVIVFNDFSAEKAKEVMNLANKGTANTFTGFTSTLNNNIAPTPNQVPHLMKAATQDPKQTSSAAMACELPIARRASLHRFLAKRKDRVTSKAPYQLNDPAKAYSKPQTGNTTTSWLGLAADM

SEQ ID NO: 60 - protein, 233 aa, Zea mays
MAASARPGERATSFAVACSLLSRFVRQNGVAAADLGLRIKGEVEQQRTPATTNSLPGAEGEEVERRKETMELFPQSVGFSIKDAAAPREEQGDKEKPKQLTIFYGGKVLVFDDFPADKAKDLMQLASKGSPVVQNVALPQPSAAAAVTTDKAVLDPVISLAAAKKPARTNASDMPIMRKASLHRFLEKRKDRLNAKTPYQTAPSDAAPVKKEPESQPWLGLGPNAVDSSLNLS

SEQ ID NO: 61 - protein, 240 aa, Glycine max
MSSSSEYLVFSSHHPANSPAEKSTFSQTCSLLSQYIKEKGTFGDLTLGMTCTAETNGSPETSCHSATTMELFPTIITQRNPTTVDFLSPQTAYPHHSEVPIMVKSSAFKSMEKEPKAAQLTIFYAGQVVVFDDFPAEKLEEITSLAGKGISQSQNTSAYAHTHNQQVNHPSFVPNISPQAPSRPLVCDLPIARKASLHRFLSKRKDRIAAKAPYQINNPNSASSKPAESMSWLGLGAQST

SEQ ID NO: 62 - protein, 244 aa, Oryza sativa
MAASARPVGVGGERATSFAMACSLLSRYVRQNGAAAAELGLGIRGEGEAPRAAPATMSLLPGEAERKKETMELFPQSAGFGQQDAITADSAADAREQEPEKRQLTIFYGGKVLVFNDFPADKAKGLMQLASKGSPVAPQNAAAPAPAAVTDNTKAPMAVPAPVSSLPTAQADAQKPARANASDMPIARKASLHRFLEKRKDRLNAKTPYQASPSDATPVKKEPESQPWLGLGPNAVVKPIERGQ

SEQ ID NO: 63 - protein, 391 aa, Zea mays
MERDFLAAIGKEQQHPRKEKAGGGAEESAYFGAAAVPAMDWSFASKPCAAPALMSFRSAAREEPSFPQFSALDGTKNTAPRMLTHQRSFGPDSTQYAALHRAQNGARVVPVSSPFSQSNPMFRVQSSPSLPNSTAFKQPPFAISNAVASSTVGSYGGTRDAVRPRTAQLTIFYAGSVNVFNNVSAEKAQELMFLASRGSSAPVACKPEAPPTLAPAKVTAPEVLLPAKQMLFQKPQHLSPPPSSVPGILQSAALPRSASSSSNLDSPAPKSSVPLAVPPVSQAPPATLIATTTAAAIMPRAVPQARKASLARFLEKRKERVTTAAPYPSAKSPLESSDTFGSGSASANANDKSSCTDIALSSNHEESLCLGGQPRSIISFSEESPSTKLQI

SEQ ID NO: 64 - protein, 386 aa, Glycine max
MEREFFGLSSKNGAWTTMKDDAVNKSRDQVRSSGMQWSFPNKVSALPQFLSFKTNQEDKPRKTILEPLASSGYMAMSTQYAFDSNQKSFLGLTNRNLSISKHAAGNKQGMTVYPLQCCDAQSEEARIFSVSNQSNQVSPVLQSNLASTGLNMVNSVIKPQPFGSKSSGTPLSILPSIGSIVGSTDLRNNSKSSTMPTQLTIFYAGSVCVYDDISPEKAKAIMLMAGNGYTPTEKMELPTVKLQPAISIPSKDDGFMISQSYPPSTFPTPLPLTSHVNSQPGGGSSSNKEISIIRQVGPSTAPTNHLESPIIGSIGSASKEKAQPVCLPQARKASLARFLEKRKGRMMRTSPYLYMSKKSPECSSSGSDSVSFSLNFSGSCSLPATN

SEQ ID NO: 65 - protein, 416 aa, Oryza sativa
MERDFLGAIGKDEEQRRHAEERKESDYFGAGGGAAAAAMDWSFASRAALMSFRSSSSAAAAAAREETRELAFPHFSALDGAKMQQASHVLARQKSFGAESHGIPQYAAAAAVHGAHRGQPPHVLNGARVIPASSPFNPNNPMFRVQSSPNLPNAVGAGGGAFKQPPFAMGNAVAGSTVGVYGTRDMPKAKAAQLTIFYAGSVNVFNNVSPEKAQELMFLASRGSLPSAPTTVARMPEAHVFPPAKVTVPEVSPTKPMMLQKPQLVSSPVPAISKPISVVSQATSLPRSASSSNVDSNVTKSSGPLVVPPTSLPPPAQPETLATTTAAAIMPRAVPQARKASLARFLEKRKERVTTVAPYPLAKSPLESSDTMGSANDNKSSCTDIALSSNRDESLSLGQPRTISFCEESPSTKLQI

SEQ ID NO: 66 - protein, 404 aa, Zea mays
MAKSGASFPESSWMERDFLAAIGKEQQHPHKEEAGAEESAYFGGAGAAAAAPAMDWSFASKPGAAPALMSFRSASFPQFSSFDGAKNPAPRILTHQRSFGPDSTHYAAAHRTQHALNGARVTPVSSPFNQNSPMFRVQSSPSLPNGTAFKQPPFAINNNAAASSTVGFYGTRDVVRPKTAQLTIFYAGSVNVFDNVSAEKAQELMLLASRGSLPSSAPVARKPEAPILAPAKVTAPEVLHATQMLFQKPQHVSPPSSAISKPIPGILQAASLPRSASSSNLDSPFPKSSVPFPVSPVSQAPRAQPATIAATTAAAIMPRAVPQARKASLARFLEKRKERVTTAAPYPSAKSPMESSDTFGSGSANDKSSCTDIALSSNHEESLCLGQPRNISFIQESPSTKLQI

SEQ ID NO: 67 - protein, 340 aa, Glycine max
MERDFMGLNLKEPLAVVKEEMNNDGCKNSGFKKGRIAQWPFSNKVSALPHLMSFKASQDDKTKNTVSDTLSSSGFMSILSQEAFDTSQKRSAGEPQMFSVPNQAISVSLGNPFLKNHFAAAGQKPLLGGIPVTTSHSVLPSAVAVAGMTESCNSVKPSAQLTIFYAGTVNIFDDISAEKAQAIMLLAGNSLSAASNMAQPNVQVPISKLGAGAGVPVSQPANTSPGSGLSSPLSVSSHTGVQSGSGLTSTDEFLAAKTTGVPNTPICNVEPPKVVSATTMLTSAVPQARKASLARFLEKRKERVMSAAPYNLNKKSEECATAEYAGVNFSATNTVLAKQG

SEQ ID NO: 68 - protein, 427 aa, Oryza sativa
MERDFLGAIWRKEEAAGKPEEHSDYRGGGGGASAAMQWQFPATKVGAASSAFMSFRSSAAAAREEDPKEAAVFDRFSLSGFRPPPRPSPGDAFDGAAAMKQRQFGFNGRQQYAAAAQHGHREQGVDSYGVAAPHHFPSPSPSPRHPVPFGHANPMLRVHSLPNVAGGSPYRNQSFSVGNSVAGSTVGVYGGPRDLQNPKVTQMTIFYDGLVNVFDNIPVEKAQELMLLASRASIPSPPSAARKSDSPISAAAKLTVPEALPARQIVVQKPEASVPLVSGVSNPITIVSQAVTLPKSFSSSNDSAGPKSGGLPLAVTPLSQASPSQPIPVATTNASAIMPRAVPQARKASLARFLEKRKERVSSVAPYPSSKSPLESSDTIGSPSTPSKSSCTDITPSTNNCEDSLCLGQPRNISFSSQEPPSTKLQI

SEQ ID NO: 69 - protein, 353 aa, Zea mays
MDWSFASKPCAAPALMSFRSAAREEPSFPQFSALDGTKNTAPRMLTHQRSFGPDSTQYAALHRAQNGARVVPVSSPFSQSNPMFRVQSSPSLPNSTAFKQPPFAISNAVASSTVGSYGGTRDAVRPRTAQLTIFYAGSVNVFNNVSAEKAQELMFLASRGSSAPVACKPEAPPTLAPAKVTAPEVLLPAKQMLFQKPQHLSPPPSSVPGILQSAALPRSASSSSNLDSPAPKSSVPLAVPPVSQAPPATLIATTTAAAIMPRAVPQARKASLARFLEKRKERVTTAAPYPSAKSPLESSDTFGSGSASANANDKSSCTDIALSSNHEESLCLGGQPRSIISFSEESPSTKLQI

SEQ ID NO: 70 - protein, 338 aa, Glycine max
MERDFMGLNLKEPLAVVKEEMNNDGCKNSGFKKGRIAQWPFSNKVSALPHLMSFKASQDDKTKNTVSDTLSSSGFMSILSQEAFDTSQKRSAGEPQMFSVPNQAISVSLGNPFLKNHFAAAGQKPLLGGIPVTTSHSVLPSAVAVAGMTESCVKPSAQLTIFYAGTVNIFDDISAEKAQAIMLLAGNSLSAASNMAQPNVQVPISKLGAGAGVPVSQPANTSPGSGLSSPLSVSSHTGVQSGSGLTSTDEFLAAKTTGVPNTPICNVEPPKVVSATTMLTSAVPQARKASLARFLEKRKERVMSAAPYNLNKKSEECATAEYAGVNFSATNTVLAKQG

SEQ ID NO: 71 - protein, 308 aa, Oryza sativa
MQQASHVLARQPPHVLNGARVIPASSPFNPNNPMFRVQSSPNLPNAVGAGGGAFKQPPFAMGNAVAGSTVGVYGTRDMPKAKAAQLTIFYAGSVNVFNNVSPEKAQELMFLASRGSLPSAPTTVARMPEAHVFPPAKVTVPEVSPTKPMMLQKPQLVSSPVPAISKPISVVSQATSLPRSASSSNVDSNVTKSSGPLVVPPTSLPPPAQPETLATTTAAAIMPRAVPQARKASLARFLEKRKERVTTVAPYPLAKSPLESSDTMGSANDNKSSCTDIALSSNRDESLSLGQPRTISFCEESPSTKLQI

SEQ ID NO: 72 - protein, 218 aa, Zea mays
MAGHAPARDKTTTGFAATCSLLSQFLKEKKGGLQGLGGLAMAPAPAAGAGAFRPPTTMNLLSALDAAKATVGEPEGHGQRTGGNPREAAGEEAQQLTIFYGGKVVVFDRFPSAKVKDLLQIVSPPGADAVVDGAGAGAAVPTQNLPRPSHDSLSADLPIARRNSLHRFLEKRKDRITAKAPYQVNSSVGAEASKAEKPWLGLGQEQEGSDGRQAGEEM

SEQ ID NO: 73 - protein, 201 aa, Glycine max
MAAGVTVKSEVLESSPPEGVCSNTVENHLVQTNLSDGSPNKSVPASGLDAVIPSANQLTIFYNGSVCVYDGIPAEKVHEIMLIAAAAAKSTEMKKIGTQTTLISPAPSRPSSPHGITNNIGSSQKSSICRLQAEFPIARRHSLQRFLEKRRDRLGSKTPYPSSPTTKVADNIENNFCADNAPELISLNRSEEEFQPTVSAS

SEQ ID NO: 74 - protein, 188 aa, Oryza sativa
MSTRAPVELDFLGLRAAAADADDRHAKSGGSSASSSSSIRGMETSAIARIGPHLLRRVIAAAGPPPPPSTAPVPEEMPGAAAAAAPMTLFYNGSVAVFDVSHDKAEAIMRMATEATKAKGLARGNAIVGNFAKEPLTRTKSLQRFLSKRKERLTSLGPYQVGGPAAVGATTSTTTKSFLAKEEEHTAS

SEQ ID NO: 75 - DNA, 20 bp, synthetic construct
  misc_feature 1..20: note = A synthetic primer
  source 1..20: mol_type = other DNA
accgagacacattcccgatt

SEQ ID NO: 76 - DNA, 20 bp, synthetic construct
  misc_feature 1..20: note = A synthetic primer
  source 1..20: mol_type = other DNA
catcaggcttgcatgccatt

SEQ ID NO: 77 - DNA, 25 bp, synthetic construct
  misc_feature 1..25: note = A synthetic primer
  source 1..25: mol_type = other DNA
acgaataagagcgtccattttagag

SEQ ID NO: 78 - DNA, 24 bp, synthetic construct
  misc_feature 1..24: note = A synthetic primer
  source 1..24: mol_type = other DNA
gtggagtggtctaaagcaaccttc

SEQ ID NO: 79 - DNA, 23 bp, synthetic construct
  misc_feature 1..23: note = A synthetic primer
  source 1..23: mol_type = other DNA
ataacgctgcggacatctacatt

SEQ ID NO: 80 - DNA, 21 bp, synthetic construct
  misc_feature 1..21: note = A synthetic primer
  source 1..21: mol_type = other DNA
tcaggaagacagagtgttccc

SEQ ID NO: 81 - DNA, 21 bp, synthetic construct
  misc_feature 1..21: note = A synthetic primer
  source 1..21: mol_type = other DNA
tgcgtttctctaagaaccgag

SEQ ID NO: 82 - skipped

SEQ ID NO: 83 - DNA, 20 bp, synthetic construct
  misc_feature 1..20: note = A synthetic primer
  source 1..20: mol_type = other DNA
ttgggtgatggttcacgtag

SEQ ID NO: 84 - DNA, 21 bp, synthetic construct
  misc_feature 1..21: note = A synthetic primer
  source 1..21: mol_type = other DNA
taccgcataatcatggtcgtc

SEQ ID NO: 85 - DNA, 21 bp, synthetic construct
  misc_feature 1..21: note = A synthetic primer
  source 1..21: mol_type = other DNA
tcatgctcattgcattagtcg

SEQ ID NO: 86 - DNA, 22 bp, synthetic construct
  misc_feature 1..22: note = A synthetic primer
  source 1..22: mol_type = other DNA
ctttgaagacgtggttggaacg

SEQ ID NO: 87 - DNA, 22 bp, synthetic construct
  misc_feature 1..22: note = A synthetic primer
  source 1..22: mol_type = other DNA
atttctcgatcgccgtcgtagt

SEQ ID NO: 88 - DNA, 25 bp, synthetic construct
  misc_feature 1..25: note = A synthetic primer
  source 1..25: mol_type = other DNA
gccaaagagctttggtcttagagtg

SEQ ID NO: 89 - DNA, 25 bp, synthetic construct
  misc_feature 1..25: note = A synthetic primer
  source 1..25: mol_type = other DNA
gtctaagcgtcaatttgtttacacc

SEQ ID NO: 90 - DNA, 25 bp, synthetic construct
  misc_feature 1..25: note = A synthetic primer
  source 1..25: mol_type = other DNA
atgtcgagttctatggaatgttctg

SEQ ID NO: 91 - DNA, 26 bp, synthetic construct
  misc_feature 1..26: note = A synthetic primer
  source 1..26: mol_type = other DNA
tcatatttcagctgctaaaccgagcc

SEQ ID NO: 92 - DNA, 21 bp, synthetic construct
  misc_feature 1..21: note = A synthetic primer
  source 1..21: mol_type = other DNA
atggagagagattttctcggg

SEQ ID NO: 93 - DNA, 23 bp, synthetic construct
  misc_feature 1..23: note = A synthetic primer
  source 1..23: mol_type = other DNA
ttaggttgcagagctgagagaag

SEQ ID NO: 94 - DNA, 20 bp, synthetic construct
  misc_feature 1..20: note = A synthetic primer
  source 1..20: mol_type = other DNA
atggagagagattttctcgg

SEQ ID NO: 95 - DNA, 20 bp, synthetic construct
  misc_feature 1..20: note = A synthetic primer
  source 1..20: mol_type = other DNA
cagatgatgagctggaggac

SEQ ID NO: 96 - DNA, 24 bp, synthetic construct
  misc_feature 1..24: note = A synthetic primer
  source 1..24: mol_type = other DNA
atggaaagagattttctgggtttg

SEQ ID NO: 97 - DNA, 29 bp, synthetic construct
  misc_feature 1..29: note = A synthetic primer
  source 1..29: mol_type = other DNA
ttatgtaggagaagtagaagagtaattca

SEQ ID NO: 98 - DNA, 22 bp, synthetic construct
  misc_feature 1..22: note = A synthetic primer
  source 1..22: mol_type = other DNA
atgtcgaaagctaccatagaac

SEQ ID NO: 99 - DNA, 30 bp, synthetic construct
  misc_feature 1..30: note = A synthetic primer
  source 1..30: mol_type = other DNA
gatagtaaggagatgttgatactaatctct

SEQ ID NO: 100 - DNA, 24 bp, synthetic construct
  misc_feature 1..24: note = A synthetic primer
  source 1..24: mol_type = other DNA
atggctgatggtgaagacattcaa

SEQ ID NO: 101 - DNA, 24 bp, synthetic construct
  misc_feature 1..24: note = A synthetic primer
  source 1..24: mol_type = other DNA
tcagaagcacttcctgtgaacaat

SEQ ID NO: 102 - DNA, 19 bp, synthetic construct
  misc_feature 1..19: note = A synthetic primer
  source 1..19: mol_type = other DNA
aagcagcgtaatcggtagg

SEQ ID NO: 103 - DNA, 21 bp, synthetic construct
  misc_feature 1..21: note = A synthetic primer
  source 1..21: mol_type = other DNA
gcacagcaatcgggtataaag

SEQ ID NO: 104 - DNA, 24 bp, synthetic construct
  misc_feature 1..24: note = A synthetic primer
  source 1..24: mol_type = other DNA
ggagaactcacgatgggagcgatt

SEQ ID NO: 105 - DNA, 24 bp, synthetic construct
  misc_feature 1..24: note = A synthetic primer
  source 1..24: mol_type = other DNA
gcgtcgtggctttcgataaccaga

SEQ ID NO: 106 - DNA, 17 bp, synthetic construct
  misc_feature 1..17: note = A synthetic primer
  source 1..17: mol_type = other DNA
gctggcggttcgacatg

SEQ ID NO: 107 - DNA, 21 bp, synthetic construct
  misc_feature 1..21: note = A synthetic primer
  source 1..21: mol_type = other DNA
gccattcctctgcgaattaga

SEQ ID NO: 108 - DNA, 26 bp, synthetic construct
  misc_feature 1..26: note = A synthetic primer
  source 1..26: mol_type = other DNA
agaaactccaaatcaagaaccagctc

SEQ ID NO: 109 - DNA, 26 bp, synthetic construct
  misc_feature 1..26: note = A synthetic primer
  source 1..26: mol_type = other DNA
ccggtttaatcgaagaacacgaagac

SEQ ID NO: 110 - protein, 4 aa, synthetic construct
  REGION 1..4: note = A synthetic protein
GWGD

SEQ ID NO: 111 - protein, 4 aa, synthetic construct
  REGION 1..4: note = A synthetic protein
DFSG

SEQ ID NO: 112 - protein, 11 aa, synthetic construct
  REGION 1..11: note = A synthetic protein
RELNSLISGGV

SEQ ID NO: 113 - protein, 10 aa, synthetic construct
  REGION 1..10: note = A synthetic protein
DTEWFFLVSM

SEQ ID NO: 114 - protein, 15 aa, synthetic construct
  REGION 1..15: note = A synthetic protein
VVNDLMIQQATVKMG

SEQ ID NO: 115 - protein, 32 aa, synthetic construct
  REGION 1..32: note = A synthetic protein
KRGRKPANGREEPLNHVEAERQRREKLNQRFY

SEQ ID NO: 116 - protein, 49 aa, synthetic construct
  REGION 1..49: note = A synthetic protein
GDNTVILGWGDGYYKGEEDKEKKKNNTNTAEQEHRKRVIRELNSLISGG

SEQ ID NO: 117 - protein, 51 aa, synthetic construct
  REGION 1..51: note = A synthetic protein
NNNTVLLGWGDGYYKGEEEKSRKKKSNPASAAEQEHRKRVIRELNSLISGG

SEQ ID NO: 118 - protein, 52 aa, synthetic construct
  REGION 1..52: note = A synthetic protein
GAGASLLGWGDGYYKGCDDADKRARQQPTPASAAEQHRKRVLRELNSLIAGG

SEQ ID NO: 119 - protein, 53 aa, synthetic construct; identical to SEQ ID NO: 12
  REGION 1..53: note = A synthetic protein

SEQ ID NO: 120 - protein, 53 aa, synthetic construct
  REGION 1..53: note = A synthetic protein
STGASLLGWGDGYYKGCDDDKRKQRSSTPAAAAEQEHRKRVLRRELNSLIAGA

SEQ ID NO: 121 - protein, 52 aa, synthetic construct; identical to SEQ ID NO: 10
  REGION 1..52: note = A synthetic protein

SEQ ID NO: 122 - protein, 52 aa, synthetic construct
  REGION 1..52: note = A synthetic protein
ATGASLLGWGDGYYKGCDDDKRKQRPLTPAAQAEQEHRKRVLRELNSLISGA

SEQ ID NO: 123 - protein, 55 aa, synthetic construct
  REGION 1..55: note = A synthetic protein
FSGASVLGWGDHYYKGEEDKAKPRQRSSSPPFSTPADQEYRKKVLRELNSLISGG

SEQ ID NO: 124 - protein, 55 aa, synthetic construct
  REGION 1..55: note = A synthetic protein
FSGASVLGWGDGYYKGEEDKANPRRRSSSPPFSTPADQEYRKKVLRELNSLISGG

SEQ ID NO: 125 - protein, 53 aa, synthetic construct; identical to SEQ ID NO: 20
  REGION 1..53: note = A synthetic protein

SEQ ID NO: 126 - protein, 54 aa, synthetic construct
  REGION 1..54: note = A synthetic protein
FASQTVLGWGDGYYKGEEDKNKRRGSSSSAANFCAEQEHRKKVLRELNSLISGV

SEQ ID NO: 127 - protein, 55 aa, synthetic construct
  REGION 1..55: note = A synthetic protein
RSGQQVLGWGDGCCREPNEEEESKVVRSYNFNNMGAEEETWQDMRKRVLQKLHRL

SEQ ID NO: 128 - protein, 55 aa, synthetic construct
  REGION 1..55: note = A synthetic protein
KAGDLVLCWGDGYCREPKEGEKSEIVRILSMGREEETHQTMRKRVLQKLHDLFGG

SEQ ID NO: 129 - DNA, 23 bp, synthetic construct
  misc_feature 1..23: note = A synthetic primer
  source 1..23: mol_type = other DNA
acggttcctctatgcctcaagtc